(19) United States
(12) Patent Application Publication
(10) Pub. No.: US 2007/0111981 A1
(43) Pub. Date: May 17, 2007

(54) New (hetero)aryl compounds with MCH antagonistic activity and medicaments comprising these compounds

(76) Inventors: Gerald Juergen Roth, Biberach (DE); Stephan Georg Mueller, Warthausen (DE); Thorsten Lehmann-Lintz, Ochsenhausen (DE); Dirk Stenkamp, Biberach (DE); Philipp Lustenberger, Basel (CH); Joerg Kley, Mittelbiberach (DE); Klaus Rudolf, Warthausen (DE); Armin Heckel, Biberach (DE); Marcus Schindler, Biberach (DE); Leo Thomas, Biberach (DE); Ralf R.H. Lotz, Schemmerhofen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/552,836

(22) Filed: Oct. 25, 2006

(30) Foreign Application Priority Data
Oct. 26, 2005 (EP) 05 110 014

(51) Int. Cl. A61K 31/541; A61K 31/5377; A61K 31/496; A61K 31/454

(52) U.S. Cl. 514/210.2; 514/227.5; 514/235.2; 514/326; 514/253.01; 544/60; 544/124; 544/360; 546/207

(57) ABSTRACT

The present invention relates to (hetero)aryl compounds of general formula I 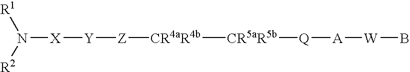
wherein the groups and radicals A, B, Q, W, X, Y, Z, R¹, R², R^{4a}, R^{4b}, R^{5a}, R^{5b}, have the meanings given in claim 1. Moreover the invention relates to pharmaceutical compositions containing at least one compound according to the invention. By virtue of their MCH-receptor antagonistic activity the pharmaceutical compositions according to the invention are suitable for the treatment of metabolic disorders and/or eating disorders, particularly obesity, bulimia, anorexia, hyperphagia and diabetes.

This application claims priority to EP 05 110 014, filed Oct. 26, 2005, which is incorporated herein in its entirety.

The present invention relates to new heteroaryl compounds, the physiologically acceptable salts thereof as well as their use as MCH antagonists and their use in preparing a pharmaceutical preparation which is suitable for the prevention and/or treatment of symptoms and/or diseases caused by MCH or causally connected with MCH in some other way. The invention also relates to the use of a compound according to the invention for influencing eating behaviour and for reducing body weight and/or for preventing any increase in body weight in a mammal. It further relates to compositions and medicaments containing a compound according to the invention and processes for preparing them. Other aspects of this invention relate to processes for preparing the compounds according to the invention.

### BACKGROUND TO THE INVENTION

The intake of food and its conversion in the body is an essential part of life for all living creatures. Therefore, deviations in the intake and conversion of food generally lead to problems and also illness. The changes in the lifestyle and nutrition of humans, particularly in industrialised countries, have promoted morbid overweight (also known as corpulence or obesity) in recent decades. In affected people, obesity leads directly to restricted mobility and a reduction in the quality of life. There is the additional factor that obesity often leads to other diseases such as, for example, diabetes, dyslipidaemia, high blood pressure, arteriosclerosis and coronary heart disease. Moreover, high body weight alone puts an increased strain on the support and mobility apparatus, which can lead to chronic pain and diseases such as arthritis or osteoarthritis. Thus, obesity is a serious health problem for society.

The term obesity means an excess of adipose tissue in the body. In this connection, obesity is fundamentally to be seen as the increased level of body fat which leads to a health risk. There is no sharp distinction between normal individuals and those suffering from obesity, but the health risk accompanying obesity is presumed to rise continuously as the level of body fat increases. For simplicity's sake, in the present invention, individuals with a Body Mass Index (BMI), which is defined as the body weight measured in kilograms divided by the height (in metres) squared, above a value of 25 and more particularly above 30, are preferably regarded as suffering from obesity.

Apart from physical activity and a change in nutrition, there is currently no convincing treatment option for effectively reducing body weight. However, as obesity is a major risk factor in the development of serious and even life-threatening diseases, it is all the more important to have access to pharmaceutical active substances for the prevention and/or treatment of obesity. One approach which has been proposed very recently is the therapeutic use of MCH antagonists (cf. interalia WO 01/21577, WO 01/82925).

Melanin-concentrating hormone (MCH) is a cyclic neuropeptide consisting of 19 amino acids. It is synthesised predominantly in the hypothalamus in mammals and from there travels to other parts of the brain by the projections of hypothalamic neurones. Its biological activity is mediated in humans through two different G-protein-coupled receptors (GPCRs) from the family of rhodopsin-related GPCRs, namely the MCH receptors 1 and 2 (MCH-1R, MCH-2R).

Investigations into the function of MCH in animal models have provided good indications for a role of the peptide in regulating the energy balance, i.e. changing metabolic activity and food intake [1,2]. For example, after intraventricular administration of MCH in rats, food intake was increased compared with control animals. Additionally, transgenic rats which produce more MCH than control animals, when given a high-fat diet, responded by gaining significantly more weight than animals without an experimentally altered MCH level. It was also found that there is a positive correlation between phases of increased desire for food and the quantity of MCH mRNA in the hypothalamus of rats. However, experiments with MCH knock-out mice are particularly important in showing the function of MCH. Loss of the neuropeptide results in lean animals with a reduced fat mass, which take in significantly less food than control animals.

The anorectic effects of MCH are presumably mediated in rodents through the G_{∀S}-coupled MCH-1R [3-6], as, unlike primates, ferrets and dogs, no second MCH receptor subtype has hitherto been found in rodents. After losing the MCH-1R, knock-out mice have a lower fat mass, an increased energy conversion and, when fed on a high fat diet, do not put on weight, compared with control animals. Another indication of the importance of the MCH system in regulating the energy balance results from experiments with a receptor antagonist (SNAP-7941) [3]. In long term trials the animals treated with the antagonist lose significant amounts of weight.

In addition to its anorectic effect, the MCH-1R antagonist SNAP-7941 also achieves additional anxiolytic and antidepressant effects in behavioural experiments on rats [3]. Thus, there are clear indications that the MCH-MCH-1R system is involved not only in regulating the energy balance but also in affectivity.

Literature:
1. Qu, D., et al., A role for melanin-concentrating hormone in the central regulation of feeding behaviour. Nature, 1996. 380(6571): p. 243-7.
2. Shimada, M., et al., Mice lacking melanin-concentrating hormone are hypophagic and lean. Nature, 1998. 396(6712): p. 670-4.
3. Borowsky, B., et al., Antidepressant, anxiolytic and anorectic effects of a melanin-concentrating hormone-I receptor antagonist. Nat Med. 2002. 8(8): p. 825-30.
4. Chen, Y., et al., Targeted disruption of the melanin-concentrating hormone receptor-1 results in hyperphagia and resistance to diet-induced obesity. Endocrinology, 2002. 143(7): p. 2469-77.
5. Marsh, D. J., et al., Melanin-concentrating hormone 1 receptor-deficient mice are lean, hyperactive, and hyperphagic and have altered metabolism. Proc Natl Acad Sci U S A, 2002. 99(5): p. 3240-5.
6. Takekawa, S., et al., T-226296: A novel, orally active and selective melanin-concentrating hormone receptor antagonist. Eur J Pharmacol, 2002. 438(3): p.129-35.

In the patent literature certain amine compounds are proposed as MCH antagonists. Thus, WO 01/21577 (Takeda) describes compounds of formula 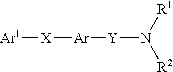
wherein Ar¹ denotes a cyclic group, X denotes a spacer, Y denotes a bond or a spacer, Ar denotes an aromatic ring which may be fused with a non-aromatic ring, R¹ and R² independently of one another denote H or a hydrocarbon group, while R¹ and R² together with the adjacent N atom may form an N-containing hetero ring and R² with Ar may also form a spirocyclic ring, R together with the adjacent N atom and Y may form an N-containing hetero ring, as MCH antagonists for the treatment of obesity.

Moreover WO 01/82925 (Takeda) also describes compounds of formula 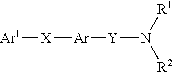
wherein Ar¹ denotes a cyclic group, X and Y represent spacer groups, Ar denotes an optionally substituted fused polycyclic aromatic ring, R¹ and R² independently of one another represent H or a hydrocarbon group, while R¹ and R² together with the adjacent N atom may form an N-containing heterocyclic ring and R² together with the adjacent N atom and Y may form an N-containing hetero ring, as MCH antagonists for the treatment of obesity, inter alia.

WO 94/22809 (Pharmacia/Famitalia) describes substituted (arylalkylaminobenzyl)-aminopropionamide derivatives and their use as anti-epileptic, neuroprotective and antidepressant agents. Among many other examples the compounds 2-[[[4-[[3-(2-fluorophenyl)propyl]amino]phenyl]methyl]amino]-propanamide and 2-[[[4-[[3-(3-fluorophenyl)propyl]amino]phenyl]methyl]amino]-propanamide are mentioned.

U.S. Pat. No. 3,209,029 describes aminoalkyl-aromatic-ethylamines as difunctional amines capable of use in condensation reactions to provide novel polyamides.

### AIM OF THE INVENTION

The aim of the present invention is to identify new (hetero)aryl compounds, particularly those which are especially effective as MCH antagonists. The invention also sets out to provide new (hetero)aryl compounds which can be used to influence the eating habits of mammals and achieve a reduction in body weight, particularly in mammals, and/or prevent an increase in body weight.

The present invention further sets out to provide new pharmaceutical compositions which are suitable for the prevention and/or treatment of symptoms and/or diseases caused by MCH or otherwise causally connected to MCH. In particular, the aim of this invention is to provide pharmaceutical compositions for the treatment of metabolic disorders such as obesity and/or diabetes as well as diseases and/or disorders which are associated with obesity and diabetes. Other objectives of the present invention are concerned with demonstrating advantageous uses of the compounds according to the invention. The invention also sets out to provide a process for preparing the amide compounds according to the invention. Other aims of the present invention will be immediately apparent to the skilled man from the foregoing remarks and those that follow.

### OBJECT OF THE INVENTION

In a first aspect the present invention relates to (hetero)aryl compounds of general formula I

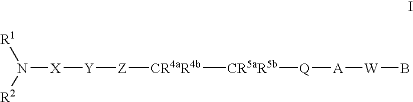

wherein

R¹, R² independently of one another denote H, C₁₋₈-alkyl or C₃₋₇-cycloalkyl, while the alkyl or cycloalkyl group may be mono- or polysubstituted by identical or different groups R¹¹, and a —CH₂— group in position 3 or 4 of a 5-, 6- or 7-membered cycloalkyl group may be replaced by —O—, —S— or —NR³—, or
R² denotes a C₁₋₃-alkylene bridge which is linked to the group Y, wherein the alkylene bridge may be sustituted with one or more C₁₋₃-alkyl-groups, and R¹ is defined as hereinbefore or denotes a group selected from C₁₋₄-alkyl-CO—, C₁₋₄-alkyl-O—CO—, (C₁₋₄-alkyl)NH—CO— and (C₁₋₄-alkyl)₂N—CO— wherein alkyl-groups may be mono- or polyfluorinated; or
R¹ and R² form a C₃₋₈-alkylene bridge, wherein a —CH₂— group not adjacent to the N atom of the R¹R²N group may be replaced by —CH═N—, —CH═CH—, —O—, —S——SO—, —(SO₂)—, —CO—, —C(═CH₂)—, —C(═N—OH)—, —C(═N—(C₁₋₄-alkyl))- or —NR¹³—,
while in the alkylene bridge defined hereinbefore one or more H atoms may be replaced by identical or different groups R¹⁴, and
the alkylene bridge defined hereinbefore may be substituted by one or two identical or different carbo- or heterocyclic groups Cy in such a way that the bond between the alkylene bridge and the group Cy is made
via a single or double bond,
via a common C atom forming a spirocyclic ring system,
via two common adjacent C and/or N atoms forming a fused bicyclic ring system or
via three or more C and/or N atoms forming a bridged ring system;

X denotes a C₁₋₄-alkylene bridge, while in the definition C₂₋₄-alkylene one or two C atoms may be monosubstituted by R¹⁰, or
a C₃₋₄-alkylene bridge, wherein a —CH₂—CH₂— group not directly adjacent to the N atom of the R¹R²N— group is replaced by —CH₂—O— or —CH₂—NR⁴—,
while the meanings given for X hereinbefore may comprise one, two or three identical or different C₁₋₄-alkyl substituents, while two alkyl groups may be joined together forming a 3 to 7-membered cyclic group; and

R⁴ denotes H or C₁₋₃-alkyl; and

R¹⁰ denotes hydroxy, hydroxy-C₁₋₃-alkyl, C₁₋₄-alkoxy or C₁₋₄-alkoxy-C₁₋₃-alkyl; and

Y is a 5- or 6-membered unsaturated or aromatic carbocyclic group which may contain 1, 2, 3 or 4 heteroatoms selected from N, O and/or S; and which cyclic group may be mono- or polysubstituted by identical or different substituents R²⁰;

Q, Z independently of one another denote a group selected from —CR^{3a}R^{3b}—, —O— and —NR^{N}—,

R^{N} independently of one another denote H, C₁₋₄-alkyl, formyl, C₁₋₃-alkylcarbonyl or C₁₋₃-alkylsulfonyl; and

R^{3a}R^{3b}R^{4a},

R^{4b}R^{5a}R^{5b} independently of one another denote H or C₁₋₄-alkyl; and

A is a 5- or 6-membered unsaturated or aromatic carbocyclic group which may contain 1, 2, 3 or 4 heteroatoms selected from N, O and/or S; which cyclic group may be mono- or polysubstituted by identical or different substituents R²⁰; and

B denotes a group Cy; and

W denotes a single bond, —CH₂—, —O—, —NR^{N}—; —O—CH₂—, —NR^{N}—CH₂—, —CH₂—O—, —CH₂—NR^{N}—, or —CH₂—CH₂—;
or

B is selected from the group consisting of halogen, CN, C₁₋₆-alkyl, C₁₋₆-alkoxy, C₂₋₆-alkenyl, C₂₋₆-alkynyl, C₃₋₆-alkenyloxy, C₃₋₆-alkynyloxy, C₃₋₇-cycloalkyl-C₁₋₃-alkyl, C₃₋₇-cycloalkenyl-C₁₋₃-alkyl, C₁₋₆-alkylcarbonyl, C₁₋₆-alkylamino or di-(C₁₋₆-alkyl)-amino, wherein one or more C atoms independently of one another may be mono- or polysubstituted by halogen and/ or monosubstituted by hydroxy, C₁₋₄-alkoxy or cyano and/ or cyclic groups may be mono- or polysubstituted by identical or different groups R²⁰; and

W denotes a single bond; and

Cy denotes a carbo- or heterocyclic group selected from one of the following meanings
a saturated 3- to 7-membered carbocyclic group,
an unsaturated 4- to 7-membered carbocyclic group,
a phenyl group,
a saturated 4- to 7-membered or unsaturated 5- to 7-membered heterocyclic group with an N, O or S atom as heteroatom,
a saturated or unsaturated 5- to 7-membered heterocyclic group with two or more N atoms or with one or two N atoms and an O or S atom as heteroatoms,
an aromatic heterocyclic 5- or 6-membered group with one or more identical or different heteroatoms selected from N, O and/or S.
while the above-mentioned saturated 6- or 7-membered groups may also be present as bridged ring systems with an imino, (C₁₋₄-alkyl)-imino, methylene, ethylene, (C₁₋₄-alkyl)-methylene or di-(C₁₋₄-alkyl)-methylene bridge, and
while the above-mentioned cyclic groups may be mono- or polysubstituted at one or more C atoms by identical or different groups R²⁰, or in the case of a phenyl group may also additionally be monosubstituted by nitro, and/or one or more NH groups may be substituted by R²¹, and
while in the above-mentioned saturated or unsaturated carbo- or heterocyclic groups a —CH₂-group may be replaced by a —C(═O)— group;

R¹¹ denotes halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N—, R¹⁸R¹⁹N—CO— or Cy, while in the above-mentioned groups one or more C atoms may be substituted independently of one another by substituents selected from halogen, OH, CN, CF₃, C₁₋₃-alkyl, C₁₋₃-alkoxy, hydroxy-C₁₋₃-alkyl;

R¹³ has one of the meanings given for R¹⁷,

R¹⁴ denotes halogen, cyano, C₁₋₆-alkyl, C₂₋₆-alkenyl, C₂₋₆-alkynyl, R¹⁵—O—, R¹⁵—O—CO—, R¹⁵—CO—, R¹⁵—CO—O—, R¹⁶R¹⁷N—, HCO—NR¹⁵—, R¹⁸R¹⁹N—CO—, R¹⁵—O—C₁₋₃-alkyl R¹⁵—O—CO—C₁₋₃-alkyl, R¹⁵—SO₂—NH, R¹⁵—SO₂—N(C₁₋₃-alkyl)—, R¹⁵—O—CO—NH—C₁₋₃-alkyl, R¹⁵—SO₂—NH—C₁₋₃-alkyl, R¹⁵—CO—C₁₋₃-alkyl, R¹⁵—CO—O—C₁₋₃-alkyl, R¹⁶R¹⁷N—C₁₋₃-alkyl, R¹⁸R¹⁹N—CO—C₁₋₃-alkyl or Cy-C₁₋₃-alkyl,

R¹⁵ denotes H, C₁₋₄-alkyl, C₃₋₇-cycloalkyl, C₃₋₇-cycloalkyl-C₁₋₃-alkyl, phenyl, phenyl-C₁₋₃-alkyl, pyridinyl or pyridinyl-C₁₋₃-alkyl,

R¹⁶ denotes H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C_{1 4}-alkyl)-amino-C₂₋₆-alkyl or cyclo-C₃₋₆-alkyleneimino-C₂₋₆-alkyl,

R¹⁷ has one of the meanings given for R¹⁶ or denotes phenyl, phenyl-C₁₋₃-alkyl, pyridinyl, C₁₋₄-alkylcarbonyl, C₃₋₇-cycloalkylcarbonyl, hydroxycarbonyl-C₁₋₃-alkyl, C₁₋₄-alkoxycarbonyl, C₁₋₄-alkylaminocarbonyl, C₁₋₄-alkoxycarbonyl-C₁₋₃-alkyl, C₁₋₄-alkylcarbonylamino-C₂₋₃-alkyl, N—(C₁₋₄-alkylcarbonyl)-N—(C₁₋₄-alkyl)-amino—C₂₋₃-alkyl, C₁₋₄-alkylsulphonyl, C₁₋₄-alkylsulphonylamino-C₂₋₃-alkyl or N—(C₁₋₄-alkylsulphonyl)-N(—C₁₋₄-alkyl)-amino-C₂₋₃-alkyl;

R¹⁸, R¹⁹ independently of one another denote H or C₁₆-alkyl wherein R¹⁸, R¹⁹ may be linked to form a C₃₋₆-alkylene bridge, wherein a —CH₂— group not adjacent to an N atom may be replaced by —O—, —S—, —SO—, —(SO₂)—, —CO—, —C(═CH₂)— or —NR¹³—;

R²⁰ denotes halogen, hydroxy, cyano, nitro, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²—C₁₋₃-alkyl or has one of the meanings given for R²²; and

R²¹ denotes C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulphonyl, aminosulphonyl, C₁₋₄-alkylaminosulphonyl, di-C₁₋₄-alkylaminosulphonyl or cyclo-C₃₋₆-alkylene-imino-sulphonyl,

R²² denotes pyridinyl, phenyl, phenyl-C₁₋₃-alkoxy, cyclo-C₃-₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylamino-carbonyl, di-(C₁₋₄-alkyl)-aminocarbonyl, cyclo-C₃₋₆-alkyl-amino-carbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulphonyl, C₁₋₄-alkyl-sulphinyl, C₁₋₄-alkyl-sulphonylamino, C₁₋₄-alkyl-sulphonyl-N—(C₁₋₄-alkyl)amino, amino, C₁₋₄-alkyl-amino, di-(C₁₋₄-alkyl)-amino, C₁₋₄-alkyl-carbonyl-amino, C₁₋₄-alkyl-carbonyl-N—(C₁₋₄-alkyl)amino, cyclo-C₃₋₆-alkyleneimino, phenyl-C₁₋₃-alkylamino, N—(C₁₋₄-alkyl)-phenyl-C₁₋₃-alkylamino, acetylamino, propionylamino, phenylcarbonyl, phenylcarbonylamino, phenylcarbonylmethylamino, hydroxy-C₂₋₃-alkylamino-carbonyl, (4-morpholinyl)carbonyl, (1-pyrrolidinyl)carbonyl, (1-piperidinyl)-carbonyl, (hexahydro-1-azepinyl)carbonyl, (4-methyl-1-piperazinyl)carbonyl, aminocarbonylamino or C₁₋₄-alkylaminocarbonylamino,

while in the above-mentioned groups and radicals, particularly in A, B, Q, W, X, Y, Z, R^{N}, R^{3a}, R^{3b}, R⁴, R^{4a}, R^{4b}, R^{5a}, R^{5b}, R¹⁰, R¹¹, R¹³ to R²², in each case one or more C atoms may additionally be mono- or polysubstituted by F and/or in each case one or two C atoms independently of one another may additionally be monosubstituted by Cl or Br and/or in each case one or more phenyl rings may additionally comprise independently of one another one, two or three substituents selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetyl-amino, aminocarbonyl, difluoromethoxy, trifluoromethoxy, amino-C₁₋₃-alkyl, C₁₋₃-alkylamino-C₁₋₃-alkyl- and di-(C₁₋₃-alkyl)-amino-C₁₋₃-alkyl and/or may be monosubstituted by nitro, and
the H atom of any carboxy group present or an H atom bound to an N atom may in each case be replaced by a group which can be cleaved in vivo, the tautomers, the diastereomers, the enantiomers, the mixtures thereof and the salts thereof;

with the proviso that the following compounds (D1) and (D2) are not included:

(D1) 2-[[[4-[[3-(2-fluorophenyl)propyl]amino]phenyl]methyl]amino]-propanamide; and

(D2) 2-[[[4-[[3-(3-fluorophenyl)propyl]amino]phenyl]methyl]amino]-propanamide.

The invention also relates to the compounds in the form of the individual optical isomers, mixtures of the individual enantiomers or racemates, in the form of the tautomers and in the form of the free bases or corresponding acid addition salts with pharmacologically acceptable acids. The subject of the invention also includes the compounds according to the invention, including their salts, wherein one or more hydrogen atoms are replaced by deuterium.

This invention also includes the physiologically acceptable salts of the (hetero)aryl compounds according to the invention as described above and hereinafter.

Also covered by this invention are compositions containing at least one (hetero)aryl compound according to the invention and/ or a salt according to the invention optionally together with one or more physiologically acceptable excipients.

Also covered by this invention are pharmaceutical compositions containing at least one (hetero)aryl compound according to the invention and/ or a salt according to the invention optionally together with one or more inert carriers and/or diluents.

This invention also relates to the use of at least one (hetero)aryl compound according to the invention and/or a salt according to the invention, including the compounds (D1) and (D2) explicitly excluded hereinbefore or one of the physiologically acceptable salts thereof, for influencing the eating behaviour of a mammal.

The invention further relates to the use of at least one (hetero)aryl compound according to the invention and/or a salt according to the invention, including the compounds (D1) and (D2) explicitly excluded hereinbefore or one of the physiologically acceptable salts thereof, for reducing the body weight and/ or for preventing an increase in the body weight of a mammal.

The invention also relates to the use of at least one (hetero)aryl compound according to the invention and/or a salt according to the invention, including the compounds (D1) and (D2) explicitly excluded hereinbefore or one of the physiologically acceptable salts thereof, for preparing a pharmaceutical composition with an MCH receptor-antagonistic activity, particularly with an MCH-1 receptor-antagonistic activity.

This invention also relates to the use of at least one (hetero)aryl compound according to the invention and/or a salt according to the invention, including the compounds (D1) and (D2) explicitly excluded hereinbefore or one of the physiologically acceptable salts thereof, for preparing a pharmaceutical composition which is suitable for the prevention and/or treatment of symptoms and/or diseases which are caused by MCH or are otherwise causally connected with MCH.

A further object of this invention is the use of at least one (hetero)aryl compound according to the invention and/or a salt according to the invention, including the compounds (D1) and (D2) explicitly excluded hereinbefore or one of the physiologically acceptable salts thereof, for preparing a pharmaceutical composition which is suitable for the prevention and/or treatment of metabolic disorders and/or eating disorders, particularly obesity, bulimia, bulimia nervosa, cachexia, anorexia, anorexia nervosa and hyperphagia.

The invention also relates to the use of at least one (hetero)aryl compound according to the invention and/or a salt according to the invention, including the compounds (D1) and (D2) explicitly excluded hereinbefore or one of the physiologically acceptable salts thereof, for preparing a pharmaceutical composition which is suitable for the prevention and/or treatment of diseases and/or disorders associated with obesity, particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis and gonitis.

In addition the present invention relates to the use of at least one (hetero)aryl compound according to the invention and/or a salt according to the invention, including the compounds (D1) and (D2) explicitly excluded hereinbefore or one of the physiologically acceptable salts thereof, for preparing a pharmaceutical composition which is suitable for the prevention and/or treatment of hyperlipidaemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia and hormonal disorders.

The invention also relates to the use of at least one (hetero)aryl compound according to the invention and/or a salt according to the invention, including the compounds (D1) and (D2) explicitly excluded hereinbefore or one of the physiologically acceptable salts thereof, for preparing a pharmaceutical composition which is suitable for the prevention and/or treatment of urinary problems, such as for example urinary incontinence, overactive bladder, urgency, nycturia and enuresis.

The invention further relates to the use of at least one (hetero)aryl compound according to the invention and/ or a salt according to the invention, including the compounds (D1) and (D2) explicitly excluded hereinbefore or one of the physiologically acceptable salts thereof, for preparing a pharmaceutical composition which is suitable for the prevention and/or treatment of dependencies and/or withdrawal symptoms.

The invention further relates to processes for preparing for preparing a pharmaceutical composition according to the invention, characterised in that at least one (hetero)aryl compound according to the invention and/ or a salt according to the invention is incorporated in one or more inert carriers and/or diluents by a non-chemical method.

The invention also relates to a pharmaceutical composition containing a first active substance which is selected from the (hetero)aryl compounds according to the invention and/or the corresponding salts, including the compounds (D1) and (D2) explicitly excluded hereinbefore or one of the physiologically acceptable salts thereof, as well as a second active substance which is selected from the group consisting of active substances for the treatment of diabetes, active substances for the treatment of diabetic complications, active substances for the treatment of obesity, preferably other than MCH antagonists, active substances for the treatment of high blood pressure, active substances for the treatment of dyslipidaemia or hyperlipidaemia, including arteriosclerosis, active substances for the treatment of arthritis, active substances for the treatment of anxiety states and active substances for the treatment of depression, optionally together with one or more inert carriers and/or diluents.

Moreover, in one aspect, the invention relates to a process for preparing (hetero)aryl compounds of formula (1-3) 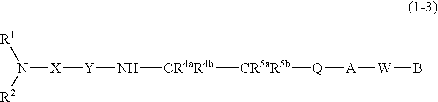
wherein R¹, R², X, Y, R^{4a}, R^{4b}, R^{5a}, R^{5b}, Q, A, W, and B are defined as hereinbefore and hereinafter, by reacting a compound of general formula (1-1) 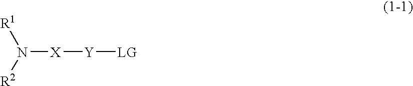
wherein R¹, R², X and Y are defined as hereinbefore and hereinafter,
with a compound of general formula (1-2) 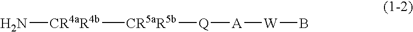
wherein R^{4a}, R^{4b}, R^{5a}, R^{5b}, Q, A, W and B are defined as hereinbefore and hereinafter,
in the presence of a palladium catalyst with or without ligands and/or copper iodide and in the presence of a base.

The starting materials and intermediate products used in the synthesis according to the invention are also a subject of this invention.

### DETAILED DESCRIPTION OF THE INVENTION

Unless otherwise specified, the groups, residues and substituents, particularly A, B, Q, W, X, Y, Z, Cy, R¹, R², R^{3a}, R^{3b}, R⁴, R^{4a}, R^{4b}, R^{5a}, R^{5b}, R¹⁰, R¹¹, R¹³ to R²², R^{N}, have the meanings given hereinbefore.

If groups, residues and/or substituents occur more than once in a compound, they may have the same or different meanings in each case.

If R¹ and R² are not joined together via an alkylene bridge, R¹ and R² independently of one another preferably denote a C₁₋₈-alkyl or C₃₋₇-cycloalkyl group which may be mono- or polysubstituted by identical or different groups R¹¹, while a —CH₂— group in position 3 or 4 of a 5-, 6- or 7-membered cycloalkyl group may be replaced by —O—, —S— or —NR³—, while one or both of the groups R¹ and R² may also represent H.

Preferred meanings of the group R¹¹ are F, Cl, Br, C₁₋₆-alkyl, C₂₋₆-alkenyl, C₂₋₆-alkynyl, R¹⁵—O—, cyano, R¹⁶R¹⁷N, C₃₋₇-cycloalkyl, cyclo-C₃₋₆-alkyleneimino, pyrrolidinyl, N—(C₁₋₄-alkyl)-pyrrolidinyl, piperidinyl, N—(C₁₋₄-alkyl)-piperidinyl, phenyl, pyridyl, pyrazolyl, thiazolyl, imidazolyl, while in the above-mentioned groups and radicals one or more C atoms may be mono- or polysubstituted independently of one another by F, C₁₋₃-alkyl, C₁₋₃-alkoxy or hydroxy-C₁₋₃-alkyl, and/or one or two C atoms may be monosubstituted independently of one another by Cl, Br, OH, CF₃ or CN, and the above-mentioned cyclic groups may be mono- or polysubstituted at one or more C atoms by identical or different radicals R²⁰, or in the case of a phenyl group may also additionally be monosubstituted by nitro, and/or one or more NH groups may be substituted by R²¹. If R¹¹ has one of the meanings R¹⁵—O—, cyano, R¹⁶R¹⁷N or cyclo-C₃₋₆-alkyleneimino, the C atom of the alkyl or cycloalkyl group substituted by R¹¹ is preferably not directly connected to a heteroatom, such as for example to the group —N—X—.

Preferably the groups R¹, R² independently of one another represent H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkylene alkyl, pyrrolidin-3-yl, N—(C₁₋₄-alkyl)-pyrrolidin-3-yl, pyrrolidinyl-C₁₋₃-alkyl, N—(C₁₋₄-alkyl)-pyrrolidinyl-C₁₋₃-alkyl, piperidin-3-yl, piperidin-4-yl, N—(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-tetrahydropyran-4-yl, tetrahydrofuran-2-ylmethyl, tetrahydrofuran-3-ylmethyl, phenyl-C₁₋₃-alkyl, pyridyl-C₁₋₃-alkyl, pyrazolyl-C₁₋₃-alkyl, thiazolyl-C₁₋₃-alkyl or imidazolyl-C₁₋₃-alkyl, while the above-mentioned groups and radicals one or more C atoms independently of one another may be mono- or polysubstituted by F. C₁₋₃-alkyl or hydroxy-C₁₋₃-alkyl, and/or one or two C atoms independently of one another may be monosubstituted by Cl, Br, OH, CF₃ or CN, and the above-mentioned cyclic groups may be mono- or polysubstituted at one or more C atoms by identical or different radicals R²⁰, in the case of a phenyl group may also additionally be monosubstituted by nitro, and/or one or more NH groups may be substituted by R²¹. Preferred substituents of the above-mentioned phenyl or pyridyl groups are selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl and di-(C₁₋₃-alkyl)-amino-C₁₋₃-alkyl, while a phenyl group may also be monosubstituted by nitro.

Particularly preferred definitions of the groups R¹ and/or R² are selected from the group consisting of H, C₁₋₄-alkyl, hydroxy-C₁₋₄-alkyl, C₃₋₅-alkenyl, C₃₋₅-alkynyl, C₃₋₇-cycloalkyl, hydroxy-C₃₋₇-cycloalkyl, dihydroxy-C₃₋₆-alkyl, C₃₋₇-cycloalkyl-C₁₋₃-alkyl, tetrahydropyran-3-yl, tetrahydropyran-4-yl, tetrahydrofuran-2-yl methyl, tetrahydrofuran-3-ylmethyl, (hydroxy-C₃₋₇-cycloalkyl)-C₁₋₃-alkyl, C₁₋₄-alkoxy-C₂₋₃-alkyl, hydroxy-C₁₋₄-alkoxy-C₂-₃-alkyl, C₁₋₄-alkoxy-C₁₋₄-alkoxy-C₂₋₃-alkyl, di-(C₁₋₃-alkyl)amino-C₂₋₃-alkyl, pyrrolidin-N-yl-C₂₋₃-alkyl and piperidin-N-yl-C₂₋₃-alkyl, while an alkyl, cycloalkyl or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl or CN.

Most particularly preferred groups R¹ and/or R² are selected from the group consisting of H, methyl, ethyl, n-propyl, i-propyl, prop-2-enyl, but-2-enyl, prop-2-ynyl, but-2-ynyl, 2-methoxyethyl, cyclopropyl, cyclopentyl, cyclohexyl, cyclopropylmethyl, cyclopentylmethyl, hydroxy-C₃₋₇-cycloalkyl, (hydroxy-C₁₋₃-alkyl)-hydroxy-C₃₋₇-cycloalkyl, dihydroxy-C₃₋₅-alkyl, 2-hydroxy- 1-(hydroxymethyl)-ethyl, 1,1-di(hydroxymethyl)-ethyl, (1-hydroxy-C₃₋₆-cycloalkyl)-methyl, tetrahydropyran-3-yl, tetrahydropyran-4-yl, tetrahydrofuran-2-ylmethyl, tetrahydrofuran-3-ylmethyl, 2-hydroxyethyl, 3-hydroxypropyl, 2-hydroxypropyl, 2-hydroxy-2-methyl-propyl, hydroxy-C₁₋₄-alkoxy-C₂₋₃-alkyl, di-(C₁₋₃-alkyl)aminoethyl, pyrrolidin-N-yl-ethyl and piperidin-N-ylethyl, while the above-mentioned groups may be mono- or polysubstituted by F and/or C₁₋₃-alkyl.

Examples of most particularly preferred groups R¹ and/or R² are therefore H, methyl, ethyl, n-propyl, i-propyl, prop-2-enyl, prop-2-ynyl, 2-methoxyethyl, cyclopropyl, cyclopentyl, cyclohexyl, cyclopropylmethyl, cyclopentylmethyl, hydroxy-cyclopentyl, hydroxy-cyclohexyl, (hydroxymethyl)-hydroxy-cyclopentyl, (hydroxymethyl)-hydroxy-cyclohexyl, 2,3-dihydroxypropyl, (1-hydroxy-cyclopropyl)-methyl, tetrahydropyran-3-yl, tetrahydropyran-4-yl, tetrahydrofuran-2-yl methyl, tetrahydrofuran-3-yl methyl, 2-hydroxyethyl, 2-hydroxypropyl, 3-hydroxypropyl, 2-hydroxy-2-methyl-propyl, hydroxyethoxyethyl and dimethylaminoethyl.

Particularly preferably, at least one of the groups R¹, R² has a meaning other than H.

In case the group R² denotes a C₁₋₃-alkylene bridge which is linked to the group Y, preferably the definition of R¹ is in accordance with a preferred definition as described hereinbefore or R¹ denotes a group selected from C₁₋₄-alkyl-CO—, C₁₋₄-alkyl-O—CO—, (C₁₋₄-alkyl)NH—CO— or (C₁₋₄-alkyl)₂N—CO— wherein alkyl-groups may be mono- or polyfluorinated. In case R² is linked to the group Y, then R² preferably denotes —CH₂— or —CH₂—CH₂—, wherein the alkylene bridge may be sustituted with one or more C₁₋₃-alkyl-groups. In case R² is linked to the group Y, then R¹ preferably denotes H or C₁₋₃-alkyl which may be mono- or polyfluorinated.

If R¹ and R² form an alkylene bridge, this is preferably a C₃-₇-alkylene bridge or a C₃₋₇-alkylene bridge, wherein a —CH₂— group not adjacent to the N atom of the R¹R²N group is replaced by —CH═N—, —CH═CH—, —O—, —S—, —(SO₂)—, —CO—, —C(═N—OH)—, —C(═N—(C₁₄-alkyl))— or —NR¹⁻³—,

while in the alkylene bridge defined hereinbefore one or more H atoms may be replaced by identical or different groups R¹⁴, and

the alkylene bridge defined hereinbefore may be substituted with a carbo- or heterocyclic group cy in such a way that the bond between the alkylene bridge and the group Cy is made
via a single or double bond,
via a common C atom forming a spirocyclic ring system,
via two common adjacent C- and/or N atoms forming a fused bicyclic ring system or
via three or more C- and/or N atoms forming a bridged ring system.

Preferably also, R¹ and R² form an alkylene bridge such that R¹R²N— denotes a group which is selected from azetidine, pyrrolidine, piperidine, azepan, 2,5-dihydro-1H-pyrrole, 1,2,3,6-tetrahydro-pyridine, 2,3,4,7-tetrahydro-1H-azepine, 2,3,6,7-tetrahydro-1H-azepine, piperazine in which the free imine function is substituted by R³, piperidin-4-one morpholine thiomorpholine, 1-oxo-thiomorpholin-4-yl, 1,1-dioxo-thiomorpholin-4-yl, 4-C₁₋₄-alkoxy-imino-piperidin-1-yl and 4-hydroxyimino-piperidin-1-yl; or

a group which is particularly preferably selected from pyrrolidine, piperidine, piperazine in which the free imine function is substituted by R¹³, and morpholine,

while according to the general definition of R¹ and R² one or more H atoms may be replaced by identical or different groups R¹⁴, and/ or the above-mentioned groups may be substituted by one or two identical or different carbo- or heterocyclic groups Cy in a manner specified according to the general definition of R¹ and R² while the group Cy may be mono- or polysubstituted by R²⁰.

Particularly preferred groups Cy are C₃₋₇-cycloalkyl, aza-C₄₋₇-cycloalkyl, particularly cyclo-C₃₋₆-alkyleneimino, as well as 1-C₁₋₄-alkyl-aza-C₄₋₇-cycloalkyl, while the group Cy may be mono- or polysubstituted by R²⁰.

The C₃₋₈-alkylene bridge formed by R¹ and R², wherein —CH₂— groups may be replaced as specified, may be substituted, as described, by one or two identical or different carbo- or heterocyclic groups Cy, which may be substituted as specified hereinbefore.

In the event that the alkylene bridge is linked to a group Cy through a single bond, Cy is preferably selected from the group consisting of C₃₋₇-cycloalkyl, cyclo-C₃₋₆-alkyleneimino, imidazol, triazol, thienyl and phenyl.

In the event that the alkylene bridge is linked to a group Cy via a common C atom forming a spirocyclic ring system, Cy is preferably selected from the group consisting of C₃₋₇-cycloalkyl, aza-C₄₋₈-cycloalkyl, oxa-C₄₋₈-cycloalkyl, 2,3-dihydro-1H-quinazolin-4-one.

In the event that the alkylene bridge is linked to a group Cy via two common adjacent C and/or N atoms forming a fused bicyclic ring system, Cy is preferably selected from the group consisting of C₄₋₇-cycloalkyl, phenyl, thienyl.

In the event that the alkylene bridge is linked to a group Cy via three or more C and/or N atoms forming a bridged ring system, Cy preferably denotes C₄₋₈-cycloalkyl or aza-C₄₋₈-cycloalkyl.

In the event that the heterocyclic group R¹R²N— is substituted by a group Cy, the group Cy is preferably linked to the group R¹R²N— through a single bond, while Cy is preferably selected from the group consisting of C₃₋₇-cycloalkyl, cyclo-C₃₋₆-alkyleneimino, imidazol, imidazolidin-2-one, and triazol, while these groups may be substituted as specified, preferably by fluorine, C₁₋₃-alkyl, hydroxy-C₁₋₃-alkyl and hydroxy. Particularly preferably the group 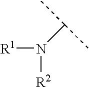
is defined according to one of the following partial formulae 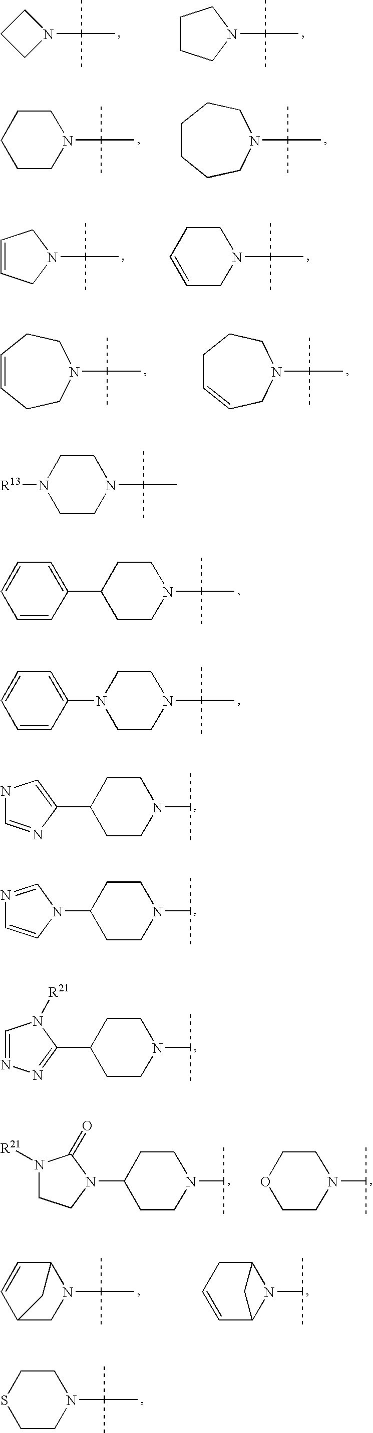 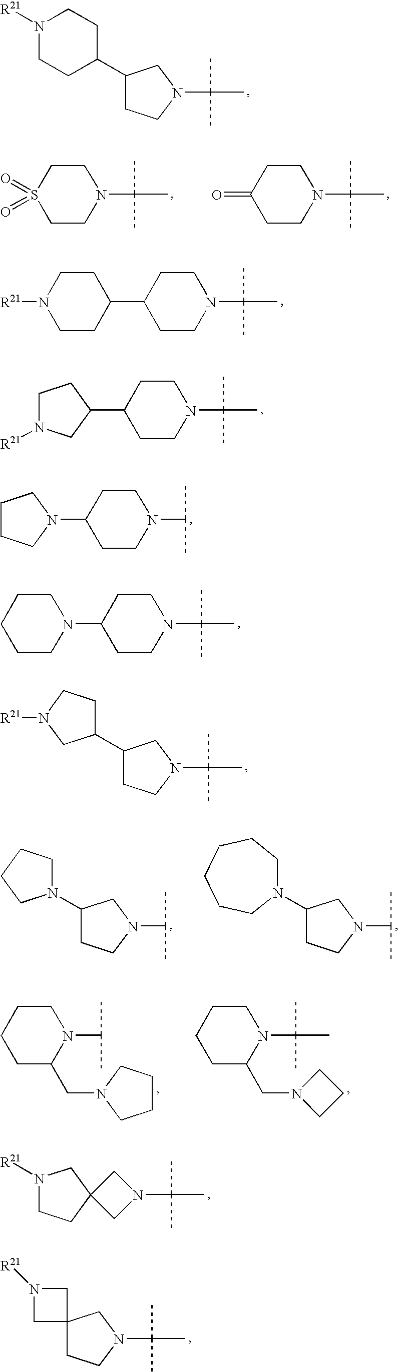 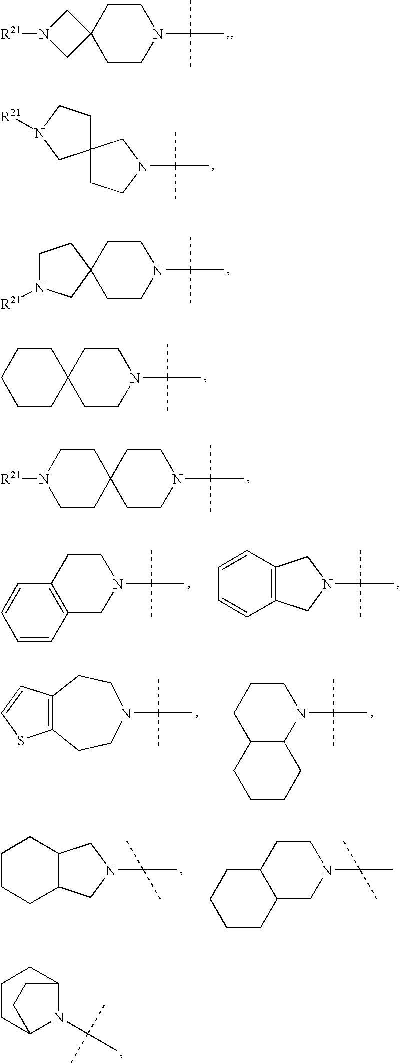
wherein one or more H atoms of the heterocycle formed by the group R¹R²N— may be replaced by identical or different groups R¹⁴, and

the heterocycle formed by the group R¹R²N— may be substituted by one or two, preferably one C₃₋₇-cycloalkyl group, while the cycloalkyl group may be mono- or polysubstituted by R²⁰, and

the ring attached to the heterocycle formed by the group R¹R²N— may be mono- or polysubstituted at one or more C atoms by R²⁰, or in the case of a phenyl ring may also additionally be monosubstituted by nitro and

wherein R¹³, R¹⁴, R²⁰, R²¹ have the meanings given hereinbefore and hereinafter.

If the heterocycle formed by the group R¹R²N— is substituted as specified by one or two cycloalkyl groups mono- or polysubstituted by R²⁰, the substituents R²⁰ independently of one another preferably denote C₁₋₄-alkyl, C₁₋₄-alkoxy-C₁₋₃-alkyl, hydroxy-C₁₋₃-alkyl, hydroxy, fluorine, chlorine, bromine or CF₃, particularly hydroxy.

Most particularly preferably the group 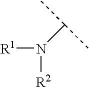
is defined according to one of the following partial formulae 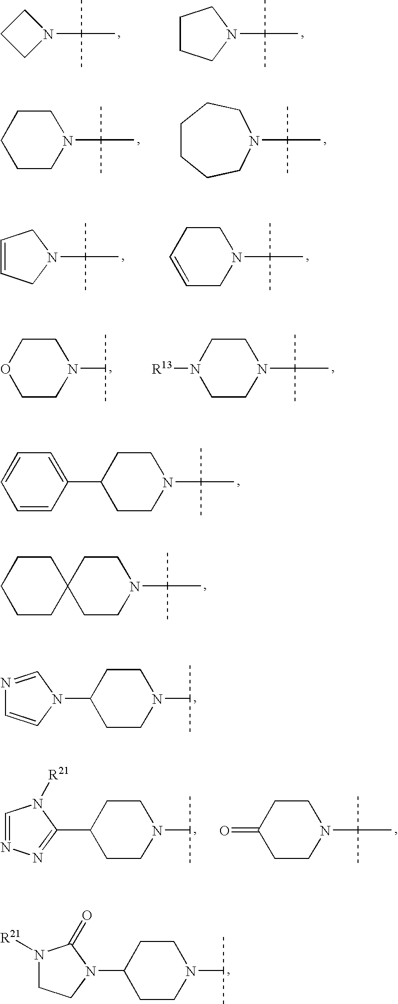
particularly 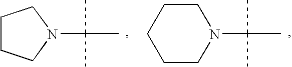
where R¹³ has the meanings given above and hereinafter, and

the heterocycle formed by the group R¹R²N- may be substituted by C₃₋₆-cycloalkyl, hydroxy-C₃₋₆-cycloalkyl or (hydroxy-C₃₋₆-cycloalkyl)-C₁₋₃-alkyl, and

the heterocycle formed by the group R¹R²N— may be mono-, di- or trisubstituted by identical or different groups R¹⁴.

The following definitions of the group R¹R²N are particularly preferred: azetidinyl, pyrrolidinyl, piperidinyl, 2,5-dihydro-1H-pyrrole, 1,2,3,6-tetrahydro-pyridine, morpholinyl,

fluoroazetidinyl, fluoropyrrolidinyl, fluoropiperidinyl, methylpyrrolidinyl, methylpiperidinyl, hydroxyazetidinyl, hydroxypyrrolidinyl, hydroxypiperidinyl, hydroxyazepanyl, (hydroxymethyl)-pyrrolidinyl, (hydroxymethyl)-piperidinyl,

3,4-dihydroxypyrrolidinyl, 3,4-dihydroxypiperidinyl, 3,5-dihydroxypiperidinyl, (hydroxymethyl)-hydroxy-pyrrolidinyl, (hydroxymethyl)-hydroxy-piperidinyl,

dimethylaminopyrrolidinyl, dimethylaminopiperidinyl, aminocarbonylpyrrolidinyl, methylaminocarbonylpyrrolidinyl, dimethylaminocarbonylpyrrolidinyl, aminocarbonylpiperidinyl, methylaminocarbonylpiperidinyl, dimethylaminocarbonylpiperidinyl, formylaminopiperidinyl, (N-formyl-N-methylamino)-piperidinyl,

methylcarbonylaminopiperidinyl, methylcarbonylaminopyrrolidinyl, N-(methylcarbonyl)-N-methyl-aminopiperidinyl, N-(methylcarbonyl)-N-methyl-aminopyrrolidinyl, ethylcarbonylamino-piperidinyl, ethylcarbonylaminopyrrolidinyl, N-(ethylcarbonyl)-N-methyl-aminopiperidinyl, N-(ethylcarbonyl)-N-methyl-aminopyrrolidinyl, cyclopropylcarbonylaminopiperidinyl, cyclopropylcarbonylaminopyrrolidinyl, N-(cyclopropylcarbonyl)-N-methyl-aminopiperidinyl, N-(cyclopropylcarbonyl)-N-methyl-aminopyrrolidinyl,

methylcarbonylaminomethylpiperidinyl, methylcarbonylaminomethylpyrrolidinyl, N-(methylcarbonyl)-N-methyl-aminomethylpiperidinyl, N-(methylcarbonyl)-N-methyl-aminomethylpyrrolidinyl,

methylsulfonylaminopyrrolidinyl, methylsulfonylaminopiperidinyl, N-(methylsulfonyl)-N-methyl-aminopyrrolidinyl, N-(methylsulfonyl)-N-methyl-aminopiperidinyl,

methoxycarbonylpyrrolidinyl, methoxycarbonylpiperidinyl, N-methyl-piperazinyl, N-(methylcarbonyl)-piperazinyl,

(methyl-4H-triazolyl)-pyrrolidinyl, (methyl-4H-triazolyl)-piperidinyl,

(methyl-imidazolidin-2-on-yl)pyrrolidinyl, (methyl-imidazolidin-2-on-yl)piperidinyl, imidazolylpyrrolidinyl, imidazolylpiperidinyl,

while in the groups mentioned a hydroxymethyl group may be mono- or disubstituted at the C atom by methyl, while two methyl substituents may be joined together, forming a cyclopropyl group, and

in one or two hydroxy groups the H atom may be replaced by a methyl group, and

the groups R¹R²N- mentioned have no further substituents or have one or two substituents selected independently of one another from fluorine, hydroxy, C₁₋₃-alkyl, hydroxy-C₁₋₃-alkyl, CF₃.

The following partial formulae are most particularly preferred definitions of the heterocyclic group 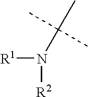
specified above: 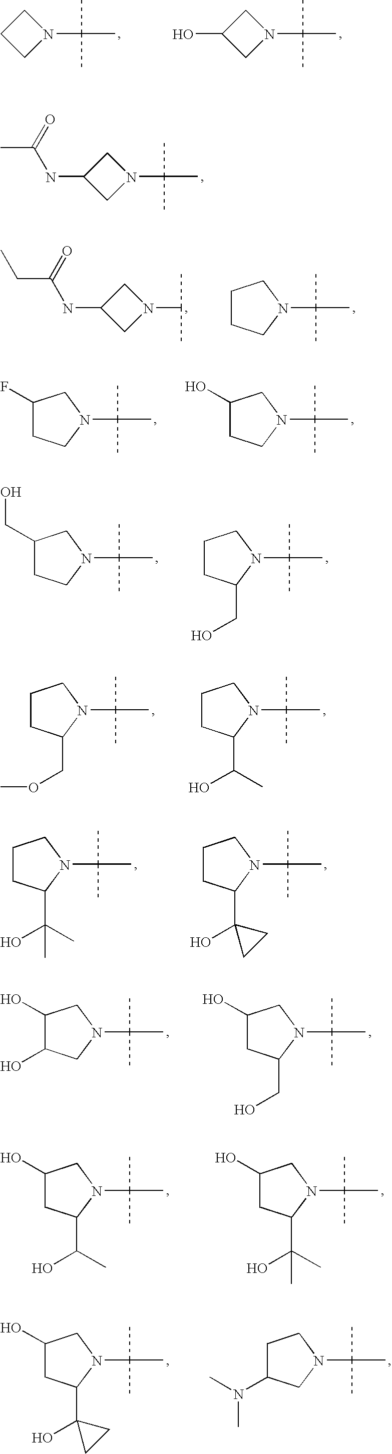 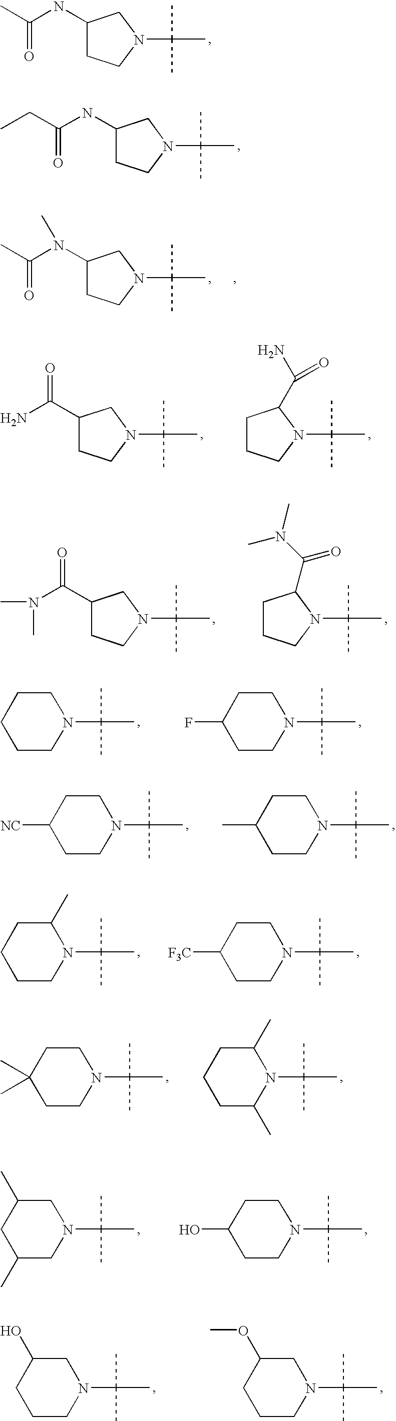 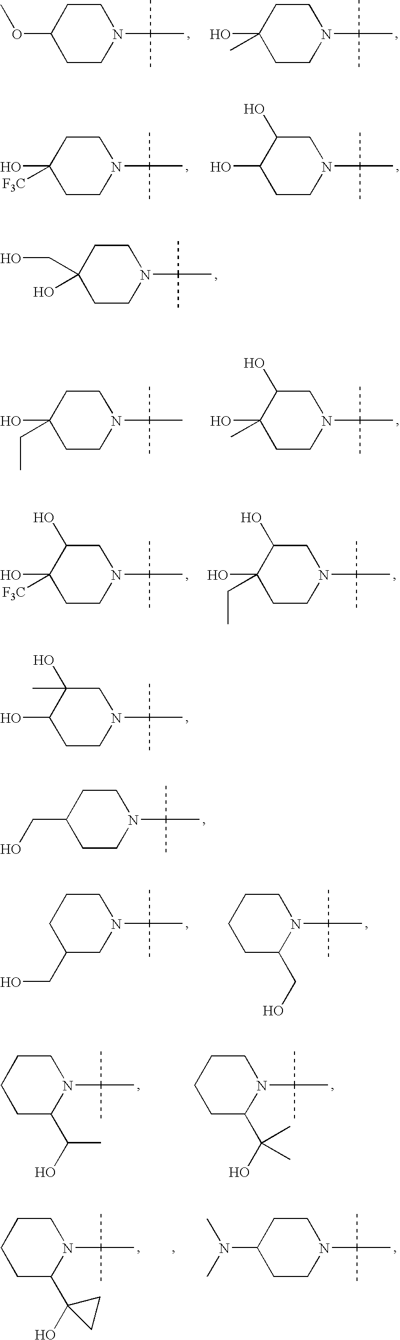 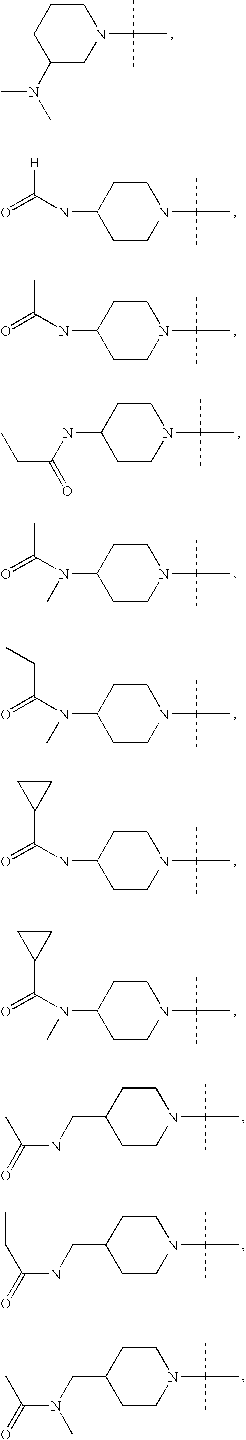 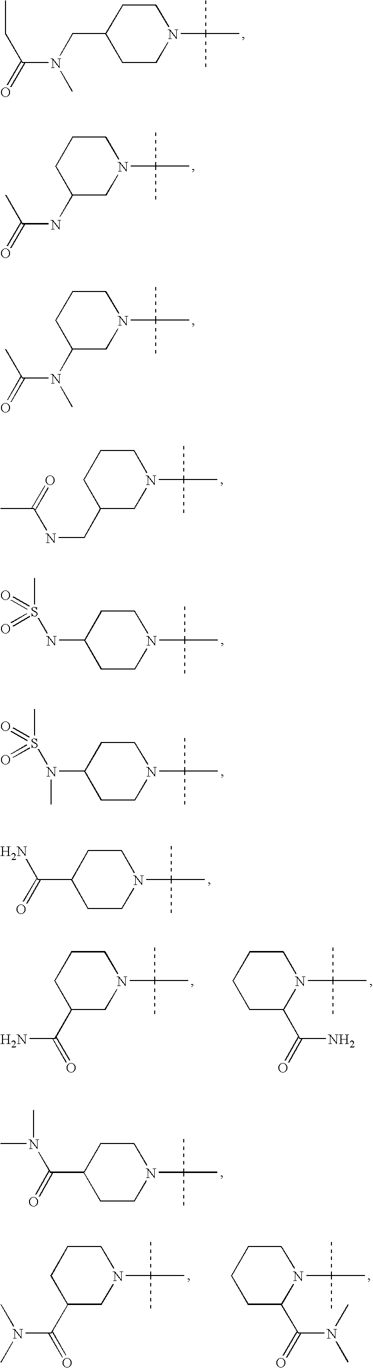 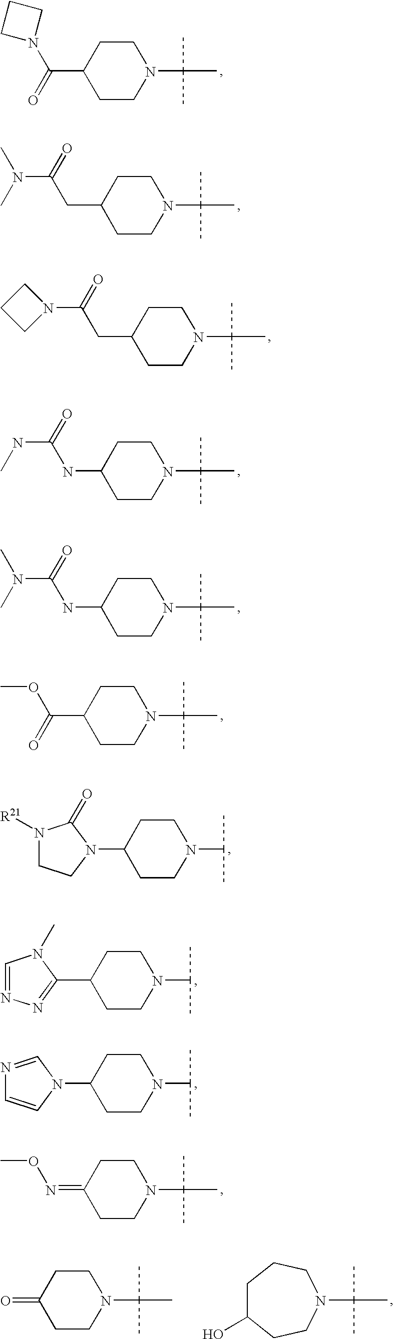 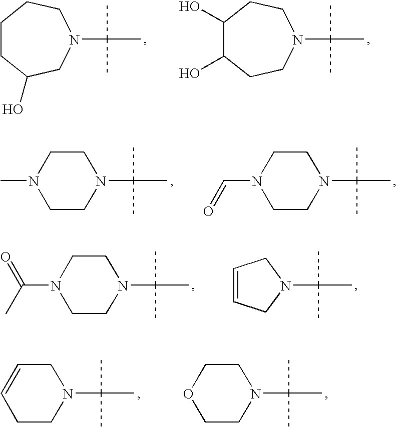
wherein the groups mentioned are not further substituted, or

wherein methyl or ethyl groups may be mono-, di- or trisubstituted by fluorine, and wherein one or more H atoms of the heterocycle formed by the group R¹R²N— which are bound to carbon may be substituted independently of one another by fluorine, chlorine, CN CF₃ C₁₃-alkyl, hydroxy-C₁₋₃-alkyl, particularly C₁₋₃-alkyl or CF₃ preferably methyl, ethyl, CF₃.

Among the above-mentioned preferred and particularly preferred meanings of R¹R²N, the following definitions of the substituent R¹⁴ are preferred: F Cl Br, cyano, C₁₄-alkyl, C₂₋₄-alkenyl, C₂₋₄-alkynyl, C₃₋₇-cycloalkyl, C₃₋₇-cycloalkyl-C₁₋₃-alkyl, hydroxy, hydroxy-C₁₋₃-alkyl, C₁₋₄-alkoxy, ω-(C₁₋₄-alkoxy)-C₁₃-alkyl, C₁₋₄-alkyl-carbonyl, carboxy, C₁₋₄-alkoxycarbonyl, hydroxy-carbonyl-C₁₋₃-alkyl, C₁₋₄-alkoxycarbonyl-C₁₋₃-alkyl, formylamino, formyl-N—(C₁₋₄-alkyl)-amino, C₁₋₄-alkyl-carbonylamino, C₁₋₄-alkyl-carbonyl-N—(C₁₋₃-alkyl)amino, C₃₋₇-cycloalkyl-carbonylamino, C₁₋₄-alkyl-aminocarbonylamino, C₁₋₄-alkyl-carbonylamino-C₁₋₃-alkyl, C₁₋₄-alkyl-carbonyl-N—(C₁₋₃-alkyl)amino-C₁₋₃-alkyl, C₃₋₇-cycloalkyl-carbonylamino-C₁₋₃-alkyl, C₁₋₄-aminocarbonylamino-C₁₋₃-alkyl, C₁₋₄-alkyl-sulfonylamino, C₁₋₄-alkyl-sulfonyl-N—(C₁₋₃-alkyl)amino, C₁₋₄-alkoxy-carbonylamino, C₁₋₄-alkoxy-carbonylamino-C₁₋₃-alkyl, amino, C₁₋₄-alkyl-amino, C₃₋₇-cycloalkyl-amino, C₃₋₇-cycloalkyl-N—(C₁₋₄-alkyl)-amino, di-(C₁₋₄-alkyl)-amino cyclo-C₃₋₆-alkyleneimino, amino-C₁₋₃-alkyl, C₁₋₄-alkyl-amino-C₁₋₃-alkyl, C₃₋₇-cycloalkyl-amino-C₁₋₃-alkyl, C₃₋₇-cycloalkyl-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, C₁₋₄-alkyl-amino-carbonyl, C₃₋₇-cycloalkyl-amino-carbonyl, C₃₋₇-cycloalkyl-N—(C₁₋₄-alkyl)-amino-carbonyl, di-(C₁₋₄-alkyl)-amino-carbonyl and (aza-C₄₋₆-cycloalkyl)-carbonyl.

Particularly preferred meanings of the substituent R¹⁴ are F, Cl, Br, C₁₋₄-alkyl, hydroxy, hydroxy-C₁₋₃-alkyl, C₁₋₄-alkoxy, ω-(C₁₋₄-alkoxy)-C₁₋₃-alkyl, C₁₋₄-alkoxycarbonyl, amino-C₁₋₃-alkyl, C₁₋₄-alkyl-amino-C₁₋₃-alkyl, C₃₋₇-cycloalkyl-amino-C₁₋₃-alkyl, C₃₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, di-(C₁₋₄-alkyl)-amino-carbonyl, (aza-C₄₋₆-cycloalkyl)-carbonyl, di-(C₁₋₄-alkyl)-amino, formylamino, formyl-N(C₁₋₄-alkyl)-amino, C₁₋₄-alkyl-carbonylamino, C₁₋₄-alkyl-carbonyl-N—(C₁₋₃-alkyl)amino, C₃₋₅-cycloalkyl-carbonylamino, C₁₋₄-alkyl-aminocarbonylamino, C₁₋₄-alkyl-carbonylamino-C₁₋₃-alkyl, N—(C₁₋₄-alkyl-carbonyl)-N—(C₁₋₃-alkyl)amino-C₁₋₃-alkyl, C₃₋₅-cycloalkyl-carbonylamino-C₁₋₃-alkyl, C₁₋₄-alkyl-sulfonylamino, and N—(C₁₋₄-alkyl-sulfonyl)-N—(C₁₋₃-alkyl)amino.

In the above-mentioned preferred meanings of R¹⁴ in each case one or more C atoms may additionally be mono- or polysubstituted by F and/or in each case one or two C atoms may independently of one another additionally be monosubstituted by Cl or Br. Thus, preferred meanings of R¹⁴ also include, for example, —CF₃, —OCF₃, CF₃—CO— and CF₃—CHOH—.

Most particularly preferred meanings of the substituent R¹⁴ are F, C₁₋₃-alkyl, C₁₋₃-alkoxy, hydroxy-C₁₋₃-alkyl, methoxymethyl, hydroxy, CF₃, C₁₋₃-alkoxycarbonyl, aminocarbonyl, di(C₁₋₃-alkyl)amino, formylamino, N-formyl-N(C₁₋₃-alkyl)amino, C₁₋₃-alkyl-carbonylamino, C₁₋₄-alkyl-carbonyl-N-methyl-amino, C₃₋₅-cycloalkyl-carbonylamino, C₁₋₃-alkyl-aminocarbonylamino, C₁₋₃-alkyl-carbonylaminomethyl, C₁₋₄-alkyl-carbonyl-N-methyl-aminomethyl, C₃₋₅-cycloalkyl-carbonylaminomethyl, C₁₋₃-alkyl-sulfonylamino, C₁₋₄-alkyl-sulfonyl-N—(C₁₋₃-alkyl)amino, CF₃—CHOH—.

Examples of most preferred meanings of R¹⁴ are F, hydroxy, methyl, ethyl, CF₃, methoxy, hydroxymethyl, 2-hydroxyethyl, methoxycarbonyl, dimethylamino, formylamino, N-formyl-N-methylamino, methylcarbonylamino, ethylcarbonylamino, methylcarbonyl-N-methyl-amino, cyclopropyl-carbonylamino, methylcarbonylaminomethyl, ethylcarbonylaminomethyl, methylcarbonyl-N-methyl-aminomethyl, cyclopropyl-carbonylaminomethyl, methylamino-carbonylamino, methylsulfonylamino, methylsulfonyl-N-methylamino.

The group X preferably denotes a —CH₂—, —CH₂—CH₂—, —CH₂—CH₂—O— or —CH₂—CH₂—NR⁴— bridging group, wherein one or two hydrogen atoms may be replaced by identical or different C₁₋₃-alkyl-groups, while two alkyl-groups may linked together to form a 3 to 6-membered cycloalkyl group; and wherein R⁴ is as defined hereinbefore or preferably denotes H or methyl.

Most preferably the group X denotes a —CH₂—, —CH₂—CH₂— or —CH₂—CH₂—O—.

In case the substituent R² denotes an alkylene bridge which is linked to the group Y, then the group X preferably denotes —CH₂— or —CH₂—CH₂—.

The group Y preferably denotes a phenyl, pyridyl, pyridazinyl, pyrimidinyl or pyrazinyl group which may be mono- or polysubstituted by identical or different substituents R²⁰.

More preferably the group Y denotes phenyl, pyridyl or pyridazinyl, which may be mono- or polysubstituted, in particular mono- or disubstituted by identical or different substituents R²⁰.

Most preferably the group Y denotes a group characterized by a subformula selected from 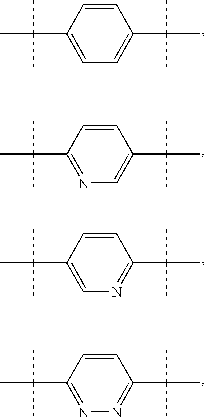
which may be mono- or disubstituted by identical or different substituents R²⁰.

Preferred substituents R²⁰ of the group Y are selected from halogen, C₁₋₃-alkyl, C₁₋₃-alkoxy, hydroxy and CF₃; in particular chlorine or bromine.

According to a first embodiment the groups Q. Z independently of one another preferably denote a group selected from —CH₂—, —O— and —NR^{N}— with the proviso that Q and Z do not both at the same time denote —CH₂—.

According to a second embodiment the groups Q and Z denote —CH₂—.

The groups R^{N} independently of each other preferably denotes H, methyl, ethyl or formyl; most preferably H.

The groups R^{4a}, R^{4b}, R^{5a , R}^{5b} preferably denote H.

Therefore according to said first embodiment preferred meanings of the bridging group -Z-CR^{4a}R^{4b}—CR^{5a}R^{5b}-Q- are selected from the group of subformulae consisting of

(a) —NR^{N}—CH₂—CH₂—CH₂—,

(b) —NR^{N}—CH₂—CH₂—NR^{N}—;

(c) —NR^{N}—CH₂—CH₂—O—,

(d) —CH₂—CH₂—CH₂—NR^{N}—,

(e) —O—CH₂—CH₂—NR^{N}—, and

(f) —O—CH₂—CH₂—O—,

(g) —O—CH₂—CH₂—CH₂—,

(h) —CH₂—CH₂—CH₂—O—,

wherein R^{N} is defined as hereinbefore. The subformulae (a), (c), (d) and (g) are particularly preferred.

According to said second embodiment the bridging group -Z-CR^{4a}R^{4b}—CR^{5a}R^{5b}-Q- is preferably the group —CH₂—CH₂—CH₂—CH₂—.

The group A preferably denotes a phenyl, pyridyl, pyridazinyl, pyrimidinyl or pyrazinyl group which may be mono- or polysubstituted by identical or different substituents R²⁰.

More preferably the group A denotes phenyl, pyridyl or pyridazinyl, which may be mono- or polysubstituted, in particular mono- or disubstituted by identical or different substituents R²⁰.

Most preferably the group A denotes a group characterized by a subformula selected from 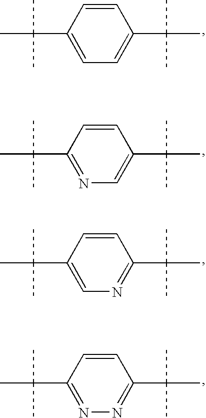 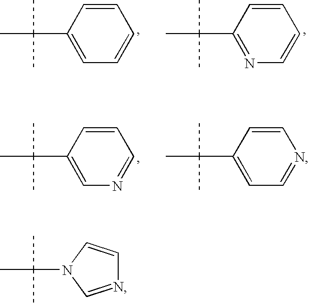
which may be mono- or disubstituted by identical or different substituents R²⁰.

Preferred substituents R²⁰ of the group Y are selected from halogen, C₁₋₃-alkyl, C₁₋₃-alkoxy, hydroxy and CF₃; in particular chlorine or bromine.

In case the group A is a phenyl group monosubstituted by R²⁰, the position of the substituent R²⁰ is preferably ortho with respect to the group Q.

In case the group B denotes a group selected from Cy and any preferred meaning thereof as given hereinafter, the group W preferably denotes a single bond, —CH₂—, —O—, —NR^{N}—, —CH₂—, —NR^{N}—CH₂—, —CH₂—O— or —CH₂—NR^{N}—, wherein R^{N} preferably denotes H or C₁₋₄-alkyl. According to this embodiment of the present invention the group W more preferably denotes a single bond, —O—, —CH₂—, —O≦CH₂— or —NH—CH₂—. According to an alternative of this embodiment, the group W preferably denotes —CH₂—CH₂—.

In case the group B does not denote a group selected from Cy, the group W denotes a single bond.

In case the group B denotes a group Cy, it is preferably selected from the group consisting of phenyl and 5- to 6-membered unsaturated or aromatic heterocyclic groups which contain 1 to 4 heteroatoms selected from N, O and S wherein the phenyl or heterocyclic group may be mono- or polysubstituted by identical or different substituents R²⁰.

More preferably in case the group B denotes a group Cy, it is selected from the group consisting of phenyl, pyridyl, pyridazinyl, pyrazinyl, pyrimidinyl, pyrrolyl, pyrazolyl, imidazolyl, triazolyl, tetrazolyl and thienyl; in particular selected from phenyl, pyridyl and 1H-imidazolyl, wherein said group B may be mono- or polysubstituted, preferably mono- or disubstituted by identical or different substituents R²⁰.

Most preferably the group B denotes a group characterized by a subformula selected from which may be mono- or polysubstituted, particularly mono- or disubstituted by identical or different substituents R²⁰.

In case the group B is a 6-membered ring, in particular a phenyl or pyridyl group, it is preferably unsubstituted or mono- or disubstituted by identical or different groups R²⁰, wherein the preferred position of a substituent is para with respect to the group A—W.

Preferred substituents R²⁰ of the group B are selected from halogen, hydroxy, nitro, C₁₋₃-alkyl, C₁₋₃-alkoxy, (C₁₋₃-alkyl)-carbonyl-, di-(C₁₋₃-alkyl)amino, aminocarbonyl, (C₁₋₃-alkyl)-carbonylamino and (C₁₋₃-alkyl)-sulfonylamino, wherein in each case one or more C atoms may additionally be mono- or polysubstituted by F. Preferred examples of fluorinated groups R²⁰ are CF₃ and —O—CF₃. Particularly preferred meanings of R²⁰ are fluorine, chlorine, methyl, methoxy and dimethylamino.

In case the group B does not denote a group Cy, it is preferably selected from the group consisting of halogen, CN, C₁₋₄-alkyl, C₁₋₆-alkoxy, C₁₋₄-alkylcarbonyl, C₁₋₄-alkylamino or di-(C₁₋₄-alkyl)-amino, wherein one or more C-atoms of said groups may additionally be mono- or polysubstituted by F; particularly selected from chlorine, bromine, iodine, CN, CF₃, methoxy, ethoxy, n-propoxy, i-propoxy, n-butoxy and methylcarbonyl.

The following are preferred definitions of other substituents according to the invention:

Preferably the substituent R¹³ has one of the meanings given for R¹⁶. Particularly preferably R¹³ denotes H, C₁₋₄-alkyl, C₃₋₇-cycloalkyl, C₃₋₇-cycloalkyl-C₁₋₃-alkyl, ω-hydroxy-C₂₋₃-alkyl, ω-(C₁₋₄-alkoxy)-C₂₋₃-alkyl, C₁₋₄-alkylcarbonyl. Most particularly preferably R¹³ denotes H, C₁₋₄-alkyl or C₁₋₃-alkylcarbonyl. The alkyl groups mentioned hereinbefore may be monosubstituted by Cl or mono- or polysubstituted by F.

Preferred meanings of the substituent R¹⁵ are H, C₁₋₄-alkyl, C₃₋₇-cycloalkyl, C₃₋₇-cycloalkyl-C₁₋₃-alkyl, while, as defined hereinbefore, in each case one or more C atoms may additionally be mono- or polysubstituted by F and/or in each case one or two C atoms independently of one another may additionally be monosubstituted by Cl or Br. Particularly preferably R¹⁵ denotes H, CF₃, methyl, ethyl, propyl or butyl.

The substituent R¹⁶ preferably denotes H, C₁₋₄-alkyl, C₃₋₇-cycloalkyl, C₃₋₇-cycloalkyl-C₁₋₃-alkyl, ωhydroxy-C₂₋₃-alkyl or ω-(C₁₋₄-alkoxy)-C₂₋₃-alkyl, while, as hereinbefore defined, in each case one or more C atoms may additionally be mono- or polysubstituted by F and/or in each case one or two C atoms independently of one another may additionally be monosubstituted by Cl or Br. More preferably R¹⁶ denotes H, CF₃, C₁₋₃-alkyl, C₃₋₆-cycloalkyl or C₃₋₆-cycloalkyl-C₁₋₃-alkyl; in particular H, methyl, ethyl, n-propyl and i-propyl.

Preferably the substituent R¹⁷ has one of the meanings given for R¹⁶ as being preferred or denotes C₁₋₄-alkylcarbonyl or C₃₋₅-cycloalkylcarbonyl. Particularly preferably R¹⁷ denotes H, C₁₋₃-alkyl, C₁₋₃-alkylcarbonyl, or C₃₋₅-cycloalkylcarbonyl.

Preferably one or both of the substituents R¹⁸ and R¹⁹ independently of one another denotes hydrogen or C₁₋₄-alkyl, particularly hydrogen or methyl.

In general the substituent R²⁰ preferably denotes halogen, hydroxy, cyano, nitro, C₁₋₄-alkyl, C₁₋₄-alkoxy, hydroxy-C₁₋₄-alkyl, (C₁₋₃-alkyl)-carbonyl-, di-(C₁₋₃-alkyl)amino, aminocarbonyl, (C₁₋₃-alkyl)-carbonylamino, (C₁₋₃-alkyl)-sulfonylamino or R²²—C₁₋₃-alkyl, while, as hereinbefore defined, in each case one or more C atoms may additionally be mono- or polysubstituted by F and/or in each case one or two C atoms independently of one another may additionally be monosubstituted by Cl or Br.

The substituent R²² preferably denotes C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, amino, C₁₋₄-alkylamino, di-(C₁₋₄-alkyl)-amino, C₁₋₄-alkyl-carbonyl-amino, aminocarbonylamino or C₁₋₄-alkylaminocarbonyl-amino, while, as hereinbefore defined, in each case one or more C atoms may additionally be mono- or polysubstituted by F and/or in each case one or two C atoms independently of one another may additionally be monosubstituted by Cl or Br. Most particularly preferred meanings for R²² are C₁₋₄-alkoxy, C₁₋₄-alkylcarbonyl, amino, C₁₋₄-alkylamino, di-(C₁₋₄-alkyl)-amino, wherein one or more H atoms may be replaced by fluorine.

Preferred definitions of the group R²¹ are C₁₋₄-alkyl, C₁₋₄-alkylcarbonyl, C₁₋₄-alkylsulphonyl, —SO₂—NH₂, —SO₂—NH—C₁₋₃-alkyl, —SO₂—N(C₁₋₃-alkyl)₂ and cyclo-C₃₋₆-alkyleneimino-sulphonyl, while, as hereinbefore defined, in each case one or more C atoms may additionally be mono- or polysubstituted by F and/or in each case one or two C atoms independently of one another may additionally be monosubstituted by Cl or Br. Most particularly preferably R²¹ denotes C₁₋₄-alkyl or CF₃.

Cy preferably denotes a C₃₋₇-cycloalkyl, particularly a C₃₋₆-cycloalkyl group, a C₅₋₇-cycloalkenyl group, pyrrolidinyl, piperidinyl, piperazinyl, morpholinyl, thiomorpholinyl, aryl or heteroaryl, and the above-mentioned cyclic groups may be mono- or polysubstituted at one or more C atoms by identical or different groups R²⁰, or in the case of a phenyl group may also additionally be monosubstituted by nitro, and/or one or more NH groups may be substituted by R²¹; and in the above-mentioned saturated or unsaturated carbo- or heterocyclic groups a —CH₂-group may be replaced by a —C(═O)— group. Most particularly preferred definitions of the group Cy are C₃₋₆-cycloalkyl, pyrrolidinyl, piperidinyl and piperidinonyl, which may be substituted as specified.

The term aryl preferably denotes phenyl or naphthyl, particularly phenyl.

The term heteroaryl preferably comprises pyridyl, pyridazinyl, indolyl, quinolinyl and benzoxazolyl.

Preferred compounds according to the invention are those wherein one or more of the groups, radicals, substituents and/or indices have one of the meanings given hereinbefore as being preferred.

Particularly preferred compounds according to the invention may be described by a general formula IIa1 to IIf9, wherein compounds of the formulae IIc1 to IIc9, in particular IIc1, IIc6 and IIc9 are even more preferred, 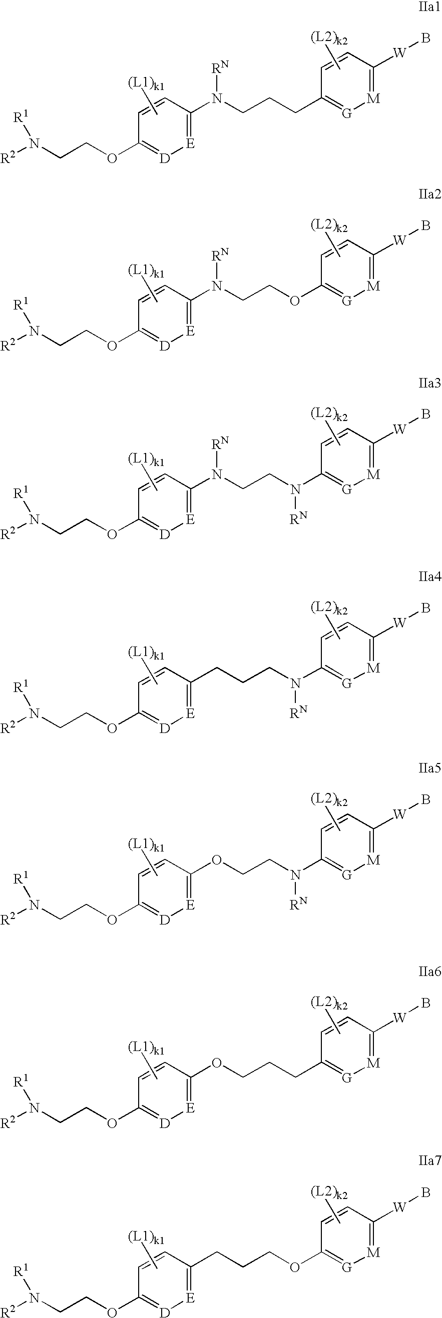 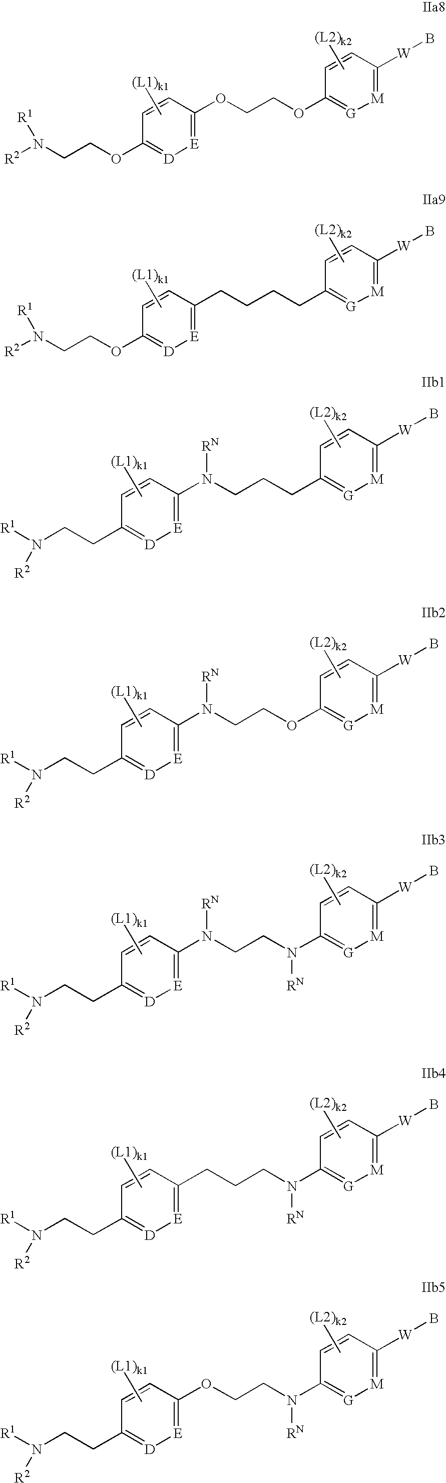 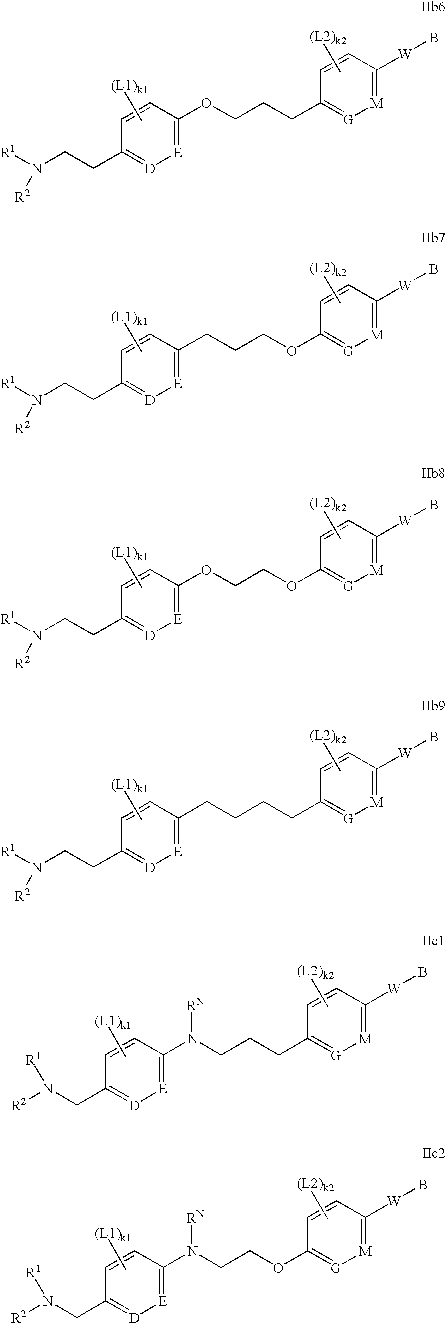 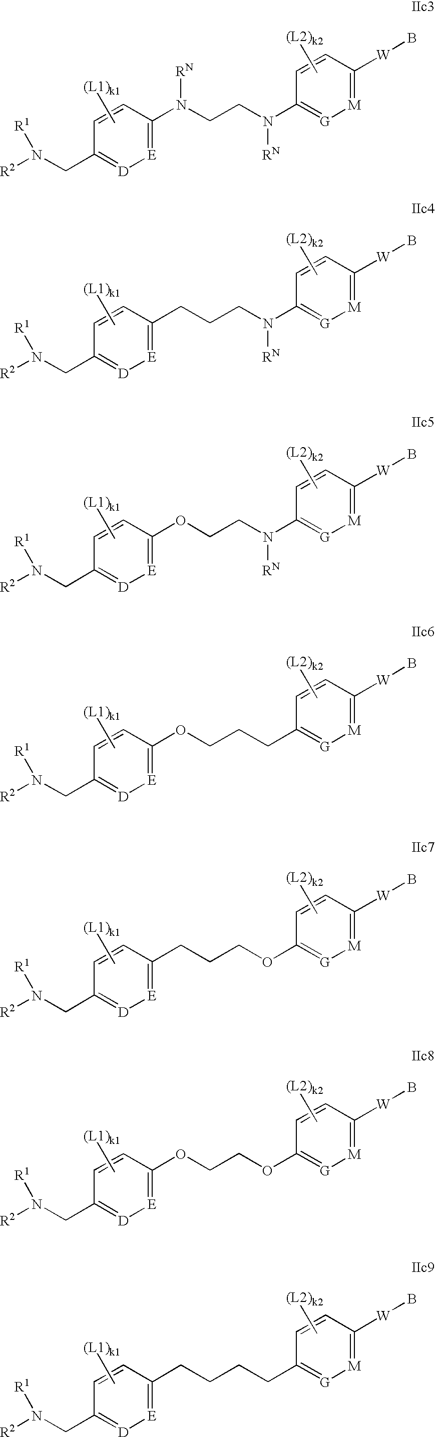 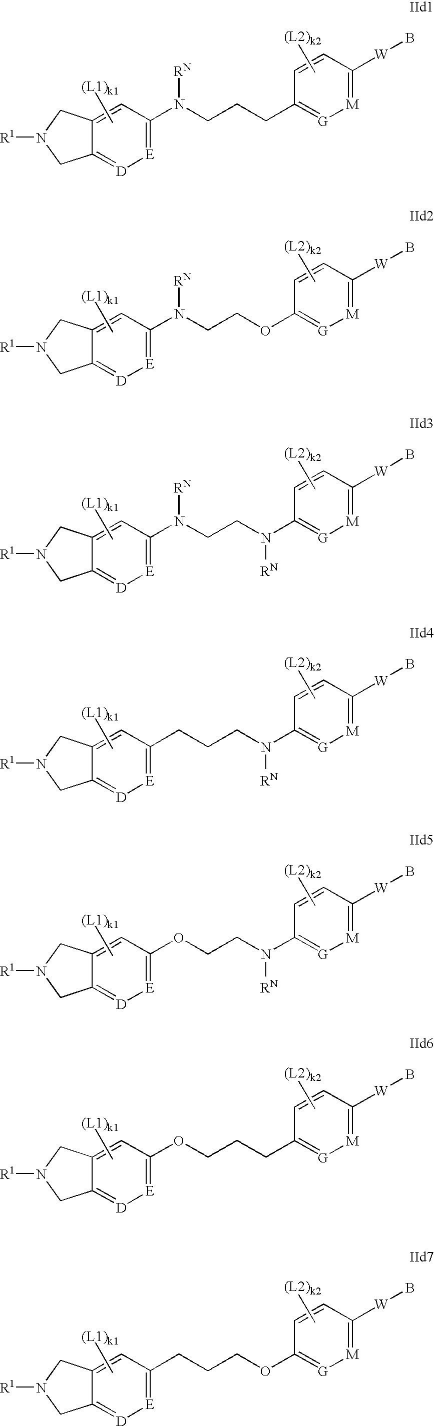 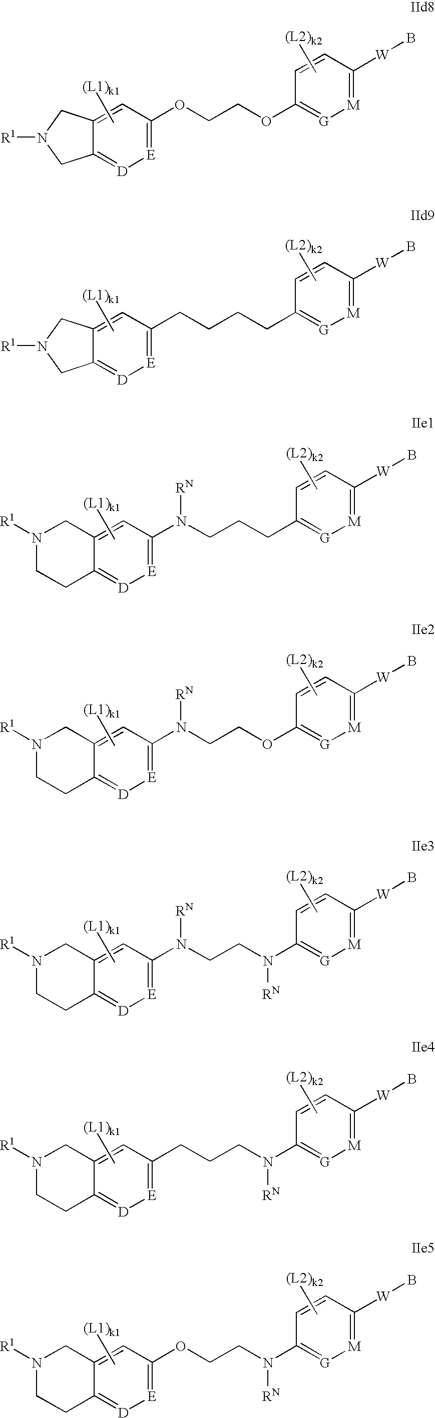 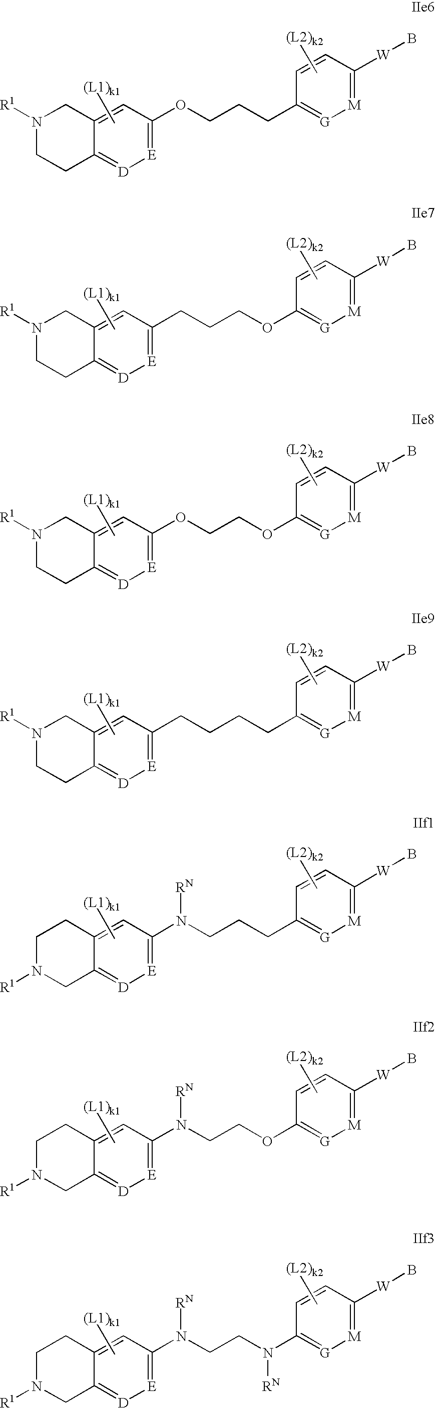 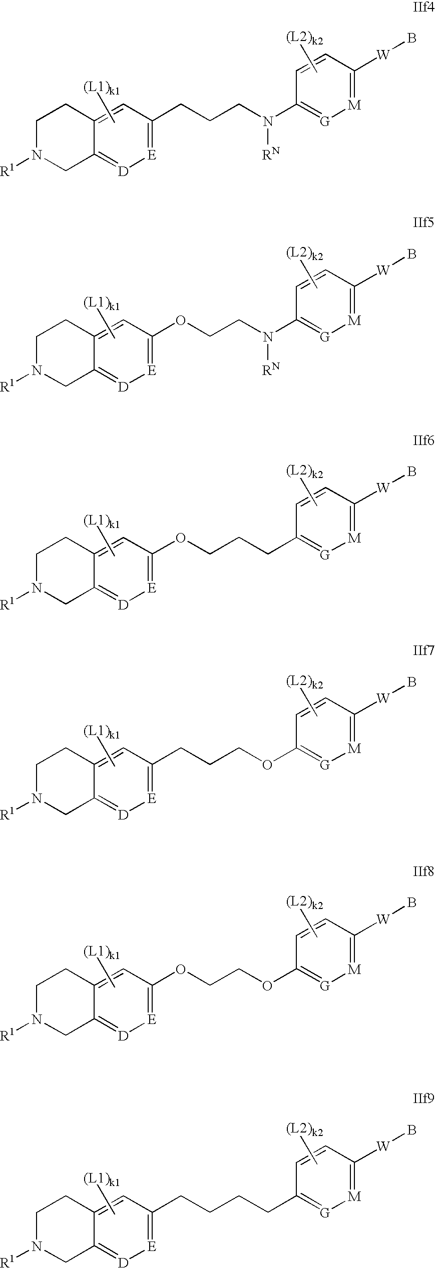
wherein

D and E independently of one another denote CH or N, wherein CH may be substituted with L1; and

G and M independently of one another denote CH or N, wherein CH may be substituted with L2; and

L1 are independently of one another selected from the meanings of R²⁰ as defined hereinbefore, in particular of the meanings of R²⁰ as a substituent of the group Y as defined hereinbefore; and

L2 are independently of one another selected from the meanings of R²⁰ as defined hereinbefore, in particular of the meanings of R²⁰ as a substituent of the group A as defined hereinbefore; and

k1, k2 independently of one another denote 0, 1 or 2; and

R¹, R², R^{N}, W and B are defined as hereinbefore, in particular possess a preferred meaning as defined hereinbefore.

According to a preferred embodiment in the formulae IIa1 to IIf9 both groups D and E denote N or both groups D and E denote CH, or D denotes CH while E denotes N; and

both groups G and M denote N or both groups G and M denote CH, or G denotes N while M denotes CH.

Even more preferably in the formulae IIa1 to IIf9 both groups D and E denote CH; and both groups G and M denote N.

In particular in the formulae IIa1 to IIf9, preferably IIc1 to IIc9, even more preferably IIc1, IIc6 and IIc9,

R¹, R² independently of one another denote C₁₋₄-alkyl, hydroxy-C₁₋₄-alkyl, C₃₋₅-alkenyl, C₃₋₅-alkynyl, C₃₋₇-cycloalkyl, hydroxy-C₃₋₇-cycloalkyl, dihydroxy-C₃₋₆-alkyl, C₃₋₇-cycloalkyl-C₁₋₃-alkyl, tetrahydropyran-3-yl, tetrahydropyran-4-yl, tetrahydrofuran-2-ylmethyl, tetrahydrofuran-3-ylmethyl, (hydroxy-C₃₋₇-cycloalkyl)-C₁₋₃-alkyl, C₁₋₄-alkoxy-C₂₋₃-alkyl, hydroxy-C₁₋₄-alkoxy-C₂₋₃-alkyl, C₁₋₄-alkoxy-C₁₋₄-alkoxy-C₂₋₃-alkyl, di-(C₁₋₃-alkyl)amino-C₂₋₃-alkyl, pyrrolidin-N-yl-C₂₋₃-alkyl and piperidin-N-yl-C₂₋₃-alkyl, while an alkyl, alkoxy, cycloalkyl or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl or CN; and one or both, preferably one of the groups R¹ and R² may also represent H; or

R¹, R² are joined together and form together with the N atom to which they are bound a heterocyclic group which is selected from azetidine, pyrrolidine, piperidine, 2,5-dihydro-1H-pyrrole, 1,2,3,6-tetrahydro-pyridine, piperazine, wherein the free imine function is substituted by R¹³ piperidin-4-one, morpholine, thiomorpholine, 1-oxo-thiomorpholine and 1,1-dioxo-thiomorpholine;
wherein one or more H atoms may be replaced by identical or different groups R¹⁴ and
the heterocyclic group defined hereinbefore may be substituted via a single bond by a carbo- or heterocyclic group Cy, while Cy is selected from the group comprising C₃₋₇-cycloalkyl, cyclo-C₃₋₆-alkyleneimino, 1H-imidazol, imidazolidin-2-one, 4H-triazol, while Cy may be mono- or polysubstituted by identical or different groups R²⁰ where R²⁰ is as hereinbefore defined and is preferably selected from fluorine, CF₃, C₁₋₃-alkyl, hydroxy-C₁₋₃-alkyl and hydroxy, and

R¹⁴ is selected from F, Cl, Br, cyano, C₁₋₄-alkyl, C₂₋₄-alkenyl, C₂₋₄-alkynyl, C₃₋₇-cycloalkyl, C₃₋₇-cycloalkyl-C₁₋₃-alkyl, hydroxy, hydroxy-C₁₋₃-alkyl, C₁₋₄-alkoxy, ω-(C₁₋₄-alkoxy)-C₁₋₃-alkyl, C₁₋₄-alkyl-carbonyl, carboxy, C₁₋₄-alkoxycarbonyl, hydroxy-carbonyl-C₁₋₃-alkyl, C₁₋₄-alkoxycarbonyl-C₁₋₃-alkyl, formylamino, N-formyl-N—(C₁₋₄-alkyl)-amino, C₁₋₄-alkyl-carbonylamino, N—(C₁₋₄-alkyl-carbonyl)-N—(C₁₋₄-alkyl)amino, C₃₋₇-cycloalkyl-carbonylamino, C₁₋₄-alkyl-aminocarbonylamino, C₁₋₄-alkyl-carbonylamino-C₁₋₃-alkyl, N—(C₁₋₄-alkyl-carbonyl)-N—(C₁₋₃-alkyl)amino-C₁₋₃-alkyl, C₃₋₇-cycloalkyl-carbonylamino-C₁₋₃-alkyl, C₁₋₄-alkyl-aminocarbonylamino-C₁₋₃-alkyl, C₁₋₄-alkyl-sulfonylamino, N—(C₁₋₄-alkyl-sulfonyl)-N—(C₁₋₃-alkyl)amino, C₁₋₄-alkoxy-carbonylamino, C₁₋₄-alkoxy-carbonylamino-C₁₋₃-alkyl, amino, C₁₋₄-alkyl-amino, C₃₋₇-cycloalkyl-amino, N—(C₃₋₇-cycloalkyl)-N—(C₁₋₄-alkyl)-amino, di-(C₁₋₄-alkyl)-amino, cyclo-C₃₋₆-alkyleneimino, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, C₁₋₄-alkyl-amino-carbonyl, C₃₋₇-cycloalkyl-amino-carbonyl, N—(C₃₋₇-cycloalkyl)-N—(C₁₋₄-alkyl)-amino-carbonyl, di-(C₁₋₄-alkyl)-amino-carbonyl, while in the above-mentioned meanings in each case one or more C atoms may additionally be mono- or polysubstituted by F and/or in each case one or two C atoms independently of one another may additionally be monosubstituted by Cl or Br; and

B denotes a group Cy, which is selected from the group consisting of phenyl, pyridyl, pyridazinyl, pyrazinyl, pyrimidinyl, 1H-imidazolyl, pyrrolyl, pyrazolyl, imidazolyl, triazolyl, tetrazolyl and thienyl; in particular selected from phenyl, pyridyl and 1H-imidazolyl, wherein the group B may be mono- or polysubstituted, preferably mono- or disubstituted by identical or different substituents R²⁰; and

W denotes a single bond, —CH₂—, —O—, —NR^{N}—, —O—CH₂—, —NR^{N}—CH₂—, —CH₂—O—, —CH₂—NR^{N}—, or —CH₂—CH₂—, wherein R^{N} preferably denotes H or C₁₋₄-alkyl; most preferably a single bond, —O—, —O—CH₂—, —NH—CH₂—, —CH₂—, or —CH₂—CH₂—;

or

B denotes a group selected from halogen, CN, C₁₋₄-alkyl, C₁₋₆-alkoxy, C₁₋₄-alkylcarbonyl, C₁₋₄-alkylamino or di-(C₁₋₄-alkyl)-amino, wherein one or more C-atoms of said groups may additionally mono- or polysubstituted by F; and

W denotes a single bond; or

R²⁰ independently of one another denote F. Cl, Br, hydroxy, cyano, nitro, C₁₋₃-alkyl, C₁₋₃-alkoxy, (C₁₋₃-alkyl)-carbonyl-, di-(C₁₋₃-alkyl)amino, aminocarbonyl, (C₁₋₃-alkyl)-carbonylamino and (C₁₋₃-alkyl)-sulfonylamino, wherein in each case one or more C atoms may additionally be mono- or polysubstituted by F; and

R^{N} independently of each other denotes H, C₁₋₃-alkyl or formyl; more preferably H or methyl; and

L1 halogen, C₁₋₃-alkyl, C₁₋₃-alkoxy, hydroxy and CF₃; and

k1 is 0 or 1; and

L2 halogen, C₁₋₃-alkyl, C₁₋₃-alkoxy, hydroxy and CF₃; and

k2 isOor1.

According to a preferred embodiment characterized by the formulae IIa1 to IIa9, in particular by the formula IIa2, the group B denotes halogen, CN, C₁₋₄-alkyl, C₁₋₆-alkoxy, C₁₋₄-alkylcarbonyl, C₁₋₄-alkylamino or di-(C₁₋₄-alkyl)-amino, wherein one or more C-atoms of said groups may additionally mono- or polysubstituted by F; and all other groups in said formulae are as defined hereinbefore.

According to a alternative preferred embodiment characterized by the formulae IIb1 to IIf9, in particular by the formula IId1 and IIc4, the group B denotes Cy, which is selected from the group consisting of phenyl, pyridyl, pyridazinyl, pyrazinyl, pyrimidinyl, 1H-imidazolyl, pyrrolyl, pyrazolyl, imidazolyl, triazolyl, tetrazolyl and thienyl; in particular selected from phenyl, pyridyl and 1H-imidazolyl, wherein the group B may be mono- or polysubstituted, preferably mono- or disubstituted by identical or different substituents R²⁰; and all other groups in said formulae are as defined hereinbefore.

In the formulae IIa1 to IIa9 the group W preferably denotes a single bond. In the formulae IIb1 to IIf9 the group W preferably denotes a single bond, —CH₂—, —O—, —NR^{N}—, —O—CH₂—, —NR^{N}—CH₂—, —CH₂—O— or —CH₂—NRN— wherein R^{N} preferably denotes H or C₁₋₄-alkyl; most preferably a single bond, —O—, —O—CH₂— or —NH—CH₂.

The compounds listed in the experimental section, including the tautomers, the diastereomers, the enantiomers, the mixtures thereof and the salts thereof, are preferred according to the invention.

Some expressions used hereinbefore and below to describe the compounds according to the invention will now be defined more fully.

The term halogen denotes an atom selected from among F, Cl, Br and 1, particularly F, Cl and Br.

The term C₁₋ₙ-alkyl, where n has a value of 3 to 8, denotes a saturated, branched or unbranched hydrocarbon group with 1 to n C atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term C₁₋ₙ-alkylene, where n may have a value of 1 to 8, denotes a saturated, branched or unbranched hydrocarbon bridge with 1 to n C atoms. Examples of such groups include methylene (—CH₂—), ethylene (—CH₂—CH₂—), 1-methyl-ethylene (—CH(CH₃)—CH₂—), 1,1-dimethyl-ethylene (—C(CH₃)₂—CH₂—), n-prop-1,3-ylene (—CH₂—CH₂—CH₂—), 1-methylprop-1,3-ylene (—CH(CH₃)—CH₂—CH₂—), 2-methylprop-1,3-ylene (—CH₂—CH(CH₃)—CH₂—), etc., as well as the corresponding mirror-symmetrical forms.

The term C₂₋ₙ-alkenyl, where n has a value of 3 to 6, denotes a branched or unbranched hydrocarbon group with 2 to n C atoms and at least one C═C-double bond. Examples of such groups include vinyl, 1-propenyl, 2-propenyl, iso-propenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 4-hexenyl, 5-hexenyl etc.

The term C₂₋ₙ-alkynyl, where n has a value of 3 to 6, denotes a branched or unbranched hydrocarbon group with 2 to n C atoms and a C═C triple bond. Examples of such groups include ethynyl, 1-propynyl, 2-propynyl, iso-propynyl, 1-butynyl, 2-butynyl, 3-butynyl, 2-methyl-1-propynyl, 1-pentynyl, 2-pentynyl, 3-pentynyl, 4-pentynyl, 3-methyl-2-butynyl, 1-hexynyl, 2-hexynyl, 3-hexynyl, 4-hexynyl, 5-hexynyl etc.

The term C₁₋ₙ-alkoxy denotes a C₁₋ₙ-alkyl-O— group, wherein C₁₋ₙ-alkyl is defined as above. Examples of such groups include methoxy, ethoxy, n-propoxy, iso-propoxy, n-butoxy, iso-butoxy, sec-butoxy, tert-butoxy, n-pentoxy, iso-pentoxy, neo-pentoxy, tert-pentoxy, n-hexoxy, iso-hexoxy etc.

The term C₁₋ₙ-alkylthio denotes a C₁₋ₙ-alkyl-S— group, wherein C₁₋ₙ-alkyl is defined as above. Examples of such groups include methylthio, ethylthio, n-propylthio, iso-propylthio, n-butylthio, iso-butylthio, sec-butylthio, tert-butylthio, n-pentylthio, iso-pentylthio, neo-pentylthio, tert-pentylthio, n-hexylthio, iso-hexylthio, etc.

The term C₁₋ₙ-alkylcarbonyl denotes a C₁₋ₙ-alkyl —C(═O)— group, wherein C₁₋ₙ-alkyl is defined as above. Examples of such groups include methylcarbonyl, ethylcarbonyl, n-propylcarbonyl, iso-propylcarbonyl, n-butylcarbonyl, iso-butylcarbonyl, sec-butylcarbonyl, tert-butylcarbonyl, n-pentylcarbonyl, iso-pentylcarbonyl, neo-pentylcarbonyl, tert-pentylcarbonyl, n-hexylcarbonyl, iso-hexylcarbonyl, etc.

The term C₃₋ₙ-cycloalkyl denotes a saturated mono-, bi-, tri- or spirocarbocyclic, preferably monocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, bicyclo[3,2,1]octyl, spiro[4,5]decyl, norpinyl, norbonyl, norcaryl, adamantyl, etc.

The term C₅₋ₙ-cycloalkenyl denotes a monounsaturated mono-, bi-, tri- or spirocarbocyclic, preferably monocarboxylic group with 5 to n C atoms. Examples of such groups include cyclopentenyl, cyclohexenyl, cycloheptenyl, cyclooctenyl, cyclononenyl, etc.

The term C₃₋ₙ-cycloalkylcarbonyl denotes a C₃₋ₙ-cycloalkyl-C(═O) group, wherein C₃₋ₙ-cycloalkyl is as hereinbefore defined.

The term aryl denotes a carbocyclic, aromatic ring system, such as for example phenyl, biphenyl, naphthyl, anthracenyl, phenanthrenyl, fluorenyl, indenyl, pentalenyl, azulenyl, biphenylenyl, etc. A particularly preferred meaning of “aryl” is phenyl.

The term cyclo-C₃₋₆-alkyleneimino denotes a 4- to 7-membered ring which comprises 3 to 6 methylene units as well as an imino group, while the bond to the residue of the molecule is made via the imino group.

The term cyclo-C₃₋₆-alkyleneimino-carbonyl denotes a cyclo-C₃₋₆-alkyleneimino ring as hereinbefore defined which is linked to a carbonyl group via the imino group.

The term heteroaryl used in this application denotes a heterocyclic, aromatic ring system which comprises in addition to at least one C atom one or more heteroatoms selected from N. O and/or S. Examples of such groups are furanyl, thiophenyl, pyrrolyl, oxazolyl, thiazolyl, imidazolyl, isoxazolyl, isothiazolyl, 1,2,3-triazolyl, 1,3,5-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinozilinyl, quinolinyl, isoquinolinyl, quinoxalinyl, naphthyridinyl, pteridinyl, carbazolyl, azepinyl, diazepinyl, acridinyl, etc. The term heteroaryl also comprises the partially hydrogenated heterocyclic, aromatic ring systems, particularly those listed above. Examples of such partially hydrogenated ring systems are 2,3-dihydrobenzofuranyl, pyrolinyl, pyrazolinyl, indolinyl, oxazolidinyl, oxazolinyl, oxazepinyl, etc. Particularly preferably heteroaryl denotes a heteroaromatic mono- or bicyclic ring system.

Terms such as C₃₋₇-cycloalkyl-C₁₋ₙ-alkyl, heteroaryl-C₁₋ₙ-alkyl, etc. refer to C₁₋ₙ-alkyl, as defined above, which is substituted with a C₃₋₇-cycloalkyl, aryl or heteroaryl group.

Many of the terms given above may be used repeatedly in the definition of a formula or group and in each case have one of the meanings given above, independently of one another. Thus, for example, in the group di-C₁₋₄-alkyl-amino, the two alkyl groups may have the same or different meanings.

The term “unsaturated”, for example in “unsaturated carbocyclic group” or “unsaturated heterocyclic group”, as used particularly in the definition of the group Cy, comprises in addition to the mono- or polyunsaturated groups, the corresponding, totally unsaturated groups, but particularly the mono- and diunsaturated groups.

The term “optionally substituted” used in this application indicates that the group thus designated is either unsubstituted or mono- or polysubstituted by the substituents specified. If the group in question is polysubstituted, the substituents may be identical or different.

The style used hereinbefore and hereinafter, according to which in a cyclic group a bond of a substituent is shown towards the centre of this cyclic group, indicates unless otherwise stated that this substituent may be bound to any free position of the cyclic group carrying an H atom.

Thus in the example 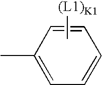
the substituent L1 where k1=1 may be bound to any of the free positions of the phenyl ring; where k1=2 selected substituents L1 may independently of one another be bound to different free positions of the phenyl ring.

The H atom of any carboxy group present or an H atom bound to an N atom (imino or amino group) may in each case be replaced by a group which can be cleaved in vivo. By a group which can be cleaved in vivo from an N atom is meant, for example, a hydroxy group, an acyl group such as the benzoyl or pyridinoyl group or a C₁₋₁₆-alkanoyl group such as the formyl, acetyl, propionyl, butanoyl, pentanoyl or hexanoyl group, an allyloxycarbonyl group, a C₁₋₁₆-alkoxycarbonyl group such as the methoxycarbonyl, ethoxycarbonyl, propoxycarbonyl, isopropoxycarbonyl, butoxycarbonyl, tert.butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulphonyl-C₂₋₄-alkoxycarbonyl, C₁₋₃-alkoxy-C₂₋₄-alkoxy-C₂₋₄-alkoxycarbonyl or RₑCO—O—(R_{f}CR_{g})—O—CO— group wherein
Rₑ denotes a C₁₋₈-alkyl, C₅₋₇-cycloalkyl, phenyl or phenyl-C₁₋₃-alkyl group,
R_{f} denotes a hydrogen atom, a C₁₋₃-alkyl, C₅₋₇-cycloalkyl or phenyl group and
R_{g} denotes a hydrogen atom, a C₁₋₃-alkyl or RₑCO—O—(R_{f}CRₕ)—O group wherein Rₑ and R_{f} are as hereinbefore defined and Rh is a hydrogen atom or a C₁₋₃-alkyl group,
   while the phthalimido group is an additional possibility for an amino group, and the above-mentioned ester groups may also be used as a group which can be converted in vivo into a carboxy group.

The residues and substituents described above may be mono- or polysubstituted by fluorine as described. Preferred fluorinated alkyl groups are fluoromethyl, difluoromethyl and trifluoromethyl. Preferred fluorinated alkoxy groups are fluoromethoxy, difluoromethoxy and trifluoromethoxy. Preferred fluorinated alkylsulphinyl and alkylsulphonyl groups are trifluoromethylsulphinyl and trifluoromethylsulphonyl.

The compounds of general formula I according to the invention may have acid groups, predominantly carboxyl groups, and/or basic groups such as e.g. amino functions. Compounds of general formula I may therefore be present as internal salts, as salts with pharmaceutically useable inorganic acids such as hydrochloric acid, sulphuric acid, phosphoric acid, sulphonic acid or organic acids (such as for example maleic acid, fumaric acid, citric acid, tartaric acid or acetic acid) or as salts with pharmaceutically useable bases such as alkali or alkaline earth metal hydroxides or carbonates, zinc or ammonium hydroxides or organic amines such as e.g. diethylamine, triethylamine, triethanolamine inter alia.

The compounds according to the invention may be obtained using methods of synthesis which are known to the one skilled in the art and described in the literature of organic synthesis. Preferably the compounds are obtained analogously to the methods of preparation explained more fully hereinafter, in particular as described in the experimental section.

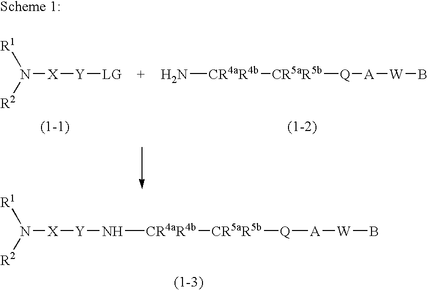

To obtain a compound of general formula (1-3) according to Scheme 1, a compound of general formula (1-1) is reacted with a compound of general formula (1-2) in the presence of a palladium catalyst with or without ligands and/or copper iodide and in the presence of a base. In principal such a reaction and its suitable reaction conditions are known as Buchwald-Hartwig amination or Goldberg reaction. The reaction is preferably carried out in an inert organic solvent solvent such as for example dioxane, DMF, DME, DMSO, toluene, benzene, acetonitrile, ethyleneglycol, isopropanol or THF, or a mixture of solvents. Suitable bases are particularly amine bases such as for example triethylamine, butylamine or N-diisopropyl-ethylamine (Hunig base), or inorganic bases such as cesium carbonate, cesium acetate, potassium carbonate, potassium tert-butoxide, sodium tert-butoxide or potassium phosphate. Preferred reaction temperatures are between −60° C. and 200° C. Typical palladium catalysts are for example tris(dibenzylideneacetone)dipalladium(0), tetrakis(triphenylphosphine)palladium(0), palladium(II)-acetate, Pd(PPh₃)₂Cl₂, Pd(CH₃CN)₂Cl₂, Pd(dppf)Cl₂ or palladium(II)-chloride. Typical ligands are for example triphenylphosphine, triphenylarsine or 2-(di-tert-butylphosphino)biphenyl. Suitable leaving groups (LG) are preferably selected from fluoride, bromide, chloride, iodide, trifluoroacetate, trifluoromethanesulfonate, methanesulfonate and toluenesulfonate and the like.

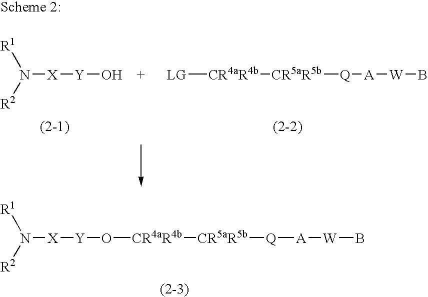

To obtain a compound of general formula (2-3) according to Scheme 2, a compound of general formula (2-1), for example a phenol (Y denotes phenyl), is reacted with a compound of general formula (2-2) in the presence of a base. Suitable bases are particularly tertiary amines such as triethylamine or Hunig base as well as alkali metal carbonates, for example potassium carbonate or sodium carbonate. The reactions are preferably carried out in an inert organic solvent like DMF, methylene chloride, acetone or DMSO, or mixtures thereof. DMF is a preferred solvent. The reaction usually takes place in a period of from 2 to 48 hours. A preferred temperature range for this reaction is from 20° C. to 120° C., preferably from 60° C. to 100° C. Preferred leaving groups (LG) are selected from fluoride, bromide, chloride, iodide, trifluoroacetate, trifluoromethanesulfonate, methanesulfonate and toluenesulfonate and the like.

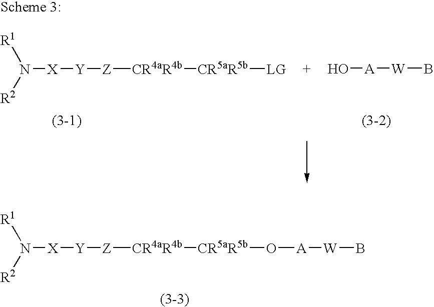

To obtain a compound of general formula (3-3) according to Scheme 3, a compound of general formula (3-1) is reacted with a compound of general formula (3-2), for example a phenol (A denotes phenyl), in the presence of a base. Suitable bases are particularly tertiary amines such as triethylamine or Hunig base as well as alkali metal carbonates, for example potassium carbonate or sodium carbonate. The reactions are advantageously carried out in an inert organic solvent like DMF, methylene chloride, acetone or DMSO, or mixtures thereof. DMF is a preferred solvent. Usually the reaction takes place in a period of from 2 to 48 hours. Preferably the reaction is carried out in in a temperature range from 20 to 120° C., preferably from 60° C. to 100° C. Preferred leaving groups (LG) are fluoride, bromide, chloride, iodide, trifluoroacetate, trifluoromethanesulfonate, methanesulfonate and toluenesulfonate and the like.

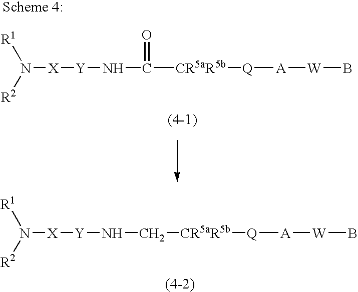

To obtain a compound of general formula (4-2) according to Scheme 4, a compound of general formula (4-1) is reacted with a reducing agent. Suitable reducing agents are selected from metal hydrides, for example lithium aluminum hydride, diisobutyl aluminum hydride (DIBAL), and boranes, preferably borane-THF-complex or borane-dimethylsulfide-complex. The reactions are preferably carried out in an inert organic solvent like methylene chloride, diethylether, toluene, benzene or THF and mixtures thereof. THF is a preferred solvent. The reaction usually takes place in a period of from 2 to 24 hours. Preferably the reaction is carried out in a temperature range from 20 to 100° C.

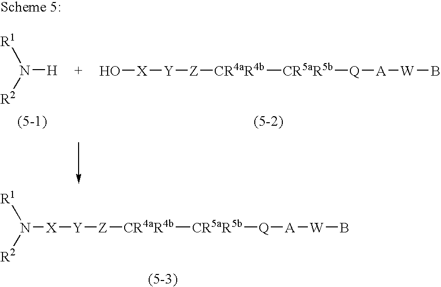

To obtain a compound of general formula (5-3) according to Scheme 5, a compound of general formula (5-2) is reacted with methanesulphonic acid chloride in the presence of a base to form the coresponding methanesulphonate derivative, followed by in situ reaction with an amine of general formula (5-1). The reaction conditions required are known to the skilled man as such. Advantageous solvents are halogenated hydrocarbons and ethers, such as for example dichloromethane, diethyl ether or THF. Suitable bases are particularly tertiary amines such as triethylamine or Hunig base as well as alkali metal carbonates, for example potassium carbonate or sodium carbonate. Suitable reaction temperatures are usually in the range from 0 to 90° C.

If the amine H—NR¹R² has another primary or secondary amino function, this is advantageously provided with a protective group beforehand, which can be cleaved again after the reaction has ended, using methods known from the literature.

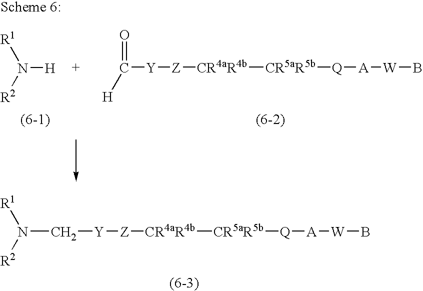

To obtain a compound of general formula (6-3) by reductive amination according to Scheme 6, a compound of general formula (6-2) is reacted with an amine of general formula (6-1) in the presence of an acid, followed by addition of a reducing agent. Advantageously the reaction is carried in an inert organic solvent such as halogenated hydrocarbons or ethers, such as for example dichloromethane, 1,2-dichloroethane, diethyl ether or THF, or mixtures thereof. Suitable acids are mineral acids, such as acetic acid or hydrochloric acid, or organic acids, such as para-toluenesulfonic acid. Suitable reducing agents are metal hydrides, especially sodium borohydride, sodium triacetoxyborohydride or sodium cyanoborhydride. Suitable reaction temperatures are usually in the range from 0 to 90° C. Typical reaction times are 1 to 24 hours.

If the amine H—NR¹R² has another primary or secondary amino function, this is advantageously provided with a protective group beforehand, which can be cleaved again after the reaction has ended, using methods known from the literature.

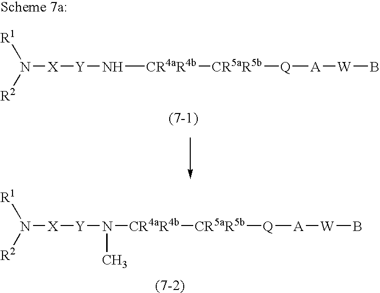

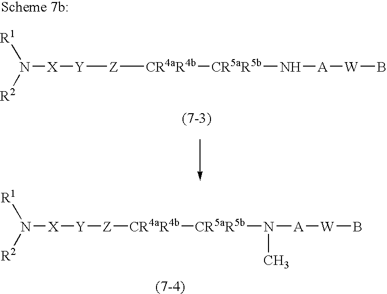

To obtain a compound of general formula (7-2) or (7-4) according to the Scheme 7a and 7b, a compound of general formula (7-1) or (7-3) is reacted with formaline in the presence of an acid, followed by addition of a reducing agent. Advantageously the reactions are carried out in an inert organic solvent such as halogenated hydrocarbons or ethers, such as for example dichloromethane, acetonitrile, diethyl ether or THF, or mixtures thereof. Suitable acids are mineral acids, such as acetic acid or hydrochloric acid, or organic acids, such as para-toluenesulfonic acid. Suitable reducing agents are metal hydrides, especially sodium borohydride, sodium triacetoxyborohydride or sodium cyanoborhydride. Suitable reaction temperatures are usually in the range from 0 to 90° C. Typical reaction times are 1 to 48 hours.

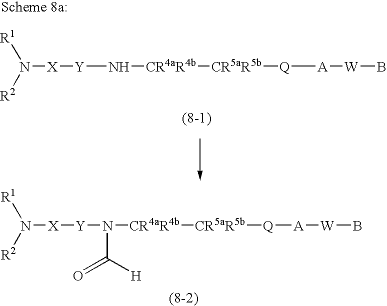

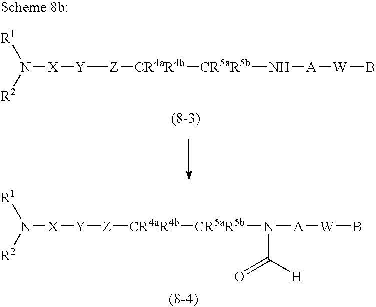

To obtain a compound of general formula (8-2) or (8-4) according to the Schemes 8a and 8b, a compound of general formula (8-1) or (8-3) is reacted with a mixture of acetic acid anhydride and formic acid. Suitable reaction temperatures are usually in the range from 0 to 200° C., preferably in the range of 20 to 130° C. Typical reaction times are 1 to 48 hours.

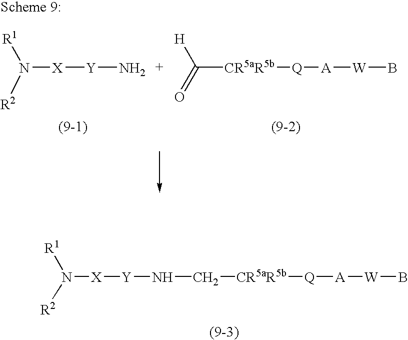

To obtain a compound of general formula (9-3) by reductive amination according to Scheme 9, a compound of general formula (9-2) is reacted with an amine or aniline of general formula (9-1) in the presence of an acid, followed by addition of a reducing agent. Advantageously the reaction is carried in an inert organic solvent such as halogenated hydrocarbons or ethers, such as for example dichloromethane, 1,2-dichloroethane, diethyl ether or THF, or mixtures thereof. Suitable acids are mineral acids, such as acetic acid or hydrochloric acid, or organic 10 acids, such as para-toluenesulfonic acid. Suitable reducing agents are metal hydrides, especially sodium borohydride, sodium triacetoxyborohydride or sodium cyanoborhydride. Suitable reaction temperatures are usually in the range from 0 to 90° C. Typical reaction times are 1 to 24 hours.

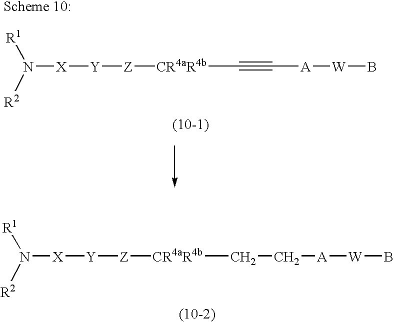

To obtain a compound of general formula (10-2) according to Scheme 10, a compound of general formula (10-1) is reacted with hydrogen in the presence of a suitable hydrogenation catalyst or any other suitable reducing agent. Suitable hydrogenation catalysts are selected from metals or metal salts like palladium/charcoal, Raney nickel, Rh(PPh₃)₃Cl (Wilkinson catalyst) or platinum(IV) oxide with or without the presence of vanadyl(IV) acetylacetonate. The reactions are preferably carried out in an inert organic solvent like ethyl acetate, diethylether, methanol, ethanol, DMF or THF and mixtures thereof with or without the presence of acids or bases like hydrochloric acid or ammonia. The reaction usually takes place in a period of from 1 to 96 hours. Preferably the reaction is carried out in a temperature range from 20 to 100° C. and in a pressure range from 1 bar to 30 bar.

Stereoisomeric compounds of formula (I) may chiefly be separated by conventional methods. The diastereomers are separated on the basis of their different physico-chemical properties, e.g. by fractional crystallisation from suitable solvents, by high pressure liquid or column chromatography, using chiral or preferably non-chiral stationary phases.

Racemates covered by general formula (I) may be separated for example by HPLC on suitable chiral stationary phases (e.g. Chiral AGP, Chiralpak AD). Racemates which contain a basic or acidic function can also be separated via the diastereomeric, optically active salts which are produced on reacting with an optically active acid, for example (+) or (−)-tartaric acid, (+) or (−)-diacetyl tartaric acid, (+) or (−)-monomethyl tartrate or (+)-camphorsulphonic acid, or an optically active base, for example with (R)-(+)-1-phenylethylamine, (S)-(−)-1-phenylethylamine or (S)-brucine.

According to a conventional method of separating isomers, the racemate of a compound of formula (I) is reacted with one of the above-mentioned optically active acids or bases in equimolar amounts in a solvent and the resulting crystalline, diastereomeric, optically active salts thereof are separated using their different solubilities. This reaction may be carried out in any type of solvent provided that it is sufficiently different in terms of the solubility of the salts. Preferably, methanol, ethanol or mixtures thereof, for example in a ratio by volume of 50:50, are used. Then each of the optically active salts is dissolved in water, carefully neutralised with a base such as sodium carbonate or potassium carbonate, or with a suitable acid, e.g. with dilute hydrochloric acid or aqueous methanesulphonic acid and in this way the corresponding free compound is obtained in the (+) or (−) form.

The (R) or (S) enantiomer alone or a mixture of two optically active diastereomeric compounds of general formula (I) may also be obtained by performing the syntheses described above with a suitable reaction component in the (R) or (S) configuration.

As already mentioned, the compounds of formula (I) may be converted into the salts thereof, particularly for pharmaceutical use into the physiologically and pharmacologically acceptable salts thereof. These salts may be present on the one hand as physiologically and pharmacologically acceptable acid addition salts of the compounds of formula (I) with inorganic or organic acids. On the other hand, in the case of acidically bound hydrogen, the compound of formula (I) may also be converted by reaction with inorganic bases into physiologically and pharmacologically acceptable salts with alkali or alkaline earth metal cations as counter-ion. The acid addition salts may be prepared, for example, using hydrochloric acid, hydrobromic acid, sulphuric acid, phosphoric acid, methanesulphonic acid, ethanesulphonic acid, toluenesulphonic acid, benzenesulphonic acid, acetic acid, fumaric acid, succinic acid, lactic acid, citric acid, tartaric acid or maleic acid. Moreover, mixtures of the above mentioned acids may be used. To prepare the alkali and alkaline earth metal salts of the compound of formula (I) with acidically bound hydrogen the alkali and alkaline earth metal hydroxides and hydrides are preferably used, while the hydroxides and hydrides of the alkali metals, particularly of sodium and potassium, are preferred and sodium and potassium hydroxide are most preferred.

The compounds according to the present invention, including the physiologically acceptable salts, are effective as antagonists of the MCH receptor, particularly the MCH-1 receptor, and exhibit good affinity in MCH receptor binding studies. Pharmacological test systems for MCH-antagonistic properties are described in the following experimental section.

As antagonists of the MCH receptor the compounds according to the invention are advantageously suitable as pharmaceutical active substances for the prevention and/or treatment of symptoms and/or diseases caused by MCH or causally connected with MCH in some other way. Generally the compounds according to the invention have low toxicity, they are well absorbed by oral route and have good intracerebral transitivity, particularly brain accessibility.

Therefore, MCH antagonists which contain at least one compound according to the invention are particularly suitable in mammals, such as for example rats, mice, guinea pigs, hares, dogs, cats, sheep, horses, pigs, cattle, monkeys and humans, for the treatment and/or prevention of symptoms and/or diseases which are caused by MCH or are otherwise causally connected with MCH.

Diseases caused by MCH or otherwise causally connected with MCH are particularly metabolic disorders, such as for example obesity, and eating disorders, such as for example bulimia, including bulimia nervosa. The indication obesity includes in particular exogenic obesity, hyperinsulinaemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity. This range of indications also includes cachexia, anorexia and hyperphagia.

Compounds according to the invention may be particularly suitable for reducing hunger, curbing appetite, controlling eating behaviour and/or inducing a feeling of satiation.

In addition, the diseases caused by MCH or otherwise causally connected with MCH also include hyperlipidaemia, cellulitis, fatty accumulation, malignant mastocytosis, systemic mastocytosis, emotional disorders, affectivity disorders, depression, anxiety states, reproductive disorders, sexual disorders, memory disorders, epilepsy, forms of dementia and hormonal disorders.

Compounds according to the invention are also suitable as active substances for the prevention and/or treatment of other illnesses and/or disorders, particularly those which accompany obesity, such as for example diabetes, diabetes mellitus, particularly type II diabetes, hyperglycaemia, particularly chronic hyperglycaemia, complications of diabetes including diabetic retinopathy, diabetic neuropathy, diabetic nephropathy, etc., insulin resistance, pathological glucose tolerance, encephalorrhagia, cardiac insufficiency, cardiovascular diseases, particularly arteriosclerosis and high blood pressure, arthritis and gonitis.

MCH antagonists and formulations according to the invention may advantageously be used in combination with a dietary therapy, such as for example a dietary diabetes treatment, and exercise.

Another range of indications for which the compounds according to the invention are advantageously suitable is the prevention and/or treatment of micturition disorders, such as for example urinary incontinence, hyperactive bladder, urgency, nycturia, enuresis, while the hyperactive bladder and urgency may or may not be connected with benign prostatic hyperplasia.

Generally speaking, the compounds according to the invention are potentially suitable for preventing and/or treating dependencies, such as for example alcohol and/or nicotine dependency, and/or withdrawal symptoms, such as for example weight gain in smokers coming off nicotine. By “dependency” is generally meant here an irresistible urge to take an addictive substance and/or to perform certain actions, particularly in order to either achieve a feeling of wellbeing or to eliminate negative emotions. In particular, the term “dependency” is used here to denote a dependency on an addictive substance. By “withdrawal symptoms” are meant here, in general, symptoms which occur or may occur when addictive substances are withdrawn from patients dependent on one or more such substances. The compounds according to the invention are potentially suitable particularly as active substances for reducing or ending tobacco consumption, for the treatment or prevention of a nicotine dependency and/or for the treatment or prevention of nicotine withdrawal symptoms, for reducing the craving for tobacco and/or nicotine and generally as an anti-smoking agent. The compounds according to the invention may also be useful for preventing or at least reducing the weight gain typically seen when smokers are coming off nicotine. The substances may also be suitable as active substances which prevent or at least reduce the craving for and/or relapse into a dependency on addictive substances. The term addictive substances refers particularly but not exclusively to substances with a psycho-motor activity, such as narcotics or drugs, particularly alcohol, nicotine, cocaine, amphetamine, opiates, benzodiazepines and barbiturates.

The dosage required to achieve such an effect is conveniently, by intravenous or sub-cutaneous route, 0.001 to 30 mg/kg of body weight, preferably 0.01 to 5 mg/kg of body weight, and by oral or nasal route or by inhalation, 0.01 to 50 mg/kg of body weight, preferably 0.1 to 30 mg/kg of body weight, in each case 1 to 3× daily.

For this purpose, the compounds prepared according to the invention may be formulated, optionally in conjunction with other active substances as described hereinafter, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, lozenges, powders, granules, solutions, emulsions, syrups, aerosols for inhalation, ointments or suppositories.

In addition to pharmaceutical compositions the invention also includes compositions containing at least one alkyne compound according to the invention and/ or a salt according to the invention optionally together with one or more physiologically acceptable excipients. Such compositions may also be for example foodstuffs which may be solid or liquid, in which the compound according to the invention is incorporated.

For the above mentioned combinations it is possible to use as additional active substances particularly those which for example potentiate the therapeutic effect of an MCH antagonist according to the invention in terms of one of the indications mentioned above and/or which make it possible to reduce the dosage of an MCH antagonist according to the invention.

Preferably one or more additional active substances are selected from among
active substances for the treatment of diabetes,
active substances for the treatment of diabetic complications,
active substances for the treatment of obesity, preferably other than MCH antagonists,
active substances for the treatment of high blood pressure,
active substances for the treatment of hyperlipidaemia, including arteriosclerosis,
active substances for the treatment of dyslipidaemia, including arteriosclerosis,
active substances for the treatment of arthritis,
active substances for the treatment of anxiety states,
active substances for the treatment of depression.

The above mentioned categories of active substances will now be explained in more detail by means of examples.

Examples of active substances for the treatment of diabetes are insulin sensitisers, insulin secretion accelerators, biguanides, insulins, α-glucosidase inhibitors, β3 adreno-receptor agonists.
Insulin sensitisers include glitazones, particularly pioglitazone and its salts (preferably hydrochloride), troglitazone, rosiglitazone and its salts (preferably maleate), JTT-501, GI-262570, MCC-555, YM-440, DRF-2593, BM-13-1258, KRP-297, R-119702 and GW-1929.
Insulin secretion accelerators include sulphonylureas, such as for example tolbutamide, chloropropamide, tolazamide, acetohexamide, glyclopyramide and its ammonium salts, glibenclamide, gliclazide, glimepiride. Further examples of insulin secretion accelerators are repaglinide, nateglinide, mitiglinide (KAD-1229) and JTT-608.
Biguanides include metformin, buformin and phenformin.
Insulins include those obtained from animals, particularly cattle or pigs, semisynthetic human insulins which are synthesised enzymatically from insulin obtained from animals, human insulin obtained by genetic engineering, e.g. from Escherichi coli or yeasts. Moreover, the term insulin also includes insulin-zinc (containing 0.45 to 0.9 percent by weight of zinc) and protamine-insulin-zinc obtainable from zinc chloride, protamine sulphate and insulin. Insulin may also be obtained from insulin fragments or derivatives (for example INS-1, etc.).
Insulin may also include different kinds, e.g. with regard to the onset time and duration of effect (“ultra immediate action type”, “immediate action type”, “two phase type”, “intermediate type”, “prolonged action type”, etc.), which are selected depending on the pathological condition of the patient.
α-Glucosidase inhibitors include acarbose, voglibose, miglitol, emiglitate.
β₃ Adreno receptor agonists include AJ-9677, BMS-196085, SB-226552, AZ40140.
Active substances for the treatment of diabetes other than those mentioned above include ergoset, pramlintide, leptin, BAY-27-9955 as well as glycogen phosphorylase inhibitors, sorbitol dehydrogenase inhibitors, protein tyrosine phosphatase 1B inhibitors, dipeptidyl protease inhibitors, glipazide, glyburide.

Active substances for the treatment of diabetes or diabetic complications furthermore include for example aldose reductase inhibitors, glycation inhibitors and protein kinase C inhibitors, DPPIV blockers, GLP-1 or GLP-2 analogues and SGLT-2 inhibitors.
Aldose reductase inhibitors are for example tolrestat, epalrestat, imirestat, zenarestat, SNK-860, zopolrestat, ARI-50i, AS-3201.
An example of a glycation inhibitor is pimagedine.
Protein Kinase C inhibitors are for example NGF, LY-333531.
DPPIV blockers are for example LAF237 (Novartis), MK431 (Merck) as well as 815541, 823093 and 825964 (all GlaxoSmithkline).
GLP-1 analogues are for example Liraglutide (NN2211) (NovoNordisk), CJC1131 (Conjuchem), Exenatide (Amylin).
SGLT-2 inhibitors are for example AVE-2268 (Aventis) and T-1095 (Tanabe, Johnson&Johnson).
Active substances other than those mentioned above for the treatment of diabetic complications include alprostadil, thiapride hydrochloride, cilostazol, mexiletine hydrochloride, ethyl eicosapentate, memantine, pimagedine (ALT-711).

Active substances for the treatment of obesity, preferably other than MCH antagonists, include lipase inhibitors and anorectics.
A preferred example of a lipase inhibitor is orlistat.
Examples of preferred anorectics are phentermine, mazindol, dexfenfluramine, fluoxetine, sibutramine, baiamine, (S)-sibutramine, SR-141716, NGD-95-1.
Active substances other than those mentioned above for the treatment of obesity include lipstatin.
Moreover, for the purposes of this application, the active substance group of anti-obesity active substances also includes the anorectics, of which the β₃ agonists, thyromimetic active substances and NPY antagonists should be emphasised. The range of substances which may be considered as preferred anti-obesity or anorectic active substances is indicated by the following additional list, by way of example: phenylpropanolamine, ephedrine, pseudoephedrine, phentermine, a cholecystokinin-A (hereinafter referred to as CCK-A) agonist, a monoamine reuptake inhibitor (such as for example sibutramine), a sympathomimetic active substance, a serotonergic active substance (such as for example dexfenfluramine, fenfluramine, a 5-HT2C agonist such as BVT.933 or APD356, or duloxetine), a dopamine antagonist (such as for example bromocriptine or pramipexol), a melanocyte-stimulating hormone receptor agonist or mimetic, an analogue of melanocyte-stimulating hormone, a cannabinoid receptor antagonist (Rimonabant, ACOMPLIA TM), an MCH antagonist, the OB protein (hereinafter referred to as leptin), a leptin analogue, a fatty acid synthase (FAS) antagonist, a leptin receptor agonist, a galanine antagonist, a GI lipase inhibitor or reducer (such as for example orlistat). Other anorectics include bombesin agonists, dehydroepiandrosterone or its analogues, glucocorticoid receptor agonists and antagonists, orexin receptor antagonists, urocortin binding protein antagonists, agonists of the Glucagon-like Peptide-1 receptor, such as for example exendin, AC 2993, CJC-1131, ZP10 or GRT0203Y, DPPIV inhibitors and ciliary neurotrophic factors, such as for example axokines. In this context mention should also be made of the forms of therapy which produce weight loss by increasing the fatty acid oxidation in the peripheral tissue, such as for example inhibitors of acetyl-CoA carboxylase.

Active substances for the treatment of high blood pressure include inhibitors of angiotensin converting enzyme, calcium antagonists, potassium channel openers and angiotensin II antagonists.
Inhibitors of angiotensin converting enzyme include captopril, enalapril, alacepril, delapril (hydrochloride), lisinopril, imidapril, benazepril, cilazapril, temocapril, trandolapril, manidipine (hydrochloride).
Examples of calcium antagonists are nifedipine, amlodipine, efonidipine, nicardipine.
Potassium channel openers include levcromakalim, L-27152, AL0671, NIP-121.
Angiotensin II antagonists include telmisartan, losartan, candesartan cilexetil, valsartan, irbesartan, CS-866, E4177.

Active substances for the treatment of hyperlipidaemia, including arteriosclerosis, include HMG-CoA reductase inhibitors, fibrate compounds.
HMG-CoA reductase inhibitors include pravastatin, simvastatin, lovastatin, atorvastatin, fluvastatin, lipantil, itavastatin, ZD-4522 and their salts.

Fibrate compounds include fenofibrate, bezafibrate, clinofibrate, clofibrate and simfibrate.

Active substances for the treatment of dyslipidaemia, including arteriosclerosis, include e.g. medicaments which raise the HDL level, such as e.g. nicotinic acid and derivatives and preparations thereof, such as e.g. niaspan, as well as agonists of the nicotinic acid receptor.

Active substances for the treatment of arthritis include NSAIDs (non-steroidal antiinflammatory drugs), particularly COX2 inhibitors, such as for example meloxicam or ibuprofen.

Active substances for the treatment of anxiety states include chlordiazepoxide, diazepam, oxozolam, medazepam, cloxazolam, bromazepam, lorazepam, alprazolam, fludiazepam.

Active substances for the treatment of depression include fluoxetine, fluvoxamine, imipramine, paroxetine, sertraline.

The dosage for these active substances is conveniently 1/5 of the lowest normal recommended dose up to 1/1 of the normal recommended dose.

In another embodiment the invention also relates to the use of at least one alkyne compound according to the invention and/ or a salt according to the invention for influencing the eating behaviour of a mammal. This use is particularly based on the fact that compounds according to the invention may be suitable for reducing hunger, curbing appetite, controlling eating behaviour and/or inducing a feeling of satiety. The eating behaviour is advantageously influenced so as to reduce food intake. Therefore, the compounds according to the invention are advantageously used for reducing body weight. Another use according to the invention is the prevention of increases in body weight, for example in people who had previously taken steps to lose weight and are interested in maintaining their lower body weight. A further use may be the prevention of weight gain in a co-medication with a substance generally causing weight gain (such a glitazones). According to this embodiment it is preferably a non-therapeutic use. Such a non-therapeutic use might be a cosmetic use, for example to alter the external appearance, or an application to improve general health. The compounds according to the invention are preferably used non-therapeutically for mammals, particularly humans, not suffering from any diagnosed eating disorders, no diagnosed obesity, bulimia, diabetes and/or no diagnosed micturition disorders, particularly urinary incontinence. Preferably, the compounds according to the invention are suitable for non-therapeutic use in people whose BMI (body mass index), defined as their body weight in kilograms divided by their height (in metres) squared, is below a level of 30, particularly below 25.

The Examples that follow are intended to illustrate the invention:

Preliminary Remarks:

As a rule, ¹H-NMR and/or mass spectra have been obtained for the compounds prepared. The R_{f} values are determined using ready-made silica gel 60 TLC plates F₂₅₄ (E. Merck, Darmstadt, Item no.1.05714) without chamber saturation or using ready-made aluminium oxide 60 F₂₅₄ TLC plates (E. Merck, Darmstadt, Item no.1.05713) without chamber saturation. The ratios given for the eluents relate to units by volume of the solvent in question. The units by volume for NH₃ relate to a concentrated solution of NH₃ in water. Silica gel made by Millipore (MATREX™, 35-70 my) is used for chromatographic purification. Alox (E. Merck, Darmstadt, aluminium oxide 90 standardised, 63-200 μm, Item no.1.01097.9050) is used for chromatographic purification.

The HPLC data given are measured under the following parameters:

mobile phase A: water:formic acid 99.9:0.1

mobile phase B: acetonitrile:formic acid 99.9:0.1

method A: analytical column: X-terra™ MS C18; 2.5 μm, 4.6 mm×30 mm; column temperature: 25° C.

gradient:

| time in min | % A | % B | flow rate in ml/min |
|---|---|---|---|
| 0.00 | 95.0 | 5.0 | 1.00 |
| 0.10 | 95.0 | 5.0 | 1.00 |
| 3.10 | 2.00 | 98.00 | 1.00 |
| 4.50 | 2.00 | 98.00 | 1.00 |
| 5.00 | 95.0 | 5.0 | 1.00 |

method B: analytical column: Zorbax column (Agilent Technologies), SB (Stable Bond)—C18; 3.5 μm; 4.6 mm×75 mm; column temperature: 30° C.

gradient:

| time in min | % A | % B | flow rate in ml/min |
|---|---|---|---|
| 0.00 | 95.0 | 5.0 | 1.60 |
| 4.50 | 10.0 | 90.0 | 1.60 |
| 5.50 | 90.0 | 10.00 | 1.60 |

method C: analytical column: Zorbax column (Agilent Technologies), SB (Stable Bond)−C18; 3.5 μm; 4.6 mm×75 mm; column temperature: 30° C.

gradient:

| time in min | % A | % B | flow rate in ml/min |
|---|---|---|---|
| 0.00 | 95.0 | 5.0 | 0.80 |
| 9.00 | 10.0 | 90.0 | 0.80 |
| 11.0 | 90.0 | 10.00 | 0.80 |

method D: analytical column: Zorbax column (Agilent Technologies), SB (Stable bond)—C18; 3.5 μm; 4.6 mm×75 mm; column temperature: RT

gradient:

| time in min | % A | % B | flow rate in ml/min |
|---|---|---|---|
| 0.00 | 95.0 | 5.0 | 1.60 |
| 4.50 | 10.0 | 90.0 | 1.60 |
| 5.00 | 10.0 | 90.0 | 1.60 |
| 5.50 | 95.0 | 5.00 | 1.60 |

method E: analytical column: Waters Symmetry—C18; 3.5 μm; 4.6 mm×75 mm; column temperature: RT

gradient:

| time in min | % A | % B | flow rate in ml/min |
|---|---|---|---|
| 0.00 | 95.0 | 5.0 | 1.60 |
| 4.00 | 50.0 | 50.0 | 1.60 |
| 4.50 | 10.00 | 90.00 | 1.60 |
| 5.00 | 10.00 | 90.00 | 1.60 |
| 5.50 | 95.0 | 5.0 | 1.60 |

method F: analytical column: Zorbax column (Agilent Technologies), SB (Stable bond)—C18; 3.5 μm; 4.6 mm×75 mm; column temperature: RT

gradient:

| time in min | % A | % B | flow rate in ml/min |
|---|---|---|---|
| 0.00 | 95.0 | 5.0 | 1.60 |
| 4.00 | 50.0 | 50.0 | 1.60 |
| 4.50 | 10.0 | 90.0 | 1.60 |
| 5.00 | 10.0 | 90.0 | 1.60 |
| 5.50 | 95.0 | 5.0 | 1.60 |

The following abbreviations for the eluent mixtures are used hereinafter when giving the R_{f} values:

(A): silica gel, methylene chloride/methanol/ammonia (9:1:0.01)

(B): silica gel, methylene chloride/methanol/ammonia (9:1:0.1)

(C): silica gel, methylene chloride/methanol (9:1)

(D): silica gel, methylene chloride/methanol/ammonia (5:2:0.01)

(D): silica gel, methylene chloride/methanol/ammonia (5:1:0.01)

(E): aluminum oxide, methylene chloride/methanol (30:1)

(F): silica gel, ethyl acetate/methanol/ammonia (95:5:0.5)

(G): silica gel, ethyl acetate/methanol/ammonia (90:10:0.5)

(H): silica gel, cyclohexane/ethyl acetate (2:1)

(I): aluminum oxide, methylene chloride

(K): aluminum oxide, methylene chloride/methanol (50:1)

(L): silica gel, methylene chloride/methanol/ammonia (5:1:0.1)

(M): silica gel, methylene chloride/methanol/ammonia (95:5:0.01)

(N): aluminum oxide, ethyl acetate/ethanol (50:1)

If there is no specific information as to the configuration, it is not clear whether there are pure enantiomers or whether partial or even total racemisation has taken place.

The following abbreviations are used above and hereinafter:

abs. absolute

Cbz benzyloxycarbonyl

conc. concentrated

DMF N,N-dimethylformamide

dppf 1,1′-bis(diphenylphosphino)ferrocene

EII electron impact ionisation

ether diethyl ether

EtOAc ethyl acetate

EtOH ethanol

Fmoc 9-fluorenylmethoxycarbonyl

HCl hydrochloric acid

MeOH methanol

Ph phenyl

RT ambient temperature (about 20° C.)

TBTU 2-(1H-benzotriazol-1-yl)-1,1,3,3-tetramethyluronium-tetrafluoroborate

THF tetrahydrofuran

Preparation of the Starting Compounds:

### EXAMPLE I.1

### 3-(4′-Chloro-biphenyl-4-yl)-propylamine

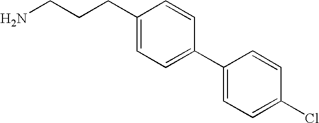

### I.1.a

### 3-(4′-Chloro-biphenyl-4-yl)-acrylamide

10.0 g (38.7 mmol) of 3-(4′-Chloro-biphenyl-4-yl)-acrylic acid are dissolved in 300 ml methylene chloride and 14.0 ml thionyl chloride are added. The mixture is stirred for 1.5 hours at reflux. After cooling the mixture is slowly poured into 200 ml of ammonia at 0° C. Stirring is continued for 30 minutes. After that time the residue is filtered off, recrystallised from methanol and dried at 85° C.

Yield: 7.60 g (76% of theory),

R_{f} value: 0.50 (silica gel, methylene chloride/methanol/ammonia=9:1:0.01)

C₁₅H₁₂ClNO

EII Mass spectrum: m/z=258/260 [M+H]⁺

### I.1.b

### 3-(4′-Chloro-biphenyl-4-yl)-propionamide

5.15 g (20.0 mmol) of 3-(4′-chloro-biphenyl-4-yl)-acrylamide are dissolved in 100 ml DMF. 1.00 g Raney nickel is added and the mixture is hydrogenated (50 psi) for 6 hours at RT. After that time the catalyst is filtered off and the filtrate evaporated. The residue is recrystallised from ethanol and the product is dried in vacuo at 80° C.

Yield: 4.40 g (85% of theory),

R_{f} value: 0.70 (silica gel, methylene chloride/methanol=9:1)

C₁₅H₁₄ClNO

EII Mass spectrum: m/z=260/262 [M+H]⁺

### I.1.c

### 3-(4′-Chloro-biphenyl-4-yl)-propylamine

3.00 g (11.6 mmol) of 3-(4′-chloro-biphenyl-4-yl)-propionamide are dissolved in 100 ml THF. Under protective gas a total of 11.6 ml (11.6 mmol) of a 1N lithium aluminum hydride solution in THF is added batchwise at −10° C. The mixture is stirred for 10 hours at RT. After that time water and a 1N NaOH-solution are added. The mixture is filtered and the filtrate evaporated. The residue is purified by silica gel column cromatography with methylene chloride/ethanol/ammonia (5:1:0.01) as eluent.

Yield: 1.20 g (42% of theory),

R_{f} value: 0.70 (aluminum oxide, methylene chloride/methanol=5:1)

C₁₅H₁₆ClN

The following compounds are synthesised analogously to the method described above:

### (I.2) [3-(4′-Chloro-biphenyl-4-yl)-propyl]-methyl-amine

### EXAMPLE II.1

### 3-[6-(4-Methoxy-phenyl)-pyridazin-3-yl]-propylamine

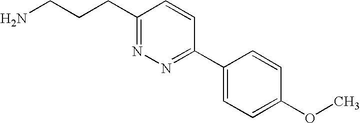

### II.1.a

### Trifluoro-methanesulfonic acid 6-(4-methoxy-phenyl)-pyridazin-3-yl ester

2.02 g (10.0 mmol) 6-(4-Methoxy-phenyl)-2H-pyridazin-3-one (Synthesis 1993, 334-342) are dissolved in 15 ml pyridine and 2.50 ml (15.0 mmol) trifluoromethanesulfonic acid anhydride are slowly added at 0° C. under argon atmosphere. The mixture is stirred for 2 hours at RT. After that time the mixture is slowly poured into ice water, the precipitate is filtered off and washed with water. Methylene chloride is added, the organic phase is separated and dried over sodium sulphate. The solvent is evaporated and dried in vacuo at 60° C.

Yield: 2.95 g (88% of theory),

R_{f} value: 0.90 (silica gel, methylene chloride/methanol=9:1)

C₁₂H₉F₃N₂O₄S

EII Mass spectrum: m/z=335 [M+H]⁺

### II.1.b

### {3-[6-(4-Methoxy-phenyl)-pyridazin-3-yl]-prop-2- ynyl}-carbamic acid tert-butyl ester

11.7 g (35.0 mmol) Trifluoro-methanesulfonic acid 6-(4-methoxy-phenyl)-pyridazin-3-yl ester and 11.0 g (70.0 mmol) prop-2-ynyl-carbamic acid tert-butyl ester are dissolved in 250 ml THF and 98 mg (1.4 mmol) bis-(triphenylphosphine)palladiumdichloride, 1.00 g (5.25 mmol) copper-(I)-iodide and finally 80 ml diisopropylamine are added at −10° C. The mixture is stirred for 3 hours at 0° C. and for additional 2 hours at RT. After that time the solvent is evaporated and purified by silica gel column chromatography with methylene chloride/ethyl acetate (5:1) as eluent. The product is dried in vacuo at 60° C.

Yield: 9.20 g (78% of theory),

R_{f} value: 0.30 (silica gel, methylene chloride/ethyl acetate=5:1)

C₁₉H₂₁ N₃O₃

### II.1.c

### {3-[6-(4-Methoxy-phenyl)-pyridazin-3-yl]-propyl]-carbamic acid tert-butyl ester

9.20 g (27.1 mmol) {3-[6-(4-Methoxy-phenyl)-pyridazin-3-yl]-prop-2-ynyl}-carbamic acid tert-butyl ester are dissolved in 500 ml ethyl acetate and 200 ml ethanol. 2.00 g Palladium on charcoal(10%) are added and the mixture is hydrogenated (50 psi) for 24 hours at RT. After that time the catalyst is filtered off and the filtrate evaporated.

Yield: 7.50 g (81% of theory),

R_{f} value: 0.60 (silica gel, methylene chloride/methanol=9:1)

C₁₉H₂₅N₃O₃

EII Mass spectrum: m/z=344 [M+H]⁺

### II.1.d

### 3-[6-(4-Methoxy-phenyl)-pyridazin-3-yl]-propylamine

7.50 g (21.8 mmol) {3-[6-(4-Methoxy-phenyl)-pyridazin-3-yl]-propyl}-carbamic acid tert-butyl ester are dissolved in 100 ml methylene chloride and 17.0 ml of trifluoroacetic acid are added. The mixture is stirred for 3 hours at RT. After that time the solvent is evaporated. The residue is taken up in methylene chloride and washed with a diluted ammonia-solution. The organic phase is dried over sodium sulphate.

Yield: 5.00 g (94% of theory),

R_{f} value: 0.10 (silica gel, methylene chloride/methanol/ammonia=5:2:0.01)

C₁₄H₁₇N₃O

EII Mass spectrum: m/z=244 [M+H]⁺

The following compounds are synthesised analogously to the method described above:
(II.2) 3-[6-(4-Chloro-phenyl)-pyridazin-3-yl]-propylamine
(II.3) 3-[6-(4-Cyano-phenyl)-pyridazin-3-yl]-propylamine (using Raney-nickel instead of palladium on charcoal for step c)
(II.4) 3-[6-(4-Fluoro-phenyl)-pyridazin-3-yl]-propylamine (using Raney-nickel instead of palladium on charcoal for step c)

### EXAMPLE III.1

### 1-(4-Iodo-benzyl)-4-methyl-piperidine

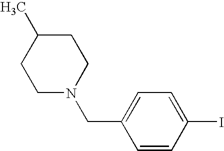

12.3 g (41.3 mmol) 1-Bromomethyl-4-iodo-benzene and 11.5 ml (82.7 mmol) triethylamine are dissolved in 125 ml methylene chloride and 4.10 ml (41.3 mmol) of 4-methyl-piperidine are added slowly. The mixture is stirred for 2 hours at ambient temperature. The organic phase is washed with water and dried over sodium sulphate. Lastly the solvent is eliminated.

Yield: 8.90 g (68% of theory),

R_{f} value: 0.70 (silica gel, cyclohexane/ethyl acetate=1:1)

C₁₃H₁₈INO

The following compounds are synthesised analogously to the method described above:
(III.2) 1-(4-bromo-benzyl)-4-trifluoromethyl-piperidine
(III.3) (4-bromo-benzyl)-dimethyl-amine
(III.4) 1-(4-bromo-benzyl)-piperidine
(III.4a) 1-(6-chloro-pyridin-3-ylmethyl)-4-trifluoromethyl-piperidin-4-ol
(III.5) 1-(6-chloro-pyridin-3-ylmethyl)-piperidine
(III.6) 1 -(6-chloro-pyridin-3-ylmethyl)-4-methyl-piperidine
(III.7) (6-chloro-pyridin-3-ylmethyl)-dimethyl-amine
(III.8) 1-(4-bromo-benzyl)-4-trifluoromethyl-piperidin-4-ol
(III.9) 1-(4-bromo-benzyl)-piperidin-4-ol
(III.10) 1 -(4-bromo-benzyl)-piperidin-3-ol
(III.11) 4-(4-bromo-benzyl)-morpholine
(III.12) 1-(4-bromo-benzyl)-4-methyl-piperidin-4-ol
(III.13) [1 -(4-bromo-benzyl)-piperidin-4-yl]-methanol
(III.14) 1-(4-bromo-benzyl)-piperidine-4-carboxylic acid amide
(III.15) N-[1 -(4-bromo-benzyl)-piperidin-4-yl]-acetamide
(III.16) (4-bromo-benzyl)-diethyl-amine
(III.17) 1-(4-bromo-benzyl)-4-methyl-piperidin-3,4-diol
(III.18) 1-(4-iodo-benzyl)-piperidin-3-ol
(III.19) 1-[(4-bromo-benzyl)-ethyl-amino]-2-methyl-propan-2-ol
(III.20) (R)-1-(4-bromo-benzyl)-piperidin-3-ol
(III.21) (S)-1-(4-bromo-benzyl)-piperidin-3-ol
(III.22) (4-bromo-benzyl)-methyl-amine
(III.23) 1-(4-bromo-benzyl)-pyrrolidine
(III.24) 1-[(4-bromo-benzyl)-(2-hydroxy-ethyl)-amino]-2-methyl-propan-2-ol
(III.25) N-[1-(4-bromo-benzyl)-pyrrolidin-3-yl]-acetamide
(III.26) 2-[(4-bromo-benzyl)-(2-hydroxy-ethyl)-amino]-ethanol
(III.27) (R)-[1-(4-bromo-benzyl)-pyrrolidin-2-yl]-methanol
(III.28) [1-(4-bromo-benzyl)-piperidin-4-yl]-dimethyl-amine
(III.29) 1-(4-bromo-benzyl)-piperidine-4-carboxylic acid methyl ester
(III.30) 1-(4-bromo-benzyl)-4-fluoro-piperidine
(III.31) (S)-1-(4-bromo-benzyl)-pyrrolidin-3-ol
(III.32) N-[1-(4-bromo-benzyl)-piperidin-4-yl]-N-methyl-acetamide
(III.33) 4-(4-bromo-benzyl)-thiomorpholine-1,1-dioxide
(III.34) (R)-1-(4-bromo-benzyl)-pyrrolidin-3-ol
(III.35) 1-(4-bromo-benzylamino)-propan-2-ol
(III.36) (S)-1-(4-bromo-benzyl)-2-methoxymethyl-pyrrolidine
(III.37) (R)-1-(4-bromo-benzyl)-2-methoxymethyl-pyrrolidine
(III.38) 1-(4-iodo-benzyl)-piperidine-3-carboxylic acid amide
(III.39) [2-(4-iodo-phenylethyl)]-dimethylamine
(III.40) N-[1-(4-bromo-benzyl)-piperidin-4-yl]-formamide
(III.41) 1-(4-bromo-benzyl)-4-(4-methyl-4H-[1,2,4]triazol-3-yl)-piperidine
(III.42) (S)-[1-(4-bromo-benzyl)-pyrrolidin-2-yl]-methanol
(III.43) [1-(4-bromo-benzyl)-pyrrolidin-3-yl]-methanol
(III.44) 2-[(4-bromo-benzyl)-methyl-amino]-ethanol
(III.45) 1-(4-bromo-benzyl)-azetidine
(III.46) N-[1-(4-bromo-benzyl)-pyrrolidin-3-yl]-N-methyl-acetamide
(III.47) (3S,4R)-1-(4-bromo-benzyl)-piperidin-3,4-diol
(III.48) (3R,4S)-1-(4-bromo-benzyl)-piperidin-3,4-diol
(III.49) 2-[2-(4-bromo-benzylamino)-ethoxy]-ethanol
(III.50) [1-(4-bromo-benzyl)-piperidin-2-yl]-methanol
(III.51) 1-(4-bromo-benzyl)-3-methoxy-piperidine
(III.52) [1-(4-bromo-benzyl)-piperidin-3-yl]-methanol
(III.53) 1-(4-bromo-benzyl)-1,2,3,6-tetrahydro-pyridine
(III.54) (3S,4S)-1-(4-bromo-benzyl)-pyrrolidine-3,4-diol
(III.55) 1-(4-bromo-benzyl)-4-methoxy-piperidine
(III.56) [1-(4-bromo-benzyl)-pyrrolidin-3-yl]-dimethyl-amine
(III.57) [2-(4-bromo-phenylethyl)]-dimethylamine
(III.58) 1-[1-(4-bromo-benzyl)-piperidin-4-yl]-3-methyl-urea
(III.59) 1-(4-bromo-benzyl)-4-methyl-piperazine
(III.60) N-[1-(4-bromo-benzyl)-piperidin-4-ylmethyl]-N-methyl-acetamide
(III.61) 1-(4-bromo-benzyl)-3,5-dimethyl-piperidine
(III.62) cyclopropanecarboxylic acid [1-(4-bromo-benzyl)-piperidin-4-yl]-amide
(III.63) N-[1-(4-bromo-benzyl)-piperidin-3-yl]-acetamide
(III.64) 1-[1-(4-bromo-benzyl)-piperidin-4-yl]-3-methyl-imidazolidin-2-one
(III.65) N-[1-(4-bromo-benzyl)-piperidin-4-yl]-propionamide
(III.66) N-[1-(4-bromo-benzyl)-piperidin-4-ylmethyl]-acetamide
(III.67) N-[1-(4-bromo-benzyl)-piperidin-4-yl]-methanesulfonamide
(III.68) 1-(4-bromo-2-methoxy-benzyl)-piperidin-4-ol
(III.69) N-[1-(4-bromo-benzyl)-piperidin-4-yl]-N-methyl-methanesulfonamide
(III.70) [1-(4-bromo-benzyl)-piperidin-3-yl]-dimethyl-amine
(III.71) 1-(4-bromo-2-fluoro-benzyl)-piperidin-4-ol
(III.72) 2-[(4-bromo-benzyl)-ethyl-amino]-ethanol
(III.73) N-[1-(4-bromo-benzyl)-piperidin-3-ylmethyl]-acetamide
(III.74) 1-(4-bromo-benzyl)-3-methoxy-piperidine
(III.75) 1-[4-(4-bromo-benzyl)-piperazin-1-yl]-ethanone
(III.76) 1-(4-bromo-benzyl)-piperidin-4-one
(III.77) N-[1-(4-bromo-benzyl)-piperidin-3-yl]-N-methyl-acetamide
(III.78) 1-(4-bromo-benzyl)-4-imidazol-1-yl-piperidine
(III.79) 1-(4-bromo-benzyl)-piperidine-4-carboxylic acid dimethylamide
(III.80) (R)-1-(4-bromo-benzylamino)-propan-2-ol
(III.81) (S)-1-(4-bromo-benzylamino)-propan-2-ol
(III.82) (S)-N-[1-(4-bromo-benzyl)-pyrrolidin-3-yl]-acetamide
(III.83) (R)-N-[1-(4-bromo-benzyl)-pyrrolidin-3-yl]-acetamide
(III.84) (R)-[1-(4-bromo-benzyl)-piperidin-3-yl]-methanol
(III.85) (S)-[1-(4-bromo-benzyl)-piperidin-3-yl]-methanol
(III.86) (S)-N-[1-(4-bromo-benzyl)-pyrrolidin-3-yl]-N-methyl-acetamide
(III.87) (R)-N-[1-(4-bromo-benzyl)-pyrrolidin-3-yl]-N-methyl-acetamide
(III.88) N-[1-(4-bromo-benzyl)-4-methyl-piperidin-4-yl]-acetamide

### EXAMPLE IV.1

### 1-(6-iodo-pyridin-3-ylmethyl)-4-trifluoromethyl-pipieridin-4-ol

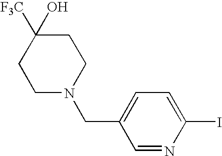

295 mg (1.00 mmol) 1-(6-chloro-pyridin-3-ylmethyl)-4-trifluoromethyl-piperidin-4-ol (educt III.4a) and 3.00 g (20.0 mmol) sodium iodide are dissolved in 5 ml of acetonitrile and 0.2 ml conc. HCl is added at RT. The mixture is stirred for 10 hours at reflux. After cooling, the solvent is evaporated, the residue is suspended in water and conc. ammonia is added. The water phase is extracted three times with ethyl acetate and the combined organic phases are dried over sodium sulphate. After evaporation of the solvent the product is purified by silica gel column chromatography with methylene chloride/methanol (9:1) as eluent.

Yield: 390 mg (100% of theory),

R_{f} value: 0.40 (silica gel, methylene chloride/methanol=9:1)

C₁₂H₁₄F₃IN₂O

EII Mass spectrum: m/z=387 [M+H]⁺

The following compounds are synthesised analogously to the method described above:
(IV.2) 1-(6-iodo-pyridin-3-ylmethyl)-piperidin (synthesized from educt III.5)
(IV.3) (6-iodo-pyridin-3-ylmethyl)-dimethyl-amine (synthesized from educt III.7)

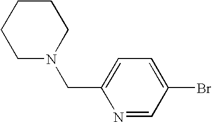

### EXAMPLE V.1

### 5-Bromo-2-piperidin-1-ylmethyl-pyridine

This compound was prepared as described in Organic Letters 2004, 6, 4905-4907.

### EXAMPLE VI.1

### 3-(6-Benzyloxy-pyridazin-3-yl)-propylamine

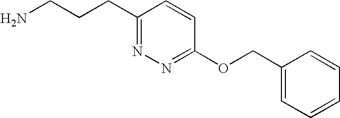

### VI1.a

### 3-Benzyloxy-6-chloro-pyridazine

33.92 g (205.5 mmol) 3,6-Dichloro-pyridazine are dissolved in 100 ml benzyl alcohol and 30.06 g (231.0 mmol) sodium benzylate are added. The mixture is stirred for 30 minutes at RT. After that time the mixture is slowly poured into ice water, the precipitate is filtered off and washed with water. The product is dried at 80° C.

Yield: 11.5 g (81% of theory),

R_{f} value: 0.60 (silica gel, cyclohexane/tehyl acetate=2:1)

C₁₁H₁₀N₂O₂

EII Mass spectrum: m/z=243/245 [M+Na]⁺

### VI.1.b

### 6-Benzyloxy-2H-pyridazin-3-one

15.5 g (70.0 mmol) 3-Benzyloxy-6-chloro-pyridazine are dissolved in 100 ml acetic acid and 6.3 g (77.0 mmol) sodium acetate are added. The mixture is stirred for 8 hours at 120° C. After that time the solvent is evaporated. The residue is taken up in methylene chloride and washed four times with 0.1N acetic acid. The organic phase is separated and the solvent is evaporated.

Yield: 40.21 g (89% of theory),

R_{f} value: 0.50 (silica gel, methylene chloride/methanol=9:1)

M.p. 170-173° C.

C₁₁H₉ClN₂O

### VI.1.c

Trifluoro-methanesulfonic acid 6-benzyloxy-pyridazin-3-yl ester 11.4 g (56.4 mmol) 6-Benzyloxy-2H-pyridazin-3-one are dissolved in 50 ml pyridine and 14.0 ml (84.6 mmol) trifluoromethanesulfonic acid anhydride are slowly added at 0° C. under argon atmosphere. The mixture is stirred for 1.5 hours at RT. After that time the mixture is slowly poured into ice water, the precipitate is filtered off and washed with water. Methylene chloride is added, the organic phase is separated and dried over sodium sulphate. Lastly the solvent is evaporated.

Yield: 17.0 g (90% of theory),

R_{f} value: 0.50 (silica gel, petrol ether/ethyl acetate=5:1)

M.p. 67-68° C.

C₁₂H₉F₃N₂O₄S

### VI1.d

### N-[3-(6-Benzyloxy-pyridazin-3-yl)-prop-2-ynyl]-2,2,2-trifluoro-acetamide

16.7 g (50.0 mmol) Trifluoro-methanesulfonic acid 6-benzyloxy-pyridazin-3-yl ester and 15.1 g (100.0 mmol) 2,2,2-trifluoro-N-prop-2-ynyl-acetamide are dissolved in 150 ml THF and 75 ml triethyl amine. 1.4 g (2.0 mmol) bis-(triphenylphosphine)palladiumdichloride and 1.40 g (7.35 mmol) copper-(I)-iodide are added at −5° C. The mixture is stirred for 20 hours at RT. After that time the solvent is evaporated. The residue is taken up in ethyl acetate and washed with water. The organic phase is dried over sodium sulphate, the solvent is evaporated. The product is washed with tert-butyl methyl ether and dried at 80° C.

Yield: 9.50 g (57% of theory),

R_{f} value: 0.50 (silica gel, methylene chloride/ethyl acetate=5:1)

M.p. 163-166° C.

C₁₆H₁₂F₃N₃O₂

### VI1.e

### N-[3-(6-Benzyloxy-pyridazin-3-yl)-propyl]-2,2,2-trifluoro-acetamide

9.50 g (28.3 mmol) N-[3-(6-Benzyloxy-pyridazin-3-yl)-prop-2-ynyl]-2,2,2-trifluoro-acetamide are dissolved in 100 ml ethyl acetate and 100 ml ethanol. 1.00 g Raney nickel are added and the mixture is hydrogenated (50 psi) for 48 hours at RT. After that time the catalyst is filtered off and the filtrate evaporated. The residue is purified by aluminum oxide column chromatography with methylene chloride/ethyl acetate (5:1) as eluent. The product is dried in vacuo at 50° C.

Yield: 5.90 g (61% of theory),

R_{f} value: 0.60 (silica gel, methylene chloride/methanol=9:1)

C₁₆H₁₆F₃N₃O₂

EII Mass spectrum: m/z=340 [M+H]⁺

### VI.1.f

### 3-(6-Benzyloxy-pyridazin-3-yl)-propylamine

5.90 g (17.4 mmol) N-[3-(6-Benzyloxy-pyridazin-3-yl)-propyl]-2,2,2-trifluoro-acetamide are dissolved in 100 ml methanol and 70.0 ml (69.6 mmol) 1N sodium hydroxide solution are added at 0° C. The mixture is stirred for 1 hour at RT. After that time the solvent is evaporated. The residue is taken up in methylene chloride and washed with water. The organic phase is dried over sodium sulphate and the solvent is evaporated.

Yield: 4.00 g (95% of theory),

R_{f} value: 0.30 (silica gel, methylene chloride/methanol/ammonia=5:1:0.01)

C₁₄H₁₇N₃O

EII Mass spectrum: m/z=244 [M+H]⁺

The following compounds are synthesised analogously to the method described above:
(VI.2) 3-(6-Methoxy-pyridazin-3-yl)-propylamine starting from 3-iodo-6-methoxy-pyridazine (see J. Org. Chem. 1963, 28, 218) in step d
(VI.3) 3-(6-Ethoxy-pyridazin-3-yl)-propylamine starting from 3-iodo-6-ethoxy-pyridazine (prepared analogously to J. Org. Chem. 1963, 28, 218) in step d
(VI.4) 3-(6-Propoxy-pyridazin-3-yl)-propylamine starting from 3-iodo-6-propoxy-pyridazine (prepared analogously to J. Org.

Chem. 1963, 28, 218) in step d
(VI.5) 3-(6-isopropoxy-pyridazin-3-yl)-propylamine starting from 3-iodo-6-isopropoxy-pyridazine (prepared analogously to J. Org. Chem. 1963, 28, 218) in step d
(VI.6) 3-[6-(4-Fluoro-benzyloxy)-pyridazin-3-yl]-propylamine starting from 3-iodo-6-(4-fluoro-benzyloxy)-pyridazine (prepared analogously to J. Org. Chem. 1963, 28, 218) in step d
(VI.7) 3-(6-Phenoxy-pyridazin-3-yl)-propylamine starting from 3-iodo-6-phenoxy-pyridazine (prepared analogously to J. Org. Chem. 1963, 28, 218) in step d

### EXAMPLE VII.1

### (4-{3-[5-(4-Chloro-phenyl)-pyridin-2-ylamino]-propyl}-phenyl)-methanol

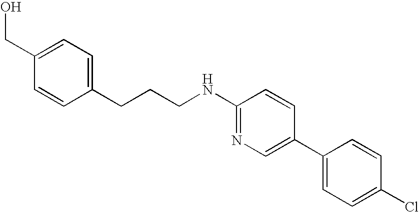

3.07 g (15.0 mmol) 5-(4-Chloro-phenyl)-pyridin-2-ylamine (described in WO 04/039780) and 2.46 g (15.0 mmol) 3-(4-hydroxymethyl-phenyl)-propionaldehyde (described in WO 04/039780) are dissolved in 50 ml methanol and 1 ml conc. acetic acid. The mixture is stirred for 1 hour at RT. After that time 1.89 g (30.0 mmol) sodium cyanoborohydride are added and the mixture is stirred for additional 16 hours at RT. After that time the solvent is evaporated. The residue is taken up in ethyl acetate and water, the organic phase is separated and washed with brine. The organic phase is dried over sodium sulphate and the solvent is evaporated. The residue is purified by silica gel column chromatography with ethyl acetate/methanol/ammonia (99:1:0.1) as eluent.

Yield: 2.60 g (49% of theory),

retention time (HPLC): 3.4 min (method B)

C₂₁H₂₁ClN₂O

EII Mass spectrum: m/z=353/355 [M+H]⁺

The following compounds are synthesised analogously to the method described above:
(VII.2) (4-{3-[6-(4-Methoxy-phenyl)-pyridazin-3-ylamino]-propyl}-phenyl)-methanol
(VII.3) (4-{2-[6-(4-Methoxy-phenyl)-pyridazin-3-ylamino]-ethoxyl}-phenyl)-methanol

### EXAMPLE VIII.1

### [4-(3-{[5-(4-Chloro-phenyl)-pyridin-2-yl]-methyl-amino}-propyl)-phenyl]-methanol

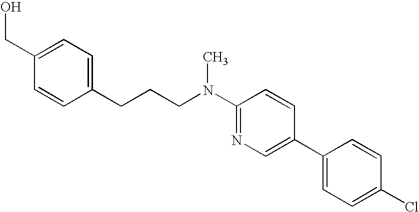

423 mg (1.20 mmol) (4-{3-[5-(4-Chloro-phenyl)-pyridin-2-ylamino]-propyl}-phenyl)-methanol (educt VII.1) and 3.00 ml (3.60 mmol) formalin (37%) are dissolved in 5 ml acetonitrile and 0.5 ml conc. acetic acid. The mixture is stirred for 1 hour at RT. After that time 150 mg (2.40 mmol) sodium cyanoborohydride are added and the mixture is stirred for additional 20 hours at RT. After that time the solvent is evaporated. The residue is taken up in water and extracted with ethyl acetate. The organic phase is dried over sodium sulphate and the solvent is evaporated. The residue is purified by silica gel column chromatography with cyclohexane/ethyl acetate (1:1) as eluent.

Yield: 190 mg (43% of theory),

retention time (HPLC): 3.3 min (method B)

C₂₂H₂₃ClN₂O

EII Mass spectrum: m/z=367/369 [M+H]⁺

### EXAMPLE IX

The following starting materials have been described in WO 2004/039764 or can be prepared analogously:
(IX.1) N-[3-Chloro-4-(2-diethylamino-ethoxy)-phenyl]-2-(2-chloro-4-trifluoromethyl-phenoxy)-acetamide
(IX.2) N-[3-Chloro-4-(2-diethylamino-ethoxy)-phenyl]-2-(2-chloro-4-trifluoromethyl-phenylamino)-acetamide
(IX.3) 2-(2-Chloro-4-trifluoromethyl-phenoxy)-N-[4-(2-diethylamino-ethoxy)-2-dimethylamino-phenyl]-acetamide
(IX.4) 2-(3-Bromo-biphenyl-4-yloxy)-N-{3-bromo-4-[2-(4-methyl-piperidin-1-yl)-ethyl phenyl}-acetamide
(IX.5) 2-(3-Bromo-biphenyl-4-yloxy)-N-[3-bromo-4-(2-diethylamino-ethyl)-phenyl]-acetamide

### EXAMPLE X.1

### 3-Chloro-4-(2-diethylamino-ethoxy)-phenol

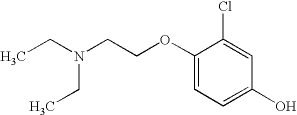

### X.1.a

### [2-(2-Chloro-4-methoxy-phenoxy)-ethyl]-diethyl-amine

5.00 g (31.5 mmol) 2-Chloro-4-methoxy-phenol, 8.50 g (32.6 mmol) (2-bromo-ethyl)-diethyl-amine hydrobromide and 8.80 g (63.7 mmol) potassium carbonate are dissolved in 200 ml acetone. The mixture is stirred for 10 hours at reflux. After that time additional 3.00 g (11.5 mmol) (2-bromo-ethyl)-diethyl-amine hydrobromide and 3.00 g (21.7 mmol) potassium carbonate are added and the mixture is refluxed for 1 hour. After cooling, the mixture is filtered, the solvent is evaporated and the residue is taken up in methylene chloride. The organic phase is washed with water and dried over sodium sulphate. Lastly the solvent is evaporated.

Yield: 6.40 g (79% of theory),

R_{f} value: 0.10 (silica gel, methylene chloride/methanol=50:1)

C₁₃H₂₀ClNO₂

EII Mass spectrum: m/z=257/259 [M+Na]⁺

### X.1.b

### 3-Chloro-4-(2-diethylamino-ethoxy)-phenol

3.00 g (11.6 mmol) [2-(2-Chloro-4-methoxy-phenoxy)-ethyl]-diethyl-amine and 30.0 g (260 mmol) pyridine hydrochloride are melted for 3 hours at 200° C. After that time the mixture is cooled to 90° C. and poured into water. The mixture is stirred for 30 minutes at RT and extracted with ethyl acetate. After drying over sodium sulphate, the solvent is evaporated.

Yield: 2.48 g (87% of theory),

R_{f} value: 0.40 (silica gel, methylene chloride/methanol=50:1)

C₁₂H₁₈ClNO₂

EII Mass spectrum: m/z=244/246 [M+H]⁺

### EXAMPLE XI.1

### Methanesulfonic acid 2-(2-chloro-4-iodo-phenoxy)-ethyl ester

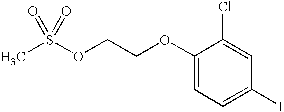

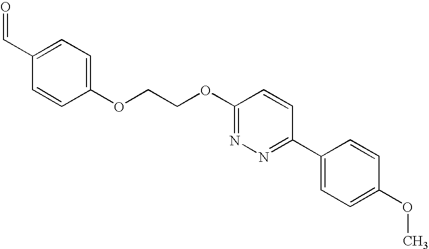

### XI.1.a

### 2-(2-Chloro-4-iodo-phenoxy)-ethanol

50.09 g (60.00 mmol) 2-(4-Bromo-2-chloro-phenoxy)-ethanol (described in WO 2004/072016), 17.98 g (120.0 mmol) sodium iodide, 1.14 g (6.00 mol) copper(I) iodide and 1.28 ml (12.0 mmol) N,N-dimethyl ethylenediamine are dissolved in 60 ml 1,4-dioxane. The mixture is stirred for 48 hours at RT. After that time 200 ml diluted ammonia solution are added and the solution is extracted three times with methylene chloride. The combined organic layers are dried over magnesium sulphate. Lastly the solvent is evaporated.

Yield: 16.2 g (90% of theory),

C₈H₈CIIO₂

EII Mass spectrum: m/z=298/300 [M]⁺

### XI.1.b

### Methanesulfonic acid 2-(2-chloro-4-iodo-phenoxy)-ethyl ester

0.20 g (0.67 mmol) 2-(2-Chloro-4-iodo-phenoxy)-ethanol, 0.14 ml (1.0 mmol) triethylamine and 0.078 ml (1.0 mmol) methane sulfonyl chloride are dissolved in 10 ml methylene chloride. The mixture is stirred for 1 hour at RT. After that time water is added. The organic phase is separated and washed with water. After drying over sodium sulphate, the solvent is evaporated.

Yield: 0.25 g (100% of theory),

R_{f} value: 0.60 (silica gel, methylene chloride/methanol=50:1)

C₉H₁₀CIIO₄S

EII Mass spectrum: m/z=376/378 [M+H]⁺

### EXAMPLE XII.1

### 4-{2-[6-(4-Methoxy-phenyl)-pyridazin-3-yloxy]-ethoxyl-benzaldehyde

70 mg (3.01 mmol) sodium metal are suspended in 2.0 ml THF and 500 mg (3.01 mmol) 4-(2-hydroxy-ethoxy)-benzaldehyde in 2.0 ml THF are slowly added. The mixture is stirred for 2 hours at 60° C. After that time 664 mg (3.01 mmol) 3-chloro-6-(4-methoxy-phenyl)-pyridazine in 2.0 ml THF are added and the mixture is stirred for 10 hours at reflux. After that time the solvent is evaporated and the residue is taken up in water. Ethyl acetate is added, the organic phase is separated and dried over sodium sulphate. The solvent is evaporated and the residue is purified by silica gel column chromatography with cyclohexane/ethyl acetate (1:1) as eluent.

Yield: 100 mg (9% of theory),

R_{f} value: 0.40 (silica gel, cyclohexane/ethyl acetate=1:1)

C₂₀H₁₈N₂O₄

EII Mass spectrum: m/z=351 [M+H]⁺

### EXAMPLE XIII.1

### {2-[4-(2-Bromo-ethoxy)-2-chloro-phenoxyl-ethyl}-diethyl-amine

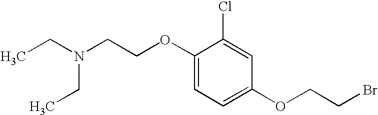

1.23 g (5.05 mmol) 3-Chloro-4-(2-diethylamino-ethoxy)-phenol (educt X.1), 1.70 ml (19.7 mmol) 1,2-dibromo-ethane and 1.70 g (12.3 mmol) potassium carbonate are dissolved in 50 ml acetonitrile. The mixture is stirred for 10 hours at 90° C. After that time additional 1.70 ml (19.7 mmol) 1,2-dibromo-ethane and 1.7 g (12.3 mmol) potassium carbonate are added and the mixture is stirred for 3 hours at 90° C. After that time the mixture is filtered, the solvent is evaporated and the residue is taken up in ethyl acetate. The organic phase is washed with water and 0.1N HCl, the aqueous phases are combined, 0.1N NaOH is added and the solution is reextracted with ethyl acetate. The combined organic layers are dried over sodium sulphate. Lastly the solvent is evaporated.

Yield: 560 mg (32% of theory),

R_{f} value: 0.05 (silica gel, methylene chloride/methanol=9:1)

C₁₄H₂₁ BrClNO₂

EII Mass spectrum: m/z=350/352 [M+H]⁺

### EXAMPLE XIV.1

### {4-[6-(3-Amino-propyl)-pyridazin-3-yl]-phenyl}-dimethyl-amine

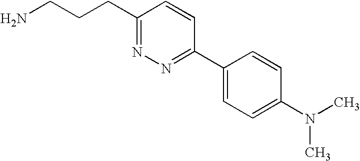

### XIV.1.a

### [3-(6-Chloro-pyridazin-3-yl)-prop-2-ynyl]-carbamic acid tert-butyl ester

19.2 g (80.0 mmol) 3-Chloro-6-iodo-pyridazine (Tetrahedron 55, 1999,15067) and 13.7 g (88.0 mmol) prop-2-ynyl-carbamic acid tert-butyl ester are dissolved in 200 ml THF and 2.50 g (4.0 mmol) bis-(triphenylphosphine)palladiumdichloride, 2.80 g (14.8 mmol) copper-(I)-iodide and finally 60 ml diisopropylamine are added at 0° C. The mixture is stirred for 2 hours at 0° C. After that time ice-water is added and the mixture is extracted with ethylacetate. The organic phase is separated and dried over sodium sulphate. The solvent is evaporated and the residue is purified by silica gel column chromatography with methylene chloride/ethyl acetate (5:1) as eluent. The product is dried in vacuo at 50° C.

Yield: 12.8 g (60% of theory),

R_{f} value: 0.50 (silica gel, methylene chloride/ethyl acetate=5:1)

C₁₂H₁₂ClN₃O₂

EII Mass spectrum: m/z=268/270 [M+H]⁺

M.p. 102-105° C.

### XIV.1.b

### [3-(6-Chloro-pyridazin-3-yl)-propyl]-carbamic acid tert-butyl ester

27.8 g (29.1 mmol) [3-(6-Chloro-pyridazin-3-yl)-prop-2-ynyl]-carbamic acid tert-butyl ester are dissolved in 250 ml ethyl acetate. 2.00 g Raney-nickel are added and the mixture is hydrogenated (25 psi) for 7 hours at RT. After that time the catalyst is filtered off and the filtrate evaporated. The residue purified by silica gel column chromatography with methylene chloride/ethyl acetate (1:1) as eluent. The product is dried in vacuo at 50° C.

Yield: 6.30 g (80% of theory),

R_{f} value: 0.50 (silica gel, methylene chloride/ethyl acetate=1:1)

C₁₂H₁₈ClN₃O₂

EII Mass spectrum: m/z=272/274 [M+H]⁺

M.p. 96-98° C.

### XIV.1.c

### {3-[6-(4-Dimethylamino-phenyl)-pyridazin-3-yl]-propyl}-carbamic acid tert-butyl ester

5.50 g (20.2 mmol) [3-(6-Chloro-pyridazin-3-yl)-propyl]-carbamic acid tert-butyl ester are dissolved in 100 ml dioxane and 1.40 g (2.00 mmol) bis-(triphenylphosphine)palladium-dichloride, 10 ml 2N sodium carbonate solution and finally 4.30 g (26.3 mmol) 4-dimethylamino-phenyl boronic acid (dissolved in 50 ml dioxane and 50 ml methanol) are added. The mixture is stirred for 4 hours at 110° C. After cooling down, water is added and the mixture is extracted with ethylacetate. The organic phase is separated and dried over sodium sulphate. The solvent is evaporated and the residue is purified by silica gel column chromatography with ethyl acetate as eluent. The product is dried in vacuo at 70° C.

Yield: 6.50 g (90% of theory),

R_{f} value: 0.30 (silica gel, petrol ether/ethyl acetate=2:1)

C₂₀H₂₈N₄O₂

EII Mass spectrum: m/z=357 [M+H]⁺

M.p. 160-164° C.

### XIV.1.d

### {4-[6-(3-Amino-propyl)-pyridazin-3-yl]-phenyl}-dimethyl-amine

6.50 g (18.2 mmol) {3-[6-(4-Dimethylamino-phenyl)-pyridazin-3-yl]-propyl}-carbamic acid tert-butyl ester are dissolved in 250 ml methylene chloride and 14.0 ml of trifluoroacetic acid are added. The mixture is stirred for 4 hours at RT. After that time the solvent is evaporated. The residue is taken up in methylene chloride and washed with 1N NaOH-solution. The organic phase is dried over sodium sulphate. After evaporation of the solvent, the product is dried in vacuo at 70° C.

Yield: 4.30 g (92% of theory),

R_{f} value: 0.20 (silica gel, methylene chloride/methanol/ammonia=5:1:0.02)

C₁₅H₂₀N₄

EII Mass spectrum: m/z=257 [M+H]⁺

M.p. 146-150° C.

The following compounds are synthesised analogously to the method described above:
(XIV.2) 3-[6-(3-Cyano-phenyl)-pyridazin-3-yl]-propylamine
(XIV.3) 3-(6-Pyridin-4-yl-pyridazin-3-yl)-propylamine
(XIV.4) 3-(6-p-Tolyl-pyridazin-3-yl)-propylamine
(XIV.5) 3-[6-(3,4-Difluoro-phenyl)-pyridazin-3-yl]-propylamine
(XIV.6) 3-[6-(2,4-Difluoro-phenyl)-pyridazin-3-yl]-propylamine

### EXAMPLE XV.1

### (4-{3-[6-(4-Methoxy-phenyl)-pyridazin-3-yl]-propoxyl}-phenyl)-methanol

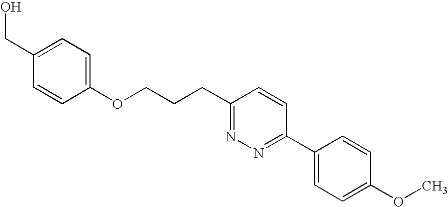

### XV1.a

### 4-{3-[6-(4-Methoxy-phenyl)-pyridazin-3-yl]-prop-2-ynyloxy}-benzoic acid ethyl ester

0.84 g (2.5 mmol) Trifluoro-methanesulfonic acid 6-(4-methoxy-phenyl)-pyridazin-3-yl ester and 1.0 g (4.9 mmol) 4-prop-2-ynyloxy-benzoic acid ethyl ester are dissolved in 30 ml THF and 88 mg (0.13 mmol) bis-(triphenylphosphine)palladiumdichloride, 47 mg (0.25 mmol) copper-(I)-iodide and finally 3.5 ml diisopropylamine are added at RT under inert gas. The mixture is stirred for 3 hours at RT and for additional 3 hours at 50° C. After that time the solvent is evaporated and purified by silica gel column chromatography with methylene chloride/methanol (95:5) as eluent. The product is washed with ether/methanol.

Yield: 0.57 g (59% of theory),

R_{f} value: 0.40 (silica gel, methylene chloride/methanol=39:1)

C₂₃H₂₀N₂O₄

EII Mass spectrum: m/z=389 [M+H]⁺

### XV.1.b

### 4-{3-[6-(4-Methoxy-phenyl)-pyridazin-3-yl]-propoxyl}-benzoic acid ethyl ester

0.55 g (1.42 mmol) 4-{3-[6-(4-Methoxy-phenyl)-pyridazin-3-yl]-prop-2-ynyloxy}-benzoic ethyl ester are dissolved in 50 ml ethyl acetate. 100 mg Raney nickel are added and the mixture is hydrogenated (3 bar) at RT until completion. After that time the catalyst is filtered off and the filtrate evaporated. The residue is purified by silica gel column chromatography with methylene chloride/ethyl acetate (9:1) as eluent and the product is dried in vacuo at 50° C.

Yield: 0.23 g (41% of theory),

R_{f} value: 0.35 (silica gel, methylene chloride/ethyl acetate=9:1)

C₂₃H₂₄N₂O₄

EII Mass spectrum: m/z=393 [M+H]⁺

### XV.1.c

### (4-{3-[6-(4-Methoxy-phenyl)-pyridazin-3-yl]-propoxy}-phenyl)-methanol

0.20 g (0.51 mmol) 4-{3-[6-(4-Methoxy-phenyl)-pyridazin-3-yl]-propoxy}-benzoic acid ethyl ester are dissolved in 10 ml THF and added to 610 ml (0.61 mmol) of a 1M solution of lithium aluminum hydride in THF at −10° C. The cooling bath is removed and the mixture is stirred for 2 hours at RT. After that time 0.1 ml water are carefully added. After 5 minutes 0.1 ml 4M NaOH solution and finally 0.5 ml water are carefully added. The mixture is stirred for 30 minutes. The solution is filtered, the solvent evaporated and the residue taken up in methylene chloride and washed with water. The organic phase is dried over sodium sulphate. After evaporation of the solvent, the product is purified by silica gel column chromatography with methylene chloride/methanol/ammonia (95:5:0.5) as eluent.

Yield: 110 mg (62% of theory),

R_{f} value: 0.45 (silica gel, methylene chloride/methanol/ammonia=9:1:0.1)

C₂₁H₂₂N₂O₃

EII Mass spectrum: m/z=351 [M+H]⁺

The following compounds are synthesised analogously to the method described above:
(XV.2) (4-{3-[6-(4-Chloro-phenyl)-pyridazin-3-yl]-propoxy}-phenyl)-methanol
(XV.3) {4-[3-(6-Phenoxy-pyridazin-3-yl)-propoxy]-phenyl}-methanol starting from 3-iodo-6-phenoxy-pyridazine (prepared analogously to J. Org. Chem. 1963, 28, 218) in step a
(XV.4) (4-{3-[6-(4-Fluoro-phenyl)-pyridazin-3-yl]-propoxy}-phenyl)-methanol
(XV.5) {4-[3-(6-Benzyloxy-pyridazin-3-yl)-propoxy]-phenyl}-methanol starting from 3-iodo-6-benzyloxy-pyridazine (prepared analogously to J. Org. Chem. 1963, 28, 218) and (4-prop-2-ynyloxy-phenyl)-methanol in step a (omitting step b)

### EXAMPLE XVI.1

### 5-Bromo-2-methyl-2,3-dihydro-1H-isoindole

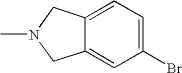

1.27 g (5.29 mmol) of 5-Bromo-2-methyl-isoindole-1,3-dione (Chem. Ber. 94, 1961, 2494) are dissolved in 60 ml THF. 2.01 ml (26.5 mmol) of borane-dimethylsulfide adduct are slowly added at 0° C. The ice-bath is removed and the mixture is stirred for 5 hours at reflux. After that time another 1.00 ml (13.2 mmol) borane-dimethylsulfide adduct are added and the mixture is stirred for 3 hours at reflux. 20 ml of methanol and 7 ml conc. HCl are slowly added. The mixture is stirred for 4 hours at 80° C. The residue is taken up in 25 ml 4N NaOH and 25 ml brine. The solution is extracted with methylene chloride, the organic phase is separated and dried over sodium sulphate. After evaporation of the solvent, the residue is purified by silica gel column cromatography with methylene chloride/methanol (95:5) as eluent.

Yield: 0.76 g (68% of theory),

R_{f} value: 0.40 (silica gel, methylene chloride/methanol=19:1) C_{g}H₁₀BrN

EII Mass spectrum: m/z=212/214 [M+H]⁺

### EXAMPLE XVII.1

### 3-(6-Phenethyl-pyridazin-3-yl)-propylamine

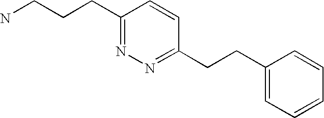

### XVII.1 .a

### 3,6-Diiodo-pyridazine

14.9 g (0.1 mol) 3,6-Dichloro-pyridazine and 120 ml (0.54 mol) hydroiodic acid are refluxed for 0.5 hours at 150° C. After that time the mixture is cooled down and poured into 0.4 N NaOH solution/ice water. The precipitate is filtered off, taken up in methylene chloride and dried over sodium sulphate. After evaporation of the solvent, the product is dried in vacuo at 50° C.

Yield: 28.3 g (85% of theory),

C₄H₂I₂N₂

EII Mass spectrum: m/z=333 [M+H]⁺

M.p. 165-168° C.

### XVII.1.b

### 3-Iodo-6-phenethyl-pyridazine

0.66 g (2.00 mmol) 3,6-Diiodo-pyridazine and 0.23 mg (0.2 mmol) tetrakis(triphenylphosphine)palladium(0) are dissolved in 5 ml THF and 5.00 ml (2.50 mmol) 0.5 N phenylethylzinc bromide in THF are added. The mixture is stirred for 3 hours at RT. After that time the mixture is poured into saturated sodium hydrogen carbonate solution and extracted with ethyl acetate. The organic phase is separated and dried over sodium sulphate. After removal of the solvent, the residue is purified by silica gel column chromatography with methylene chloride/ethyl acetate (20:1) as eluent. The product is dried in vacuo at 50° C.

Yield: 0.30 g (48% of theory),

R_{f} value: 0.50 (silica gel, methylene chloride/ethyl acetate=19:1) C₁₂H₁₁IN₂

EII Mass spectrum: m/z=311 [M+H]⁺

M.p. 120-122° C.

### XVII.1.c

### [3-(6-Phenethyl-pyridazin-3-yl)-prop-2-ynyl]-carbamic acid tert-butyl ester

Prepared according to procedure II.1.b from 5.90 g (19.0 mmol) 3-iodo-6-phenethyl-pyridazine and 3.87 g (25.0 mmol) prop-2-ynyl-carbamic acid tert-butyl ester.

Yield: 6.00 g (94% of theory),

R_{f} value: 0.80 (silica gel, methylene chloride/methanol=9:1)

C₂₀H₂₃N₃O₂

### XVII.1.d

### [3-(6-Phenethyl-pyridazin-3-yl)-propyl]-carbamic acid tert-butyl ester

Prepared according to procedure II.1.c from 6.00 g (17.8 mmol) [3-(6-phenethyl-pyridazin-3-yl)-prop-2-ynyl]-carbamic acid tert-butyl ester using 2.00 g Raney nickel as hydrogenation catalyst.

Yield: 5.50 g (91% of theory),

R_{f} value: 0.80 (silica gel, methylene chloride/methanol=9:1)

C₂₀H₂₇N₃O₂

### XVII.1.e

### 3-(6-Phenethyl-pyridazin-3-yl)-propylamine

Prepared according to procedure II.1.d from 5.50 g (16.1 mmol) [3-(6-phenethyl-pyridazin-3-yl)-propyl]-carbamic acid tert-butyl ester.

Yield: 2.20 g (57% of theory),

R_{f} value: 0.40 (silica gel, methylene chloride/methanol/ammonia=5:1:0.02)

C₁₅H₁₉N₃

The following compounds are synthesised analogously to the method described above:
(XVII.2) 3-(6-Benzyl-pyridazin-3-yl)-propylamine 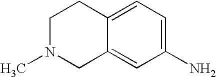 using 3,6-dichloro-pyridazine and benzylzinc bromide as starting materials in step (b)

### EXAMPLE XVIII.1

### 2-Methyl-1,2,3,4-tetrahydro-isoquinolin-7-ylamine

1.53 g (40.3 mmol) Lithium aluminum hydride are dissolved in 100 ml THF and cooled to −15° C. 2.00 g (8.05 mmol) of 7-amino-3,4-dihydro-1 H-isoquinoline-2-carboxylic acid tert-butyl ester dissolved in 100 ml THF are slowly added at −5° C. The ice-bath is removed and the mixture is stirred for 12 hours at reflux. After that time the mixture is cooled to room temperature and 22.7 g (80.5 mmol) potassium sodium tartrate tetrahydrate are added and the mixture is stirred for 3 hours at rt. After that time 1 ml water is added, the mixture is filtered over celite and the filtrate is evaporated.

Yield: 1.40 g (100% of theory),

C₁₀H₁₄N₂

EII Mass spectrum: m/z=163 [M+H]⁺

### EXAMPLE XIX.1

### 3-[6-(4-Methoxy-Phenyl)-pyridazin-3-yl]-propionaldehyde

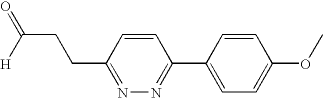

### XIX.1.a

### 3-(2-[1,3]Dioxolan-2-yl-ethyl)-6-(4-methoxy-phenyl)-pyridazine

2.70 g (8.00 mmol) Trifluoro-methanesulfonic acid 6-(4-methoxy-phenyl)-pyridazin-3-yl ester (example II.1.b) and 490 mg (0.43 mmol) tetrakis(triphenylphosphine)palladium(0) are dissolved in 20 ml THF and 20.0 ml (10.0 mmol) 0.5 N (1,3-dioxolan-2-ylethyl)zinc bromide in THF are added. The mixture is stirred for 20 hours at reflux. After that time the mixture is poured into saturated sodium hydrogen carbonate solution and extracted with ethyl acetate. The organic phase is separated and dried over sodium sulphate. After removal of the solvent, the residue is purified by silica gel column chromatography with ethyl acetate as eluent. The product is dried in vacuo at 50° C.

Yield: 2.20 g (96% of theory),

R_{f} value: 0.50 (silica gel, ethyl acetate)

C₁₆H₁₈N₂O₃

EII Mass spectrum: m/z=287 [M+H]⁺

M.p. 107-110° C.

### XIX.1.b

### 3-[6-(4-Methoxy-phenyl)-pyridazin-3-yl]-propionaldehyde

0.90 g (3.1 mmol) 3-(2-[1,3]Dioxolan-2-yl-ethyl)-6-(4-methoxy-phenyl)-pyridazine are dissolved in 15 ml 4 N HCl. The mixture is stirred for 2 hours at RT. After that time ethyl acetate is added and the mixture is neutralized by addition of sodium hydrogen carbonate. The organic phase is separated and dried over sodium sulphate. After evaporation of the solvent, the product is dried in vacuo at 50° C.

Yield: 0.70 g (92% of theory),

R_{f} value: 0.40 (silica gel, methylene chloride/methanol=9:1)

C₁₄H₁₄N₂O₂

EII Mass spectrum: m/z=243 [M+H]⁺

The following compounds are synthesised analogously to the method described above:
(XIX.2) 3-(6-Phenoxy-pyridazin-3-yl)-propionaldehyde starting from 3-iodo-6-phenoxy-pyridazine (prepared analogously to J. Org. Chem. 1963, 28, 218) in step a

### EXAMPLE XX.1

### 4-{3-[6-(4-Methoxy-phenyl)-pyridazin-3-yl]-propylamino}-benzaldehyde

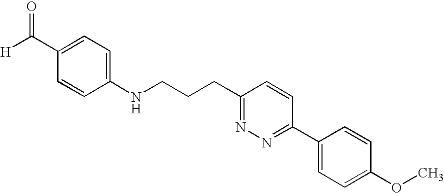

### XX.1.a

(4-Dimethoxymethyl-phenyl)-{3-[6-(4-methoxy-phenyl)-pyridazin-3-yl]-propyl]-amine 200 mg (0.82 mmol) 3-[6-(4-Methoxy-phenyl)-pyridazin-3-yl]-propylamine (educt II.1) and 0.14 ml (0.82 mmol) 4-bromo-benzaldehyde dimethyl actetal are dissolved in 3.0 ml of dioxane and 10 mg (0.03 mmol) 2-(di-tert-butylphosphino)biphenyl, 22 mg (0.03 mmol) tris(dibenzylideneaceton)dipalladium(0) and 110 mg (1.2 mmol) sodium tert-butoxide are added. The mixture is stirred for 5 hours at 60° C. in a sealed tube under argon atmosphere. After cooling, the solvent is removed. The residue is purified by silica gel column chromatography with methylene chloride/methanol/ammonia (92:8:0.1) as eluent.

Yield: 190 mg (59% of theory),

R_{f} value: 0.85 (silica gel, methylene chloride/methanol/ammonia=9:1:0.1)

retention time (HPLC): 3.1 min (method A)

C₂₃H₂₇N₃O₃

EII mass spectrum: m/z=394 [M+H]⁺

### XX.1.b

4-{3-[6-(4-Methoxy-phenyl)-pyridazin-3-yl]-propylamino}-benzaldehyde 150 mg (0.38 mmol) (4-Dimethoxymethyl-phenyl)-{3-[6-(4-methoxy-phenyl)-pyridazin-3-yl]-propyl}-amine are dissolved in 10 ml THF and 1 ml 1 N HCl is added. The mixture is stirred for 4 hours at RT. After that time ethyl acetate is added and the mixture is neutralized by addition of sodium carbonate solution. The organic phase is separated and dried over sodium sulphate. After evaporation of the solvent, the product is dried in vacuo at 50° C.

Yield: 0.11 g (83% of theory),

R_{f} value: 0.60 (silica gel, methylene chloride/methanol/ammonia=9:1:0.1)

C₂₁H₂₁ N₃O₂

EII Mass spectrum: m/z=348 [M+H]⁺

### EXAMPLE XXI

The following compounds are synthesised analogously to the method described in WO 2001/27081 (example XX):
(XXI.1) 1-(4-amino-benzyl)-azetidin-3-ol
(XXI.2) 1-(4-amino-benzyl)-piperidine-4-carboxylic acid dimethylamide

### EXAMPLE XXII.1

### [6-(3-Amino-propyl)-pyridazin-3-yl]-benzyl-amine

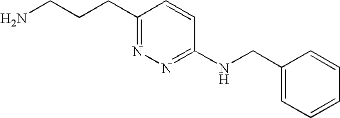

### XXII.1.a

### [3-(6-Benzylamino-pyridazin-3-yl)-propyl]-carbamic acid tert-butyl ester

0.27 g (1.0 mmol) [3-(6-Chloro-pyridazin-3-yl)-propyl]-carbamic acid tert-butyl ester (example XIV.1.b) are dissolved in 1.1 ml benzylamine and stirred for 5 hours at 140° C. After cooling down, the solvent is evaporated and the residue is purified by silica gel column chromatography with methylene chloride/methanol (9:1) as eluent. The product is dried in vacuo at 50° C.

Yield: 0.18 g (53% of theory),

R_{f} value: 0.70 (silica gel, methylene chloride/methanol (9:1)

### XXII.1.b

### [6-(3-Amino-propyl)-pyridazin-3-yl]-benzyl-amine

1.15 g (3.36 mmol) [3-(6-Benzylamino-pyridazin-3-yl)-propyl]-carbamic acid tert-butyl ester are dissolved in 50 ml methylene chloride and 3.0 ml of trifluoroacetic acid are added. The mixture is stirred for 12 hours at RT. After that time the solvent is evaporated. The residue is taken up in methylene chloride and washed with 1N NaOH-solution. The organic phase is dried over sodium sulphate. After evaporation of the solvent, the product is dried in vacuo at 70° C.

Yield: 0.75 g (92% of theory),

R_{f} value: 0.10 (silica gel, methylene chloride/methanol/ammonia=5:1:0.02) C₁₄H₁₈N₄

EII Mass spectrum: m/z=243 [M+H]⁺

The following compounds are synthesised analogously to the method described above:
(XXII.2) [6-(3-Amino-propyl)-pyridazin-3-yl]-benzyl-methyl-amine

### EXAMPLE XXIII.1

### 3-[6-(Pyridin-3-yloxy)-pyridazin-3-yl]-propylamine

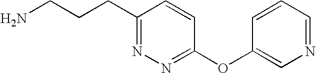

### XXIII.1.a

### [3-(6-Benzylamino-pyridazin-3-yl)-propyl]-carbamic acid tert-butyl ester

2.70 g (10.0 mmol) [3-(6-Chloro-pyridazin-3-yl)-propyl]-carbamic acid tert-butyl ester (example XIV.1.b), 1.30 g (13.7 mmol) 3-hydroxy-pyridine, 4.25 g (1.00 mmol) potassium phosphate, 0.425 g (1.00 mmol) di-tert-butyl-(2′,4′,6′-triisopropyl-biphenyl-2-yl)-phosphane and 460 mg (0.50 mmol) tris(dibenzylideneacetone)dipalladium(0) are dissolved in 30 ml dioxane under argon atmosphere. The mixture is stirred for 25 hours at 100° C. After that time the mixture is cooled down, filtered through celite and the solvent is removed. The residue is purified by silica gel column chromatography with methylene chloride/ethyl acetate (1:1) as eluent.

Yield: 1.80 g (55% of theory),

R_{f} value: 0.30 (silica gel, ethyl acetate)

C₁₇H₂₂N₄O₃

EII Mass spectrum: m/z=331 [M+H]⁺

### XXIII.1.b

### 3-[6-(Pyridin-3-yloxy)-pyridazin-3-yl]-propylamine

1.80 g (5.45 mmol) [3-(6-Benzylamino-pyridazin-3-yl)-propyl]-carbamic acid tert-butyl ester are dissolved in 50 ml methylene chloride and 4.0 ml of trifluoroacetic acid are added. The mixture is stirred for 12 hours at RT. After that time the solvent is evaporated. The residue is taken up in methylene chloride and washed with 1N NaOH-solution. The organic phase is dried over sodium sulphate. After evaporation of the solvent, the product is dried in vacuo at 50° C.

Yield: 0.80 g (64% of theory),

R_{f} value: 0.30 (silica gel, methylene chloride/methanol/ammonia=5:2:0.01)

### EXAMPLE XXIV.1

### 4-[3-(6-Phenoxy-pyridazin-3-yl)-propoxy]-benzaldehyde

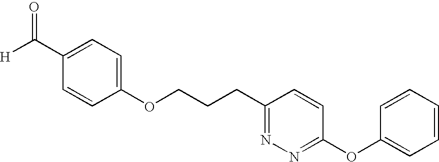

0.20 g (0.60 mmol) {4-[3-(6-Phenoxy-pyridazin-3-yl)-propoxy]-phenyl}-methanol (educt XV.3) are dissolved in 10 ml methylene chloride and 0.36 g (3.0 mmol) manganese dioxide are added. The mixture is stirred for 3 hours at RT. After that time, the mixture is filtered through celite and the solvent is removed.

Yield: 170 mg (86% of theory),

C₂₀H₁₈N₂O₃

EII mass spectrum: m/z=335 [M+H]⁺

The following compounds are synthesised analogously to the method described above:
(XXIV.2) 4-[3-(6-Benzyloxy-pyridazin-3-yl)-propoxy]-benzaldehyde using {4-[3-(6-benzyloxy-pyridazin-3-yl)-propoxy]-phenyl}-methanol (educt XV.5) as starting material

### EXAMPLE XXV

The following starting materials can be prepared analogously to procedures described in WO 2004/039780:
(XXV. 1) 1-(4-{4-[6-(4-Methoxy-phenyl)-pyridazin-3-yl]-but-3-ynyl}-benzyl)-piperidin-4-ol
(XXV.2) 1-{4-[4-(6-Benzyloxy-pyridazin-3-yl)-but-3-ynyl]-benzyl}-piperidin-4-ol

### EXAMPLE XXVI.1

### (6-{4-[5-(4-Chloro-phenyl)-pyridin-2-yl]-butyl}-pyridazin-3-yl)-methanol

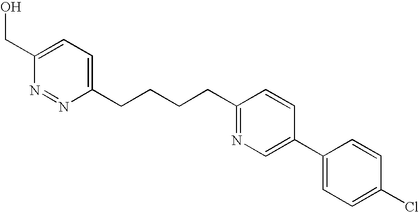

### XXVI.1.a

### 3-(6-Chloro-pyridazin-3-yl)-prop-2-yn-1-ol

42.0 g (175 mmol) 3-Chloro-6-iodo-pyridazine (Tetrahedron 55, 1999, 15067) and 11.2 ml (192 mmol) propargyl alcohol are dissolved in 400 ml THF and 1.23 g (1.75 mmol) bis-(triphenylphosphine)palladiumdichloride, 665 mg (3.49 mmol) copper-(I)-iodide and finally 49.4 ml diisopropylamine are added at 0° C. under inert gas. The mixture is stirred for 0.5 hours at 0° C. and for an additional hour at RT. After that time, ethyl acetate is added and the solution is washed with diluted ammonia solution twice. The organic phase is separated and dried over magnesium sulphate. The product is taken up in ethyl acetate/acetonitrile and filtered through charcoal. Finally, the solvent is removed in vacuo.

Yield: 19.5 g (66% of theory),

C₇H₅ClN₂O

EII Mass spectrum: m/z=169 [M+H]⁺

### XXVI.1.b

### 3-(6-Chloro-pyridazin-3-yl)-propan-1-ol

19.4 g (115 mmol) 3-(6-Chloro-pyridazin-3-yl)-prop-2-yn-1-ol are dissolved in 400 ml THF. 4.00 g Platinum(IV) oxide and 3.05 g vanadyl(IV) acetylacetonate are added and the mixture is hydrogenated (15 psi) at RT for 5 hours. After that time, the catalyst is filtered off and the filtrate evaporated. The residue is purified by silica gel column chromatography with ethyl acetate as eluent.

Yield: 9.80 g (49% of theory),

R_{f} value: 0.30 (silica gel, ethyl acetate)

C₇H₉ClN₂O

EII Mass spectrum: m/z=173/175 [M+H]⁺

### XXVI.1.c

### 3-(6-Chloro-pyridazin-3-yl)-propionaldehyde

5.30 ml (61.8 mmol) Oxalyl chloride are dissolved in 250 ml methylene chloride under inert atmosphere. The solution is cooled to −60° C. and 8.77 ml (124 mmol) anhydrous DMSO in 30 ml methylene chloride are added at −60° C. and stirred for additional 10 minutes at −60° C. After that time, 8.20 g (47.5 mmol) {3-(6-chloro-pyridazin-3-yl)-propan-1-ol dissolved in 100 ml methylene chloride are added. The mixture is stirred for 45 minutes at −55° C. After that time, 16.0 ml (115 mmol) triethylamine are carefully added, the cooling bath is removed and the mixture is stirred for 12 hours at RT. Methylene chloride is added and the organic phase is washed with water twice. The organic phase is dried over magnesium sulphate, the solvent is removed and the residue is purified by silica gel column chromatography with ethyl acetate as eluent.

Yield: 3.60 g (44% of theory),

R_{f} value: 0.50 (silica gel, ethyl acetate)

C₇H₇ClN₂O

EII mass spectrum: m/z=171/173 [M+H]⁺

### XXVI.1.d

### 3-But-3-ynyl-6-chloro-pyridazine

3.60 g (21.1 mmol) 3-(6-Chloro-pyridazin-3-yl)-propionaldehyde are dissolved in 150 ml methanol and 5.83 g (42.2 mmol) potassium carbonate and finally 4.87 g (25.3 mmol) dimethyl 1-diazo-2-oxopropylphosphonate are added. The mixture is stirred for 12 hours at RT. After that time, ethyl acetate is added and the organic phase is washed with water twice.

The organic phase is dried over magnesium sulphate, the solvent is removed and the residue is purified by silica gel column chromatography with petrol ether/ethyl acetate (1:1) as eluent.

Yield: 2.00 g (57% of theory),

R_{f} value: 0.60 (silica gel, petrol ether/ethyl acetate=1:1)

C₈H₇ClN₂

EII mass spectrum: m/z=167/169 [M+H]⁺

### XXVI.1.e

### 3-Chloro-6-{4-[5-(4-chloro-phenyl)-pyridin-2-yl]-but-3-ynyl}-pyridazine

1.0 g (6.0 mmol) 3-But-3-ynyl-6-chloro-pyridazine and 1.9 g (6.0 mmol) 5-(4-chloro-phenyl)-2-iodo-pyridine (described in WO 2004/039780) are dissolved in 20 ml THF and 98 mg (0.12 mmol) PdCl₂(dppf), 23 mg (0.12 mmol) copper-(I)-iodide and finally 1.7 ml diisopropylamine are added at RT under inert gas. The mixture is stirred for 3 hours at RT. After that time, methanol is added and the precipitate is filtered off. The filtrate is reduced in vacuo, methanol is added and the precpitate is filtered off. The combined precpitates are dried at RT.

Yield: 2.1 g (89% of theory),

R_{f} value: 0.50 (silica gel, petrol ether/ethyl acetate=2:8)

C₁₉H₁₃Cl₂N₃

EII Mass spectrum: m/z=354/356/358 [M+H]⁺

### XXVI.1.f

6-{4-[5-(4-Chloro-phenyl)-pyridin-2-yl]-but-3-ynyl]-pyridazine-3-carboxylic acid methyl ester 2.00 g (5.65 mmol) 3-Chloro-6-a4-[5-(4-chloro-phenyl)-pyridin-2-yl]-but-3-ynyl}-pyridazine are dissolved in 20 ml methanol and 20 ml DMF and 101 mg (0.452 mmol) palladium(II) acetate, 250 mg (0.452 mmol) dppf and 1.6 ml triethylamine are added under inert gas. The mixture is transferred to an autoclave and CO is added (4 bar). The mixture is shaken for 4 hours at 50° C. After cooling down, the precipitate is filtered off. The filtrate is reduced in vacuo and the residue is purified by silica gel column chromatography with ethyl acetate as eluent. The product and the precipitate are combined, dissolved in methylene chloride and some methanol and filtered through silica gel. Finally, the solvent is removed in vacuo.

Yield: 1.00 g (47% of theory),

C₂₁ H₁₆ClN₃O₂

EII Mass spectrum: m/z=378/380 [M+H]⁺

### XXVI.1.q

### 6-{4-[5-(4-Chloro-phenyl)-pyridin-2-yl]-butyl}-pyridazine-3-carboxylic acid methyl ester

600 mg (1.59 mmol) 6-{4-[5-(4-Chloro-phenyl)-pyridin-2-yl]-but-3-ynyl}-pyridazine-3-carboxylic acid methyl ester-are dissolved in 60 ml ethyl acetate. 200 mg Raney nickel are added and the mixture is hydrogenated (3 bar) at RT until completion. After that time, methanol is added, the catalyst is filtered off and the filtrate evaporated. The residue is purified by silica gel column chromatography with ethyl acetate as eluent.

Yield: 400 mg (66% of theory),

C₂₁H₂₀ClN₃O₂

EII Mass spectrum: m/z=382/384 [M+H]⁺

### XXVI.1.h

### 6-{4-[5-(4-Chloro-phenyl)-pyridin-2-yl]-butyl}-pyridazine-3-carboxylic acid

500 mg (1.31 mmol) 6-{4-[5-(4-Chloro-phenyl)-pyridin-2-yl]-butyl}-pyridazine-3-carboxylic acid methyl ester are dissolved in 25 ml methanol and 4.0 ml 1N NOH are added. The mixture is stirred for 2 hours at RT. After that time, 4.0 ml 1N HCl are added. The solvent is almost removed in vacuo and the precipitate is filtered off. The precipitate is washed with water and dried at 40° C.

Yield: 480 mg (100% of theory),

C₂₀H₁₈ClN₃O₂

EII Mass spectrum: m/z=368/370 [M+H]⁺

### XXVI.1.i

### (6-{4-[5-(4-Chloro-phenyl)-pyridin-2-yl]-butyl}-pyridazin-3-yl)-methanol

480 mg (1.31 mmol) 6-{4-[5-(4-Chloro-phenyl)-pyridin-2-yl]-butyl}-pyridazine-3-carboxylic acid are dissolved in 30 ml THF and 233 mg (1.44 mmol) 1,1′-carbonyl-diimidazole are added. The mixture is stirred for 1 hour at 50° C. After cooling down, the mixture is added to a solution of 148 mg (3.92 mmol) sodium borohydride in 40 ml water. The mixture is stirred for 30 minutes. The mixture is acidified by addition of 1 N potassium hydrogensulphate solution and stirred for 20 minutes. After that time, the mixture is neutralized by addition of sodium hydrogencarbonate solution. The aqueous phase is extracted with ethyl acetate twice. The organic phase is washed with water twice and dried over sodium sulphate. After evaporation of the solvent, the product is purified by silica gel column chromatography with ethyl acetate/methanol (9:1) as eluent.

Yield: 250 mg (54% of theory),

C₂₀H₂₀ClN₃O

EII Mass spectrum: m/z=354/356 [M+H]⁺

Preparation of the End Compounds:

### EXAMPLE 1.1

### [3-(4′-Chloro-biphenyl-4-yl)-propyl]-[4-(4-methyl-piperidin-1-ylmethyl)-phenyl]-amine

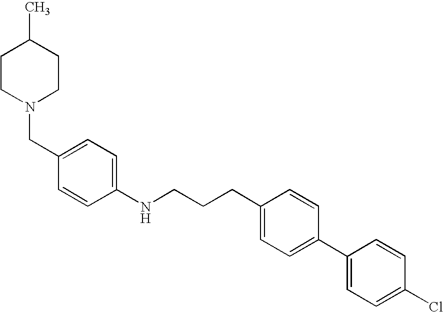

246 mg (1.00 mmol) 3-(4′-Chloro-biphenyl-4-yl)-propylamine (educt I.1) and 315 mg (1.00 mmol) 1-(4-iodo-benzyl)-4-methyl-piperidine (educt III.1) are dissolved in 1.5 ml of isopropanol and 112 ml (2.00 mmol) ethyleneglycol, 425 mg (2.00 mmol) potassium phosphate and 10 mg (0.05 mmol) copper-(I)-iodide are added. The mixture is stirred for 15 hours at 80° C. in a sealed tube under argon atmosphere. After cooling, water and ethyl acetate are added. The organic phase is separated and dried over sodium sulphate. After evaporation of the solvent, the residue is purified by silica gel column chromatography with methylene chloride/ethanol/ammonia (5:1:0.01) as eluent.

Yield: 190 mg (44% of theory),

R_{f} value: 0.40 (silica gel, methylene chloride/methanol/ammonia=9:1:0.01)

M.p. 69-71° C.

C₂₈H₃₃ClN₂

EII mass spectrum: m/z=433/435 [M+H]⁺

### EXAMPLE 2

The following compounds of general formula II-1 are prepared analogously to Example 1.1, the educts used being shown in the column headed “Educts”:

| | |
|---|---|
| | (II-1) |
| 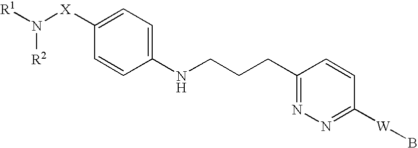 | |

| Exam- | | | | mass | M.p. | |
|---|---|---|---|---|---|---|
| ple | R¹R²N-X- | -W-B | Educts | spectrum | [° C.] | R_{f}-value |
| 2.1 | 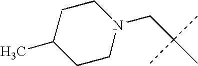 | 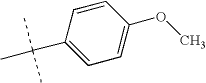 | II.1 III.1 | 431 [M + H]⁺ | 133-135 | 0.50 (A) |
| 2.2 | 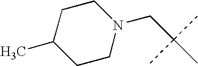 | 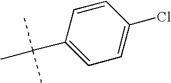 | II.1 III.1 | 435/437 [M + H]⁺ | 129-131 | 0.60 (A) |
| 2.3 | 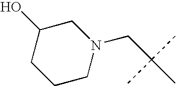 | 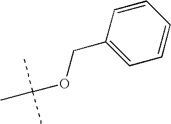 | VI.1 III.18 | 433 [M + H]⁺ | n.d. | 0.40 (A) |
| 2.4 | 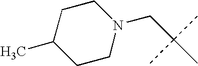 | 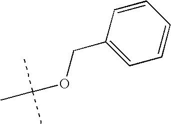 | VI.1 III.1 | 431 [M + H]⁺ | n.d. | 0.50 (A) |
| 2.5 | 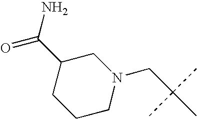 | 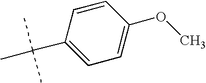 | II.1 III.38 | 460 [M + H]⁺ | n.d. | 0.45 (B) |
| 2.6 | 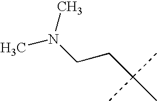 | 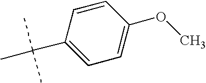 | II.1 III.39 | 391 [M + H]⁺ | n.d. | 0.55 (B) |

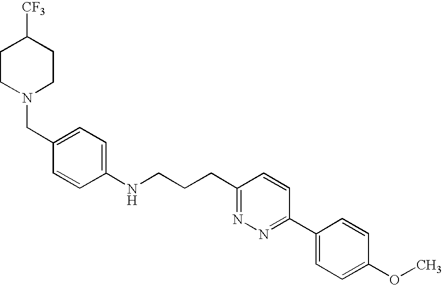

### EXAMPLE 3.1

### {3-[6-(4-Methoxy-phenyl)-pyridazin-3-yl]-propyl}-[4-(4-methyl)-phenyl]-amine

243 mg (1.00 mmol) 3-[6-(4-Methoxy-phenyl)-pyridazin-3-yl]-propylamine (educt II.1) and 322 mg (1.00 mmol) 1-(4-bromo-benzyl)-4-trifluoromethyl-piperidine (educt III.2) are dissolved in 2.0 ml of dioxane and 12 mg (0.04 mmol) 2-(di-tert-butylphosphino)biphenyl, 18 mg (0.02 mmol) tris(dibenzylideneaceton)dipalladium(0) and 135 mg (1.4 mmol) sodium tert-butoxide are added. The mixture is stirred for 26 hours at 80° C. in a sealed tube under argon atmosphere. After cooling, water is added. The precipitate is filtered off and purified by silica gel column chromatography with methylene chloride/methanol/ammonia (9:1:0.1) as eluent.

Yield: 290 mg (60% of theory),

R_{f} value: 0.50 (silica gel, methylene chloride/methanol/ammonia=9:1:0.1)

retention time (HPLC): 2.4 min (method A)

C₂₇H₃₁F₃N₄O

EII mass spectrum: m/z=485 [M+H]⁺

The following compounds of general formula III-1 are prepared analogously to Example 3.1, the educts used being shown in the column headed “Educts”:

| | |
|---|---|
| | (III-1) |
| 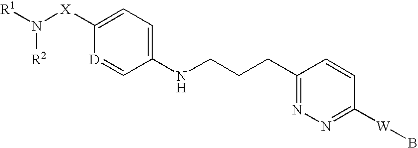 | |

| | | | | | | | ret. time | |
|---|---|---|---|---|---|---|---|---|
| Exam- | | | | | mass | M.p. | (HPLC) | |
| ple | R¹R²N-X- | -W-B | D | Educts | spectrum | [° C.] | [min]* | R_{f}-value |
| 3.2 | 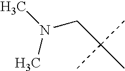 | 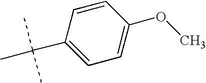 | CH | II.1 III.3 | 377 [M + H]⁺ | n.d. | n.d. | 0.50 (A) |
| 3.3 | 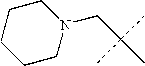 | 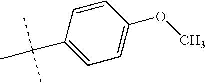 | CH | II.1 III.4 | 417 [M + H]⁺ | 85 | n.d. | 0.40 (A) |
| 3.4 | 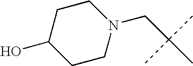 | 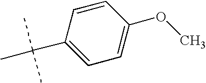 | CH | II.1 III.9 | 433 [M + H]⁺ | 145-148 | 2.2 | 0.50 (D) |
| 3.5 | 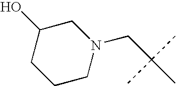 | 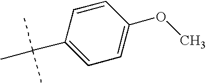 | CH | II.1 III.10 | 433 [M + H]⁺ | 140-142 | 2.2 | 0.50 (D) |
| 3.6 | 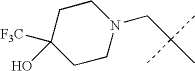 | 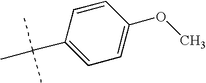 | CH | II.1 III.8 | 501 [M + H]⁺ | 173-176 | 2.3 | 0.50 (A) |
| 3.7 | 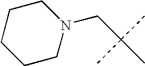 | 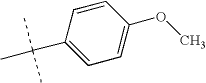 | N | II.1 V.1 | 418 [M + H]⁺ | 160-162 | 2.2 | 0.60 (D) |
| 3.8 | 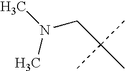 | 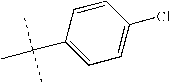 | CH | II.2 III.3 | 381/383 [M + H]⁺ | 126-130 | 2.4 | 0.30 (A) |
| 3.9 | 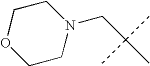 | 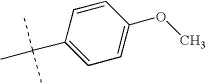 | CH | II.1 III.11 | 419 [M + H]⁺ | n.d. | 2.3 | 0.60 (B) |
| 3.10 | 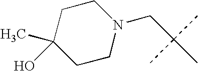 | 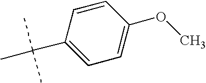 | CH | II.1 III.12 | 447 [M + H]⁺ | n.d. | 2.2 | 0.45 (B) |
| 3.11 | 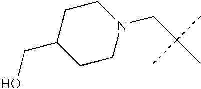 | 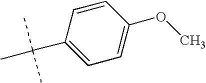 | CH | II.1 III.13 | 447 [M + H]⁺ | n.d. | 2.2 | 0.35 (B) |
| 3.12 | 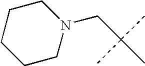 | 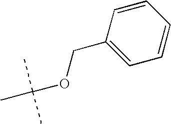 | CH | VI.1 III.4 | 417 [M + H]⁺ | n.d. | 2.4 | 0.50 (A) |
| 3.13 | 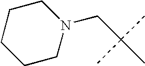 | 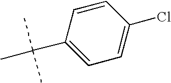 | CH | II.2 III.4 | 421/423 [M + H]⁺ | n.d. | 2.6 | 0.50 (A) |
| 3.14 | 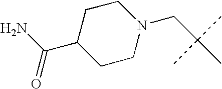 | 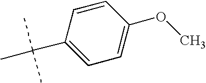 | CH | II.1 III.14 | 460 [M + H]⁺ | n.d. | 2.2 | 0.80 (B) |
| 3.15 | 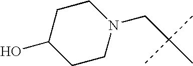 | 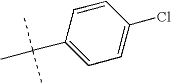 | CH | II.2 III.9 | 437/439 [M + H]⁺ | 120-125 | 2.4 | 0.30 (A) |
| 3.16 | 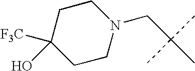 | 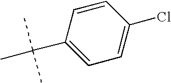 | CH | II.2 III.8 | 505/507 [M + H]⁺ | n.d. | 2.6 | 0.50 (A) |
| 3.17 | 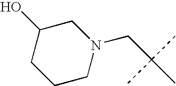 | 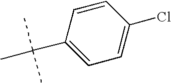 | CH | II.2 III.10 | 437/439 [M + H]⁺ | 90-95 | 2.5 | 0.50 (A) |
| 3.18 | 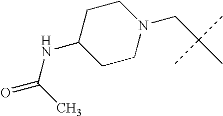 | 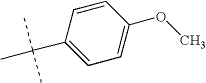 | CH | II.1 III.15 | 474 [M + H]⁺ | n.d. | 2.2 | 0.80 (B) |
| 3.19 | 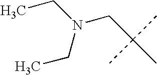 | 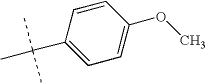 | CH | II.1 III.16 | 405 [M + H]⁺ | n.d. | 2.2 | 0.85 (B) |
| 3.20 | 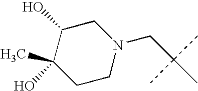 | 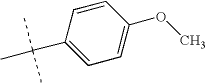 | CH | II.1 III.17 | 463 [M + H]⁺ | n.d. | 2.2 | 0.45 (B) |
| 3.21 | 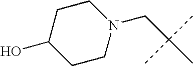 | 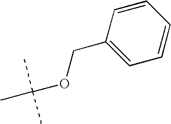 | CH | VI.1 III.9 | 433 [M + H]⁺ | 70-73 | 2.4 | 0.20 (A) |
| 3.22 | 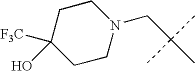 | 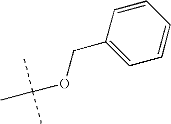 | CH | VI.1 III.8 | 501 [M + H]⁺ | 145-147 | 2.6 | 0.40 (A) |
| 3.23 | 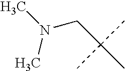 | 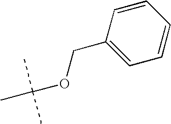 | CH | VI.1 III.3 | 377 [M + H]⁺ | n.d. | 2.4 | 0.40 (A) |
| 3.24 | 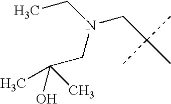 | 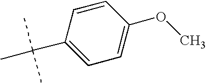 | CH | II.1 III.19 | 449 [M + H]⁺ | n.d. | 2.5 | 0.80 (L) |
| 3.25 | 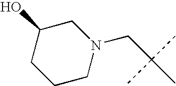 | 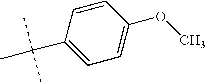 | CH | II.1 III.20 | 433 [M + H]⁺ | n.d. | 2.4 | 0.65 (L) |
| 3.26 | 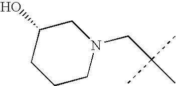 | 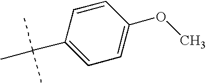 | CH | II.1 III.21 | 433 [M + H]⁺ | n.d. | 2.4 | 0.65 (L) |
| 3.27 | 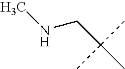 | 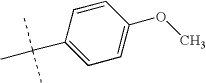 | CH | II.1 III.22 | 363 [M + H]⁺ | n.d. | 2.3 | 0.30 (L) |
| 3.28 | 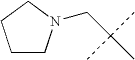 | 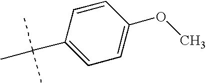 | CH | II.1 III.23 | 403 [M + H]⁺ | n.d. | 2.4 | 0.50 (L) |
| 3.29 | 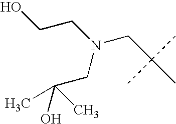 | 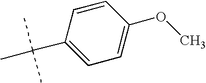 | CH | II.1 III.24 | 465 [M + H]⁺ | n.d. | 2.4 | 0.80 (L) |
| 3.30 | 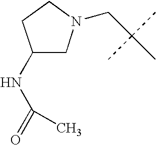 | 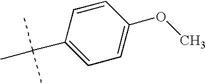 | CH | II.1 III.25 | 460 [M + H]⁺ | n.d. | 2.3 | 0.50 (L) |
| 3.31 | 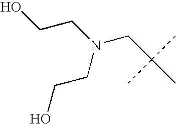 | 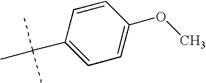 | CH | II.1 III.26 | 437 [M + H]⁺ | n.d. | 2.3 | 0.65 (L) |
| 3.32 | 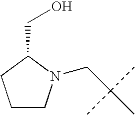 | 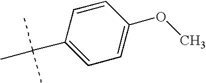 | CH | II.1 III.27 | 433 [M + H]⁺ | n.d. | 2.4 | 0.80 (L) |
| 3.33 | 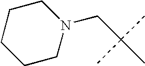 | 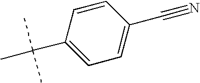 | CH | II.3 III.4 | 412 [M + H]⁺ | 180-182 | 2.4 | 0.40 (A) |
| 3.34 | 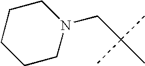 | 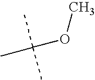 | CH | VI.2 III.4 | 341 [M + H]⁺ | 64-66 | 2.0 | 0.40 (A) |
| 3.35 | 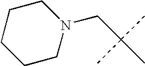 | 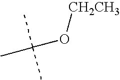 | CH | VI.3 III.4 | 447 [M + H]⁺ | 64-67 | 2.2 | 0.40 (A) |
| 3.36 | 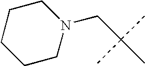 | 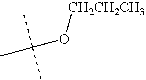 | CH | VI.4 III.4 | 369 [M + H]⁺ | 69-72 | 2.4 | 0.40 (A) |
| 3.37 | 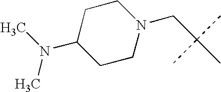 | 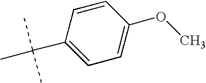 | CH | II.1 III.28 | 460 [M + H]⁺ | n.d. | 2.3 | 0.40 (B) |
| 3.38 | 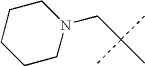 | 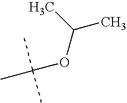 | CH | VI.5 III.4 | 369 [M + H]⁺ | n.d. | 2.2 | 0.40 (A) |
| 3.39 | 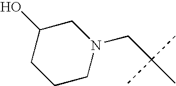 | 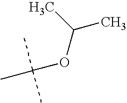 | CH | VI.5 III.10 | 385 [M + H]⁺ | n.d. | 2.1 | 0.40 (A) |
| 3.40 | 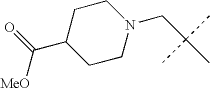 | 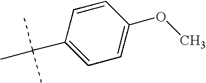 | CH | II.1 III.29 | 475 [M + H]⁺ | n.d. | 2.4 | 0.60 (B) |
| 3.41 | 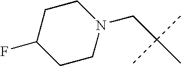 | 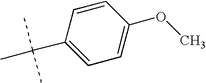 | CH | II.1 III.30 | n.d. | n.d. | 2.4 | 0.70 (B) |
| 3.42 | 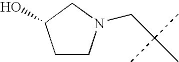 | 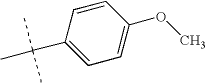 | CH | II.1 III.31 | 419 [M + H]⁺ | n.d. | 2.3 | 0.45 (B) |
| 3.43 | 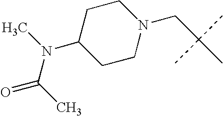 | 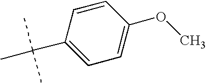 | CH | II.1 III.32 | 488 [M + H]⁺ | n.d. | 2.3 | 0.60 (B) |
| 3.44 | 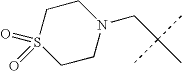 | 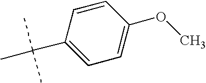 | CH | II.1 III.33 | 467 [M + H]⁺ | n.d. | 2.3 | 0.70 (B) |
| 3.45 | 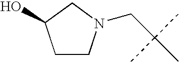 | 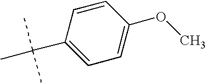 | CH | II.1 III.34 | 467 [M + H]⁺ | n.d. | 2.3 | 0.70 (B) |
| 3.46 | 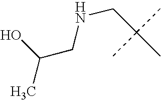 | 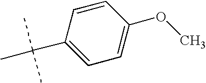 | CH | II.1 III.35 | 407 [M + H]⁺ | n.d. | 2.3 | 0.35 (B) |
| 3.47 | 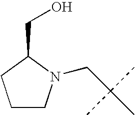 | 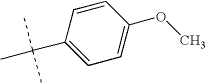 | CH | II.1 III.36 | 447 [M + H]⁺ | n.d. | 2.4 | 0.30 (B) |
| 3.48 | 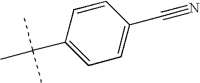 | 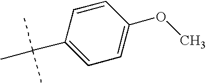 | CH | II.1 III.37 | 447 [M + H]⁺ | n.d. | 2.4 | 0.30 (B) |
| 3.49 | 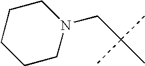 | 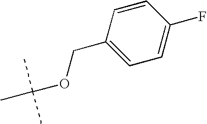 | CH | VI.6 III.4 | 435 [M + H]⁺ | 95-98 | 2.6 | 0.50 (A) |
| 3.50 | 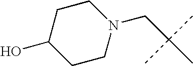 | 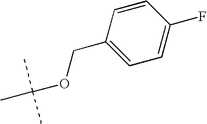 | CH | VI.6 III.9 | 451 [M + H]⁺ | 80-83 | 2.4 | 0.30 (B) |
| 3.51 | 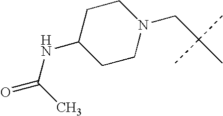 | 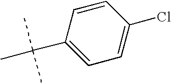 | CH | II.2 III.15 | 478/480 [M + H]⁺ | n.d. | 2.5 | 0.45 (A) |
| 3.52 | 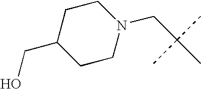 | 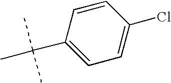 | CH | II.2 III.13 | 447 [M + H]⁺ | n.d. | 2.5 | 0.45 (A) |
| 3.53 | 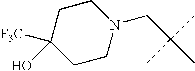 | 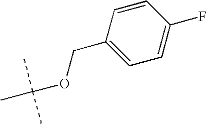 | CH | VI.6 III.8 | 519 [M + H]⁺ | 132-135 | 2.6 | 0.50 (A) |
| 3.54 | 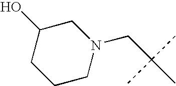 | 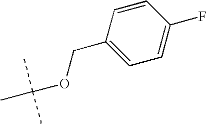 | CH | VI.6 III.10 | 451 [M + H]⁺ | n.d. | 2.4 | 0.40 (A) |
| 3.55 | 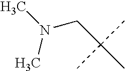 | 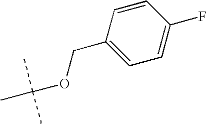 | CH | VI.6 III.3 | 395 [M + H]⁺ | 69-70 | 2.4 | 0.30 (A) |
| 3.56 | 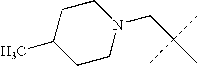 | 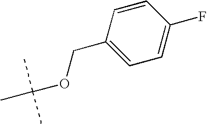 | CH | VI.6 III.1 | 449 [M + H]⁺ | 75-78 | 2.6 | 0.40 (A) |
| 3.57 | 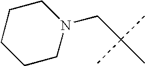 | 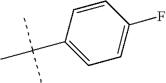 | CH | II.4 III.4 | 405 [M + H]⁺ | 112-115 | 2.4 | 0.40 (A) |
| 3.58 | 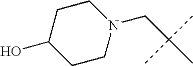 | 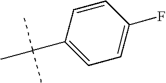 | CH | II.4 III.9 | 421 [M + H]⁺ | 142-144 | 2.3 | 0.40 (A) |
| 3.59 | 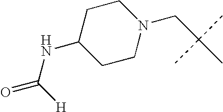 | 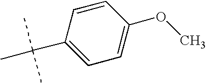 | CH | II.1 III.40 | 460 [M + H]⁺ | n.d. | 2.2 | 0.40 (B) |
| 3.60 | 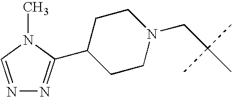 | 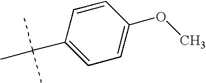 | CH | II.1 III.41 | 498 [M + H]⁺ | n.d. | 2.2 | 0.25 (B) |
| 3.61 | 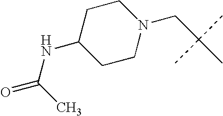 | 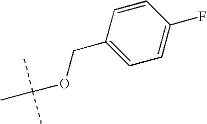 | CH | VI.6 III.15 | 492 [M + H]⁺ | 188-192 | 2.4 | 0.60 (D) |
| 3.62 | 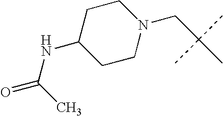 | 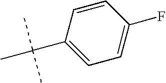 | CH | II.4 III.15 | 462 [M + H]⁺ | 150-153 | 2.3 | 0.40 (A) |
| 3.63 | 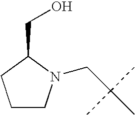 | 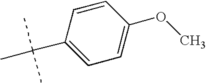 | CH | II.1 III.42 | 433 [M + H]⁺ | n.d. | 2.4 | 0.30 (B) |
| 3.64 | 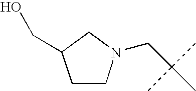 | 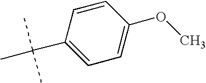 | CH | II.1 III.43 | 433 [M + H]⁺ | n.d. | 2.3 | 0.40 (B) |
| 3.65 | 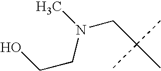 | 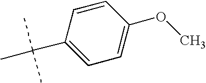 | CH | II.1 III.44 | 407 [M + H]⁺ | n.d. | 2.3 | 0.35 (B) |
| 3.66 | 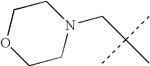 | 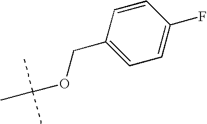 | CH | VI.6 III.11 | 437 [M + H]⁺ | 50 | 2.5 | 0.40 (A) |
| 3.67 | 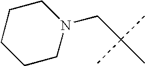 | 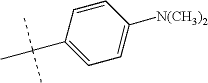 | CH | XIV.1 III.4 | 430 [M + H]⁺ | 120-125 | 2.4 | 0.50 (A) |
| 3.68 | 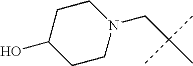 | 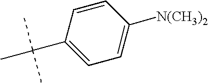 | CH | XIV.1 III.9 | 446 [M + H]⁺ | 160-163 | 2.2 | 0.40 (D) |
| 3.69 | 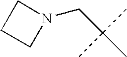 | 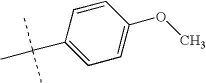 | CH | II.1 III.45 | 389 [M + H]⁺ | n.d. | 2.2 | 0.40 (B) |
| 3.70 | 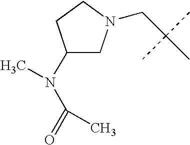 | 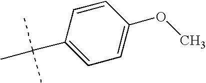 | CH | II.1 III.46 | 474 [M + H]⁺ | n.d. | 2.3 | 0.60 (B) |
| 3.71 | 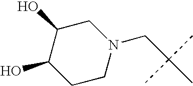 | 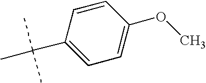 | CH | II.1 III.47 | 449 [M + H]⁺ | n.d. | 2.2 | 0.40 (B) |
| 3.72 | 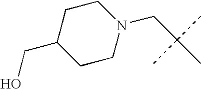 | 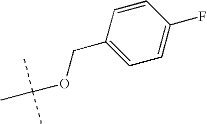 | CH | VI.6 III.13 | 465 [M + H]⁺ | n.d. | 2.4 | 0.20 (A) |
| 3.73 | 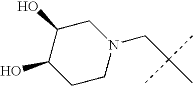 | 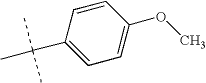 | CH | II.1 III.48 | 449 [M + H]⁺ | n.d. | 2.2 | 0.40 (B) |
| 3.74 | 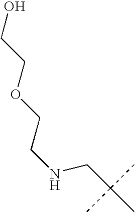 | 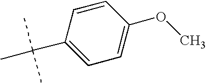 | CH | II.1 III.49 | 447 [M + H]⁺ | n.d. | 2.2 | 0.25 (B) |
| 3.75 | 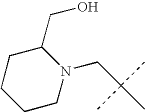 | 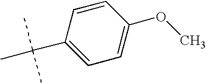 | CH | II.1 III.50 | 447 [M + H]⁺ | n.d. | 2.3 | 0.30 (B) |
| 3.76 | 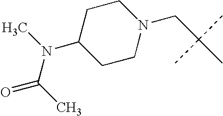 | 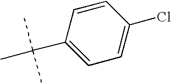 | CH | II.2 III.32 | 492/494 [M + H]⁺ | n.d. | 2.5 | 0.30 (B) |
| 3.77 | 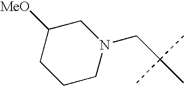 | 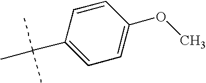 | CH | II.1 III.51 | 447 [M + H]⁺ | n.d. | 2.2 | 0.65 (B) |
| 3.78 | 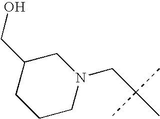 | 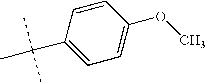 | CH | II.1 III.52 | 447 [M + H]⁺ | n.d. | 2.3 | 0.40 (B) |
| 3.79 | 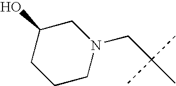 | 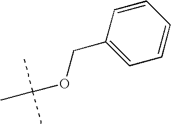 | CH | VI.1 III.20 | 433 [M + H]⁺ | n.d. | 2.4 | 0.30 (B) |
| 3.80 | 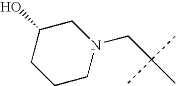 | 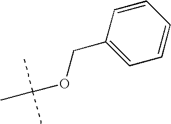 | CH | VI.1 III.21 | 433 [M + H]⁺ | n.d. | 2.4 | 0.30 (B) |
| 3.81 | 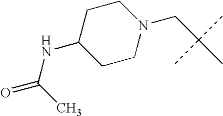 | 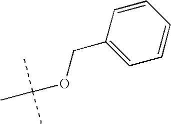 | CH | II.4 III.21 | 474 [M + H]⁺ | n.d. | 2.4 | 0.30 (B) |
| 3.82 | 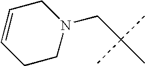 | 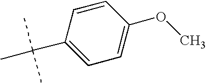 | CH | II.1 III.53 | 415 [M + H]⁺ | n.d. | 2.4 | 0.50 (B) |
| 3.83 | 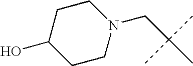 | 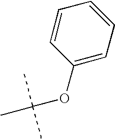 | CH | VI.7 III.9 | 419 [M + H]⁺ | 120-125 | 2.3 | 0.50 (A) |
| 3.84 | 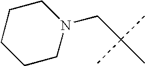 | 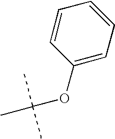 | CH | VI.7 III.4 | 403 [M + H]⁺ | 90-92 | 2.4 | 0.40 (A) |
| 3.85 | 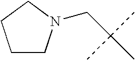 | 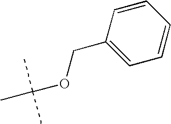 | CH | II.4 III.23 | 403 [M + H]⁺ | n.d. | 2.4 | 0.30 (B) |
| 3.86 | 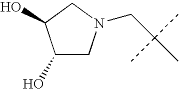 | 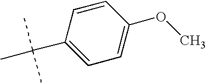 | CH | II.1 III.54 | 435 [M + H]⁺ | n.d. | 2.2 | 0.10 (B) |
| 3.87 | 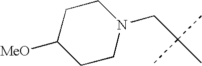 | 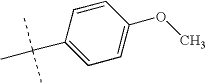 | CH | II.1 III.55 | 447 [M + H]⁺ | n.d. | 2.3 | 0.50 (B) |
| 3.88 | 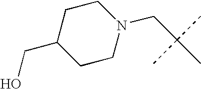 | 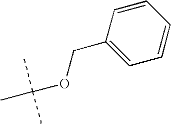 | CH | II.4 III.13 | 447 [M + H]⁺ | n.d. | 2.4 | 0.35 (B) |
| 3.89 | 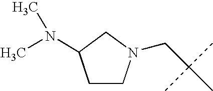 | 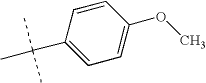 | CH | II.1 III.56 | 446 [M + H]⁺ | n.d. | 2.4 | 0.50 (B) |
| 3.90 | 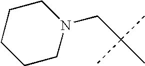 | 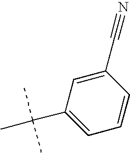 | CH | XIV.2 III.4 | 412 [M + H]⁺ | 120-122 | 2.4 | 0.30 (B) |
| 3.91 | 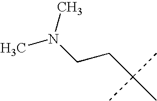 | 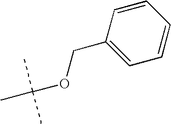 | CH | II.4 III.57 | 391 [M + H]⁺ | n.d. | 2.2 | 0.40 (B) |
| 3.92 | 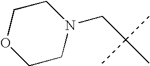 | 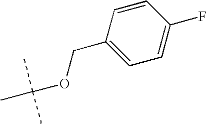 | CH | VI.6 III.11 | 419 [M + H]⁺ | n.d. | 2.4 | 0.40 (B) |
| 3.93 | 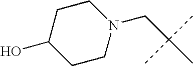 | 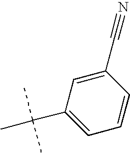 | CH | XIV.2 III.9 | 428 [M + H]⁺ | 120-122 | 2.2 | 0.60 (D) |
| 3.94 | 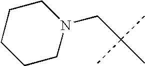 | 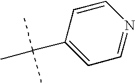 | CH | XIV.3 III.4 | 388 [M + H]⁺ | 135-138 | 1.8 | 0.50 (D) |
| 3.95 | 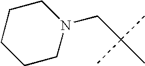 | 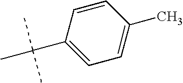 | CH | XIV.4 III.4 | 401 [M + H]⁺ | 128-130 | 2.5 | 0.50 (A) |
| 3.96 | 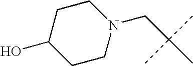 | 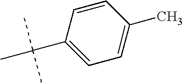 | CH | XIV.4 III.9 | 417 [M + H]⁺ | 133-135 | 2.3 | 0.20 (A) |
| 3.97 | 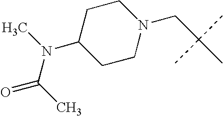 | 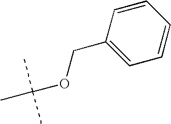 | CH | II.4 III.32 | 488 [M + H]⁺ | n.d. | 2.4 | 0.55 (B) |
| 3.98 | 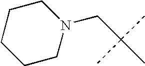 | 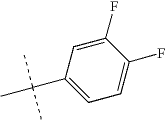 | CH | XIV.5 III.4 | 423 [M + H]⁺ | 104-106 | 2.5 | 0.40 (A) |
| 3.99 | 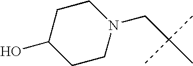 | 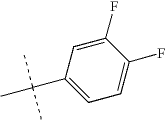 | CH | XIV.5 III.9 | 439 [M + H]⁺ | 80-82 | 2.54 | 0.40 (D) |
| 3.100 | 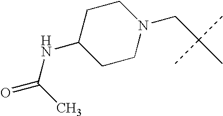 | 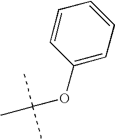 | CH | VI.7 III.21 | 460 [M + H]⁺ | 145-148 | 2.3 | 0.60 (D) |
| 3.101 | 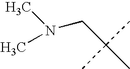 | 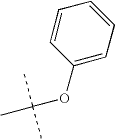 | CH | VI.7 III.3 | 363 [M + H]⁺ | n.d. | 2.3 | 0.30 (A) |
| 3.102 | 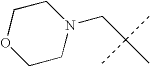 | 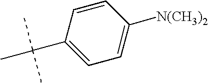 | CH | XIV.1 III.11 | 432 [M + H]⁺ | 143-145 | 2.3 | 0.70 (A) |
| 3.103 | 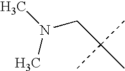 | 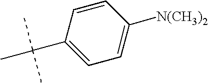 | CH | XIV.1 III.3 | 390 [M + H]⁺ | 135-137 | 2.2 | 0.40 (A) |
| 3.104 | 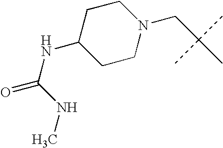 | 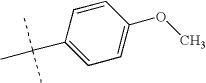 | CH | II.1 III.58 | 489 [M + H]⁺ | n.d. | 2.3 | 0.25 (B) |
| 3.105 | 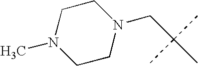 | 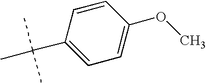 | CH | II.1 III.59 | 432 [M + H]⁺ | n.d. | 2.2 | 0.35 (B) |
| 3.106 | 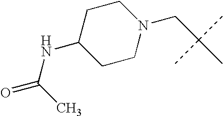 | 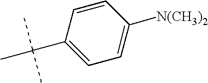 | CH | XIV.1 III.21 | 487 [M + H]⁺ | 176-178 | 2.3 | 0.80 (D) |
| 3.107 | 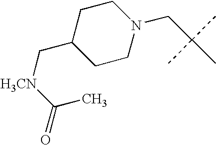 | 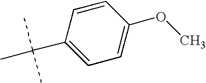 | CH | II.1 III.60 | 502 [M + H]⁺ | n.d. | 2.3 | 0.35 (B) |
| 3.108 | 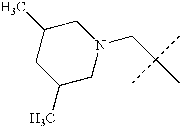 | 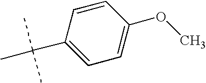 | CH | II.1 III.61 | 445 [M + H]⁺ | n.d. | 2.7 | 0.45 (B) |
| 3.109 | 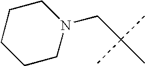 | 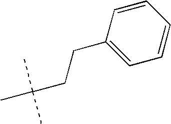 | CH | XVII.1 III.4 | 415 [M + H]⁺ | 80-85 | 2.5 | 0.40 (A) |
| 3.110 | 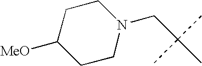 | 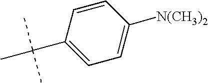 | CH | XIV.1 III.55 | 460 [M + H]⁺ | 120-122 | 2.3 | 0.40 (A) |
| 3.111 | 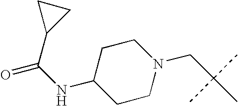 | 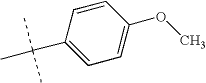 | CH | II.1 III.62 | 500 [M + H]⁺ | n.d. | 2.4 | 0.30 (B) |
| 3.112 | 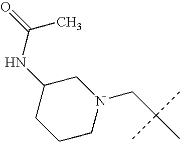 | 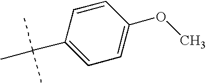 | CH | II.1 III.63 | 474 [M + H]⁺ | n.d. | 2.3 | 0.35 (B) |
| 3.113 | 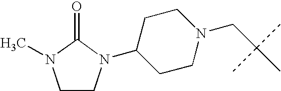 | 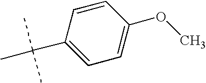 | CH | II.1 III.64 | 515 [M + H]⁺ | n.d. | 2.4 | 0.45 (B) |
| 3.114 | 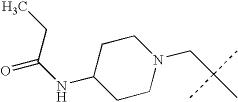 | 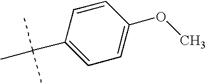 | CH | II.1 III.65 | 488 [M + H]⁺ | n.d. | 2.4 | 0.45 (B) |
| 3.115 | 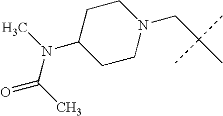 | 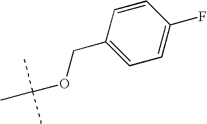 | CH | VI.6 III.32 | 506 [M + H]⁺ | 125-128 | 2.5 | 0.40 (A) |
| 3.116 | 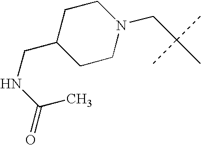 | 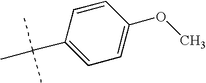 | CH | II.1 III.66 | 488 [M + H]⁺ | n.d. | 2.4 | 0.35 (B) |
| 3.117 | 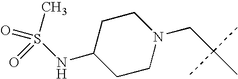 | 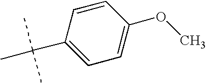 | CH | II.1 III.67 | 510 [M + H]⁺ | n.d. | 2.3 | 0.40 (B) |
| 3.118 | 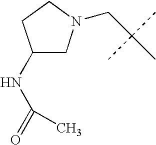 | 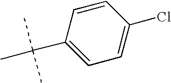 | CH | II.2 III.25 | 464/466 [M + H]⁺ | n.d. | 2.5 | 0.40 (B) |
| 3.119 | 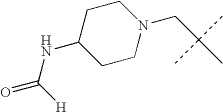 | 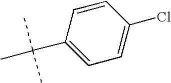 | CH | II.2 III.40 | 464/466 [M + H]⁺ | n.d. | 2.5 | 0.30 (B) |
| 3.120 | 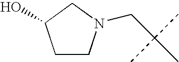 | 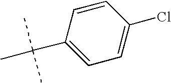 | CH | II.2 III.31 | 423/425 [M + H]⁺ | n.d. | 2.5 | 0.30 (B) |
| 3.121 | 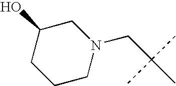 | 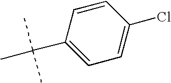 | CH | II.2 III.20 | 437/439 [M + H]⁺ | n.d. | 2.5 | 0.50 (B) |
| 3.122 | 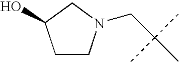 | 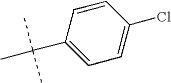 | CH | II.2 III.34 | 423/425 [M + H]⁺ | n.d. | 2.6 | 0.30 (B) |
| 3.123 | 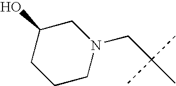 | 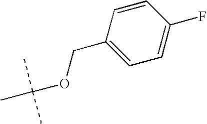 | CH | VI.6 III.20 | 451 [M + H]⁺ | 125-128 | 2.5 | 0.40 (A) |
| 3.124 | 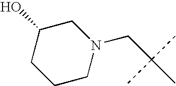 | 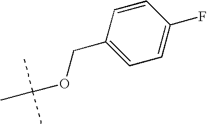 | CH | VI.6 III.21 | 451 [M + H]⁺ | 125-128 | 2.5 | 0.40 (A) |
| 3.125 | 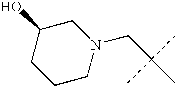 | 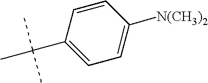 | CH | XIV.1 III.20 | 446 [M + H]⁺ | 123-125 | 2.3 | 0.60 (A) |
| 3.126 | 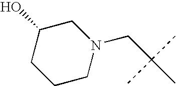 | 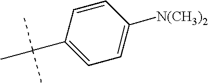 | CH | XIV.1 III.21 | 446 [M + H]⁺ | 125 | 2.3 | 0.60 (A) |
| 3.127 | 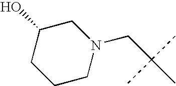 | 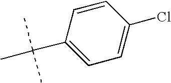 | CH | II.2 III.21 | 437/439 [M + H]⁺ | n.d. | 2.5 | 0.50 (B) |
| 3.128 | 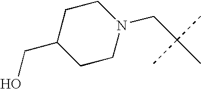 | 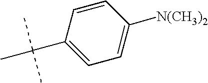 | CH | XIV.1 III.13 | 460 [M + H]⁺ | 135-138 | 2.2 | 0.30 (A) |
| 3.129 | 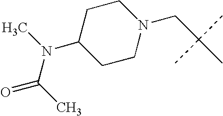 | 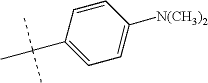 | CH | XIV.1 III.32 | 501 [M + H]⁺ | 140-142 | 2.3 | 0.40 (A) |
| 3.130 | 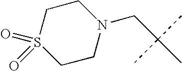 | 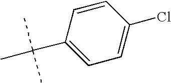 | CH | II.2 III.33 | 471/473 [M + H]⁺ | n.d. | 2.7 | 0.55 (B) |
| 3.131 | 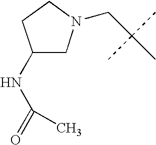 | 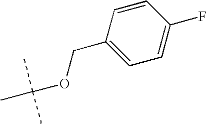 | CH | VI.6 III.25 | 478 [M + H]⁺ | 150-153 | 2.5 | 0.30 (A) |
| 3.132 | 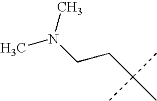 | 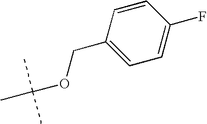 | CH | VI.6 III.57 | 409 [M + H]⁺ | 75-78 | 2.4 | 0.30 (A) |
| 3.133 | 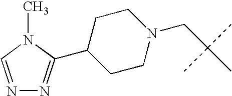 | 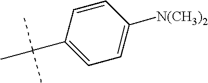 | CH | XIV.1 III.41 | 511 [M + H]⁺ | 155-160 | 2.2 | 0.60 (D) |
| 3.134 | 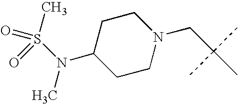 | 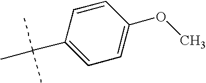 | CH | II.1 III.69 | 524 [M + H]⁺ | n.d. | 2.5 | 0.45 (B) |
| 3.135 | 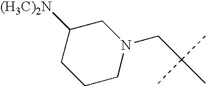 | 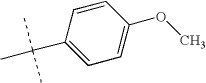 | CH | II.1 III.70 | 460 [M + H]⁺ | n.d. | 2.2 | 0.30 (B) |
| 3.136 | 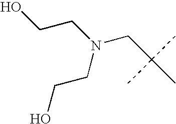 | 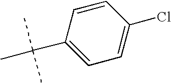 | CH | II.2 III.26 | 441/443 [M + H]⁺ | n.d. | 2.5 | 0.35 (B) |
| 3.137 | 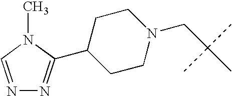 | 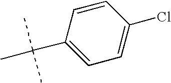 | CH | II.2 III.41 | 502/504 [M + H]⁺ | n.d. | 2.6 | 0.40 (B) |
| 3.138 | 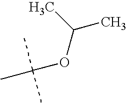 | 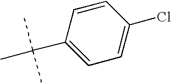 | CH | II.2 III.35 | 411/413 [M + H]⁺ | n.d. | 2.5 | 0.40 (B) |
| 3.139 | 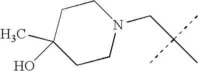 | 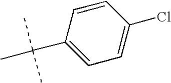 | CH | II.2 III.12 | 451/453 [M + H]⁺ | n.d. | 2.5 | 0.40 (B) |
| 3.140 | 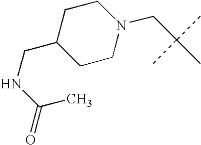 | 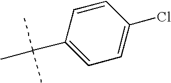 | CH | II.2 III.66 | 492/494 [M + H]⁺ | n.d. | 2.6 | 0.30 (B) |
| 3.141 | 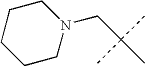 | 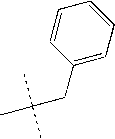 | CH | XVII.2 III.4 | 401 [M + H]⁺ | 104-106 | 2.5 | 0.40 (A) |
| 3.142 | 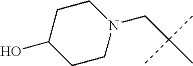 | 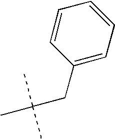 | CH | XVII.2 III.9 | 417 [M + H]⁺ | n.d. | 2.2 | 0.20 (A) |
| 3.143 | 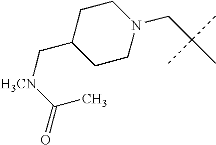 | 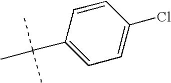 | CH | II.2 III.60 | 506/508 [M + H]⁺ | n.d. | 2.4 | 0.60 (B) |
| 3.144 | 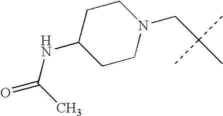 | 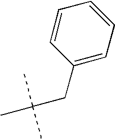 | CH | XVII.2 III.21 | 458 [M + H]⁺ | 128-130 | 2.2 | 0.40 (A) |
| 3.145 | 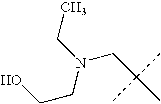 | 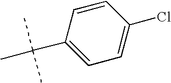 | CH | II.2 III.72 | 425/427 [M + H]⁺ | n.d. | 2.5 | 0.40 (B) |
| 3.146 | 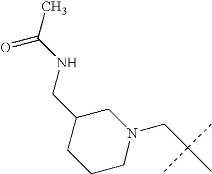 | 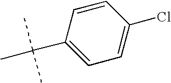 | CH | II.2 III.73 | 492/494 [M + H]⁺ | n.d. | 2.5 | 0.30 (B) |
| 3.147 | 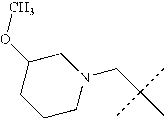 | 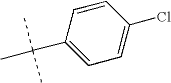 | CH | II.2 III.74 | 451/453 [M + H]⁺ | n.d. | 2.5 | 0.40 (M) |
| 3.148 | 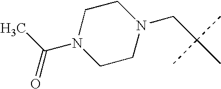 | 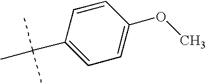 | CH | II.1 III.75 | 460 [M + H]⁺ | n.d. | 2.2 | 0.60 (B) |
| 3.149 | 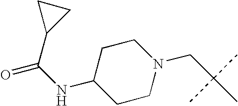 | 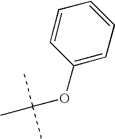 | CH | VI.7 III.62 | 486 [M + H]⁺ | 168-173 | 2.4 | 0.40 (N) |
| 3.150 | 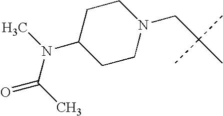 | 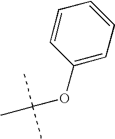 | CH | VI.7 III.32 | 474 [M + H]⁺ | 120-123 | 2.4 | 0.50 (N) |
| 3.151 | 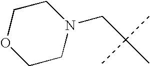 | 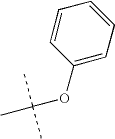 | CH | VI.7 III.11 | 405 [M + H]⁺ | 116-120 | 2.3 | 0.70 (N) |
| 3.152 | 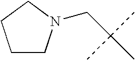 | 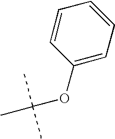 | CH | VI.7 III.23 | 389 [M + H]⁺ | 61-64 | 2.4 | 0.20 (A) |
| 3.153 | 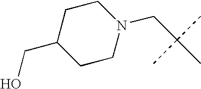 | 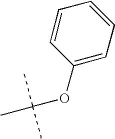 | CH | VI.7 III.13 | 433 [M + H]⁺ | n.d. | 2.3 | 0.60 (D) |
| 3.154 | 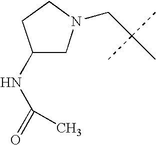 | 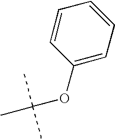 | CH | VI.7 III.25 | 446 [M + H]⁺ | n.d. | 2.1 | 0.30 (A) |
| 3.155 | 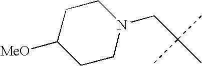 | 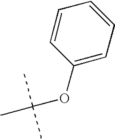 | CH | VI.7 III.55 | 433 [M + H]⁺ | n.d. | 2.5 | 0.40 (A) |
| 3.156 | 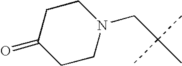 | 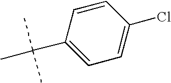 | CH | II.2 III.76 | 435/437 [M + H]⁺ | n.d. | 2.5 | 0.55 (B) |
| 3.157 | 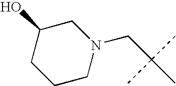 | 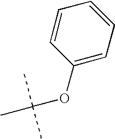 | CH | VI.7 III.20 | 419 [M + H]⁺ | 95-98 | 2.3 | 0.30 (A) |
| 3.158 | 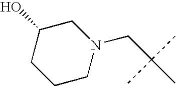 | 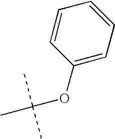 | CH | VI.7 III.21 | 419 [M + H]⁺ | 95-98 | 2.3 | 0.30 (A) |
| 3.159 | 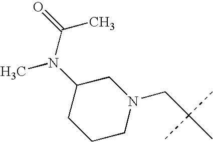 | 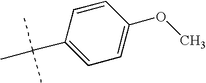 | CH | II.1 III.77 | 488 [M + H]⁺ | n.d. | 2.3 | 0.40 (B) |
| 3.160 | 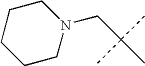 | 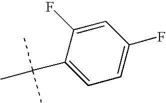 | CH | XIV.6 III.4 | 423 [M + H]⁺ | n.d. | 2.5 | 0.40 (B) |
| 3.161 | 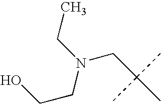 | 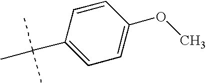 | CH | II.1 III.72 | 421 [M + H]⁺ | n.d. | 2.4 | 0.50 (B) |
| 3.162 | 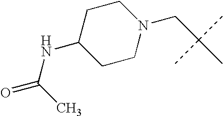 | 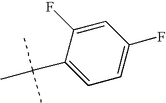 | CH | XIV.6 III.21 | 480 [M + H]⁺ | n.d. | 2.4 | n.d. |
| 3.163 | 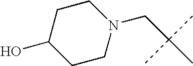 | 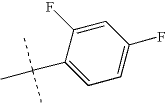 | CH | XIV.6 III.9 | 439 [M + H]⁺ | n.d. | 2.3 | n.d. |
| 3.164 | 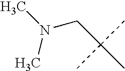 | 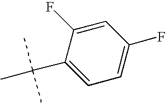 | CH | XIV.6 III.3 | 383 [M + H]⁺ | n.d. | 2.3 | n.d. |
| 3.165 | 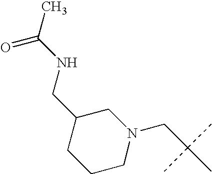 | 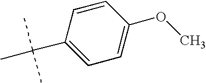 | CH | II.1 III.73 | 488 [M + H]⁺ | n.d. | 2.1 | 0.40 (B) |
| 3.166 | 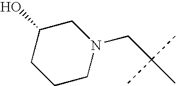 | 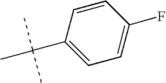 | CH | II.4 III.21 | 421 [M + H]⁺ | n.d. | 2.2 | 0.30 (B) |
| 3.167 | 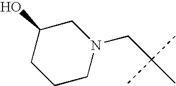 | 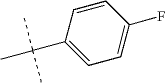 | CH | II.4 III.20 | 421 [M + H]⁺ | n.d. | 2.2 | 0.30 (B) |
| 3.168 | 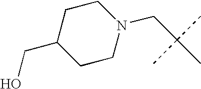 | 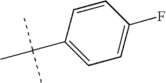 | CH | II.4 III.13 | 435 [M + H]⁺ | n.d. | 2.2 | 0.25 (B) |
| 3.169 | 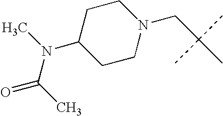 | 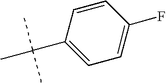 | CH | II.4 III.32 | 476 [M + H]⁺ | n.d. | 2.2 | 0.30 (B) |
| 3.170 | 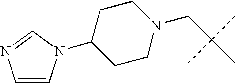 | 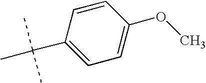 | CH | II.1 III.78 | 483 [M + H]⁺ | n.d. | 2.1 | 0.40 (B) |
| 3.171 | 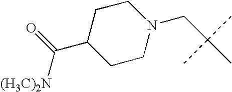 | 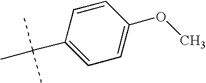 | CH | II.1 III.79 | 488 [M + H]⁺ | n.d. | 2.3 | 0.50 (B) |
| 3.172 | 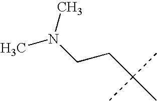 | 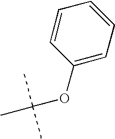 | CH | VI.7 III.75 | 377 [M + H]⁺ | 48-50 | 1.9 | 0.80 (D) |
| 3.173 | 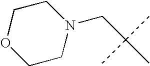 | 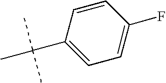 | CH | II.4 III.11 | 407 [M + H]⁺ | n.d. | 2.2 | n.d. |
| 3.174 | 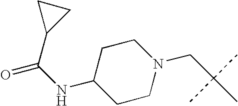 | 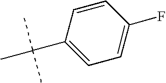 | CH | II.4 III.62 | 488 [M + H]⁺ | n.d. | 2.2 | n.d. |
| 3.175 | 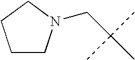 | 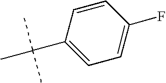 | CH | II.4 III.23 | 391 [M + H]⁺ | n.d. | 2.1 | n.d. |
| 3.176 | 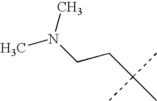 | 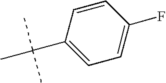 | CH | II.4 III.75 | 379 [M + H]⁺ | n.d. | 2.0 | n.d. |
| 3.177 | 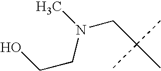 | 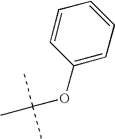 | CH | VI.7 III.44 | 393 [M + H]⁺ | n.d. | 2.2 | 0.60 (D) |
| 3.178 | 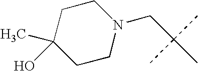 | 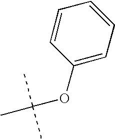 | CH | VI.7 III.12 | 433 [M + H]⁺ | n.d. | 2.2 | 0.60 (D) |
| 3.179 | 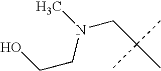 | 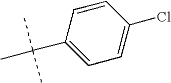 | CH | II.2 III.44 | 411/413 [M + H]⁺ | n.d. | 2.4 | 0.50 (B) |
| 3.180 | 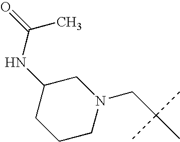 | 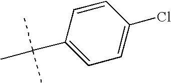 | CH | II.2 III.63 | 478/480 [M + H]⁺ | n.d. | 2.5 | 0.55 (B) |
| 3.181 | 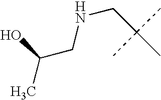 | 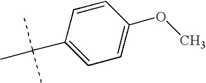 | CH | II.1 III.80 | 407 [M + H]⁺ | n.d. | 2.3 | 0.35 (B) |
| 3.182 | 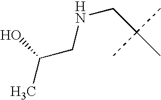 | 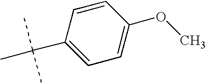 | CH | II.1 III.81 | 407 [M + H]⁺ | n.d. | 2.3 | 0.35 (B) |
| 3.183 | 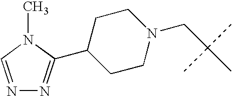 | 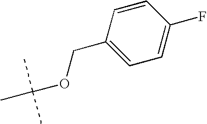 | CH | VI.6 III.41 | 516 [M + H]⁺ | 132-135 | 2.3 | 0.80 (D) |
| 3.184 | 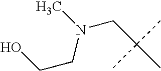 | 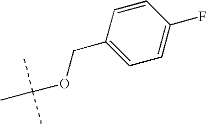 | CH | VI.6 III.44 | 425 [M + H]⁺ | n.d. | 2.2 | 0.40 (A) |
| 3.185 | 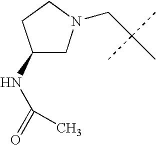 | 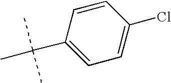 | CH | II.2 III.82 | 464/466 [M + H]⁺ | n.d. | 2.3 | n.d. |
| 3.186 | 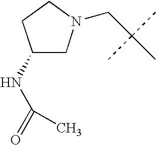 | 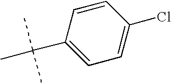 | CH | II.2 III.83 | 464/466 [M + H]⁺ | n.d. | 2.3 | n.d. |
| 3.187 | 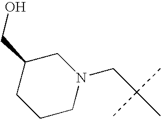 | 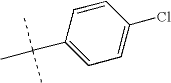 | CH | II.2 III.84 | 451/453 [M + H]⁺ | n.d. | 2.4 | 0.55 (B) |
| 3.188 | 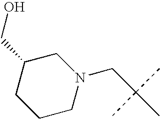 | 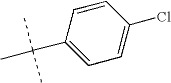 | CH | II.2 III.85 | 451/453 [M + H]⁺ | n.d. | 2.4 | 0.55 (B) |
| 3.189 | 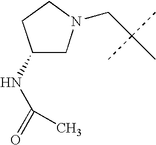 | 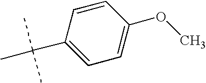 | CH | II.1 III.83 | 460 [M + H]⁺ | n.d. | 2.1 | n.d. |
| 3.190 | 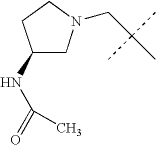 | 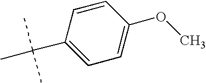 | CH | II.1 III.82 | 460 [M + H]⁺ | n.d. | 2.1 | n.d. |
| 3.191 | 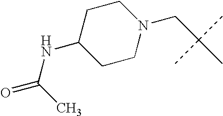 | 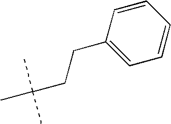 | CH | XVII.1 III.21 | 472 [M + H]⁺ | 166-168 | 2.1 | 0.30 (A) |
| 3.192 | 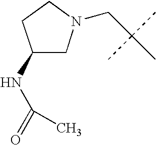 | 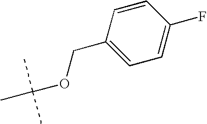 | CH | VI.6 III.82 | 478 [M + H]⁺ | n.d. | 2.3 | 0.45 (B) |
| 3.193 | 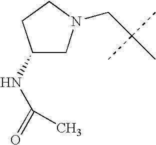 | 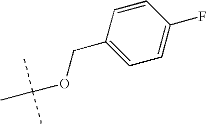 | CH | VI.6 III.83 | 478 [M + H]⁺ | n.d. | 2.3 | 0.45 (B) |
| 3.194 | 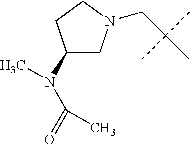 | 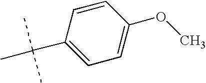 | CH | II.1 III.86 | 474 [M + H]⁺ | n.d. | 2.2 | n.d. |
| 3.195 | 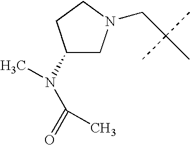 | 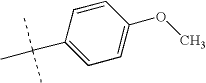 | CH | II.1 III.87 | 474 [M + H]⁺ | n.d. | 2.2 | n.d. |
| 3.196 | 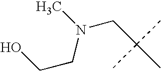 | 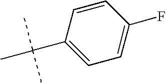 | CH | II.4 III.44 | 395 [M + H]⁺ | n.d. | 2.3 | n.d. |
| 3.197 | 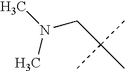 | 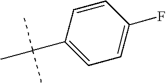 | CH | II.4 III.3 | 365 [M + H]⁺ | n.d. | 2.1 | n.d. |
| 3.198 | 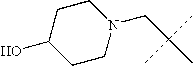 | 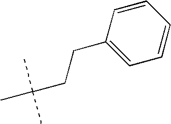 | CH | XVII.1 III.9 | 431 [M + H]⁺ | n.d. | 2.2 | 0.30 (A) |
| 3.199 | 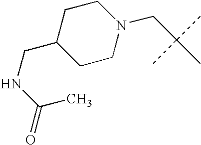 | 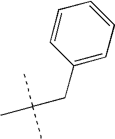 | CH | XVII.2 III.66 | 472 [M + H]⁺ | n.d. | 2.1 | 0.50 (B) |
| 3.200 | 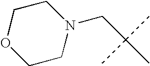 | 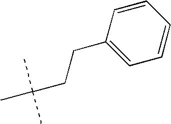 | CH | XVII.1 III.11 | 417 [M + H]⁺ | n.d. | 2.3 | 0.50 (A) |
| 3.201 | 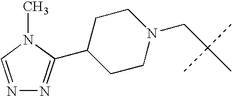 | 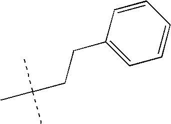 | CH | XVII.1 III.41 | 496 [M + H]⁺ | 156-158 | 2.1 | 0.20 (A) |
| 3.202 | 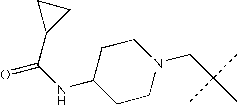 | 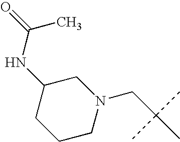 | CH | XVII.1 III.62 | 498 [M + H]⁺ | 138-142 | 2.3 | 0.40 (A) |
| 3.203 | 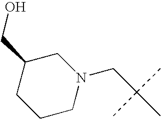 | 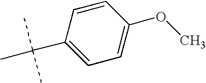 | CH | II.1 III.84 | 447 [M + H]⁺ | n.d. | 2.2 | 0.25 (B) |
| 3.204 | 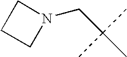 | 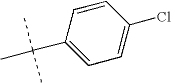 | CH | II.2 III.45 | 393/395 [M + H]⁺ | n.d. | 2.3 | 0.65 (B) |
| 3.205 | 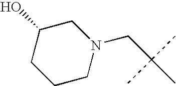 | 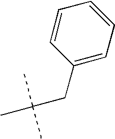 | CH | XVII.2 III.21 | 417 [M + H]⁺ | n.d. | 2.2 | 0.45 (B) |
| 3.206 | 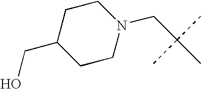 | 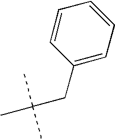 | CH | XVII.2 III.13 | 431 [M + H]⁺ | n.d. | 2.1 | 0.30 (B) |
| 3.207 | 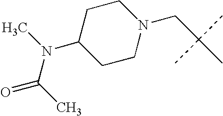 | 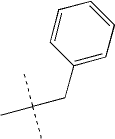 | CH | XVII.2 III.32 | 472 [M + H]⁺ | n.d. | 2.3 | 0.40 (B) |
| 3.208 | 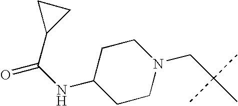 | 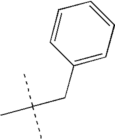 | CH | XVII.2 III.62 | 484 [M + H]⁺ | n.d. | 2.5 | 0.50 (B) |
| 3.209 | 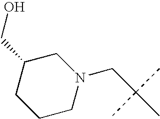 | 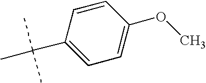 | CH | II.1 III.85 | 447 [M + H]⁺ | n.d. | 2.2 | n.d. |
| 3.210 | 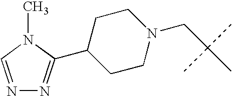 | 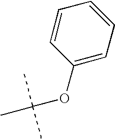 | CH | VI.7 III.41 | 484 [M + H]⁺ | n.d. | 2.3 | 0.50 (D) |
| 3.211 | 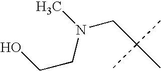 | 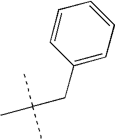 | CH | XVII.2 III.44 | 391 [M + H]⁺ | n.d. | 2.0 | 0.60 (B) |
| 3.212 | 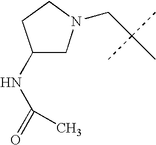 | 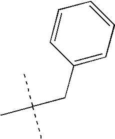 | CH | XVII.2 III.25 | 444 [M + H]⁺ | n.d. | 2.0 | 0.55 (B) |
| 3.213 | 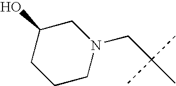 | 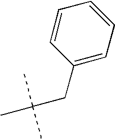 | CH | XVII.2 III.20 | 417 [M + H]⁺ | n.d. | 2.2 | 0.50 (B) |
| 3.214 | 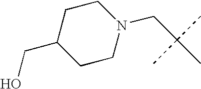 | 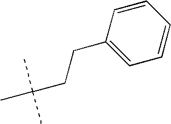 | CH | XVII.1 III.13 | 445 [M + H]⁺ | 110-113 | 2.2 | 0.30 (A) |
| 3.215 | 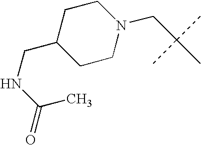 | 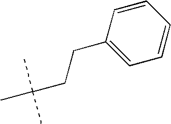 | CH | XVII.1 III.66 | 486 [M + H]⁺ | 111-115 | 2.2 | 0.30 (A) |
| 3.216 | 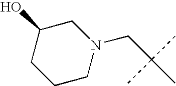 | 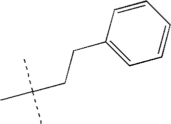 | CH | XVII.1 III.20 | 431 [M + H]⁺ | 118-121 | 2.2 | 0.30 (A) |
| 3.217 | 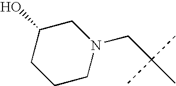 | 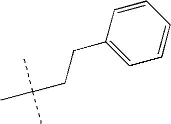 | CH | XVII.1 III.21 | 431 [M + H]⁺ | 119-121 | 2.2 | 0.30 (A) |
| 3.218 | 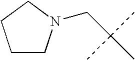 | 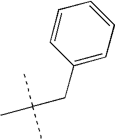 | CH | XVII.2 III.23 | 387 [M + H]⁺ | n.d. | 2.2 | 0.60 (B) |
| 3.219 | 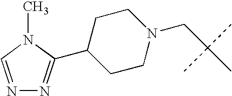 | 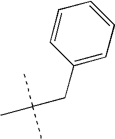 | CH | XVII.2 III.41 | 482 [M + H]⁺ | n.d. | 2.3 | 0.40 (B) |
| 3.220 | 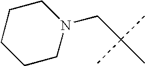 | 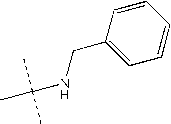 | CH | XII.1 III.4 | 416 [M + H]⁺ | 98-102 | 2.2 | 0.70 (D) |
| 3.221 | 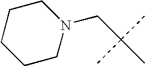 | 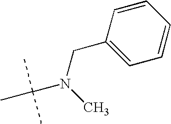 | CH | XII.2 III.4 | 430 [M + H]⁺ | n.d. | 2.1 | 0.30 (A) |
| 3.222 | 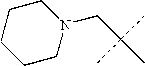 | 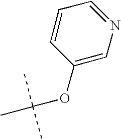 | CH | XIII.1 III.4 | 404 [M + H]⁺ | n.d. | 2.1 | 0.50 (D) |
| 3.223 | 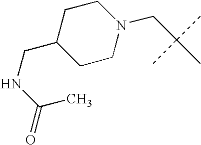 | 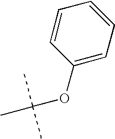 | CH | VI.7 III.66 | 474 [M + H]⁺ | n.d. | 2.1 | n.d. |
| 3.224 | 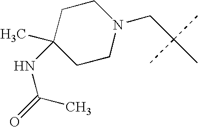 | 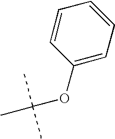 | CH | VI.7 III.88 | 474 [M + H]⁺ | n.d. | 2.1 | n.d. |
| 3.225 | 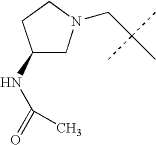 | 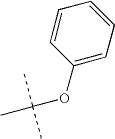 | CH | VI.7 III.82 | 446 [M + H]⁺ | n.d. | 2.1 | n.d. |
| 3.226 | 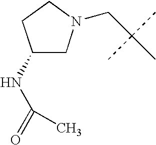 | 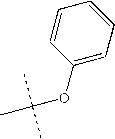 | CH | VI.7 III.83 | 446 [M + H]⁺ | n.d. | 2.1 | n.d. |
| 3.227 | 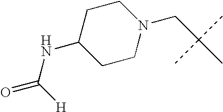 | 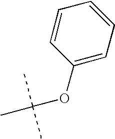 | CH | VI.7 III.40 | 446 [M + H]⁺ | n.d. | 2.1 | n.d. |
| 3.228 | 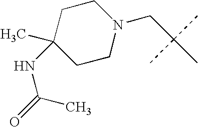 | 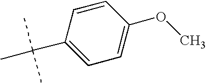 | CH | II.1 III.88 | 488 [M + H]⁺ | n.d. | 2.1 | n.d. |
| 3.229 | 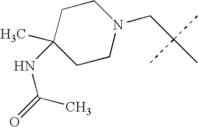 | 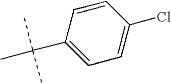 | CH | II.2 III.88 | 492/494 [M + H]⁺ | n.d. | 2.3 | n.d. |
| 3.230 | 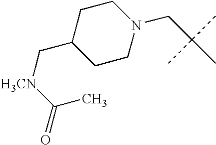 | 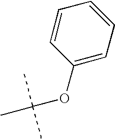 | CH | VI.7 III.60 | 488 [M + H]⁺ | n.d. | 2.2 | n.d. |
| 3.231 | 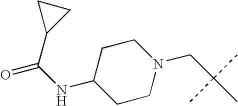 | 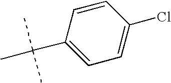 | CH | II.2 III.62 | 504/506 [M + H]⁺ | n.d. | 2.3 | n.d. |
| 3.232 | 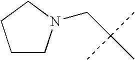 | 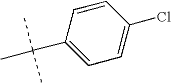 | CH | II.2 III.23 | 407/409 [M + H]⁺ | n.d. | 2.3 | n.d. |

*HPLC method A

The following compounds are prepared analogously:

### 3.233

### {3-[6-(4-Methoxy-phenyl)-pyridazin-3-yl]-propyl}-(2-methyl-2,3-dihydro-1H-isoindol-5-yl)-amine

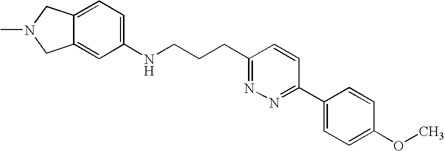 from educts II.1/XVI.1

R_{f} value: 0.60 (silica gel, methylene chloride/methanol/ammonia=9:1:0.1)

retention time (HPLC): 2.3 min (method A)

C₂₃H₂₆N₄O

EII mass spectrum: m/z=375 [M+H]⁺

### 3.234

### 1-(4-{3-[6-(4-Chloro-phenyl)-pyridazin-3-yl]-propylamino}-2-methoxy-benzyl)-piperidin-4-ol

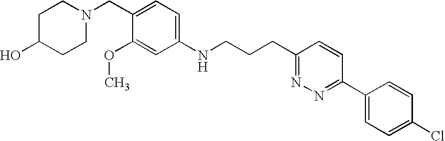 from educts II.2/III.68

R_{f} value: 0.20 (silica gel, methylene chloride/methanol/ammonia=9:1:0.1)

retention time (HPLC): 2.5 min (method A)

C₂₆H₃₁ ClN₄O₂

EII mass spectrum: m/z=467/469 [M+H]⁺

### 3.235

### 1-(4-{3-[6-(4-Chloro-phenyl)-pyridazin-3-yl]-propylamino}-2-fluoro-benzyl)-pipieridin-4-ol

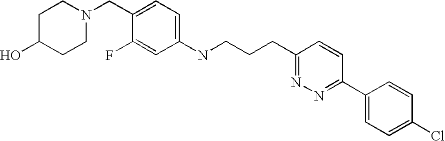 from educts II.2/III.71

R_{f} value: 0.30 (silica gel, methylene chloride/methanol/ammonia=9:1:0.1)

retention time (HPLC): 2.5 min (method A)

C₂₅H₂₈ClFN₄O

EII mass spectrum: m/z=455/457 [M+H]⁺

### 3.236

### {3-[6-(4-Methoxy-phenyl)-pyridazin-3-yl]-propyl}-(2-methyl-1,2,3,4-tetrahydro-isoquinolin-6-yl)amine

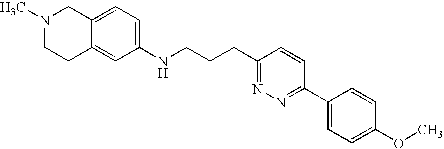 from educt II.1 and 6-bromo-2-methyl-1,2,3,4-tetrahydro-isoquinoline (J. Chem. Soc., Perkin Transactions 1, 1976, 757)

R_{f} value: 0.70 (silica gel, methylene chloride/methanol/ammonia=9:1:0.01)

retention time (HPLC): 2.1 min (method A)

M.p. 163-166° C.

C₂₄H₂₈N₄O

EII mass spectrum: m/z=389 [M+H]⁺

### EXAMPLE 4

The following compounds of general formula IV-1 are prepared analogously to Example 3.1, the educts used being shown in the column headed “Educts”:

| | |
|---|---|
| | (IV-1) |
| 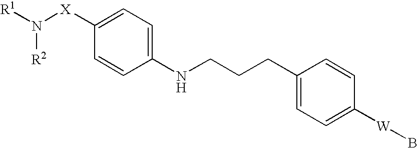 | |

| Exam- | | | | mass | M.p. | |
|---|---|---|---|---|---|---|
| ple | R¹R²N-X- | -W-B | Educts | spectrum | [° C.] | R_{f}-value |
| 4.1 | 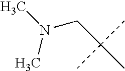 | 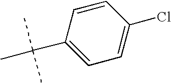 | I.1 III.3 | 379/381 [M + H]⁺ | 85-90 | 0.70 (D) |
| 4.2 | 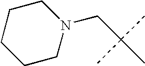 | 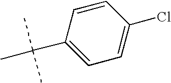 | I.1 III.4 | 419/421 [M + H]⁺ | 100-103 | 0.40 (A) |
| 4.3 | 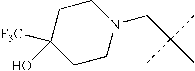 | 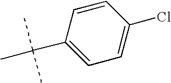 | I.1 III.8 | 503/505 [M + H]⁺ | n.d. | 0.60 (A) |

### EXAMPLE 5.1

### 1-{6-[3-(4′-Chloro-biphenyl-4-yl)-propylamino]-pyridin-3-ylmethyl}-4-methyl-piperidin-4-ol

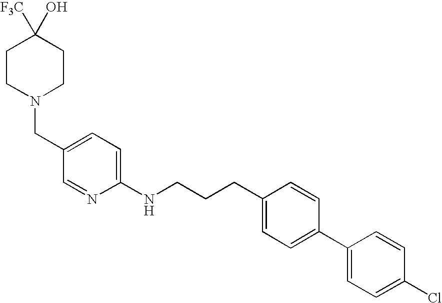

400 mg (1.63 mmol) 3-(4′-Chloro-biphenyl-4-yl)-propylamine (educt I.1) and 386 mg (1.00 mmol) 1-(6-iodo-pyridin-3-ylmethyl)-4-trifluoromethyl-piperidin-4-ol (educt IV. 1) are dissolved in 1.0 ml of DMF and 190 mg (1.00 mmol) copper-(I)-iodide and 480 mg (2.5 mmol) cesium acetate are added. The mixture is stirred for 20 hours at 90° C. in a sealed tube under argon atmosphere. After cooling, ethyl acetate and water are added. The organic phase is separated and dried over sodium sulphate. The solvent is evaporated and the product is purified by silica gel column chromatography with methylene chloride/methanol/ammonia (9:1:0.01) as eluent.

Yield: 120 mg (24% of theory),

R_{f} value: 0.50 (silica gel, methylene chloride/methanol/ammonia=9:1:0.01)

M.p. 170-172° C.

C₂₇H₂₉ClF₃N₃O

EII mass spectrum: m/z=504/506 [M+H]⁺

The following compounds of general formula V-1 are prepared analogously to Example 5.1, the educts used being shown in the column headed “Educts”:

| | |
|---|---|
| | (V-1) |
| 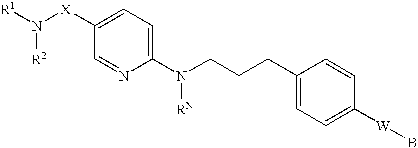 | |

| Exam- | | | | | mass | M.p. | |
|---|---|---|---|---|---|---|---|
| ple | R¹R²N-X- | -W-B | R^{N} | Educts | spectrum | [° C.] | R_{f}-value |
| 5.2 | 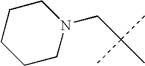 | 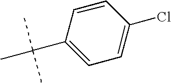 | —H | I.1 IV.2 | 420/422 [M + H]⁺ | 102-105 | 0.80 (E) |
| 5.3 | 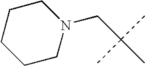 | 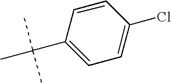 | —CH₃ | I.2 IV.2 | 434/436 [M + H]⁺ | n.d. | 0.50 (A) |
| 5.4 | 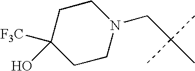 | 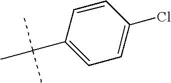 | —CH₃ | I.2 IV.1 | 447/449 [M + H]⁺ | n.d. | 0.40 (A) |

### EXAMPLE 6.1

### [3-(4′-Chloro-biphenyl-4-yl)-propyl]-[5-(4-methyl-piperidin-1-ylmethyl)-pyridin-2-yl]-amine

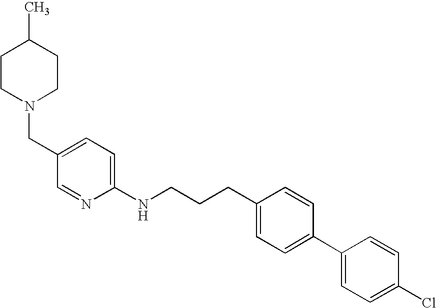

140 mg (0.57 mmol) 3-(4′-Chloro-biphenyl-4-yl)-propylamine (educt 1.1) and 128 mg (0.57 mmol) 1-(6-chloro-pyridin-3-ylmethyl)-4-methyl-piperidin (educt III.6) are dissolved in 3.0 ml of toluene and 6 mg (0.02 mmol) 2-(di-tert-butylphosphino)biphenyl, 1.3 mg (0.006 mmol) palladium(II) acetate and 77 mg (0.80 mmol) sodium tert-butoxide are added. The mixture is stirred for 15 hours at 110° C. in a sealed tube under argon atmosphere. After cooling, water and ethyl acetate are added. The organic phase is separated and dried over sodium sulphate. The solvent is evaporated and the product is purified by silica gel column chromatography with methylene chloride/methanol/ammonia (9:1:0.01) as eluent.

Yield: 18 mg (7% of theory),

R_{f} value: 0.40 (silica gel, methylene chloride/methanol/ammonia=9:1:0.01)

C₂₇H₃₂ClN₃

EII mass spectrum: m/z=434/436 [M+H]⁺

### EXAMPLE 7

The following compounds of general formula VII-1 are prepared analogously to Example 5.1, the educts used being shown in the column headed “Educts”:

| | |
|---|---|
| | (VII-1) |
| 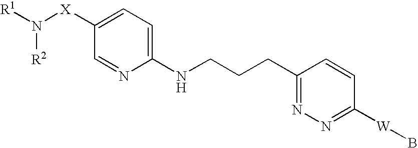 | |

| Exam- | | | | mass | M.p. | |
|---|---|---|---|---|---|---|
| ple | R¹R²N-X- | -W-B | Educts | spectrum | [° C.] | R_{f}-value |
| 7.1 | 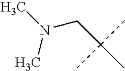 | 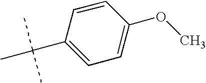 | II.1 IV.3 | 378 [M + H]⁺ | 120-122 | 0.40 (E) |
| 7.2 | 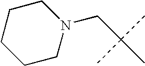 | 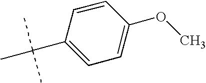 | II.1 IV.2 | 418 [M + H]⁺ | 140-145 | 0.50 (E) |

### EXAMPLE 8.1

### 1-(4-{3-[5-(4-Chloro-phenyl)-pyridin-2-ylamino]-propyl}-benzyl)-pipieridin-4-ol

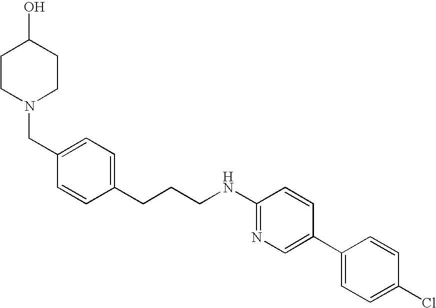

176 mg (0.50 mmol) (4-{3-[5-(4-Chloro-phenyl)-pyridin-2-ylamino]-propyl}-phenyl)-methanol (educt VII.1) and 0.10 ml (0.60 mmol) N-ethyl diisopropylamine are dissolved in 5.0 ml of THF and 0.05 ml (0.60 mmol) methane sulfonyl chloride are added at RT. After stirring for 2 hours at RT, 101 mg (1.00 mmol) 4-hydroxy-piperidine are added and the mixture is stirred for additional 10 hours at RT. After that time, water and ethyl acetate are added. The organic phase is separated, washed with water and dried over sodium sulphate. The solvent is evaporated and the product is purified by silica gel column chromatography with ethyl acetate/methanol/ammonia (9:1:0.1) as eluent.

Yield: 124 mg (57% of theory),

R_{f} value: 0.40 (silica gel, ethyl acetate/methanol/ammonia=9:1:0.1)

C₂₆H₃₀ClN₃O

EII mass spectrum: m/z=436/438 [M+H]⁺

The following compounds of general formula VIII-1 are prepared analogously to Example 8.1, the educts used being shown in the column headed “Educts”:

| | |
|---|---|
| | (VIII-1) |
| 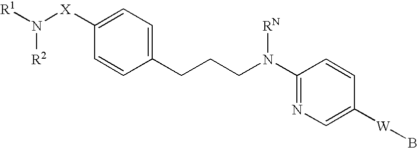 | |

| | | | | | | | ret. time | |
|---|---|---|---|---|---|---|---|---|
| Exam- | | | | | mass | M.p. | (HPLC) | |
| ple | R¹R²N-X- | -W-B | R^{N} | Educts | spectrum | [° C.] | [min]* | R_{f}-value |
| 8.2 | 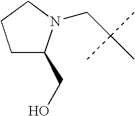 | 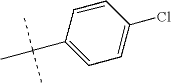 | —H | VII.1 | 436/438 [M + H]⁺ | n.d. | 2.7 (B) | 0.50 (F) |
| 8.3 | 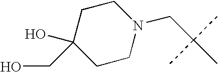 | 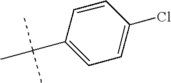 | —H | VII.1 | 466/468 [M + H]⁺ | n.d. | 2.7 (B) | 0.10 (F) |
| 8.4 | 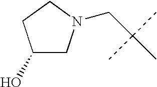 | 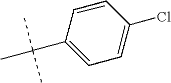 | —H | VII.1 | 422/424 [M + H]⁺ | n.d. | 2.7 (B) | 0.50 (G) |
| 8.5 | 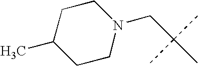 | 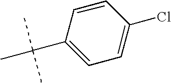 | —H | VII.1 | 434 [M + H]⁺ | n.d. | 7.4 (C) | n.d. |
| 8.6 | 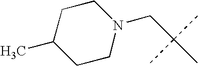 | 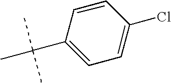 | —CH₃ | VIII.1 | 448/450 [M + H]⁺ | n.d. | 2.9 (B) | 0.25 (H) |

### EXAMPLE 9

The following compounds of general formula IX-1 are prepared analogously to Example 8.1, the educts used being shown in the column headed “Educts”:

| | | | | | | | (IX-1) |
|---|---|---|---|---|---|---|---|
| 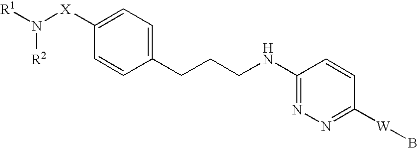 | | | | | | | |
| | | | | | | ret. time | |
| Exam- | | | | mass | M.p. | (HPLC) | |
| ple | R¹R²N-X- | -W-B | Educts | spectrum | [° C.] | [min]* | R_{f}-value |
| 9.1 | 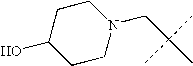 | 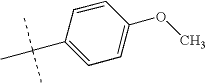 | VII.2 | 433 [M + H]⁺ | n.d. | 2.9 | n.d. |
| 9.2 | 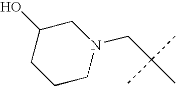 | 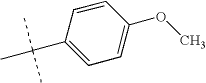 | VII.2 | 433 [M + H]⁺ | n.d. | 3.0 | n.d. |

*HPLC method B

The following compounds of general formula IX-2 are prepared analogously to Example 8.1, the educts used being shown in the column headed “Educts”:

| | | | | | | | (IX-2) |
|---|---|---|---|---|---|---|---|
| 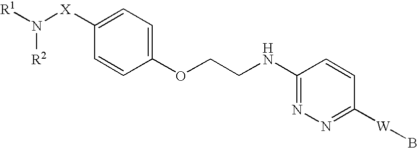 | | | | | | | |
| | | | | | | ret. time | |
| Exam- | | | | mass | M.p. | (HPLC) | |
| ple | R¹R²N-X- | -W-B | Educts | spectrum | [° C.] | [min]* | R_{f}-value |
| 9.3 | 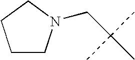 | 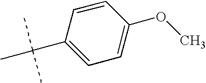 | VII.3 | 405 [M + H]⁺ | n.d. | 2.6 | n.d. |

*HPLC method A

### EXAMPLE 10.1

### [3-Chloro-4-(2-diethylamino-ethoxy)-phenyl]-[2-(2-chloro-4-trifluoromethyl-phenoxy)-ethyl]-amine

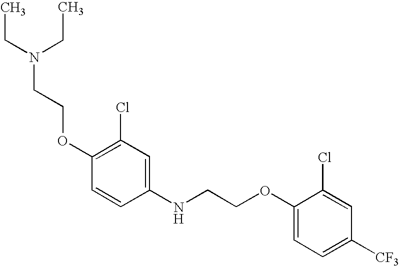

0.40 g (0.84 mmol) N-[3-Chloro-4-(2-diethylamino-ethoxy)-phenyl]-2-(2-chloro-4-trifluoromethyl-phenoxy)-acetamide (educt IX.1) are dissolved in 20 ml of THF and 4.0 ml of 1M borane-THF complex are added at RT. After stirring for 3 hours at RT, the solvent is evaporated. The residue is taken up in methanol and 0.5 ml conc. HCl are added. After stirring for 10 minutes at 1 00° C. the solvent is evaporated. The residue is taken up in 50 ml methylene chloride and 5.0 g sodium carbonate are added. The solution is filtered and the filtrate is evaporated. The residue is purified by aluminum oxide column chromatography with methylene chloride/methanol (8:2) as eluent.

Yield: 330 mg (85% of theory),

R_{f} value: 0.70 (aluminum oxide, methylene chloride/methanol=50:1)

C₂₁H₂₅Cl₂F₃N₂O₂

EII mass spectrum: m/z=465/467/469 [M+H]⁺

The following compounds of general formula X-1 are prepared analogously to Example 10.1, the educts used being shown in the column headed “Educts”:

| | |
|---|---|
| | (X-1) |
| 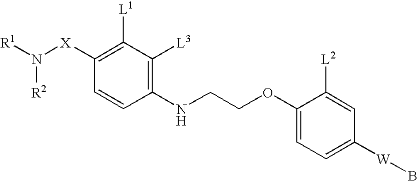 | |

| Exam- | | | | | | | mass | M.p. | |
|---|---|---|---|---|---|---|---|---|---|
| ple | R¹R²N-X- | -W-B | L¹ | L² | L³ | Educts | spectrum | [° C.] | R_{f}-value |
| 10.2 | 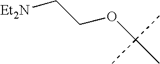 | 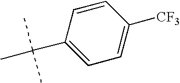 | —H | —Cl | —NMe₂ | IX.3 | 474/476 [M + H]⁺ | n.d. | 0.75 (K) |
| 10.3 | 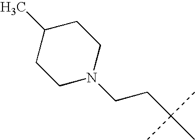 | 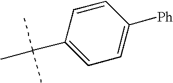 | —Br | —Br | —H | IX.4 | 571/573/ 575 [M + H]⁺ | n.d. | 0.65 (I) |
| 10.4 | 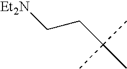 | 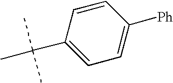 | —Br | —Br | —H | IX.5 | 545/547/ 549 [M + H]⁺ | n.d. | 0.55 (K) |

### EXAMPLE 11

The following compounds of general formula XI-1 are prepared analogously to Example 10.1, the educts used being shown in the column headed “Educts”:

| | |
|---|---|
| | (XI-1) |
| 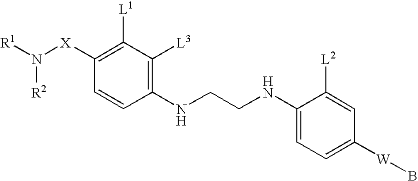 | |

| Exam- | | | | | | | mass | M.p. | |
|---|---|---|---|---|---|---|---|---|---|
| ple | R¹R²N-X- | -W-B | L¹ | L² | L³ | Educts | spectrum | [° C.] | R_{f}-value |
| 11.1 | 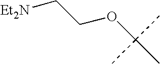 | 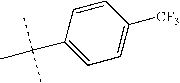 | —Cl | —Cl | —H | IX.2 | 464/468/ 470 [M + H]⁺ | n.d. | 0.45 (K) |

### EXAMPLE 12.1

### (2-{2-Chloro-4-[2-(2-chloro-4-iodo-phenoxy)-ethoxy]-phenoxyl}-ethyl)-diethyl-amine

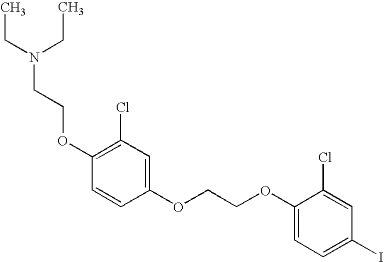

0.16 g (0.66 mmol) 3-Chloro-4-(2-diethylamino-ethoxy)-phenol (educt X.1) and 97 mg (0.70 mmol) potassium carbonate are dissolved in 20 ml of DMF and stirred for 45 minutes at 60° C. After that time 0.26 g (0.69 mmol) methanesulfonic acid 2-(2-chloro-4-iodo-phenoxy)-ethyl ester (educt XI.1) are added and the mixture is strirred for 10 hours at 80° C. After cooling the mixture is filtered and the filtrate is poured into 100 ml water. The solution is extracted with ethyl acetate and the organic phase is dried over sodium sulphate. The solvent is evaporated and the residue is purified by silica gel column chromatography with methylene chloride/methanol (9:1) as eluent.

Yield: 90 mg (26% of theory),

R_{f} value: 0.35 (silica gel, methylene chloride/methanol=9:1)

C₂₀H₂₄Cl₂NO₃

EII mass spectrum: m/z=524/526 [M+H]⁺

### EXAMPLE 13.1

### 1-(4-{2-[6-(4-Methoxy-phenyl)-pyridazin-3-yloxyl-ethoxy}-benzyl)-4-methyl-pipieridin-4-ol

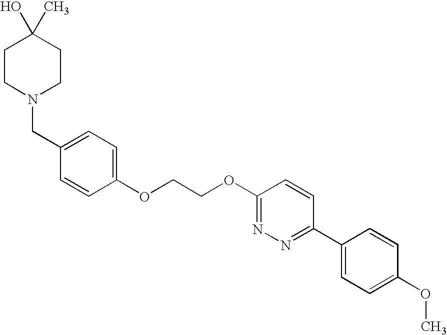

100 mg (0.285 mmol) 4-{2-[6-(4-Methoxy-phenyl)-pyridazin-3-yloxy]-ethoxy}-benzaldehyde (educt XII.1) and 35 mg (0.30 mmol) 4-methyl-piperidin-4-ol are dissolved in 10 ml of THF and 0.20 ml conc. acetic acid are added. After 10 minutes 180 mg (0.855 mmol) sodium triacetoxyborohydride are added and the mixture is stirred for 20 hours at RT. After that time the mixture is filtered and the solvent is evaporated. The residue is purified by silica gel column chromatography with methylene chloride/methanol/ammonia (10:1:0.1) as eluent.

Yield: 50 mg (39% of theory),

R_{f} value: 0.25 (silica gel, methylene chloride/methanol/ammonia=10:1:0.1)

C₂₆H₃₁ N₃O₄

EII mass spectrum: m/z=450 [M+H]⁺

The following compounds of general formula XIII-1 are prepared analogously to Example 13.1, the educts used being shown in the column headed “Educts”:

| | | | | | | | (XIII-1) |
|---|---|---|---|---|---|---|---|
| 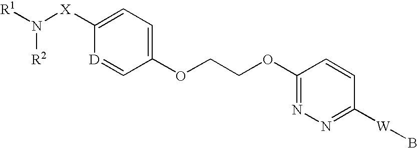 | | | | | | | |
| | | | | | | ret. time | |
| Exam- | | | | | mass | (HPLC) | |
| ple | R¹R²N-X- | -W-B | D | Educts | spectrum | [min]* | R_{f}-value |
| 13.2 | 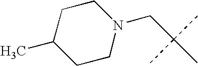 | 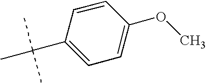 | CH | XII.1 | 434 [M + H]⁺ | 2.6 | n.d. |
| 13.3 | 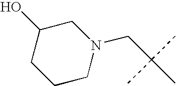 | 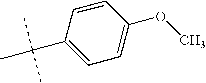 | CH | XII.1 | 436 [M + H]⁺ | 2.4 | n.d. |
| 13.4 | 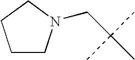 | 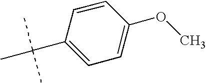 | CH | XII.1 | 406 [M + H]⁺ | 2.5 | n.d. |
| 13.5 | 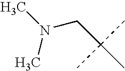 | 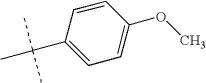 | CH | XII.1 | 380 [M + H]⁺ | 2.4 | n.d. |

*HPLC method A

The following compounds of general formula XIII-2 are prepared analogously to Example 13.1, the educts used being shown in the column headed “Educts”:

| | | | | | | | (XIII-2) |
|---|---|---|---|---|---|---|---|
| 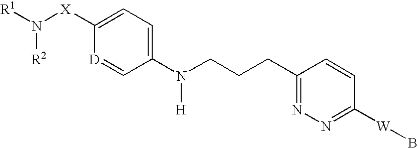 | | | | | | | |
| | | | | | | ret. time | |
| Exam- | | | | | mass | (HPLC) | |
| ple | R¹R²N-X- | -W-B | D | Educts | spectrum | [min]* | R_{f}-value |
| 13.6 | 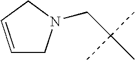 | 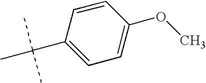 | CH | XX.1 | 401 [M + H]⁺ | 2.2 | 0.65 (B) |

*HPLC method A

### EXAMPLE 14.1

3-Chloro-4-{2-[3-chloro-4-(2-diethylamino-ethoxy)-phenoxy]-ethoxy}-benzonitrile

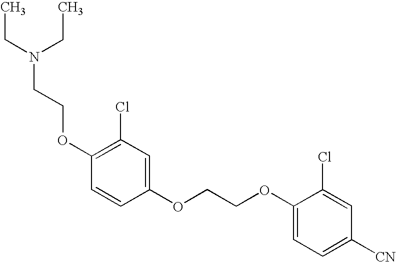

100 mg (0.285 mmol) {2-[4-(2-Bromo-ethoxy)-2-chloro-phenoxy]-ethyl}-diethyl-amine (educt XIII.1), 45 mg (0.29 mmol) 3-chloro-4-hydroxy-benzonitrile and 100 mg (0.724 mmol) potassium carbonate are dissolved in 5 ml of DMF. The mixture is stirred for 2 hours at 80° C. After that time the mixture is filtered and the solvent is evaporated. The residue is taken up in methylene chloride/methanol and washed with water and 0.1 N HCl. The organic phase is dried over sodium sulphate and the solvent is evaporated.

Yield: 38 mg (29% of theory),

R_{f} value: 0.45 (silica gel, methylene chloride/methanol=9:1)

C₂₁H₂₄Cl₂N₂O₃

EII mass spectrum: m/z=423/425 [M+H]⁺

The following compounds of general formula IVX-1 are prepared analogously to Example 14.1, the educts used being shown in the column headed “Educts”:

| | | | | | | | | (IVX-1) |
|---|---|---|---|---|---|---|---|---|
| 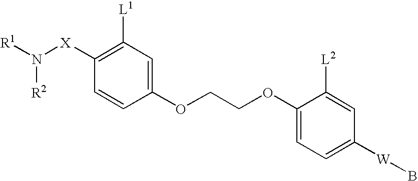 | | | | | | | | |
| | | | | | | | ret. time | |
| Exam- | | | | | | mass | (HPLC) | |
| ple | R¹R²N-X- | -W-B | L¹ | L² | Educts | spectrum | [min]* | R_{f}-value |
| 14.2 | 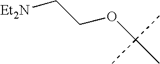 | 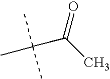 | —Cl | —CH₃ | XIII.1 | 420/422 [M + H]⁺ | 2.6 | 0.45 (C) |
| 14.3 | 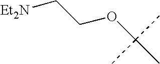 | 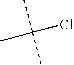 | —Cl | —Cl | XIII.1 | 432/434/ 436 [M + H]⁺ | 3.0 | 0.45 (C) |

*HPLC method A

### EXAMPLE 15.1

### [3-(4′-Chloro-biphenyl-4-yl)-propyl]-(4-dimethylaminomethyl-phenyl)-methyl-amine

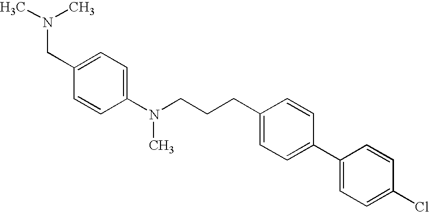

100 mg (0.264 mmol) [3-(4′-Chloro-biphenyl-4-yl)-propyl]-(4-dimethylaminomethyl-phenyl)-amine (compound 4.1) and 0.097 ml (1.30 mmol) formalin (37%) are dissolved in 5 ml of acetonitrile. The mixture is stirred for 30 minutes. After that time 0.037 ml (0.65 mmol) conc. acetic acid and 25 mg (0.39 mmol) sodium cyanoborohydride are added. The mixture is stirred for 10 hours at RT. After that time the solvent is evaporated. The residue is taken up in ethyl acetate and washed with diluted sodium hydrogen carbonate solution. The organic phase is dried over sodium sulphate and the solvent is evaporated. The residue is purified by silica gel column chromatography with methylene chloride/methanol/ammonia (9:1:0.01) as eluent.

Yield: 37 mg (23% of theory),

R_{f} value: 0.30 (silica gel, methylene chloride/methanol/ammonia=9:1:0.01)

C₂₅H₂₉ClN₂

EII mass spectrum: m/z=393/395 [M+H]⁺

The following compounds of general formula XV-1 are prepared analogously to Example 15.1, the educts used being shown in the column headed “Educts”:

(XV-1)

| Exam- | | | | mass | M.p. | |
|---|---|---|---|---|---|---|
| ple | R¹R²N-X- | -W-B | Educts | spectrum | [° C.] | R_{f}-value |
| 15.2 | 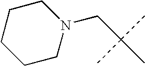 | 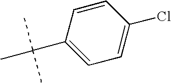 | 4.2 | 433/435 [M + H]⁺ | n.d. | 0.50 (A) |
| 15.3 | 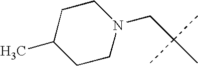 | 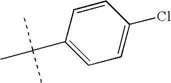 | 1.1 | 447/449 [M + H]⁺ | n.d. | 0.40 (A) |

### EXAMPLE 16

The following compounds of general formula XVI-1 are prepared analogously to Example 15.1, the educts used being shown in the column headed “Educts”:

| | |
|---|---|
| | (XVI-1) |
| 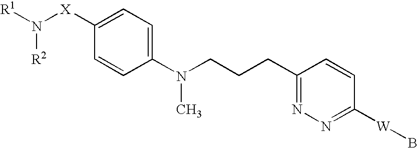 | |

| | | | | | ret. time | |
|---|---|---|---|---|---|---|
| Exam- | | | | mass | (HPLC) | |
| ple | R¹R²-N-X- | -W-B | Educts | spectrum | [min]* | R_{f}-value |
| 16.1 | 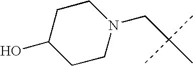 | 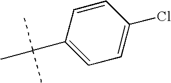 | 3.15 | 451/453 ]M + H]⁺ | 2.5 | 0.40 (A) |
| 16.2 | 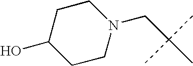 | 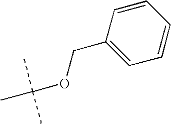 | 3.21 | 447 [M + H⁺ | 2.4 | 0.45 (A) |

*HPLC method A

### EXAMPLE 17.1

### N-{3-[6-(4-Methoxy-phenyl)-pyridazin-3-yl]-propyl}-N-(4-piperidin-1-ylmethyl-phenyl)-formamide

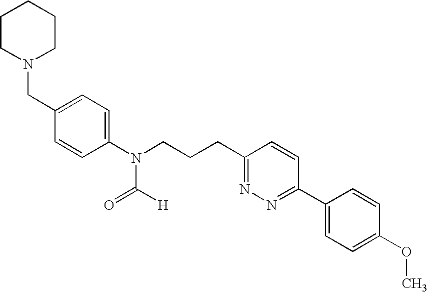

0.045 ml Acetic acid anhydride (0.48 mmol) are added to 2.0 ml formic acid (0.264 mmol) and strirred for 1.5 hours at RT. After that time 100 mg (0.24 mmol) {3-[6-(4-Methoxy-phenyl)-pyridazin-3-yl]-propyl}-(4-piperidin-1-ylmethyl-phenyl)-amine (compound 3.3) are added and the mixture is stirred for 96 hours at RT and for 8 hours at 130° C. After that time the solvent is evaporated. The residue is purified by silica gel column chromatography with methylene chloride/methanol/ammonia (9:1:0.01) as eluent.

Yield: 65 mg (40% of theory),

R_{f} value: 0.70 (silica gel, methylene chloride/methanol/ammonia=9:1:0.01)

C₂₇H₃₂N₄O₂

EII mass spectrum: m/z=445 [M+H]⁺

### EXAMPLE 18

The following compounds of general formula XVIII-1 are prepared analogously to Example 8.1, the educts used being shown in the column headed “Educts”:

(XVIII-1)

| | | | | | | ret. time | |
|---|---|---|---|---|---|---|---|
| Exam- | | | | mass | M.p. | (HPLC) | R_{f}- |
| ple | R¹R²N-X- | -W-B | Educts | spectrum | [° C.] | [min]* | value |
| 18.1 | 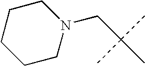 | 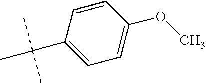 | XV.1 | 418 [M + H]⁺ | n.d. | 2.4 | 0.45 (B) |
| 18.2 | 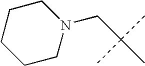 | 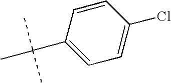 | XV.2 | 422 [M + H]⁺ | n.d. | 2.6 | 0.40 (C) |
| 18.3 | 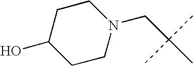 | 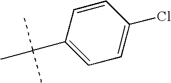 | XV.2 | 438/440 [M + H]⁺ | n.d. | 2.5 | 0.40 (B) |
| 18.4 | 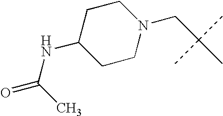 | 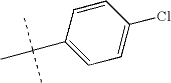 | XV.2 | 480/482 [M + H]⁺ | n.d. | 2.5 | 0.35 (B) |
| 18.5 | 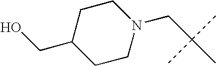 | 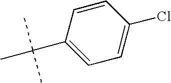 | XV.2 | 452/454 [M + H]⁺ | n.d. | 2.5 | 0.30 (B) |
| 18.6 | 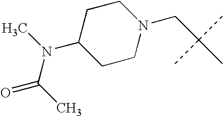 | 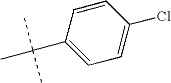 | XV.2 | 493/495 [M + H]⁺ | n.d. | 2.5 | n.d. |
| 18.7 | 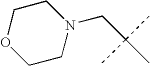 | 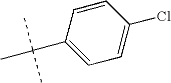 | XV.2 | 424/426 [M + H]⁺ | n.d. | 2.5 | n.d. |
| 18.8 | 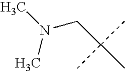 | 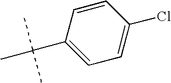 | XV.2 | 382/384 [M + H]⁺ | n.d. | 2.6 | n.d. |
| 18.9 | 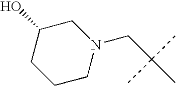 | 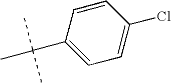 | XV.2 | 438/440 [M + H]⁺ | n.d. | 2.5 | n.d. |
| 18.10 | 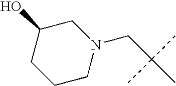 | 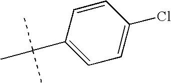 | XV.2 | 438/440 [M + H]⁺ | n.d. | 2.5 | n.d. |
| 18.11 | 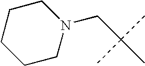 | 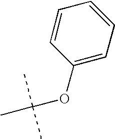 | XV.3 | 404 [M + H]⁺ | n.d. | 2.3 | n.d. |
| 18.12 | 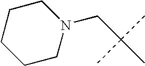 | 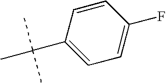 | XV.4 | 406 [M + H]⁺ | n.d. | 2.3 | n.d. |
| 18.13 | 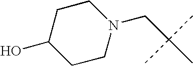 | 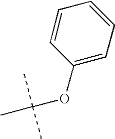 | XV.3 | 420 [M + H]⁺ | n.d. | 2.3 | n.d. |
| 18.14 | 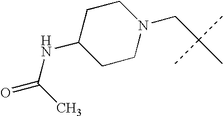 | 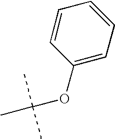 | XV.3 | 461 [M + H]⁺ | n.d. | 2.2 | n.d. |
| 18.15 | 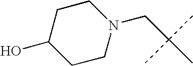 | 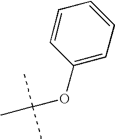 | XV.4 | 422 [M + H]⁺ | n.d. | 2.3 | n.d. |
| 18.16 | 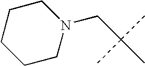 | 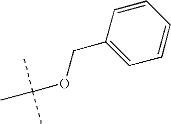 | XV.5 | 418 [M + H]⁺ | n.d. | 2.5 | 0.75 (B) |
| 18.17 | 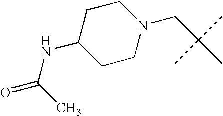 | 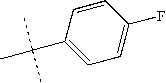 | XV.4 | 463 [M + H]⁺ | n.d. | 2.3 | n.d. |

*HPLC method A

### EXAMPLE 19.1

### {3-[6-(4-Methoxy-phenyl)-pyridazin-3-yl]-propyl}-(2-methyl-1,2,3,4-tetrahydro-isoquinolin-7-yl)-amine

540 mg (1.10 mmol) 3-[6-(4-Methoxy-phenyl)-pyridazin-3-yl]-propionaldehyde (educt XIX.1) and 178 mg (1.10 mmol) 2-methyl-1,2,3,4-tetrahydro-isoquinolin-7-ylamine (educt XVIII.1) are dissolved in 20 ml of 1,2-dichloroethane and 0.25 ml conc. acetic acid are added. Finally 466 mg (2.2 mmol) sodium triacetoxyborohydride are added and the mixture is stirred for 4 hours at RT. After that time saturated sodium hydrogen carbonate solution is added and the mixture is extracted with methylene chloride. The organic phase is dried over sodium sulphate and the solvent is evaporated. The residue is purified by silica gel column chromatography with methylene chloride/methanol/ammonia (9:1:0.01) as eluent.

Yield: 150 mg (35% of theory),

R_{f} value: 0.60 (silica gel, methylene chloride/methanol/ammonia=9:1:0.01)

M.p. 130-133° C.

C₂₄H₂₈N₄O

EII mass spectrum: m/z=389 [M+H]⁺

The following compounds of general formula XIX-1 are prepared analogously to Example 19.1, the educts used being shown in the column headed “Educts”:

| | |
|---|---|
| | (XIX-1) |
| 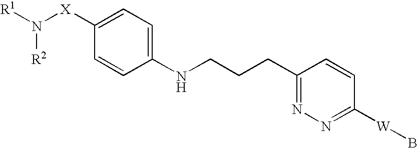 | |

| | | | | | | ret. time | |
|---|---|---|---|---|---|---|---|
| Exam- | | | | mass | M.p. | (HPLC) | R_{f}- |
| ple | R¹R²N-X- | -W-B | Educts | spectrum | [° C.] | [min]* | value |
| 19.2 | 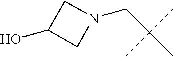 | 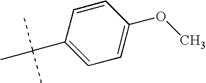 | XIX.1 XXI.1 | 405 [M + H⁺ | 143-145 | 2.2 | 0.30 (D) |
| 19.3 | 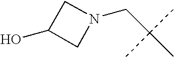 | 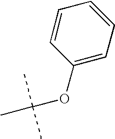 | XIX.2 XXI.2 | 391 [M + H⁺ | n.d. | 2.2 | n.d. |
| 19.4 | 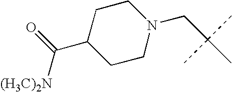 | 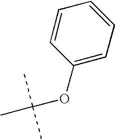 | XIX.2 XXI.2 | 474 [M + H⁺ | n.d. | 2.3 | n.d. |

*HPLC method A

### EXAMPLE 20

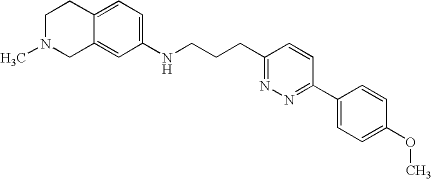

The following compounds of general formula XX-1 are prepared analogously to Example 13.1, the educts used being shown in the column headed “Educts”

| | |
|---|---|
| | (XX-1) |
| 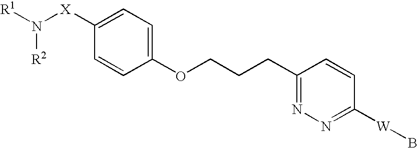 | |

| | | | | | | ret. time | |
|---|---|---|---|---|---|---|---|
| Exam- | | | | mass | M.p. | (HPLC) | R_{f}- |
| ple | R¹R²N-X- | -W-B | Educts | spectrum | [° C.] | [min]* | value |
| 20.1 | 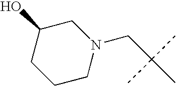 | 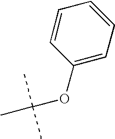 | XXIV.1 | 420 [M + H]⁺ | n.d. | 2.3 | n.d. |
| 20.2 | 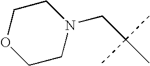 | 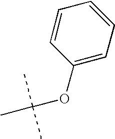 | XXIV.1 | 406 [M + H]⁺ | n.d. | 2.3 | n.d. |
| 20.3 | 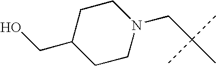 | 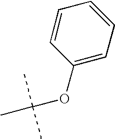 | XXIV.1 | 434 [M + H]⁺ | n.d. | 2.3 | n.d. |
| 20.4 | 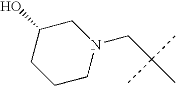 | 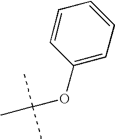 | XXIV.1 | 420 [M + H]⁺ | n.d. | 2.4 | n.d. |
| 20.5 | 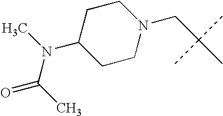 | 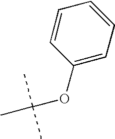 | XXIV.1 | 475 [M + H]⁺ | n.d. | 2.3 | n.d. |
| 20.6 | 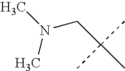 | 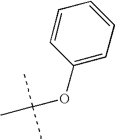 | XXIV.1 | 364 [M + H]⁺ | n.d. | 2.3 | n.d. |
| 20.7 | 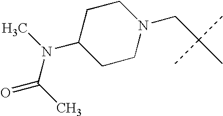 | 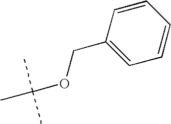 | XXIV.2 | 489 [M + H]⁺ | n.d. | 2.6 | 0.55 (C) |

*HPLC method A

### EXAMPLE 21.1

### 1-(4-{3-[6-(4-Hydroxy-phenyl)-pyridazin-3-yl]-propylamino}-benzyl)-4-methyl-piperidin-4-ol

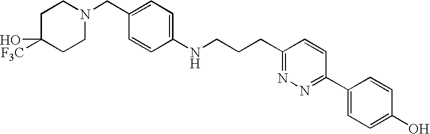

150 mg (0.30 mmol) 1-(4-{3-[6-(4-Methoxy-phenyl)-pyridazin-3-yl]-propylamino}-benzyl)-4-methyl-piperidin-4-ol (educt 3.6) are dissolved in 10 ml methylene chloride and 0.33 ml (0.33 mmol) of a 1N solution of boron tribromide in methylene chloride are added at −65° C. under argon atmosphere. The mixture is stirred for 30 minutes at −65° C. and for 3 hours at RT. After that time another equivalent of boron tribromide solution (0.33 ml) is added and the mixture is stirred for 4 days at RT. After that time diluted ammonia solution is added. The organic phase is separated and dried over sodium sulphate and the solvent is evaporated. The residue is purified by silica gel column chromatography with methylene chloride/methanol/ammonia (9:1:0.01) as eluent. The product is dried in vacuo at 80° C.

Yield: 85 mg (58% of theory),

R_{f} value: 0.40 (silica gel, methylene chloride/methanol/ammonia=9:1:0.01)

retention time (HPLC): 2.2 min (method A)

M.p. 200-204° C.

C₂₆H₂₉F₃N₄O₂

EII mass spectrum: m/z=487 [M+H]⁺

### EXAMPLE 22.1

### (4-Aminomethyl-phenyl)-{3-[6-(4-methoxy-phenyl)-pyridazin-3-yl]-propyl}-amine

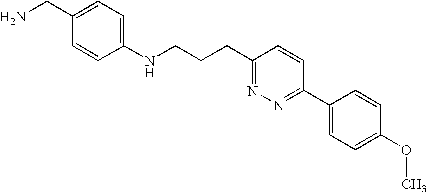

### 22.1.a

### 4-{3-[6-(4-Methoxy-phenyl)-pyridazin-3-yl]-propylamino}-benzonitrile

250 mg (1.00 mmol) 3-[6-(4-Methoxy-phenyl)-pyridazin-3-yl]-propylamine (educt II.1) and 164 mg (0.90 mmol) 4-bromo benzonitrile are dissolved in 2.0 ml of dioxane and 12 mg (0.04 mmol) 2-(di-tert-butylphosphino)biphenyl, 19 mg (0.021 mmol) tris(dibenzylideneaceton)dipalladium(0) and 122 mg (1.3 mmol) sodium tert-butoxide are added. The mixture is stirred for 24 hours at 80° C. in a sealed tube under argon atmosphere. After cooling, the solvent is removed. The residue is purified by silica gel column chromatography with methylene chloride/methanol/ammonia (9:1:0.1) as eluent.

Yield: 220 mg (71% of theory),

R_{f} value: 0.50 (silica gel, methylene chloride/methanol/ammonia=9:1:0.1)

retention time (HPLC): 3.0 min (method A)

C₂₁H₂₀N₄O

EII mass spectrum: m/z=345 [M+H]⁺

### 22.1.b

(4-Aminomethyl-phenyl)-{3-[6-(4-methoxy-phenyl)-pyridazin-3-yl]-propyl}-amine 0.22 g (0.58 mmol) 4-{3-[6-(4-Methoxy-phenyl)-pyridazin-3-yl]-propylamino}-benzonitrile are dissolved in 5 ml methylene chloride and 12 ml ammonia solution in methanol. 60 mg Raney nickel are added and the mixture is hydrogenated (3 bar) at RT for 2 days. After that time the catalyst is filtered off and the filtrate evaporated. The residue is purified by silica gel column chromatography with methylene chloride/ethyl acetate/ammonia (9:1:0.1) as eluent.

Yield: 55 mg (27% of theory),

R_{f} value: 0.35 (silica gel, methylene chloride/ethyl acetate/ammonia=9:1:0.1)

retention time (HPLC): 2.2 min (method A)

C₂₁H₂₄N₄O

EII Mass spectrum: m/z=349 [M+H]⁺

### EXAMPLE 23.1

### [4-(4-Methyl-piperidin-1-ylmethyl)-phenyl]-[3-(6-phenyl-pyridazin-3-yl)-propyl]-amine

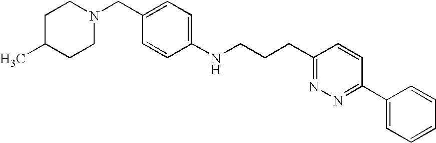

0.15 g (0.35 mmol) {3-[6-(4-Chloro-phenyl)-pyridazin-3-yl]-propyl)-[4-(4-methyl-piperidin-1-ylmethyl)-phenyl]-amine (educt 1.1) are dissolved in 10 ml ethanol and 50 mg 10% palladium/charcoal are added. The mixture is hydrogenated (50 psi) at RT for 24 hours. After that time the catalyst is filtered off and the filtrate evaporated. The residue is purified by silica gel column chromatography with methylene chloride/ethyl acetate/ammonia (9:1:0.01) as eluent.

Yield: 32 mg (23% of theory),

R_{f} value: 0.40 (silica gel, methylene chloride/ethyl acetate/ammonia=9:1:0.01)

retention time (HPLC): 2.5 min (method A)

C₂₆H₃₂N₄

EII Mass spectrum: m/z=401 [M+H]⁺

### EXAMPLE 24.1

### 1-(4-{4-[6-(4-Methoxy-phenyl)-pyridazin-3-yl]-butyl]-benzyl)-piperidin-4-ol

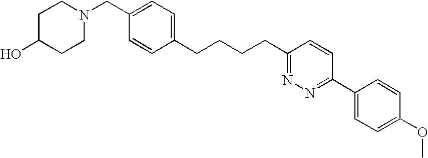

0.080 g (0.19 mmol) 1-(4-{4-[6-(4-Methoxy-phenyl)-pyridazin-3-yl]-but-3-ynyl}-benzyl)-piperidin-4-ol (educt XXV.1) are dissolved in 15 ml ethanol and 10 mg Rh(PPh₃)₃Cl (Wilkinson catalyst) are added. The mixture is hydrogenated (50 psi) for 3 hours at RT. After that time the catalyst is filtered off and the filtrate evaporated. The residue is purified by HPLC chromatography (Zorbax column, Agilent Technologies, SB (Stable Bond)-C18; 5 μm; 30 mm×100 mm; column temperature: 30° C.) using a water/acetonitrile/formic acid gradient.

Yield: 8 mg (10% of theory),

retention time (HPLC): 3.2 min (method B)

C₂₇H₃₃N₃O₂

EII Mass spectrum: m/z=432 [M+H]⁺

### 24.2

### 1-{4-[4-(6-Benzyloxy-pyridazin-3-yl)-butyl]-benzyl}-piperidin-4-ol

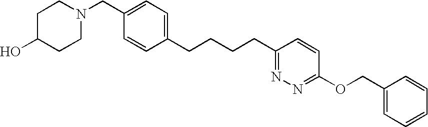 prepared analogously to example 24.1 from 1-{4-[4-(6-benzyloxy-pyridazin-3-yl)-but-3-ynyl]-benzyl}-piperidin-4-ol (educt XXV.2)

retention time (HPLC): 3.2 min (method B)

C₂₇H₃₃N₃O₂

EII mass spectrum: m/z=432 [M+H]⁺

### EXAMPLE 25

The following compounds of general formula XXV-1 are prepared analogously to Example 8.1, the educts used being shown in the column headed “Educts”:

| | |
|---|---|
| | (XXV-1) |
| 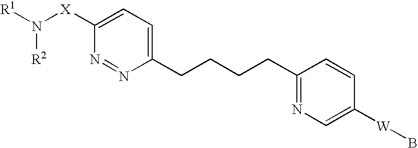 | |

| | | | | | | ret. time | |
|---|---|---|---|---|---|---|---|
| | | | | | | (HPLC) | |
| Exam- | | | | mass | M.p. | [min] | R_{f}- |
| ple | R¹R²N-X- | -W-B | Educts | spectrum | [° C.] | (method) | value |
| 25.1 | 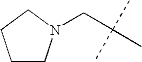 | 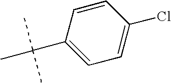 | XXVI.1 | 407/409 [M + H]⁺ | n.d. | 3.2 (F) | n.d. |
| 25.2 | 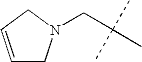 | 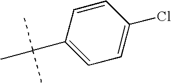 | XXVI.1 | 405/407 [M + H]⁺ | n.d. | 3.2 (D) | n.d. |
| 25.3 | 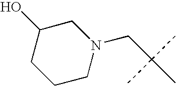 | 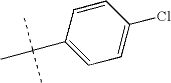 | XXVI.1 | 437/439 [M + H]⁺ | n.d. | 3.1 (D) | n.d. |
| 25.4 | 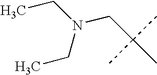 | 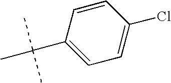 | XXVI.1 | 409/411 [M + H]⁺ | n.d. | 2.7 (E) | n.d. |
| 25.5 | 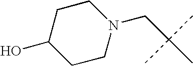 | 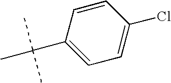 | XXVI.1 | 437/439 [M + H]⁺ | n.d. | 3.2 (D) | n.d. |

The following compounds of general formula (A-1) can be prepared analogously to the foregoing examples:

| | |
|---|---|
| | (A-1) |
| 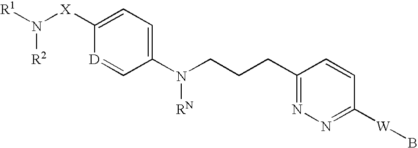 | |

| Example | R¹R²N-X- | D | R^{N} | -W-B |
|---|---|---|---|---|
| A.1 | 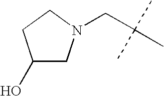 | CH | —H | 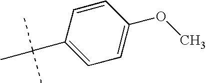 |
| A.2 | 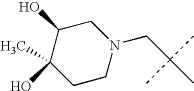 | CH | —H | 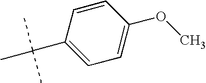 |
| A.3 | 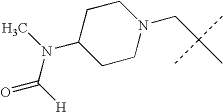 | CH | —H | 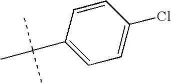 |
| A.4 | 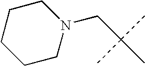 | CH | —H | 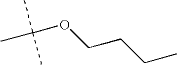 |
| A.5 | 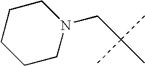 | CH | —H | 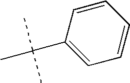 |
| A.6 | 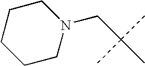 | CH | —H | 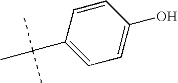 |
| A.7 | 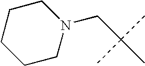 | CH | —H | 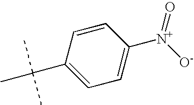 |
| A.8 | 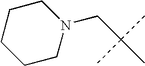 | CH | —H | 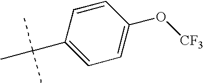 |
| A.9 | 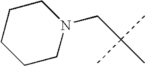 | CH | —H | 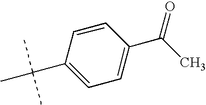 |
| A.10 | 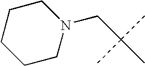 | CH | —H | 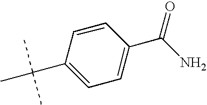 |
| A.11 | 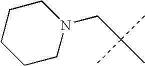 | CH | —H | 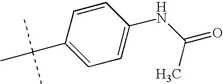 |
| A.12 | 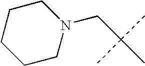 | CH | —H | 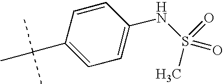 |
| A.13 | 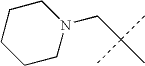 | CH | —H | 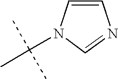 |
| A.14 | 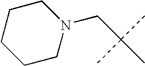 | CH | —H | 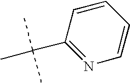 |
| A.15 | 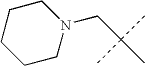 | CH | —H | 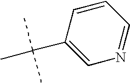 |
| A.16 | 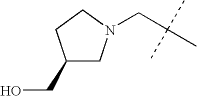 | CH | —H | 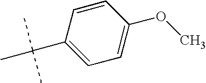 |
| A.17 | 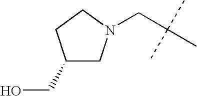 | CH | —H | 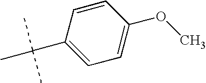 |
| A.18 | 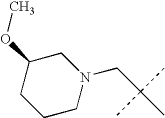 | CH | —H | 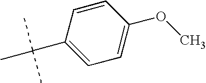 |
| A.19 | 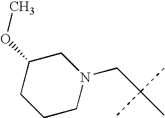 | CH | —H | 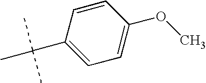 |
| A.20 | 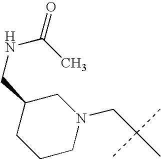 | CH | —H | 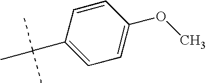 |
| A.21 | 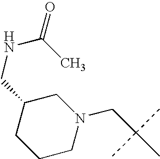 | CH | —H | 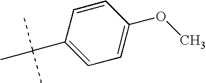 |
| A.22 | 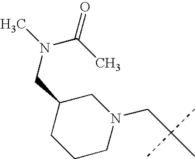 | CH | —H | 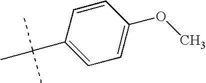 |
| A.23 | 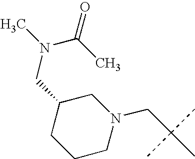 | CH | —H | 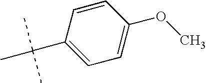 |
| A.24 | 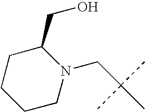 | CH | —H | 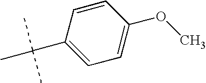 |
| A.25 | 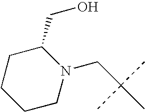 | CH | —H | 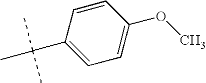 |
| A.26 | 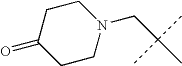 | CH | —H | 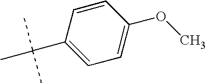 |
| A.27 | 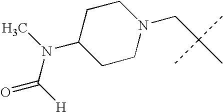 | CH | —H | 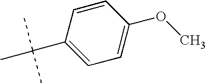 |
| A.28 | 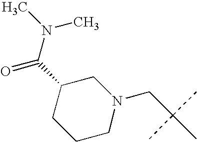 | CH | —H | 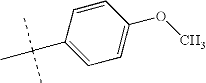 |
| A.29 | 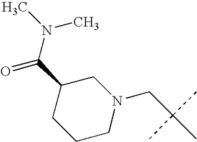 | CH | —H | 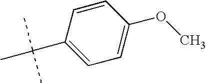 |
| A.30 | 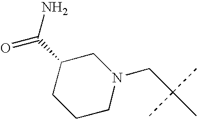 | CH | —H | 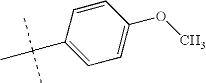 |
| A.31 | 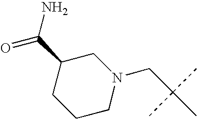 | CH | —H | 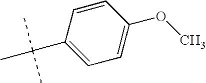 |
| A.32 | 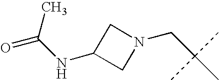 | CH | —H | 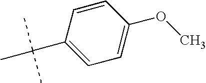 |
| A.33 | 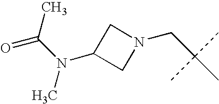 | CH | —H | 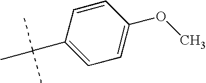 |
| A.34 | 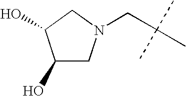 | CH | —H | 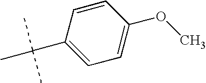 |
| A.35 | 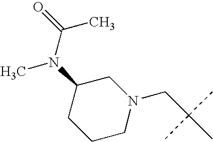 | CH | —H | 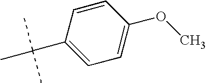 |
| A.36 | 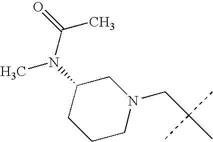 | CH | —H | 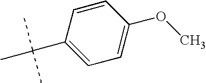 |
| A.37 | 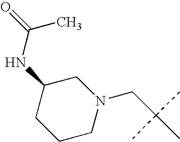 | CH | —H | 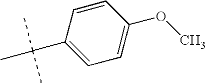 |
| A.38 | 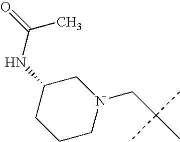 | CH | —H | 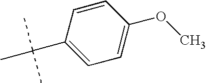 |
| A.39 | 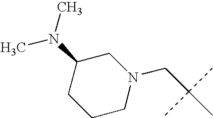 | CH | —H | 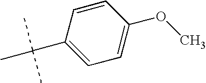 |
| A.40 | 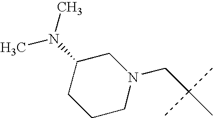 | CH | —H | 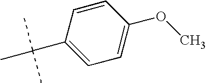 |
| A.41 | 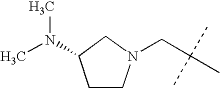 | CH | —H | 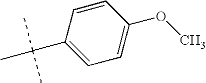 |
| A.42 | 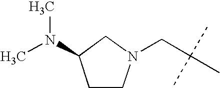 | CH | —H | 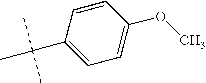 |
| A.43 | 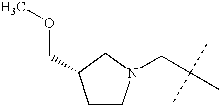 | CH | —H | 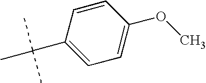 |
| A.44 | 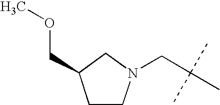 | CH | —H | 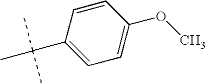 |
| A.45 | 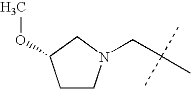 | CH | —H | 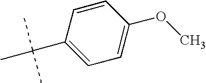 |
| A.46 | 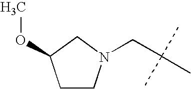 | CH | —H | 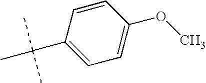 |
| A.47 | 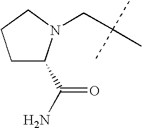 | CH | —H | 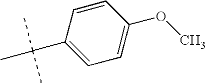 |
| A.48 | 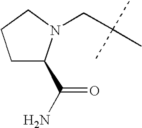 | CH | —H | 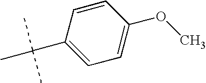 |
| A.49 | 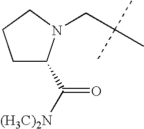 | CH | —H | 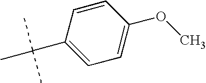 |
| A.50 | 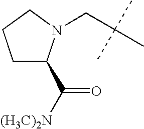 | CH | —H | 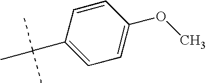 |
| A.51 | 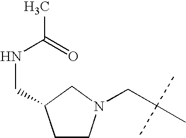 | CH | —H | 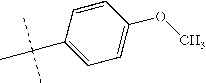 |
| A.52 | 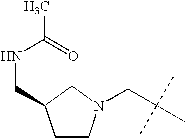 | CH | —H | 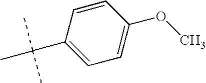 |
| A.53 | 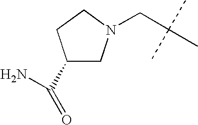 | CH | —H | 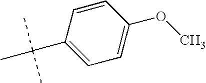 |
| A.54 | 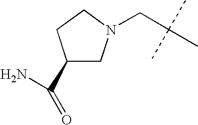 | CH | —H | 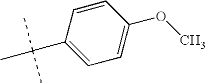 |
| A.55 | 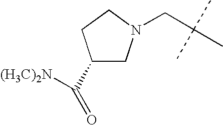 | CH | —H | 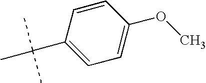 |
| A.56 | 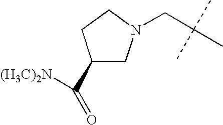 | CH | —H | 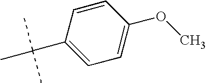 |
| A.57 | 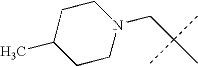 | CH | —H | 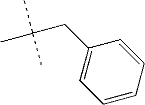 |
| A.58 | 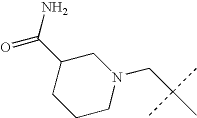 | CH | —H | 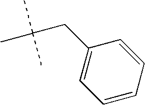 |
| A.59 | 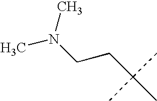 | CH | —H | 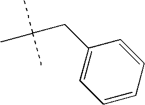 |
| A.60 | 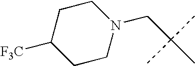 | CH | —H | 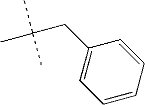 |
| A.61 | 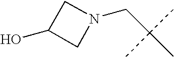 | CH | —H | 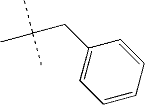 |
| A.62 | 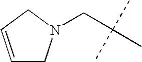 | CH | —H | 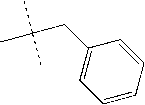 |
| A.63 | 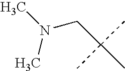 | CH | —H | 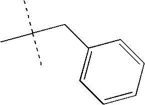 |
| A.64 | 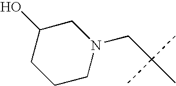 | CH | —H | 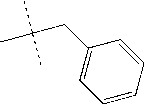 |
| A.65 | 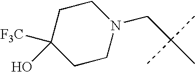 | CH | —H | 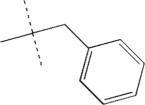 |
| A.66 | 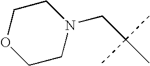 | CH | —H | 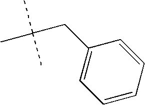 |
| A.67 | 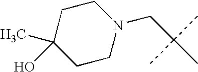 | CH | —H | 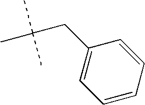 |
| A.68 | 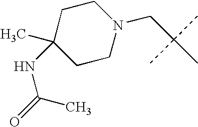 | CH | —H | 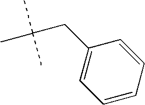 |
| A.69 | 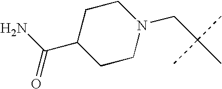 | CH | —H | 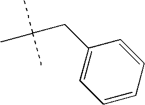 |
| A.70 | 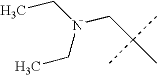 | CH | —H | 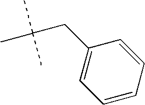 |
| A.71 | 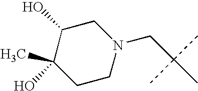 | CH | —H | 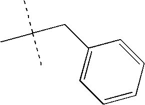 |
| A.72 | 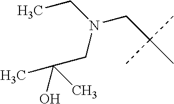 | CH | —H | 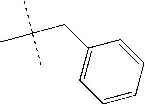 |
| A.73 | 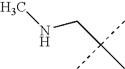 | CH | —H | 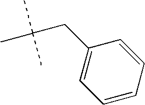 |
| A.74 | 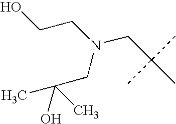 | CH | —H | 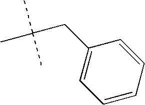 |
| A.75 | 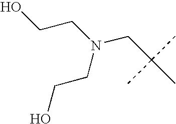 | CH | —H | 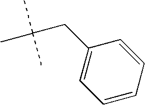 |
| A.76 | 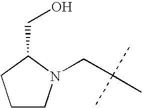 | CH | —H | 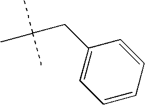 |
| A.77 | 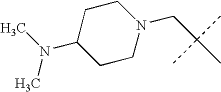 | CH | —H | 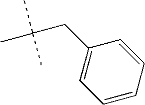 |
| A.78 | 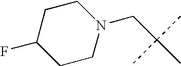 | CH | —H | 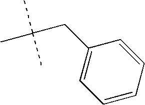 |
| A.79 | 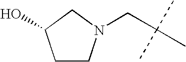 | CH | —H | 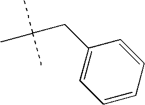 |
| A.80 | 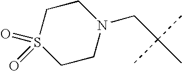 | CH | —H | 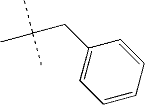 |
| A.81 | 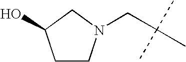 | CH | —H | 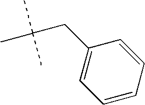 |
| A.82 | 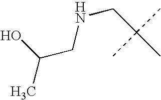 | CH | —H | 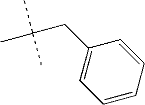 |
| A.83 | 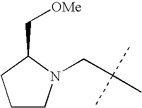 | CH | —H | 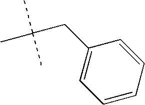 |
| A.84 | 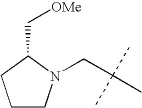 | CH | —H | 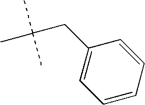 |
| A.85 | 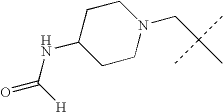 | CH | —H | 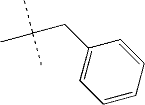 |
| A.86 | 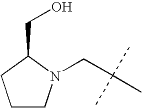 | CH | —H | 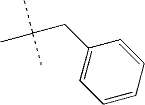 |
| A.87 | 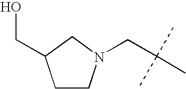 | CH | —H | 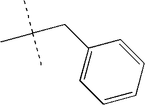 |
| A.88 | 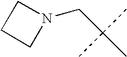 | CH | —H | 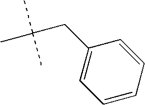 |
| A.89 | 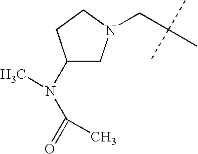 | CH | —H | 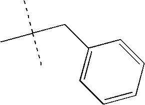 |
| A.90 | 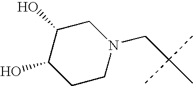 | CH | —H | 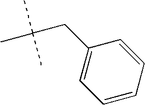 |
| A.91 | 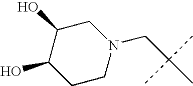 | CH | —H | 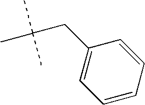 |
| A.92 | 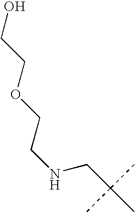 | CH | —H | 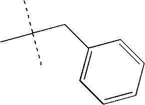 |
| A.93 | 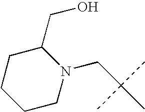 | CH | —H | 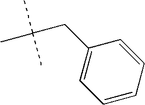 |
| A.94 | 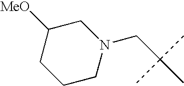 | CH | —H | 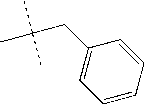 |
| A.95 | 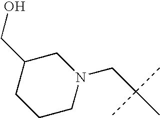 | CH | —H | 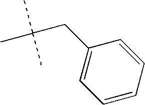 |
| A.96 | 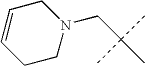 | CH | —H | 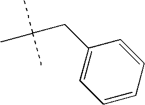 |
| A.97 | 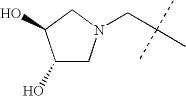 | CH | —H | 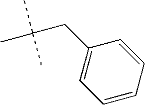 |
| A.98 | 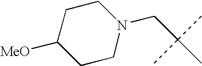 | CH | —H | 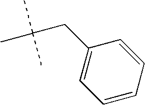 |
| A.99 | 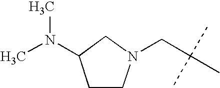 | CH | —H | 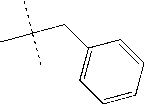 |
| A.100 | 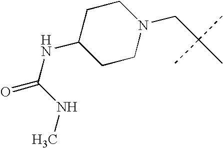 | CH | —H | 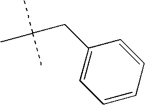 |
| A.101 | 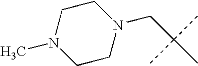 | CH | —H | 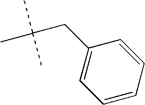 |
| A.102 | 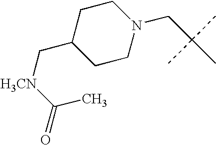 | CH | —H | 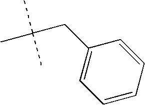 |
| A.103 | 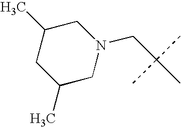 | CH | —H | 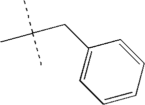 |
| A.104 | 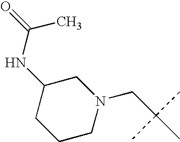 | CH | —H | 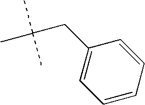 |
| A.105 | 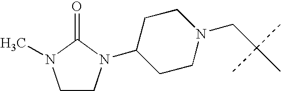 | CH | —H | 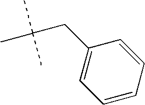 |
| A.106 | 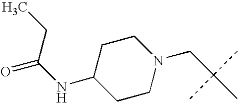 | CH | —H | 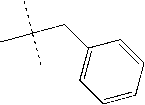 |
| A.107 | 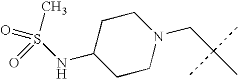 | CH | —H | 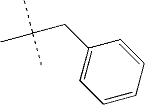 |
| A.108 | 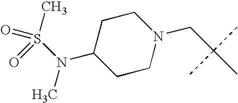 | CH | —H | 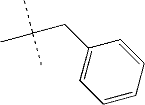 |
| A.109 | 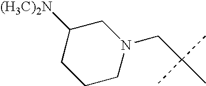 | CH | —H | 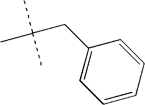 |
| A.110 | 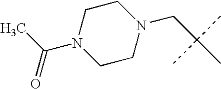 | CH | —H | 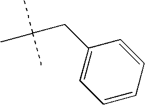 |
| A.111 | 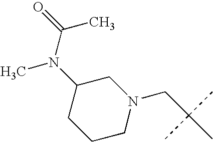 | CH | —H | 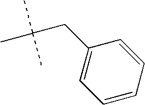 |
| A.112 | 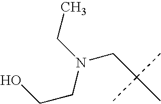 | CH | —H | 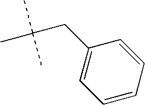 |
| A.113 | 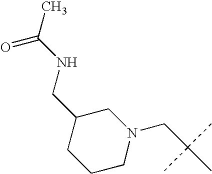 | CH | —H | 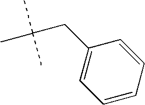 |
| A.114 | 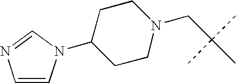 | CH | —H | 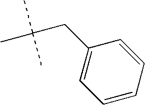 |
| A.115 | 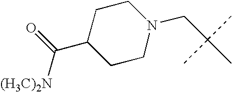 | CH | —H | 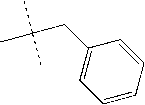 |
| A.116 | 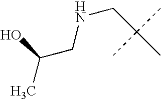 | CH | —H | 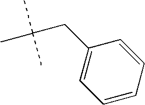 |
| A.117 | 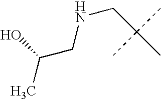 | CH | —H | 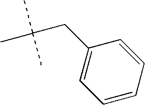 |
| A.118 | 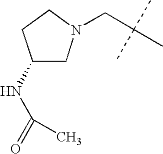 | CH | —H | 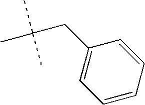 |
| A.119 | 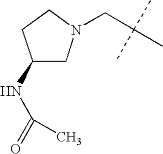 | CH | —H | 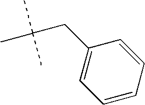 |
| A.120 | 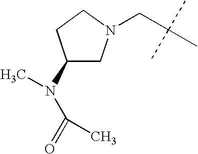 | CH | —H | 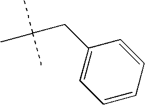 |
| A.121 | 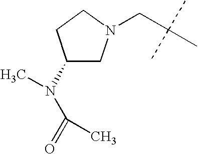 | CH | —H | 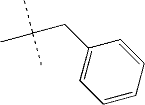 |
| A.122 | 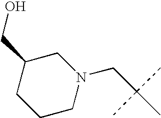 | CH | —H | 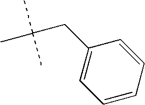 |
| A.123 | 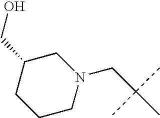 | CH | —H | 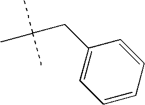 |
| A.124 | 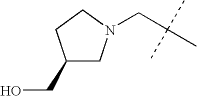 | CH | —H | 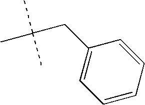 |
| A.125 | 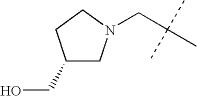 | CH | —H | 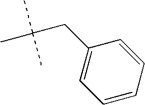 |
| A.126 | 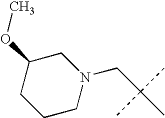 | CH | —H | 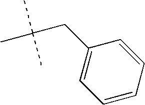 |
| A.127 | 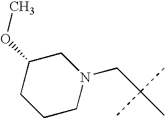 | CH | —H | 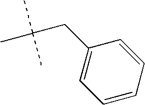 |
| A.128 | 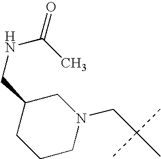 | CH | —H | 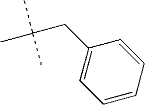 |
| A.129 | 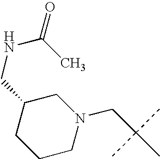 | CH | —H | 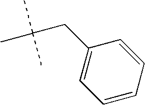 |
| A.130 | 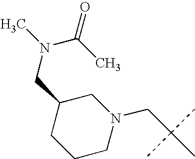 | CH | —H | 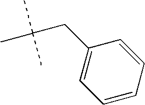 |
| A.131 | 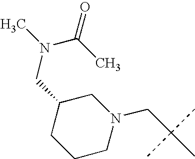 | CH | —H | 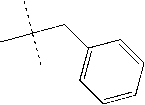 |
| A.132 | 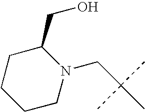 | CH | —H | 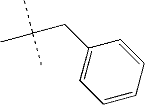 |
| A.133 | 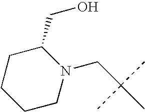 | CH | —H | 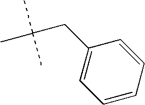 |
| A.134 | 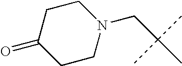 | CH | —H | 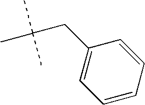 |
| A.135 | 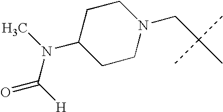 | CH | —H | 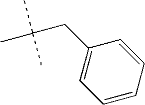 |
| A.136 | 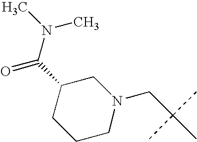 | CH | —H | 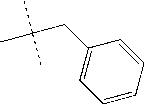 |
| A.137 | 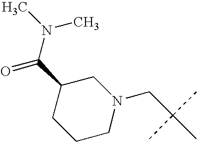 | CH | —H | 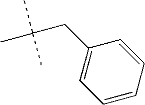 |
| A.138 | 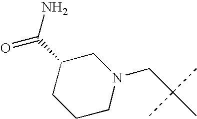 | CH | —H | 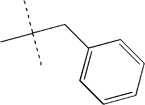 |
| A.139 | 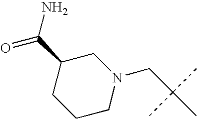 | CH | —H | 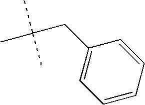 |
| A.140 | 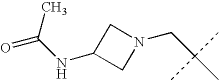 | CH | —H | 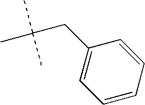 |
| A.141 | 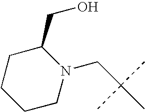 | CH | —H | 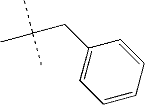 |
| A.142 | 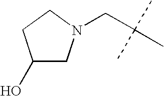 | CH | —H | 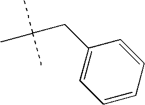 |
| A.143 | 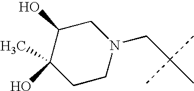 | CH | —H | 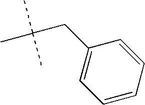 |
| A.144 | 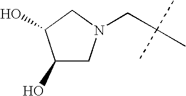 | CH | —H | 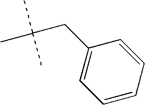 |
| A.145 | 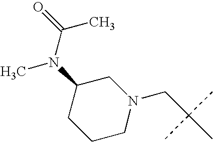 | CH | —H | 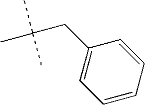 |
| A.146 | 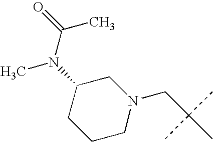 | CH | —H | 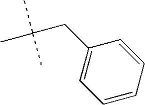 |
| A.147 | 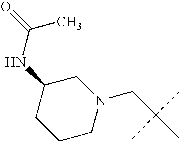 | CH | —H | 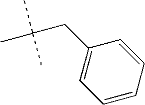 |
| A.148 | 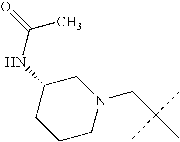 | CH | —H | 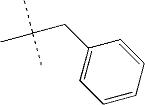 |
| A.149 | 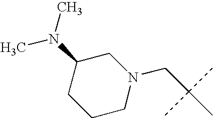 | CH | —H | 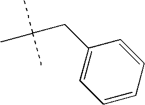 |
| A.150 | 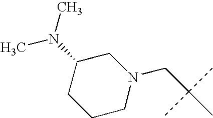 | CH | —H | 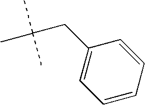 |
| A.151 | 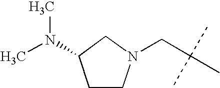 | CH | —H | 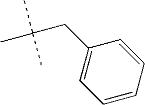 |
| A.152 | 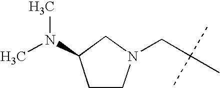 | CH | —H | 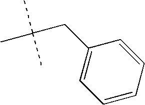 |
| A.153 | 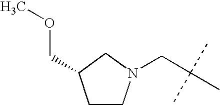 | CH | —H | 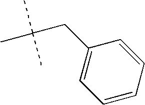 |
| A.154 | 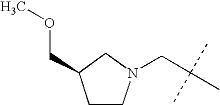 | CH | —H | 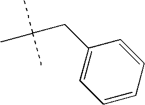 |
| A.155 | 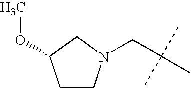 | CH | —H | 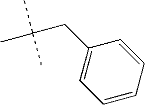 |
| A.156 | 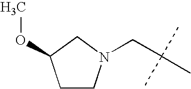 | CH | —H | 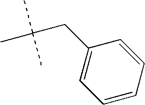 |
| A.157 | 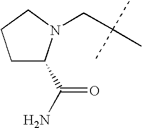 | CH | —H | 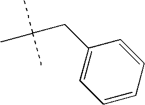 |
| A.158 | 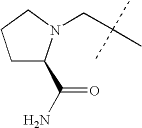 | CH | —H | 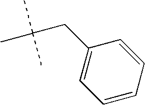 |
| A.159 | 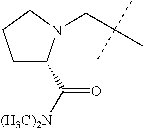 | CH | —H | 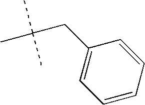 |
| A.160 | 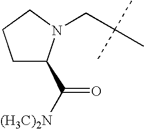 | CH | —H | 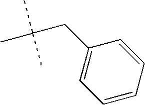 |
| A.161 | 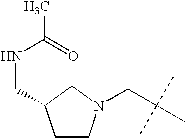 | CH | —H | 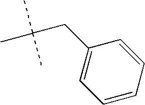 |
| A.162 | 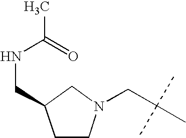 | CH | —H | 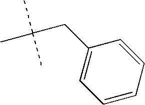 |
| A.163 | 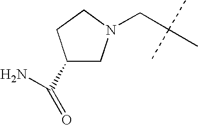 | CH | —H | 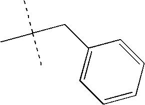 |
| A.164 | 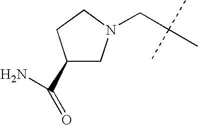 | CH | —H | 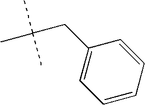 |
| A.165 | 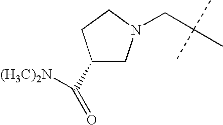 | CH | —H | 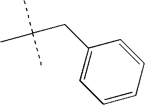 |
| A.166 | 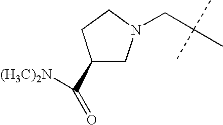 | CH | —H | 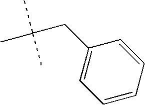 |
| A.167 | 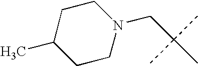 | CH | —H | 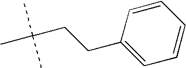 |
| A.168 | 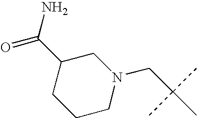 | CH | —H | 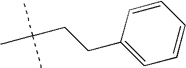 |
| A.169 | 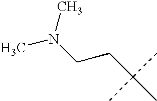 | CH | —H | 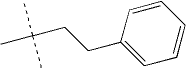 |
| A.170 | 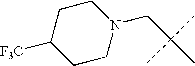 | CH | —H | 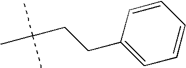 |
| A.171 | 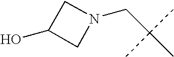 | CH | —H | 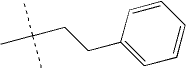 |
| A.172 | 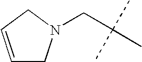 | CH | —H | 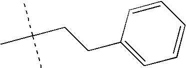 |
| A.173 | 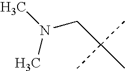 | CH | —H | 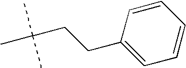 |
| A.174 | 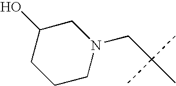 | CH | —H | 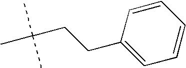 |
| A.175 | 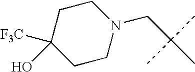 | CH | —H | 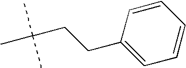 |
| A.176 | 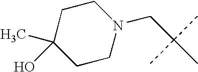 | CH | —H | 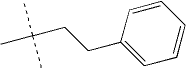 |
| A.177 | 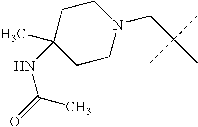 | CH | —H | 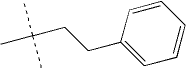 |
| A.178 | 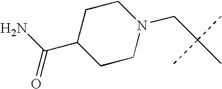 | CH | —H | 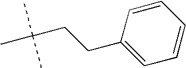 |
| A.179 | 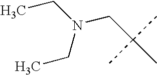 | CH | —H | 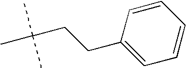 |
| A.180 | 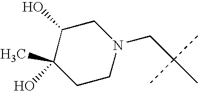 | CH | —H | 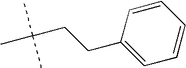 |
| A.181 | 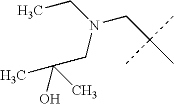 | CH | —H | 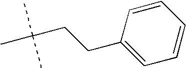 |
| A.182 | 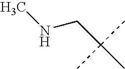 | CH | —H | 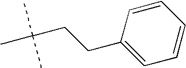 |
| A.183 | 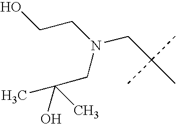 | CH | —H | 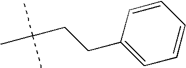 |
| A.184 | 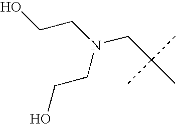 | CH | —H | 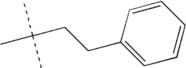 |
| A.185 | 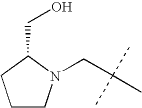 | CH | —H | 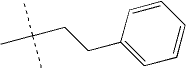 |
| A.186 | 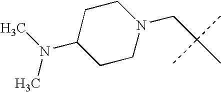 | CH | —H | 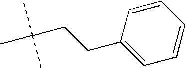 |
| A.187 | 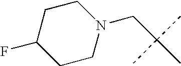 | CH | —H | 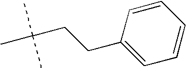 |
| A.188 | 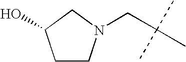 | CH | —H | 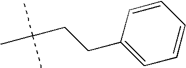 |
| A.189 | 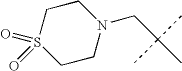 | CH | —H | 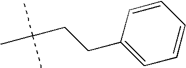 |
| A.190 | 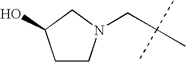 | CH | —H | 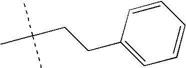 |
| A.191 | 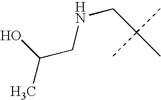 | CH | —H | 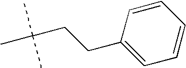 |
| A.192 | 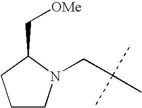 | CH | —H | 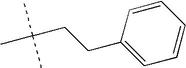 |
| A.193 | 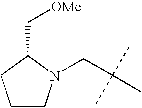 | CH | —H | 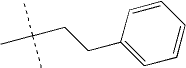 |
| A.194 | 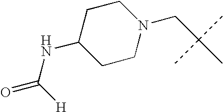 | CH | —H | 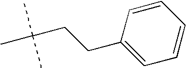 |
| A.195 | 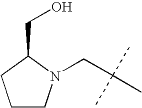 | CH | —H | 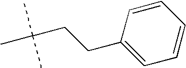 |
| A.196 | 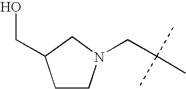 | CH | —H | 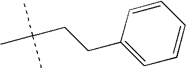 |
| A.197 | 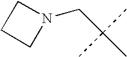 | CH | —H | 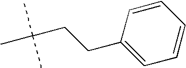 |
| A.198 | 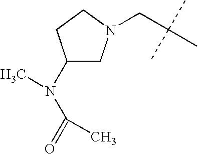 | CH | —H | 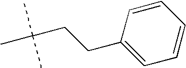 |
| A.199 | 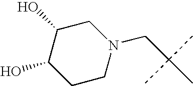 | CH | —H | 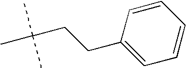 |
| A.200 | 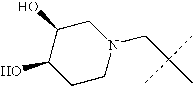 | CH | —H | 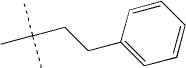 |
| A.201 | 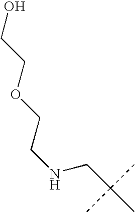 | CH | —H | 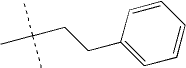 |
| A.202 | 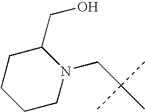 | CH | —H | 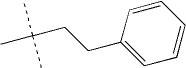 |
| A.203 | 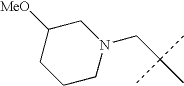 | CH | —H | 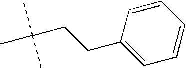 |
| A.204 | 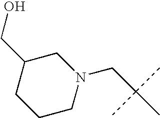 | CH | —H | 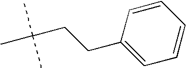 |
| A.205 | 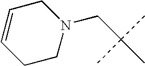 | CH | —H | 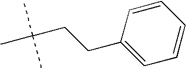 |
| A.206 | 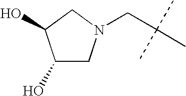 | CH | —H | 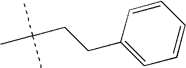 |
| A.207 | 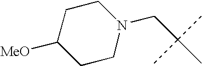 | CH | —H | 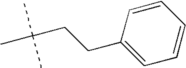 |
| A.208 | 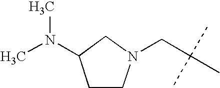 | CH | —H | 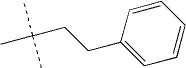 |
| A.209 | 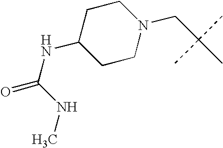 | CH | —H | 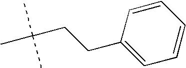 |
| A.210 | 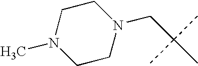 | CH | —H | 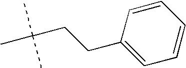 |
| A.211 | 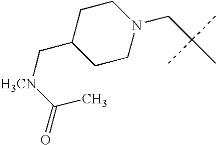 | CH | —H | 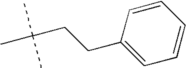 |
| A.212 | 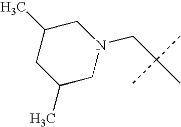 | CH | —H | 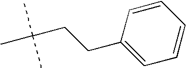 |
| A.213 | 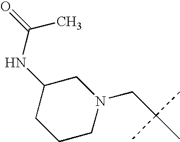 | CH | —H | 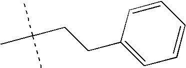 |
| A.214 | 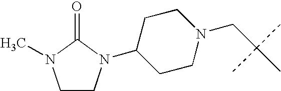 | CH | —H | 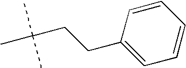 |
| A.215 | 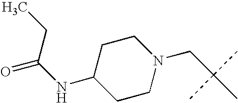 | CH | —H | 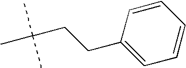 |
| A.216 | 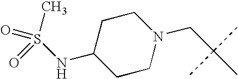 | CH | —H | 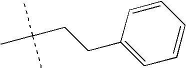 |
| A.217 | 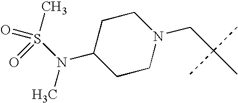 | CH | —H | 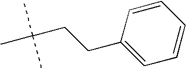 |
| A.218 | 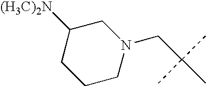 | CH | —H | 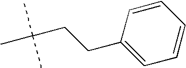 |
| A.219 | 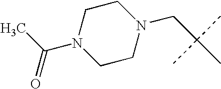 | CH | —H | 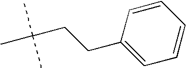 |
| A.220 | 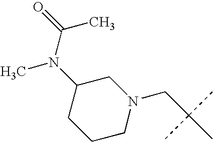 | CH | —H | 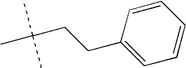 |
| A.221 | 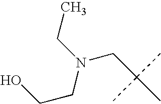 | CH | —H | 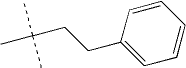 |
| A.222 | 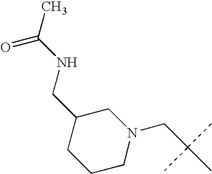 | CH | —H | 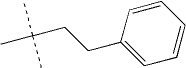 |
| A.223 | 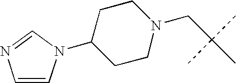 | CH | —H | 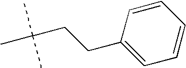 |
| A.224 | 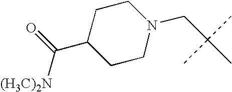 | CH | —H | 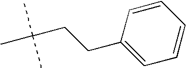 |
| A.225 | 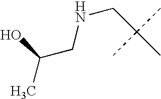 | CH | —H | 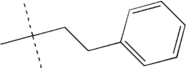 |
| A.226 | 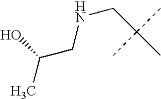 | CH | —H | 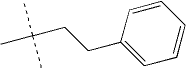 |
| A.227 | 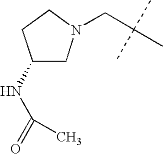 | CH | —H | 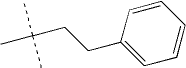 |
| A.228 | 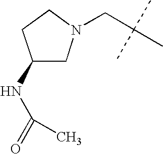 | CH | —H | 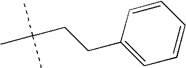 |
| A.229 | 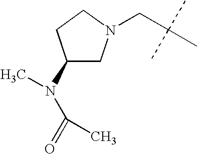 | CH | —H | 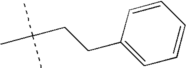 |
| A.230 | 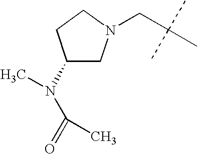 | CH | —H | 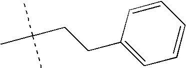 |
| A.231 | 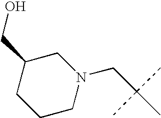 | CH | —H | 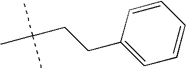 |
| A.232 | 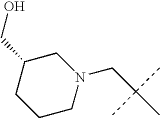 | CH | —H | 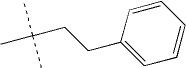 |
| A.233 | 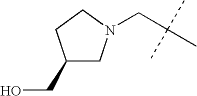 | CH | —H | 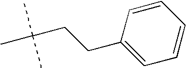 |
| A.234 | 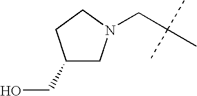 | CH | —H | 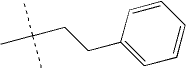 |
| A.235 | 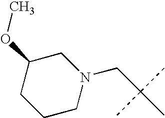 | CH | —H | 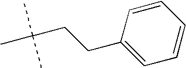 |
| A.236 | 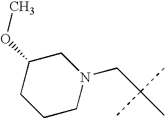 | CH | —H | 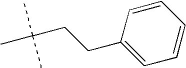 |
| A.237 | 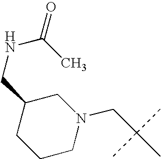 | CH | —H | 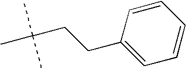 |
| A.238 | 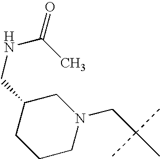 | CH | —H | 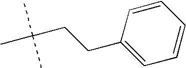 |
| A.239 | 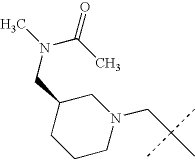 | CH | —H | 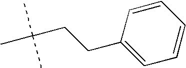 |
| A.240 | 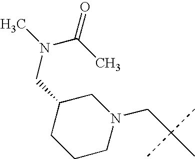 | CH | —H | 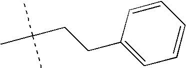 |
| A.241 | 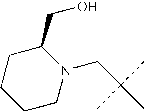 | CH | —H | 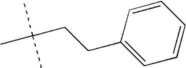 |
| A.242 | 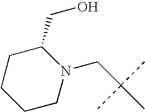 | CH | —H | 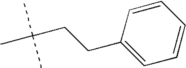 |
| A.243 | 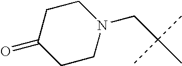 | CH | —H | 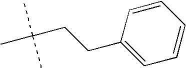 |
| A.244 | 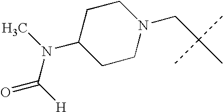 | CH | —H | 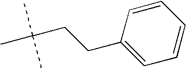 |
| A.245 | 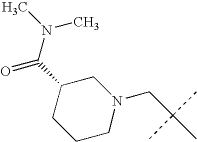 | CH | —H | 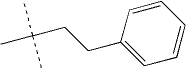 |
| A.246 | 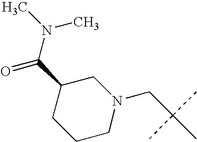 | CH | —H | 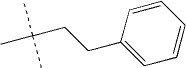 |
| A.247 | 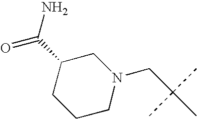 | CH | —H | 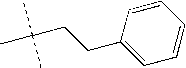 |
| A.248 | 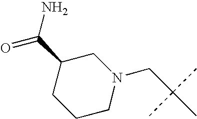 | CH | —H | 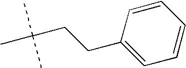 |
| A.249 | 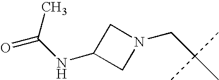 | CH | —H | 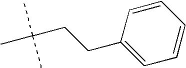 |
| A.250 | 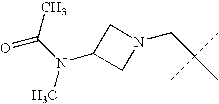 | CH | —H | 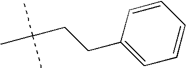 |
| A.251 | 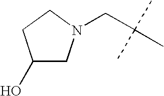 | CH | —H | 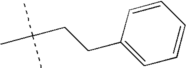 |
| A.252 | 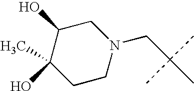 | CH | —H | 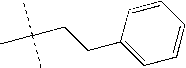 |
| A.253 | 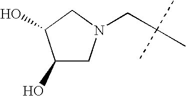 | CH | —H | 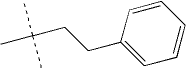 |
| A.254 | 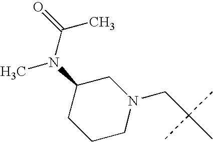 | CH | —H | 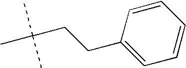 |
| A.255 | 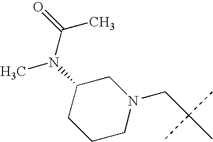 | CH | —H | 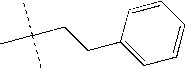 |
| A.256 | 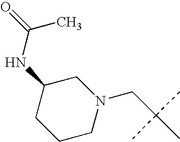 | CH | —H | 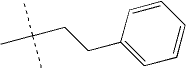 |
| A.257 | 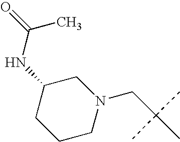 | CH | —H | 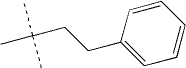 |
| A.258 | 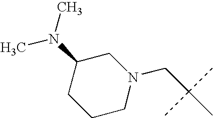 | CH | —H | 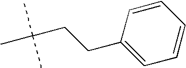 |
| A.259 | 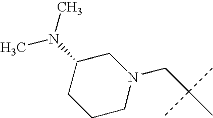 | CH | —H | 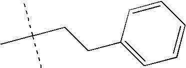 |
| A.260 | 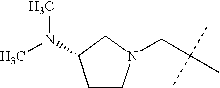 | CH | —H | 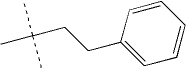 |
| A.261 | 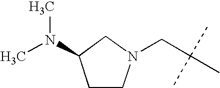 | CH | —H | 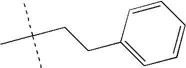 |
| A.262 | 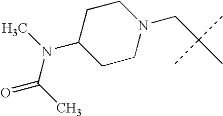 | CH | —H | 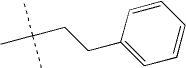 |
| A.263 | 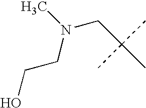 | CH | —H | 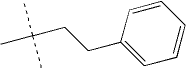 |
| A.264 | 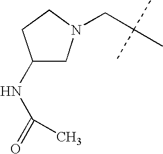 | CH | —H | 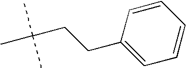 |
| A.265 | 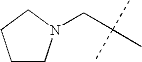 | CH | —H | 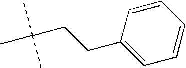 |
| A.266 | 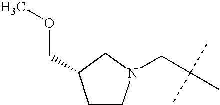 | CH | —H | 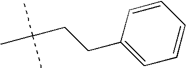 |
| A.267 | 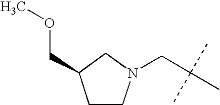 | CH | —H | 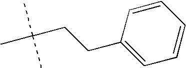 |
| A.268 | 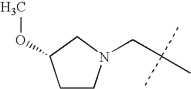 | CH | —H | 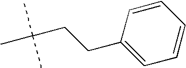 |
| A.269 | 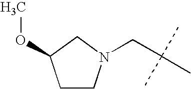 | CH | —H | 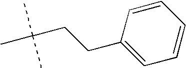 |
| A.270 | 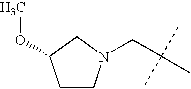 | CH | —H | 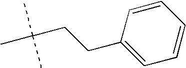 |
| A.271 | 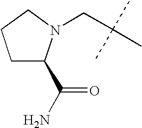 | CH | —H | 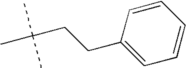 |
| A.272 | 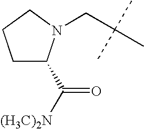 | CH | —H | 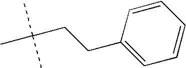 |
| A.273 | 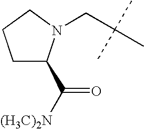 | CH | —H | 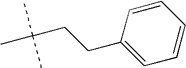 |
| A.274 | 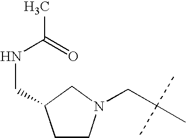 | CH | —H | 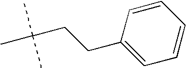 |
| A.275 | 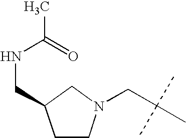 | CH | —H | 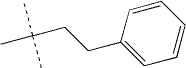 |
| A.276 | 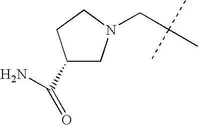 | CH | —H | 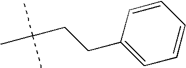 |
| A.277 | 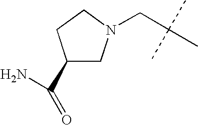 | CH | —H | 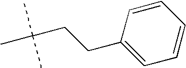 |
| A.278 | 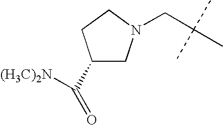 | CH | —H | 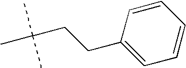 |
| A.279 | 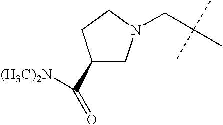 | CH | —H | 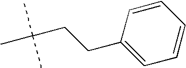 |
| A.280 | 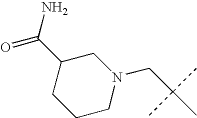 | CH | —H | 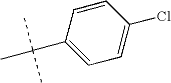 |
| A.281 | 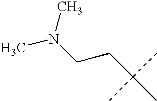 | CH | —H | 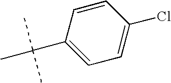 |
| A.282 | 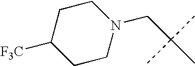 | CH | —H | 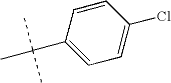 |
| A.283 | 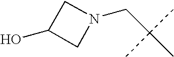 | CH | —H | 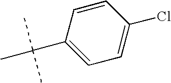 |
| A.284 | 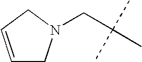 | CH | —H | 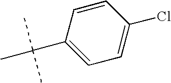 |
| A.285 | 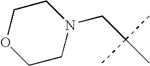 | CH | —H | 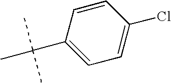 |
| A.286 | 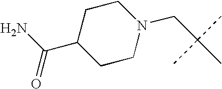 | CH | —H | 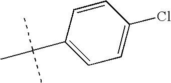 |
| A.287 | 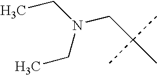 | CH | —H | 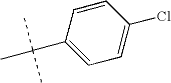 |
| A.288 | 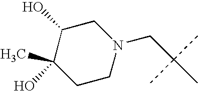 | CH | —H | 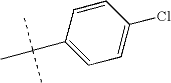 |
| A.289 | 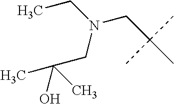 | CH | —H | 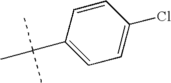 |
| A.290 | 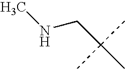 | CH | —H | 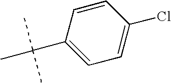 |
| A.291 | 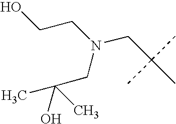 | CH | —H | 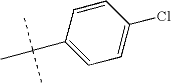 |
| A.292 | 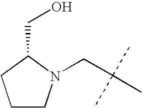 | CH | —H | 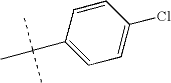 |
| A.293 | 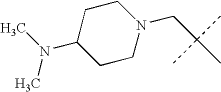 | CH | —H | 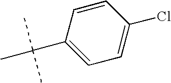 |
| A.294 | 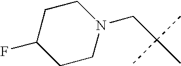 | CH | —H | 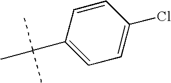 |
| A.295 | 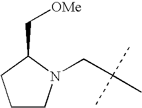 | CH | —H | 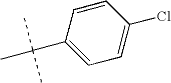 |
| A.296 | 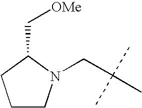 | CH | —H | 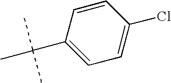 |
| A.297 | 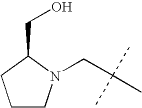 | CH | —H | 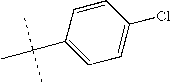 |
| A.298 | 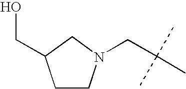 | CH | —H | 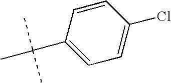 |
| A.299 | 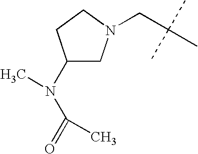 | CH | —H | 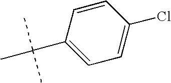 |
| A.300 | 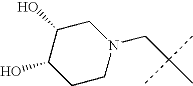 | CH | —H | 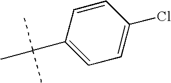 |
| A.301 | 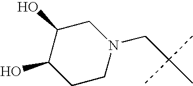 | CH | —H | 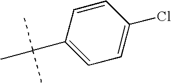 |
| A.302 | 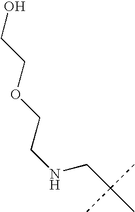 | CH | —H | 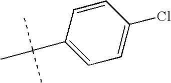 |
| A.303 | 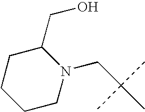 | CH | —H | 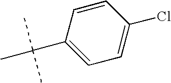 |
| A.304 | 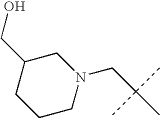 | CH | —H | 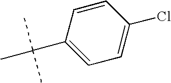 |
| A.305 | 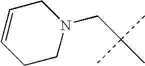 | CH | —H | 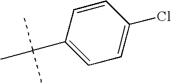 |
| A.306 | 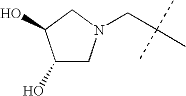 | CH | —H | 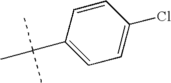 |
| A.307 | 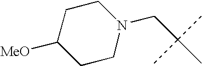 | CH | —H | 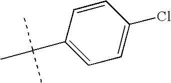 |
| A.308 | 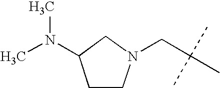 | CH | —H | 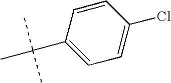 |
| A.309 | 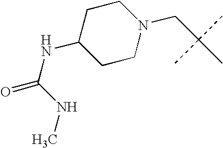 | CH | —H | 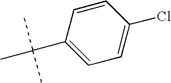 |
| A.310 | 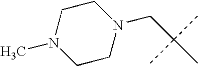 | CH | —H | 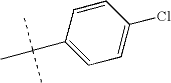 |
| A.311 | 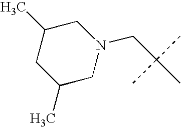 | CH | —H | 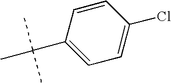 |
| A.312 | 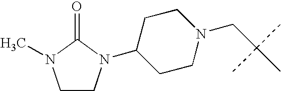 | CH | —H | 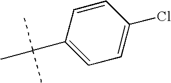 |
| A.313 | 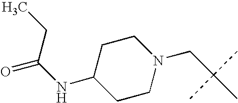 | CH | —H | 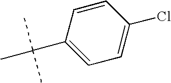 |
| A.314 | 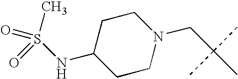 | CH | —H | 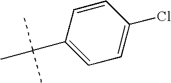 |
| A.315 | 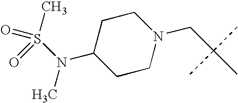 | CH | —H | 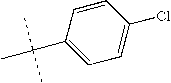 |
| A.316 | 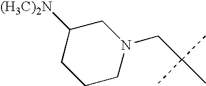 | CH | —H | 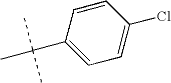 |
| A.317 | 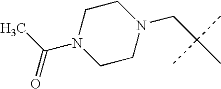 | CH | —H | 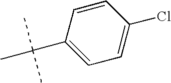 |
| A.318 | 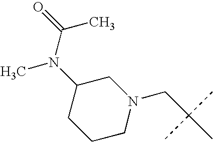 | CH | —H | 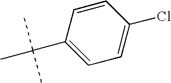 |
| A.319 | 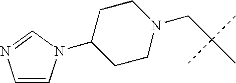 | CH | —H | 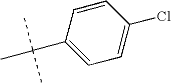 |
| A.320 | 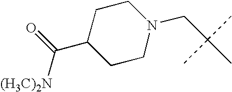 | CH | —H | 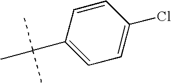 |
| A.321 | 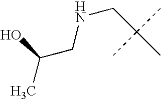 | CH | —H | 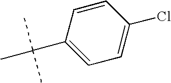 |
| A.322 | 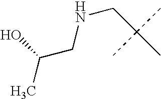 | CH | —H | 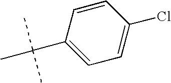 |
| A.323 | 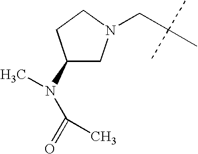 | CH | —H | 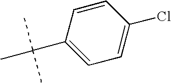 |
| A.324 | 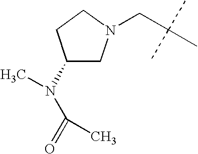 | CH | —H | 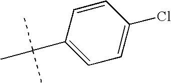 |
| A.325 | 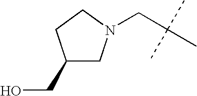 | CH | —H | 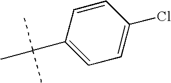 |
| A.326 | 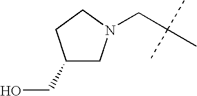 | CH | —H | 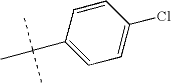 |
| A.327 | 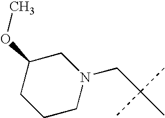 | CH | —H | 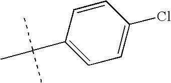 |
| A.328 | 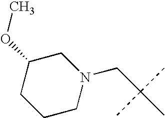 | CH | —H | 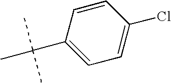 |
| A.329 | 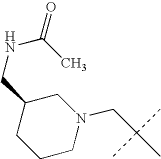 | CH | —H | 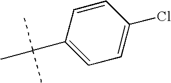 |
| A.330 | 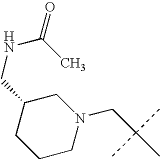 | CH | —H | 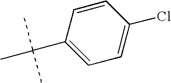 |
| A.331 | 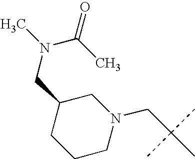 | CH | —H | 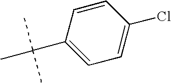 |
| A.332 | 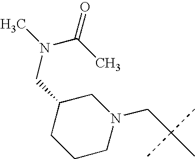 | CH | —H | 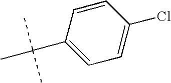 |
| A.333 | 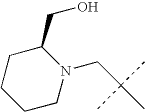 | CH | —H | 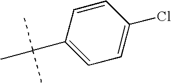 |
| A.334 | 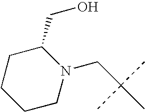 | CH | —H | 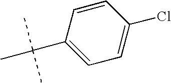 |
| A.335 | 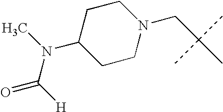 | CH | —H | 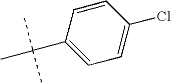 |
| A.336 | 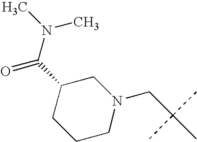 | CH | —H | 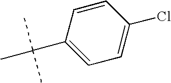 |
| A.337 | 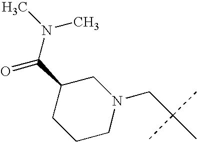 | CH | —H | 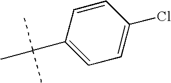 |
| A.338 | 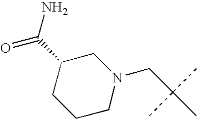 | CH | —H | 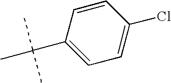 |
| A.339 | 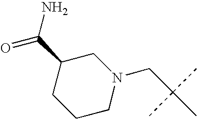 | CH | —H | 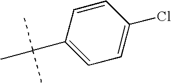 |
| A.340 | 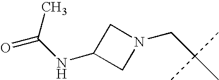 | CH | —H | 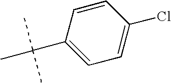 |
| A.341 | 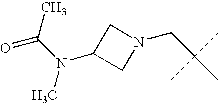 | CH | —H | 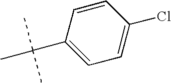 |
| A.342 | 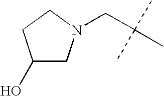 | CH | —H | 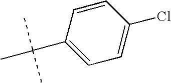 |
| A.343 | 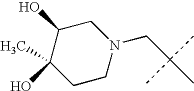 | CH | —H | 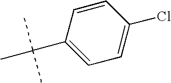 |
| A.344 | 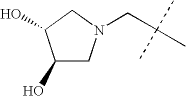 | CH | —H | 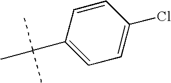 |
| A.345 | 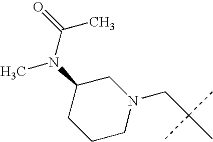 | CH | —H | 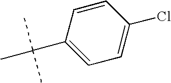 |
| A.346 | 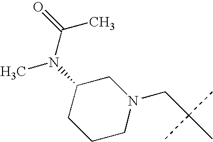 | CH | —H | 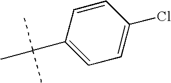 |
| A.347 | 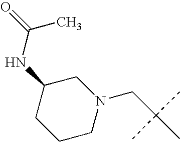 | CH | —H | 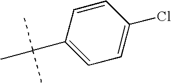 |
| A.348 | 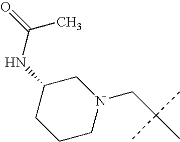 | CH | —H | 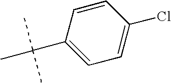 |
| A.349 | 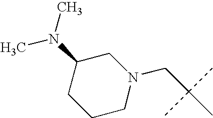 | CH | —H | 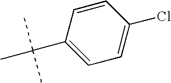 |
| A.350 | 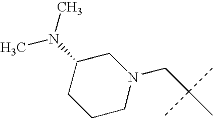 | CH | —H | 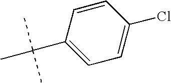 |
| A.351 | 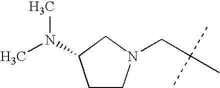 | CH | —H | 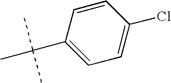 |
| A.352 | 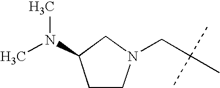 | CH | —H | 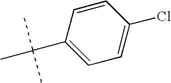 |
| A.353 | 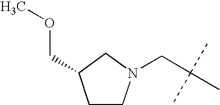 | CH | —H | 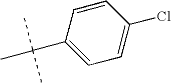 |
| A.354 | 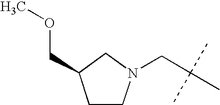 | CH | —H | 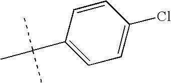 |
| A.355 | 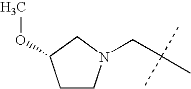 | CH | —H | 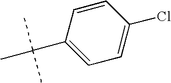 |
| A.356 | 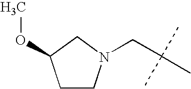 | CH | —H | 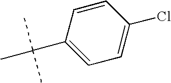 |
| A.357 | 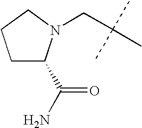 | CH | —H | 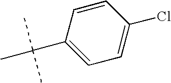 |
| A.358 | 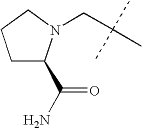 | CH | —H | 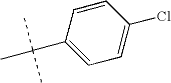 |
| A.359 | 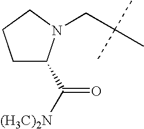 | CH | —H | 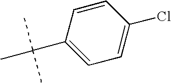 |
| A.360 | 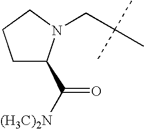 | CH | —H | 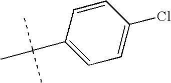 |
| A.361 | 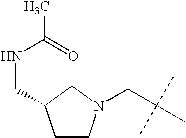 | CH | —H | 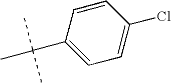 |
| A.362 | 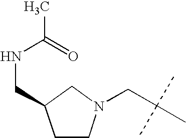 | CH | —H | 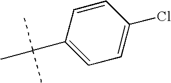 |
| A.363 | 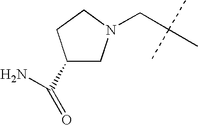 | CH | —H | 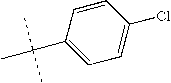 |
| A.364 | 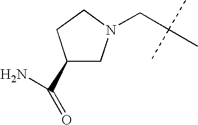 | CH | —H | 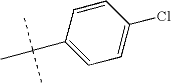 |
| A.365 | 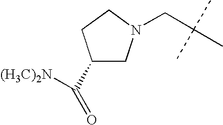 | CH | —H | 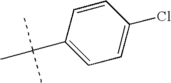 |
| A.366 | 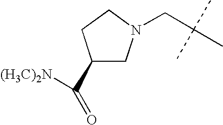 | CH | —H | 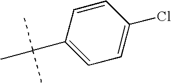 |
| A.367 | 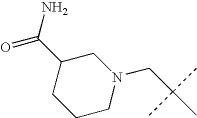 | CH | —H | 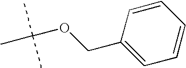 |
| A.368 | 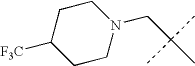 | CH | —H | 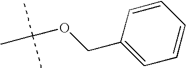 |
| A.369 | 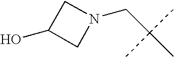 | CH | —H | 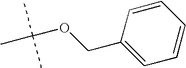 |
| A.370 | 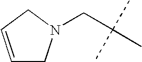 | CH | —H | 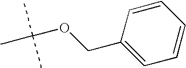 |
| A.371 | 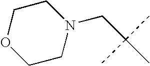 | CH | —H | 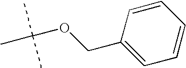 |
| A.372 | 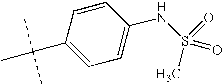 | CH | —H | 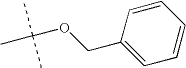 |
| A.373 | 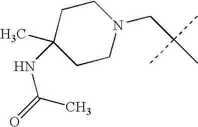 | CH | —H | 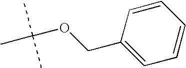 |
| A.374 | 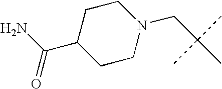 | CH | —H | 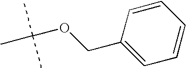 |
| A.375 | 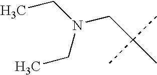 | CH | —H | 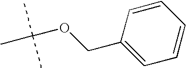 |
| A.376 | 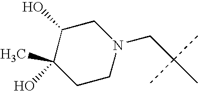 | CH | —H | 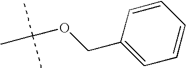 |
| A.377 | 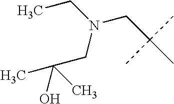 | CH | —H | 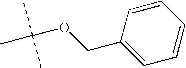 |
| A.378 | 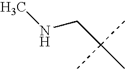 | CH | —H | 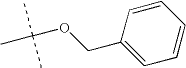 |
| A.379 | 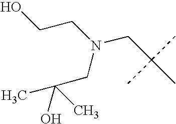 | CH | —H | 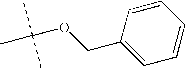 |
| A.380 | 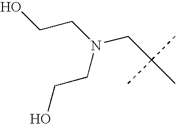 | CH | —H | 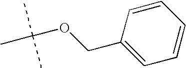 |
| A.381 | 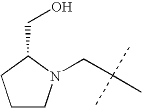 | CH | —H | 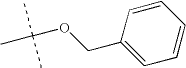 |
| A.382 | 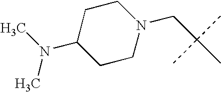 | CH | —H | 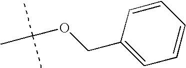 |
| A.383 | 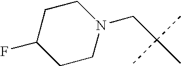 | CH | —H | 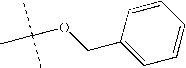 |
| A.384 | 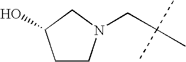 | CH | —H | 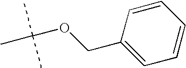 |
| A.385 | 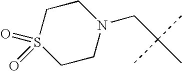 | CH | —H | 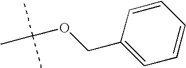 |
| A.386 | 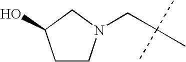 | CH | —H | 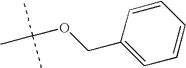 |
| A.387 | 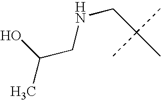 | CH | —H | 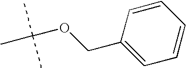 |
| A.388 | 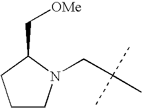 | CH | —H | 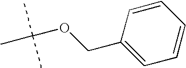 |
| A.389 | 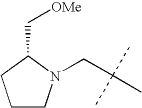 | CH | —H | 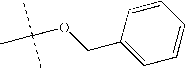 |
| A.390 | 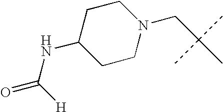 | CH | —H | 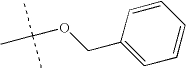 |
| A.391 | 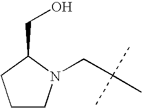 | CH | —H | 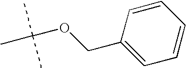 |
| A.392 | 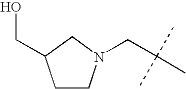 | CH | —H | 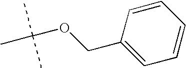 |
| A.393 | 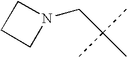 | CH | —H | 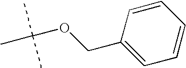 |
| A.394 | 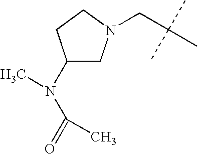 | CH | —H | 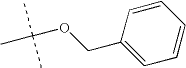 |
| A.395 | 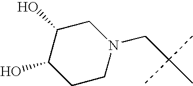 | CH | —H | 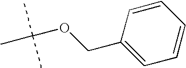 |
| A.396 | 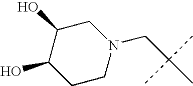 | CH | —H | 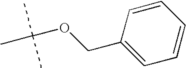 |
| A.397 | 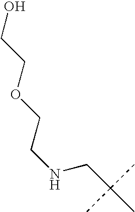 | CH | —H | 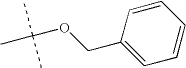 |
| A.398 | 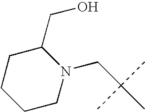 | CH | —H | 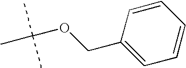 |
| A.399 | 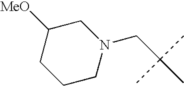 | CH | —H | 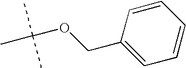 |
| A.400 | 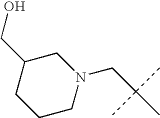 | CH | —H | 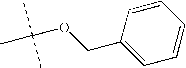 |
| A.401 | 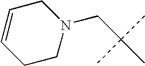 | CH | —H | 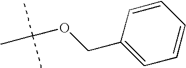 |
| A.402 | 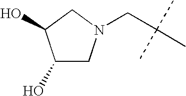 | CH | —H | 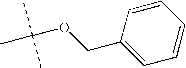 |
| A.403 | 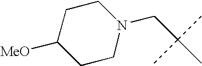 | CH | —H | 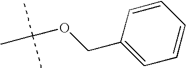 |
| A.404 | 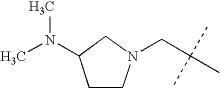 | CH | —H | 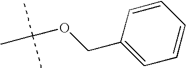 |
| A.405 | 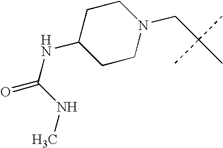 | CH | —H | 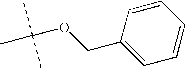 |
| A.406 | 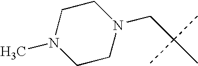 | CH | —H | 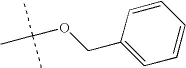 |
| A.407 | 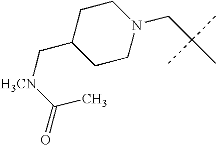 | CH | —H | 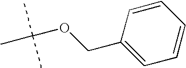 |
| A.408 | 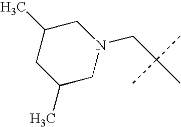 | CH | —H | 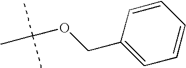 |
| A.409 | 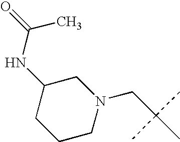 | CH | —H | 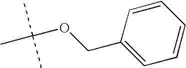 |
| A.410 | 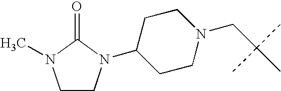 | CH | —H | 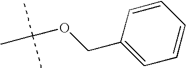 |
| A.411 | 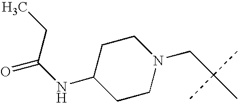 | CH | —H | 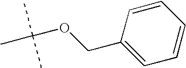 |
| A.412 | 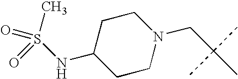 | CH | —H | 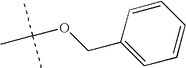 |
| A.413 | 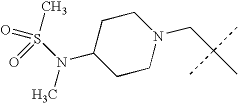 | CH | —H | 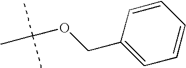 |
| A.414 | 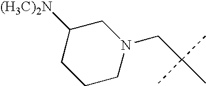 | CH | —H | 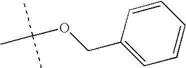 |
| A.415 | 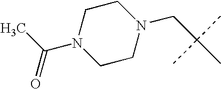 | CH | —H | 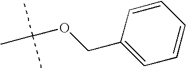 |
| A.416 | 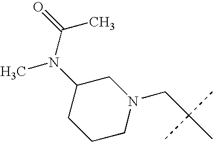 | CH | —H | 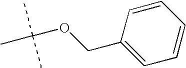 |
| A.417 | 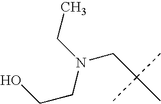 | CH | —H | 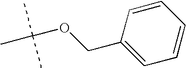 |
| A.418 | 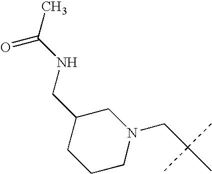 | CH | —H | 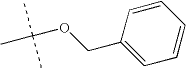 |
| A.419 | 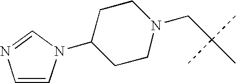 | CH | —H | 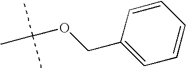 |
| A.420 | 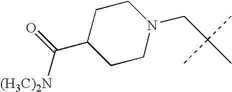 | CH | —H | 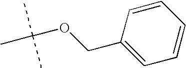 |
| A.421 | 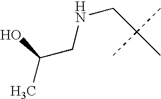 | CH | —H | 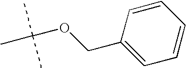 |
| A.422 | 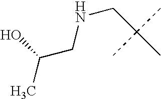 | CH | —H | 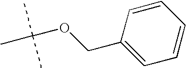 |
| A.423 | 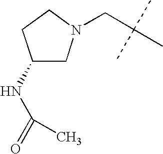 | CH | —H | 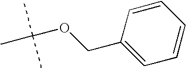 |
| A.424 | 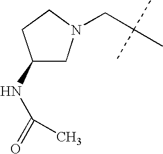 | CH | —H | 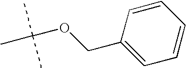 |
| A.425 | 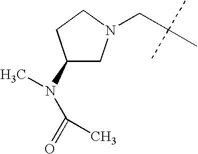 | CH | —H | 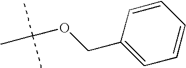 |
| A.426 | 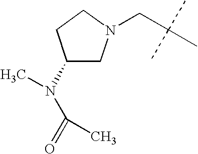 | CH | —H | 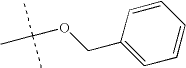 |
| A.427 | 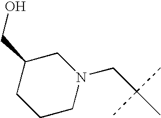 | CH | —H | 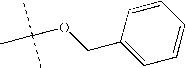 |
| A.428 | 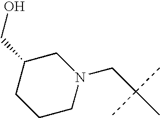 | CH | —H | 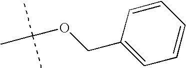 |
| A.429 | 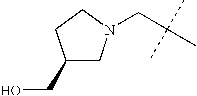 | CH | —H | 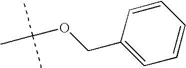 |
| A.430 | 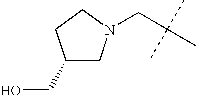 | CH | —H | 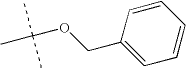 |
| A.431 | 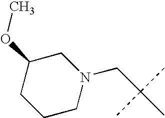 | CH | —H | 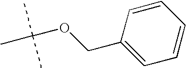 |
| A.432 | 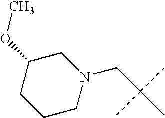 | CH | —H | 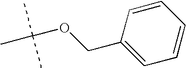 |
| A.433 | 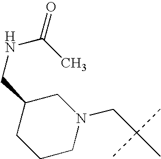 | CH | —H | 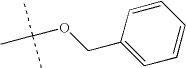 |
| A.434 | 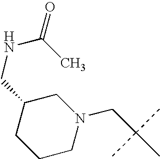 | CH | —H | 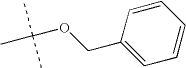 |
| A.435 | 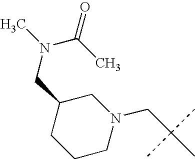 | CH | —H | 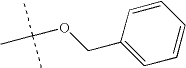 |
| A.436 | 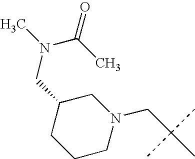 | CH | —H | 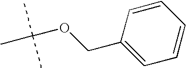 |
| A.437 | 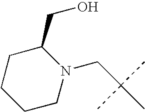 | CH | —H | 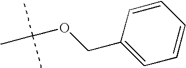 |
| A.438 | 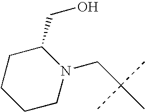 | CH | —H | 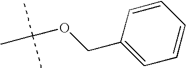 |
| A.439 | 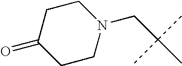 | CH | —H | 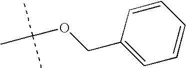 |
| A.440 | 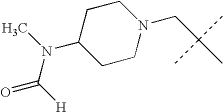 | CH | —H | 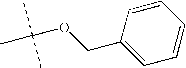 |
| A.441 | 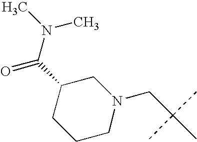 | CH | —H | 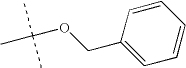 |
| A.442 | 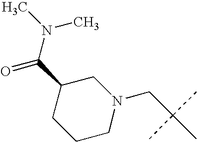 | CH | —H | 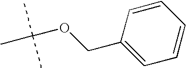 |
| A.443 | 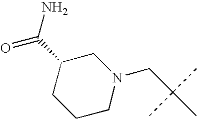 | CH | —H | 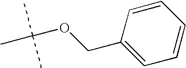 |
| A.444 | 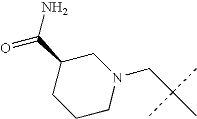 | CH | —H | 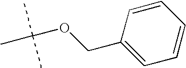 |
| A.445 | 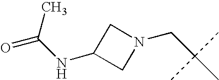 | CH | —H | 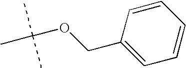 |
| A.446 | 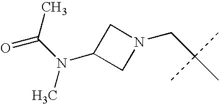 | CH | —H | 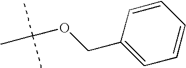 |
| A.447 | 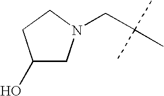 | CH | —H | 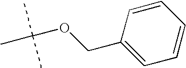 |
| A.448 | 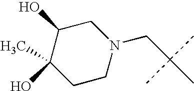 | CH | —H | 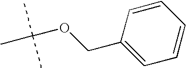 |
| A.449 | 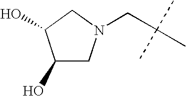 | CH | —H | 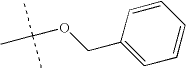 |
| A.450 | 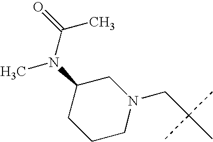 | CH | —H | 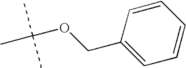 |
| A.451 | 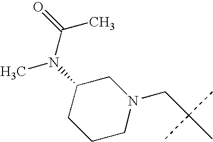 | CH | —H | 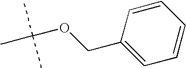 |
| A.452 | 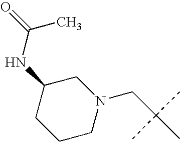 | CH | —H | 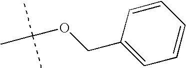 |
| A.453 | 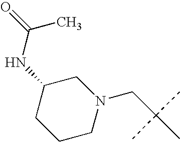 | CH | —H | 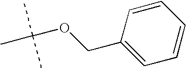 |
| A.454 | 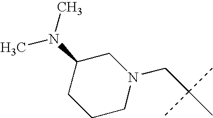 | CH | —H | 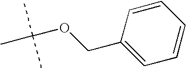 |
| A.455 | 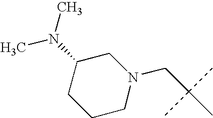 | CH | —H | 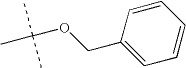 |
| A.456 | 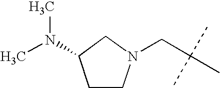 | CH | —H | 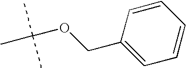 |
| A.457 | 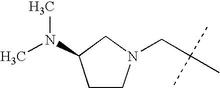 | CH | —H | 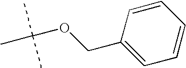 |
| A.458 | 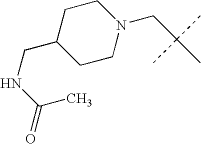 | CH | —H | 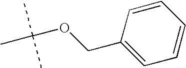 |
| A.459 | 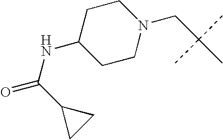 | CH | —H | 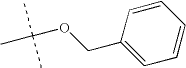 |
| A.460 | 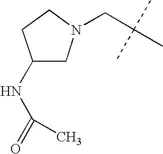 | CH | —H | 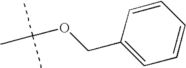 |
| A.461 | 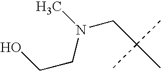 | CH | —H | 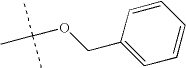 |
| A.462 | 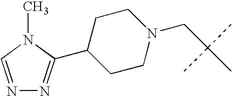 | CH | —H | 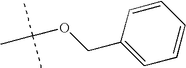 |
| A.463 | 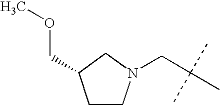 | CH | —H | 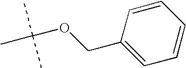 |
| A.464 | 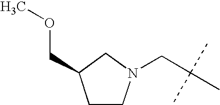 | CH | —H | 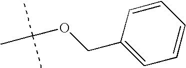 |
| A.465 | 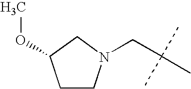 | CH | —H | 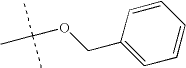 |
| A.466 | 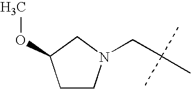 | CH | —H | 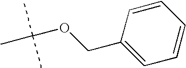 |
| A.467 | 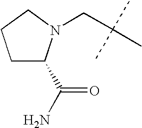 | CH | —H | 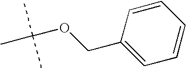 |
| A.468 | 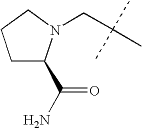 | CH | —H | 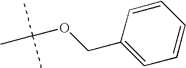 |
| A.469 | 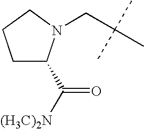 | CH | —H | 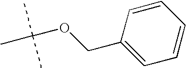 |
| A.470 | 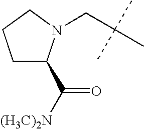 | CH | —H | 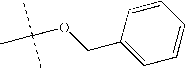 |
| A.471 | 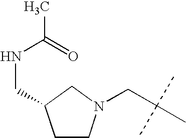 | CH | —H | 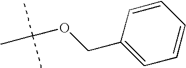 |
| A.472 | 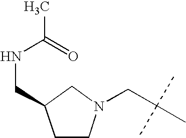 | CH | —H | 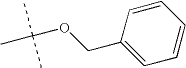 |
| A.473 | 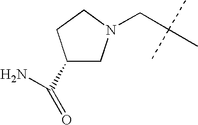 | CH | —H | 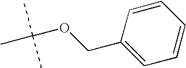 |
| A.474 | 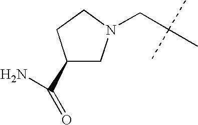 | CH | —H | 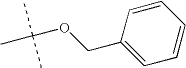 |
| A.475 | 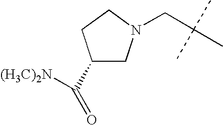 | CH | —H | 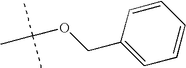 |
| A.476 | 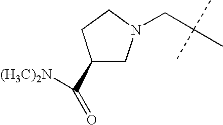 | CH | —H | 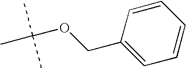 |
| A.477 | 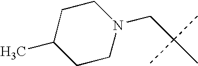 | CH | —H | 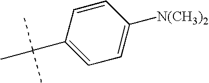 |
| A.478 | 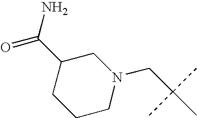 | CH | —H | 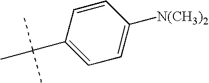 |
| A.479 | 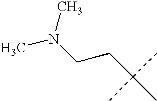 | CH | —H | 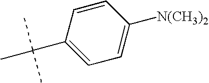 |
| A.480 | 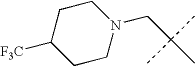 | CH | —H | 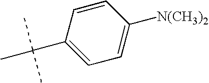 |
| A.481 | 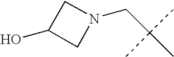 | CH | —H | 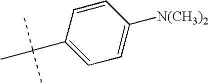 |
| A.482 | 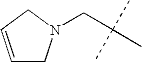 | CH | —H | 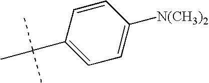 |
| A.483 | 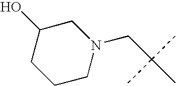 | CH | —H | 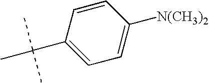 |
| A.484 | 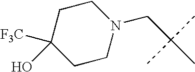 | CH | —H | 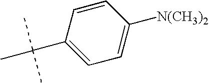 |
| A.485 | 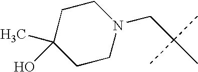 | CH | —H | 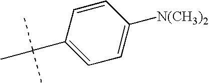 |
| A.486 | 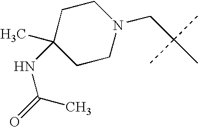 | CH | —H | 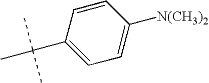 |
| A.487 | 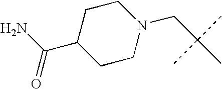 | CH | —H | 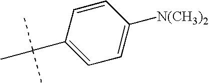 |
| A.488 | 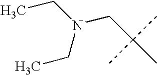 | CH | —H | 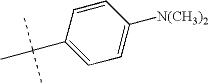 |
| A.489 | 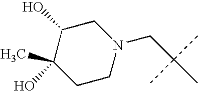 | CH | —H | 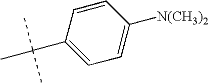 |
| A.490 | 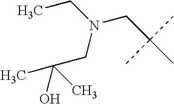 | CH | —H | 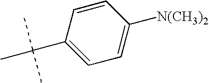 |
| A.491 | 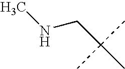 | CH | —H | 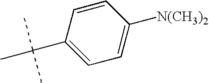 |
| A.492 | 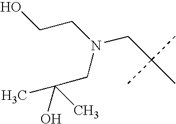 | CH | —H | 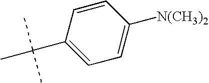 |
| A.493 | 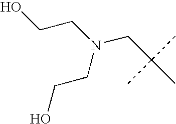 | CH | —H | 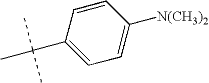 |
| A.494 | 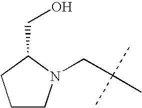 | CH | —H | 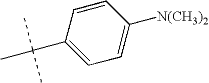 |
| A.495 | 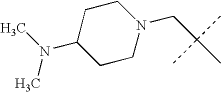 | CH | —H | 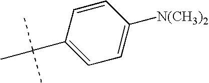 |
| A.496 | 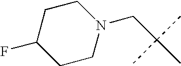 | CH | —H | 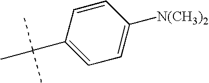 |
| A.497 | 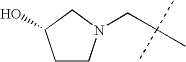 | CH | —H | 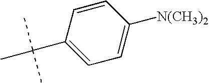 |
| A.498 | 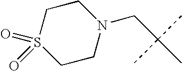 | CH | —H | 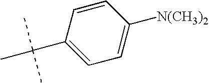 |
| A.499 | 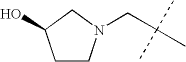 | CH | —H | 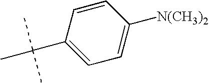 |
| A.500 | 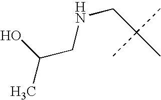 | CH | —H | 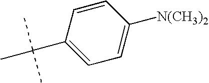 |
| A.501 | 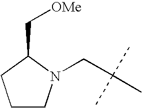 | CH | —H | 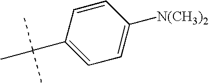 |
| A.502 | 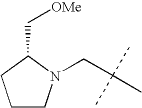 | CH | —H | 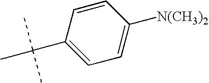 |
| A.503 | 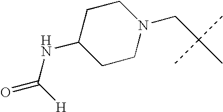 | CH | —H | 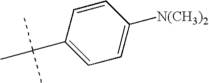 |
| A.504 | 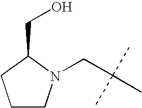 | CH | —H | 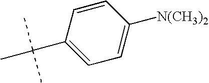 |
| A.505 | 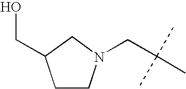 | CH | —H | 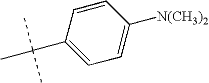 |
| A.506 | 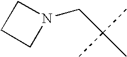 | CH | —H | 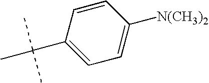 |
| A.507 | 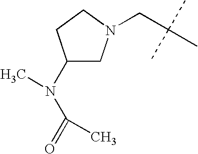 | CH | —H | 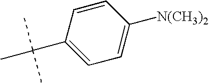 |
| A.508 | 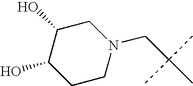 | CH | —H | 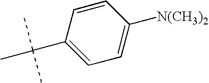 |
| A.509 | 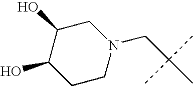 | CH | —H | 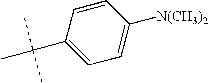 |
| A.510 | 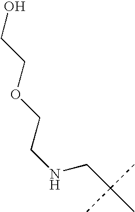 | CH | —H | 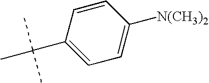 |
| A.511 | 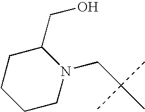 | CH | —H | 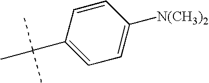 |
| A.512 | 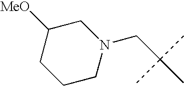 | CH | —H | 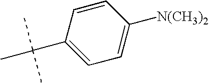 |
| A.513 | 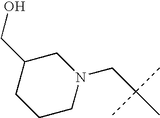 | CH | —H | 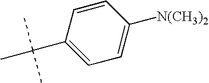 |
| A.514 | 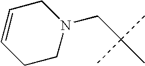 | CH | —H | 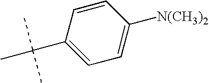 |
| A.515 | 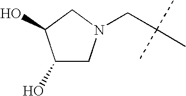 | CH | —H | 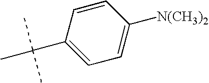 |
| A.516 | 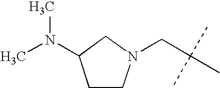 | CH | —H | 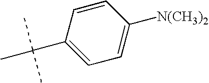 |
| A.517 | 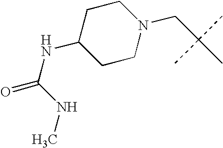 | CH | —H | 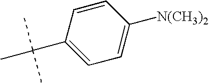 |
| A.518 | 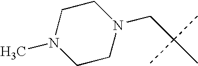 | CH | —H | 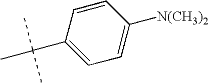 |
| A.519 | 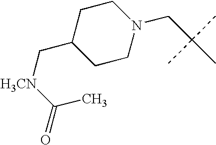 | CH | —H | 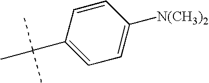 |
| A.520 | 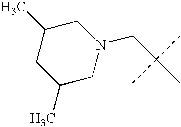 | CH | —H | 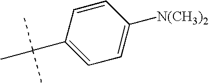 |
| A.521 | 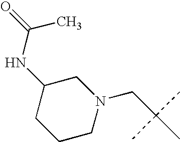 | CH | —H | 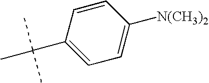 |
| A.522 | 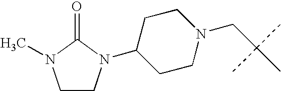 | CH | —H | 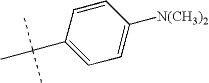 |
| A.523 | 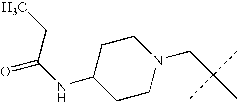 | CH | —H | 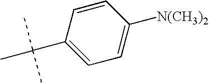 |
| A.524 | 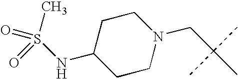 | CH | —H | 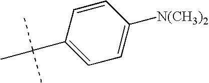 |
| A.525 | 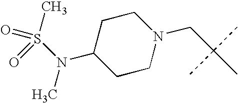 | CH | —H | 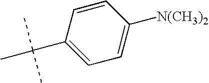 |
| A.526 | 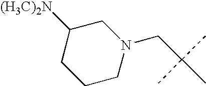 | CH | —H | 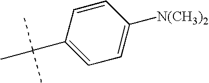 |
| A.527 | 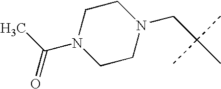 | CH | —H | 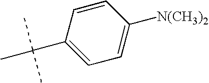 |
| A.528 | 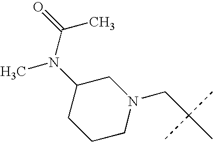 | CH | —H | 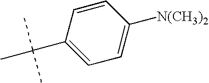 |
| A.529 | 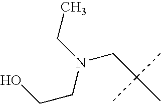 | CH | —H | 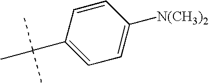 |
| A.530 | 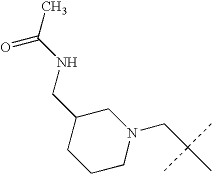 | CH | —H | 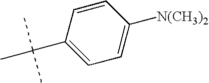 |
| A.531 | 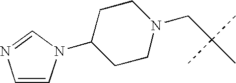 | CH | —H | 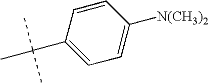 |
| A.532 | 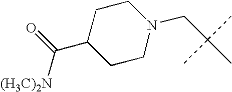 | CH | —H | 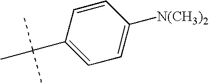 |
| A.533 | 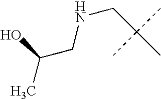 | CH | —H | 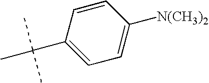 |
| A.534 | 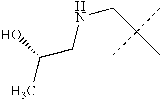 | CH | —H | 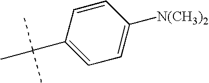 |
| A.535 | 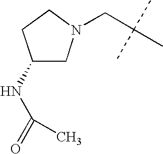 | CH | —H | 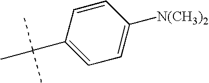 |
| A.536 | 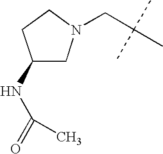 | CH | —H | 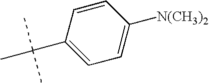 |
| A.537 | 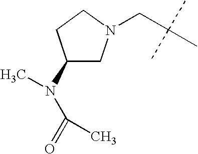 | CH | —H | 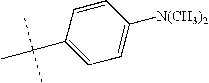 |
| A.538 | 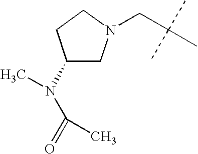 | CH | —H | 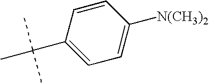 |
| A.539 | 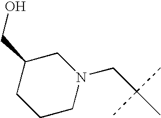 | CH | —H | 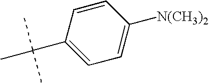 |
| A.540 | 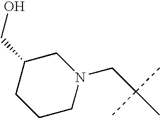 | CH | —H | 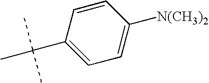 |
| A.541 | 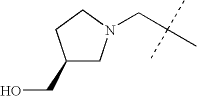 | CH | —H | 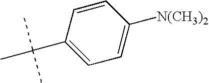 |
| A.542 | 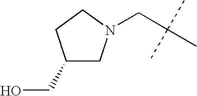 | CH | —H | 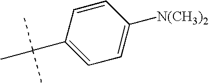 |
| A.543 | 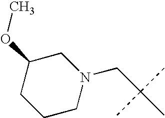 | CH | —H | 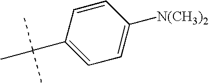 |
| A.544 | 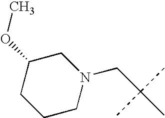 | CH | —H | 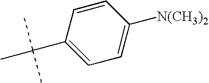 |
| A.545 | 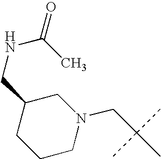 | CH | —H | 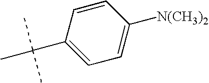 |
| A.546 | 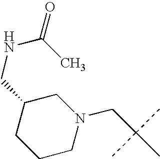 | CH | —H | 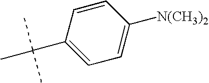 |
| A.547 | 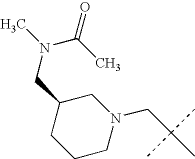 | CH | —H | 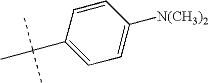 |
| A.548 | 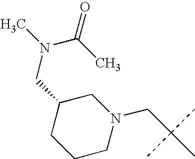 | CH | —H | 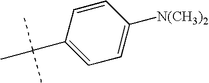 |
| A.549 | 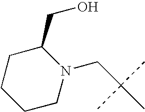 | CH | —H | 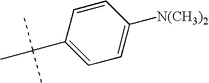 |
| A.550 | 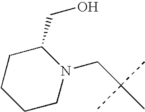 | CH | —H | 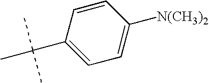 |
| A.551 | 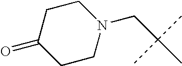 | CH | —H | 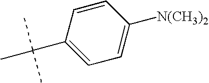 |
| A.552 | 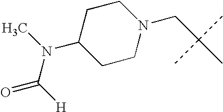 | CH | —H | 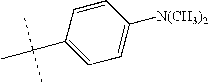 |
| A.553 | 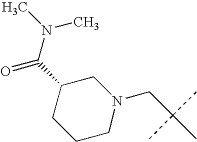 | CH | —H | 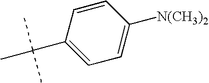 |
| A.554 | 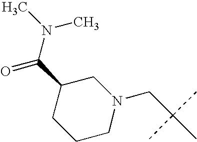 | CH | —H | 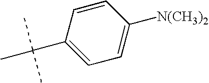 |
| A.555 | 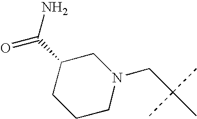 | CH | —H | 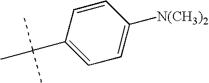 |
| A.556 | 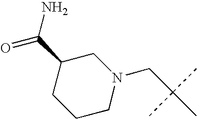 | CH | —H | 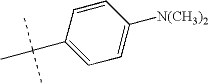 |
| A.557 | 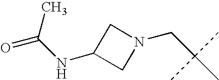 | CH | —H | 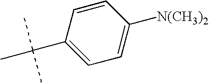 |
| A.558 | 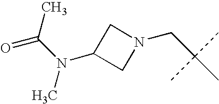 | CH | —H | 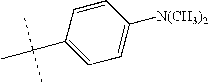 |
| A.559 | 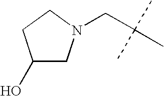 | CH | —H | 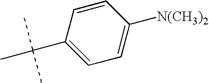 |
| A.560 | 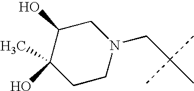 | CH | —H | 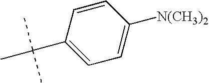 |
| A.561 | 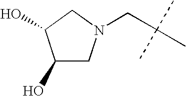 | CH | —H | 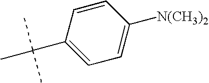 |
| A.562 | 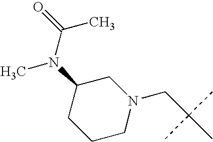 | CH | —H | 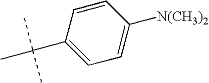 |
| A.563 | 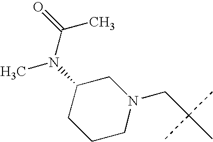 | CH | —H | 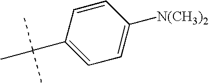 |
| A.564 | 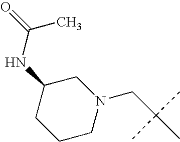 | CH | —H | 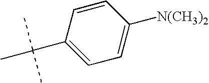 |
| A.565 | 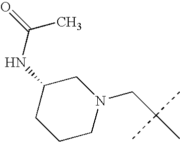 | CH | —H | 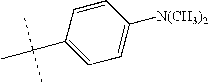 |
| A.566 | 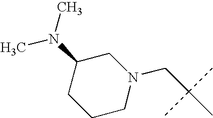 | CH | —H | 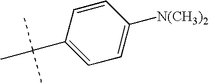 |
| A.567 | 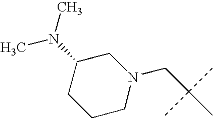 | CH | —H | 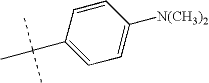 |
| A.568 | 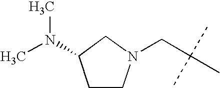 | CH | —H | 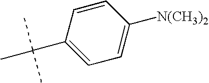 |
| A.569 | 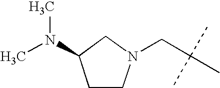 | CH | —H | 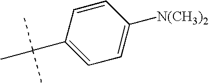 |
| A.570 | 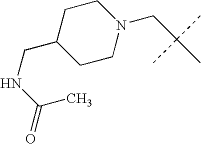 | CH | —H | 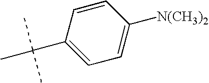 |
| A.571 | 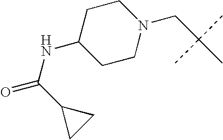 | CH | —H | 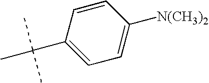 |
| A.572 | 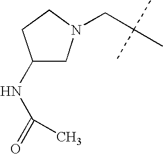 | CH | —H | 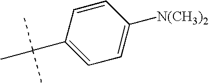 |
| A.573 | 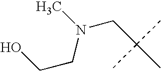 | CH | —H | 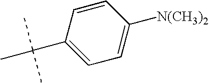 |
| A.574 | 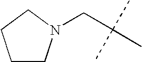 | CH | —H | 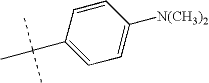 |
| A.575 | 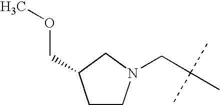 | CH | —H | 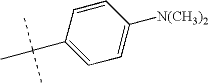 |
| A.576 | 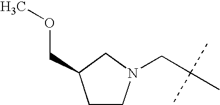 | CH | —H | 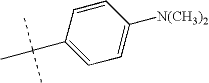 |
| A.577 | 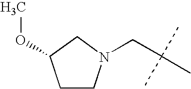 | CH | —H | 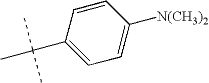 |
| A.578 | 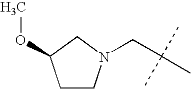 | CH | —H | 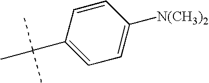 |
| A.579 | 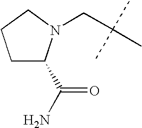 | CH | —H | 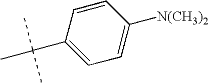 |
| A.580 | 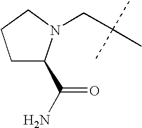 | CH | —H | 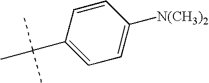 |
| A.581 | 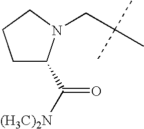 | CH | —H | 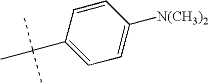 |
| A.582 | 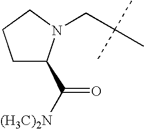 | CH | —H | 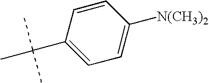 |
| A.583 | 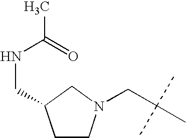 | CH | —H | 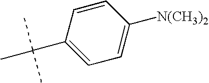 |
| A.584 | 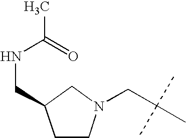 | CH | —H | 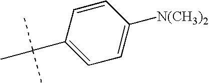 |
| A.585 | 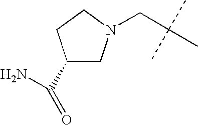 | CH | —H | 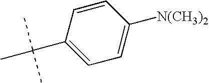 |
| A.586 | 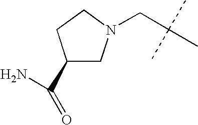 | CH | —H | 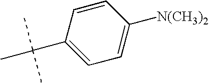 |
| A.587 | 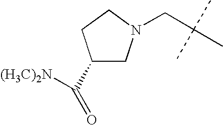 | CH | —H | 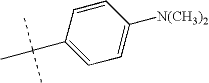 |
| A.588 | 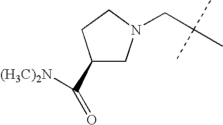 | CH | —H | 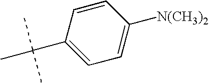 |
| A.589 | 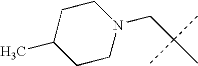 | CH | —H | 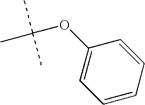 |
| A.590 | 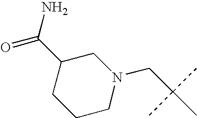 | CH | —H | 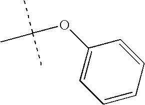 |
| A.591 | 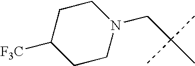 | CH | —H | 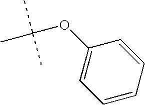 |
| A.592 | 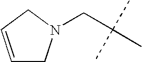 | CH | —H | 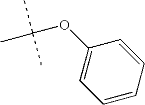 |
| A.593 | 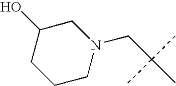 | CH | —H | 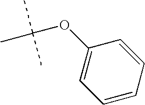 |
| A.594 | 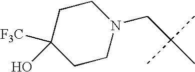 | CH | —H | 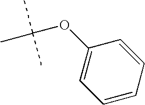 |
| A.595 | 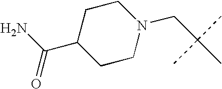 | CH | —H | 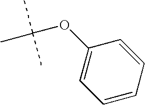 |
| A.596 | 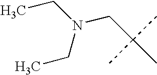 | CH | —H | 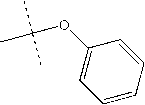 |
| A.597 | 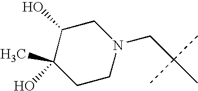 | CH | —H | 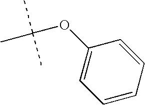 |
| A.598 | 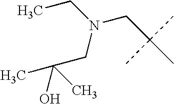 | CH | —H | 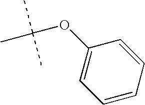 |
| A.599 | 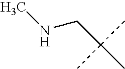 | CH | —H | 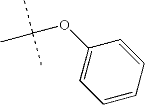 |
| A.600 | 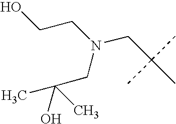 | CH | —H | 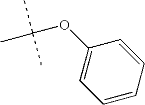 |
| A.601 | 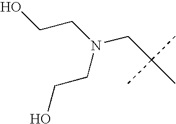 | CH | —H | 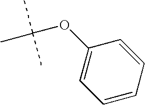 |
| A.602 | 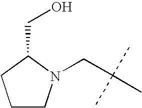 | CH | —H | 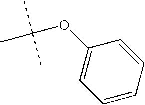 |
| A.603 | 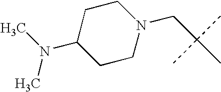 | CH | —H | 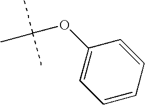 |
| A.604 | 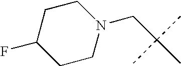 | CH | —H | 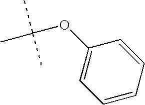 |
| A.605 | 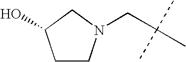 | CH | —H | 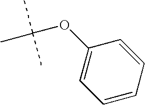 |
| A.606 | 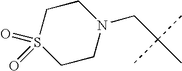 | CH | —H | 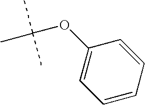 |
| A.607 | 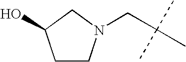 | CH | —H | 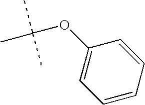 |
| A.608 | 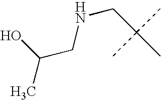 | CH | —H | 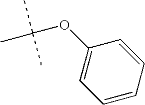 |
| A.609 | 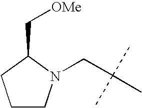 | CH | —H | 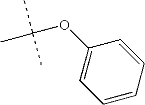 |
| A.610 | 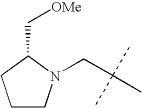 | CH | —H | 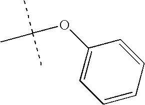 |
| A.611 | 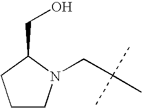 | CH | —H | 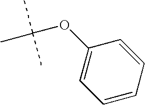 |
| A.612 | 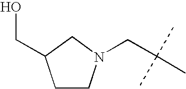 | CH | —H | 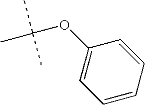 |
| A.613 | 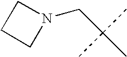 | CH | —H | 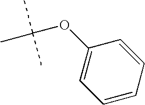 |
| A.614 | 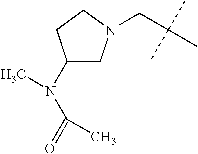 | CH | —H | 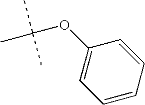 |
| A.615 | 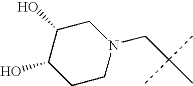 | CH | —H | 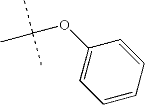 |
| A.616 | 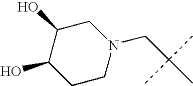 | CH | —H | 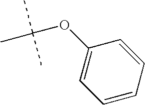 |
| A.617 | 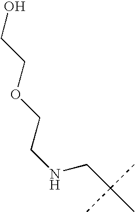 | CH | —H | 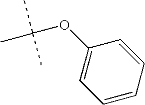 |
| A.618 | 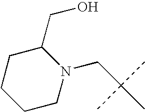 | CH | —H | 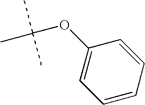 |
| A.619 | 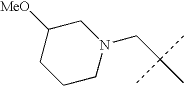 | CH | —H | 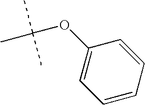 |
| A.620 | 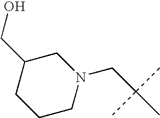 | CH | —H | 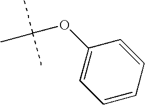 |
| A.621 | 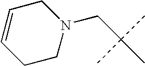 | CH | —H | 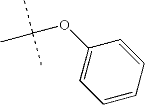 |
| A.622 | 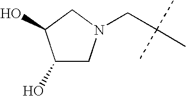 | CH | —H | 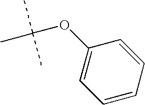 |
| A.623 | 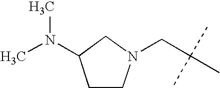 | CH | —H | 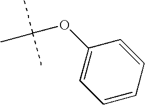 |
| A.624 | 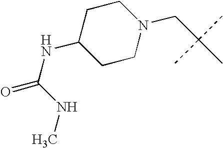 | CH | —H | 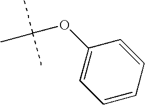 |
| A.625 | 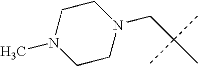 | CH | —H | 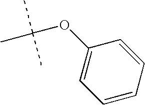 |
| A.626 | 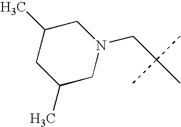 | CH | —H | 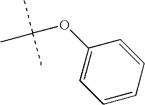 |
| A.627 | 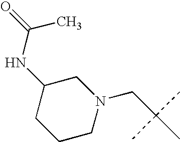 | CH | —H | 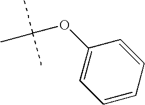 |
| A.628 | 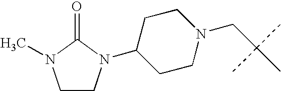 | CH | —H | 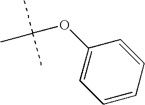 |
| A.629 | 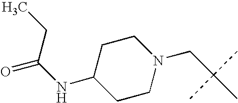 | CH | —H | 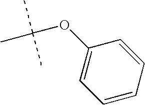 |
| A.630 | 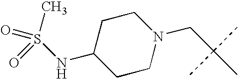 | CH | —H | 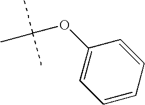 |
| A.631 | 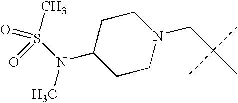 | CH | —H | 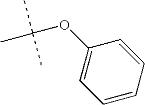 |
| A.632 | 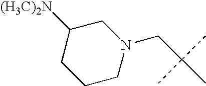 | CH | —H | 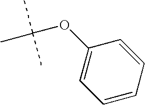 |
| A.633 | 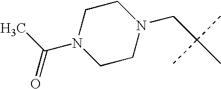 | CH | —H | 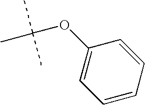 |
| A.634 | 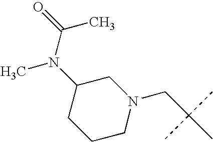 | CH | —H | 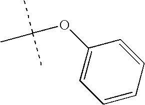 |
| A.635 | 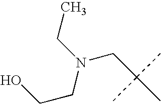 | CH | —H | 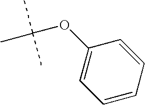 |
| A.636 | 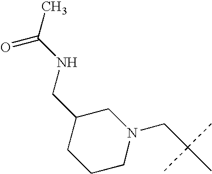 | CH | —H | 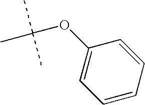 |
| A.637 | 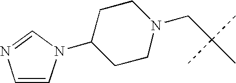 | CH | —H | 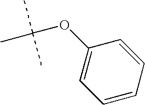 |
| A.638 | 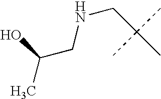 | CH | —H | 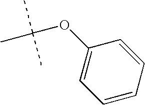 |
| A.639 | 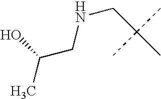 | CH | —H | 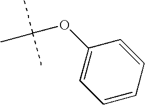 |
| A.640 | 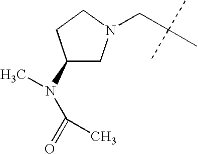 | CH | —H | 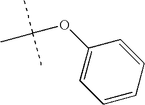 |
| A.641 | 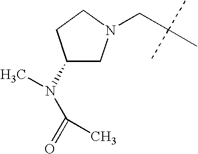 | CH | —H | 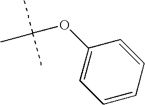 |
| A.642 | 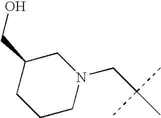 | CH | —H | 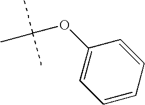 |
| A.643 | 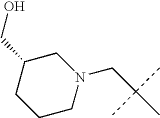 | CH | —H | 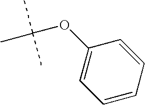 |
| A.644 | 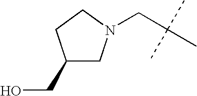 | CH | —H | 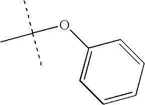 |
| A.645 | 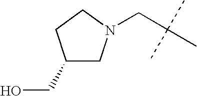 | CH | —H | 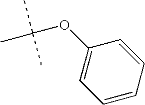 |
| A.646 | 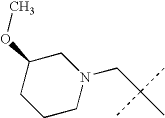 | CH | —H | 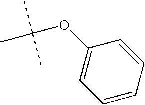 |
| A.647 | 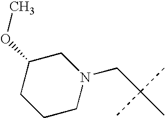 | CH | —H | 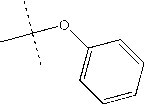 |
| A.648 | 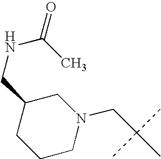 | CH | —H | 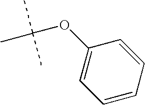 |
| A.649 | 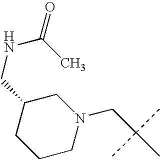 | CH | —H | 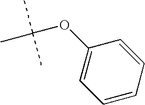 |
| A.650 | 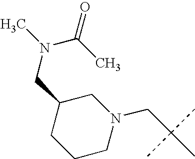 | CH | —H | 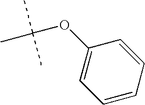 |
| A.651 | 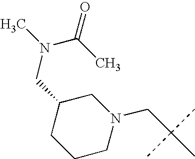 | CH | —H | 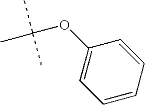 |
| A.652 | 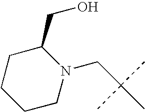 | CH | —H | 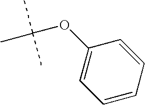 |
| A.653 | 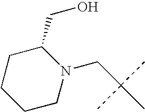 | CH | —H | 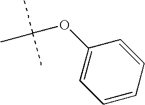 |
| A.654 | 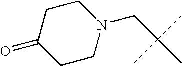 | CH | —H | 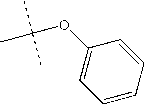 |
| A.655 | 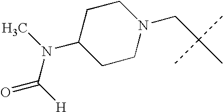 | CH | —H | 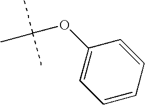 |
| A.656 | 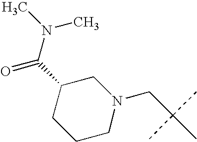 | CH | —H | 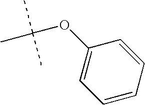 |
| A.657 | 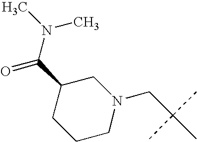 | CH | —H | 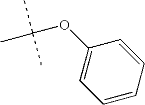 |
| A.658 | 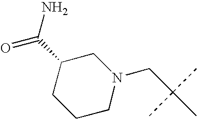 | CH | —H | 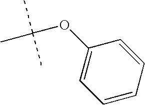 |
| A.659 | 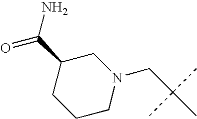 | CH | —H | 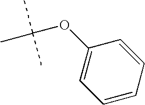 |
| A.660 | 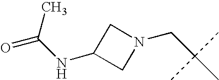 | CH | —H | 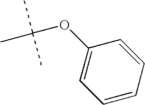 |
| A.661 | 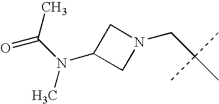 | CH | —H | 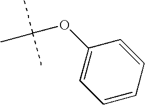 |
| A.662 | 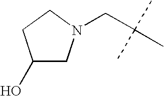 | CH | —H | 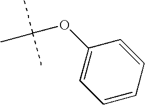 |
| A.663 | 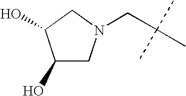 | CH | —H | 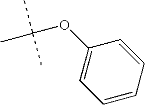 |
| A.664 | 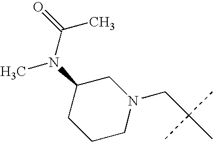 | CH | —H | 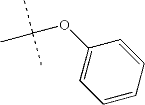 |
| A.665 | 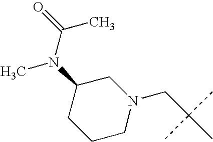 | CH | —H | 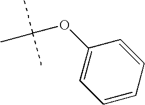 |
| A.666 | 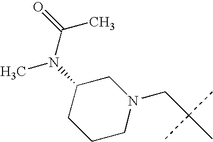 | CH | —H | 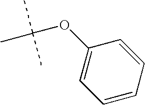 |
| A.667 | 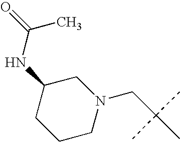 | CH | —H | 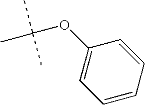 |
| A.668 | 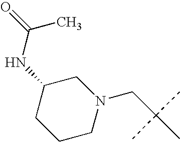 | CH | —H | 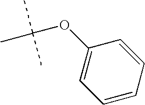 |
| A.669 | 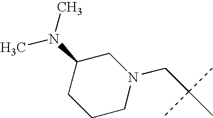 | CH | —H | 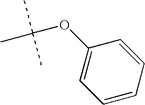 |
| A.670 | 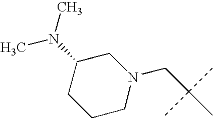 | CH | —H | 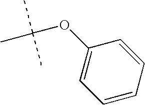 |
| A.671 | 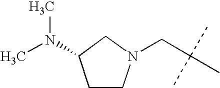 | CH | —H | 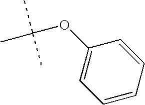 |
| A.672 | 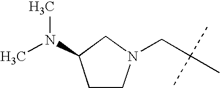 | CH | —H | 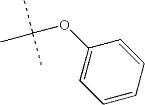 |
| A.673 | 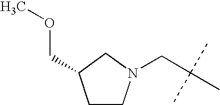 | CH | —H | 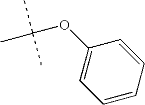 |
| A.674 | 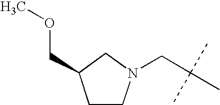 | CH | —H | 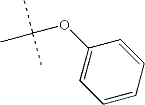 |
| A.675 | 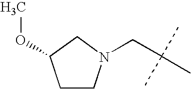 | CH | —H | 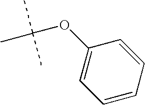 |
| A.676 | 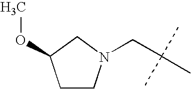 | CH | —H | 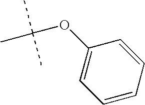 |
| A.677 | 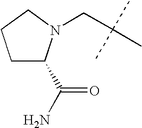 | CH | —H | 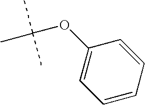 |
| A.678 | 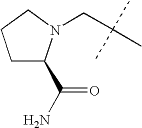 | CH | —H | 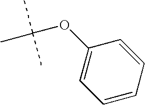 |
| A.679 | 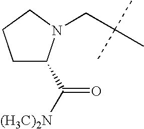 | CH | —H | 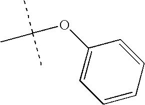 |
| A.680 | 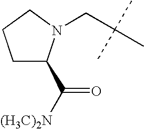 | CH | —H | 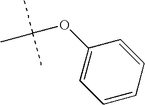 |
| A.681 | 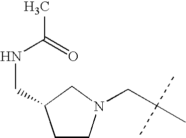 | CH | —H | 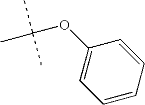 |
| A.682 | 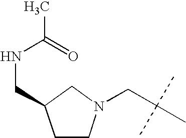 | CH | —H | 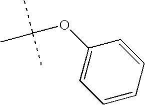 |
| A.683 | 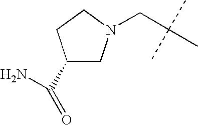 | CH | —H | 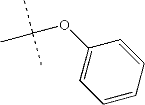 |
| A.684 | 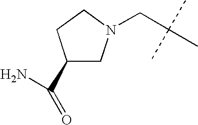 | CH | —H | 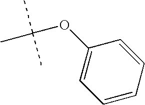 |
| A.685 | 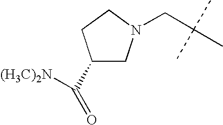 | CH | —H | 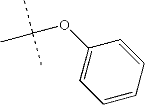 |
| A.686 | 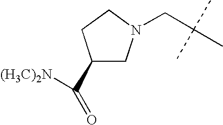 | CH | —H | 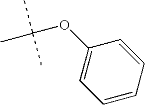 |
| A.687 | 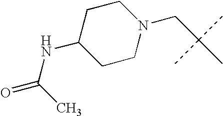 | CH | —H | 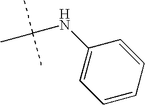 |
| A.688 | 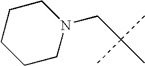 | CH | —H | 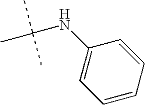 |
| A.689 | 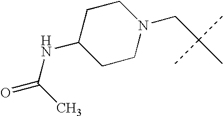 | CH | —H | 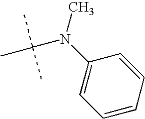 |
| A.690 | 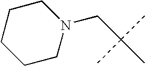 | CH | —H | 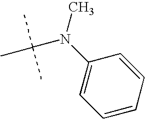 |
| A.691 | 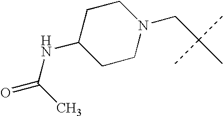 | CH | —H | 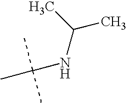 |
| A.692 | 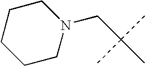 | CH | —H | 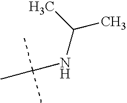 |
| A.693 | 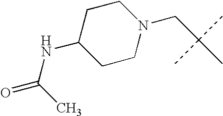 | CH | —H | 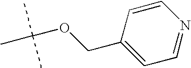 |
| A.694 | 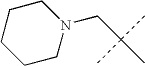 | CH | —H | 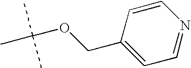 |
| A.695 | 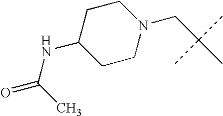 | CH | —H | 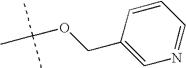 |
| A.696 | 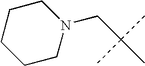 | CH | —H | 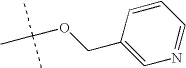 |
| A.697 | 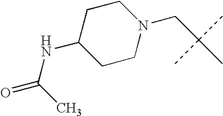 | CH | —H | 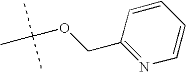 |
| A.698 | 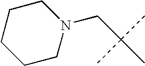 | CH | —H | 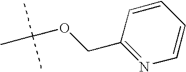 |
| A.699 | 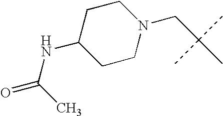 | CH | —H | 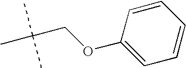 |
| A.700 | 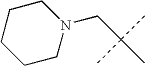 | CH | —H | 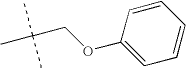 |
| A.701 | 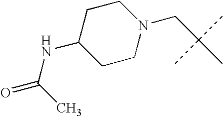 | CH | —H | 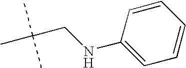 |
| A.702 | 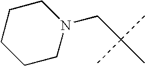 | CH | —H | 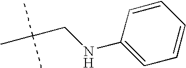 |
| A.703 | 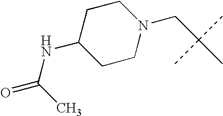 | CH | —H | 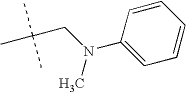 |
| A.704 | 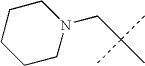 | CH | —H | 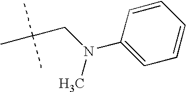 |
| A.705 | 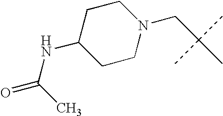 | CH | —H | 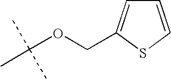 |
| A.706 | 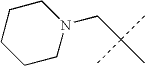 | CH | —H | 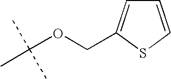 |
| A.707 | 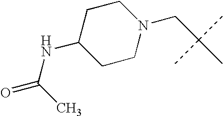 | CH | —H | 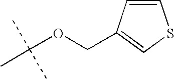 |
| A.708 | 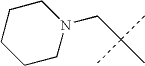 | CH | —H | 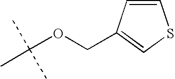 |
| A.709 | 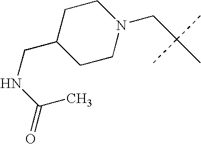 | CH | —H | 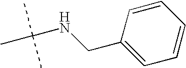 |
| A.710 | 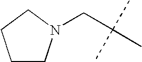 | CH | —H | 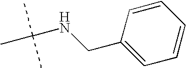 |
| A.711 | 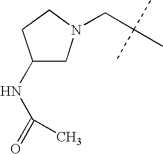 | CH | —H | 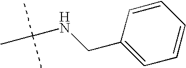 |
| A.712 | 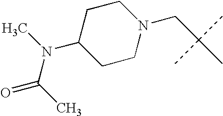 | CH | —H | 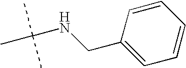 |
| A.713 | 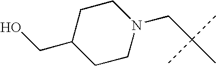 | CH | —H | 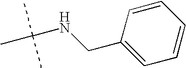 |
| A.714 | 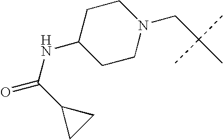 | CH | —H | 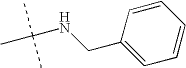 |
| A.715 | 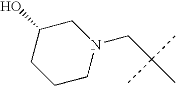 | CH | —H | 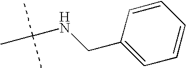 |
| A.716 | 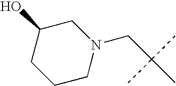 | CH | —H | 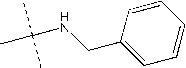 |
| A.717 | 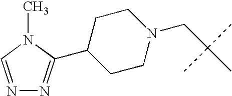 | CH | —H | 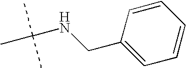 |
| A.718 | 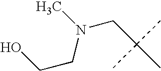 | CH | —H | 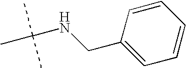 |
| A.719 | 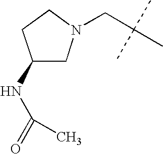 | CH | —H | 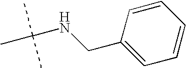 |
| A.720 | 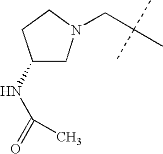 | CH | —H | 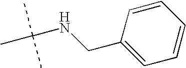 |
| A.721 | 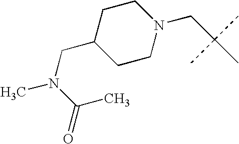 | CH | —H | 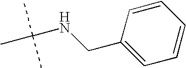 |
| A.722 | 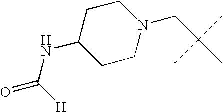 | CH | —H | 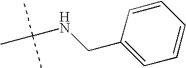 |
| A.723 | 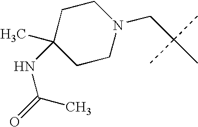 | CH | —H | 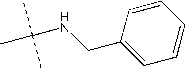 |
| A.724 | 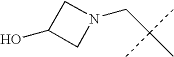 | CH | —H | 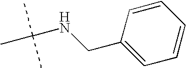 |
| A.725 | 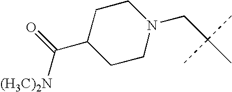 | CH | —H | 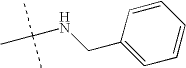 |

The following compounds of general formula (B-1) can be prepared analogously to the foregoing examples:

| | |
|---|---|
| | (B-1) |
| 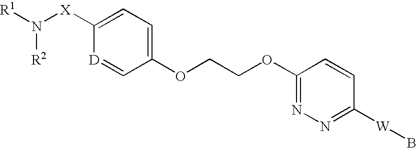 | |

| Example | R¹R²N-X- | D | -W-B |
|---|---|---|---|
| B.1 | 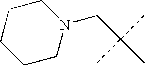 | CH | 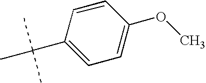 |
| B.2 | 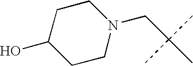 | CH | 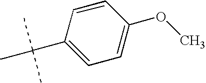 |

The following compounds of general formula (C-1) can be prepared analogously to the foregoing examples:

| | |
|---|---|
| | (C-1) |
| 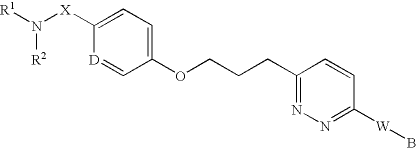 | |

| Example | R¹R²N-X- | D | -W-B |
|---|---|---|---|
| C.1 | 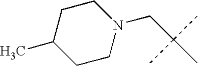 | CH | 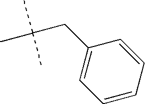 |
| C.2 | 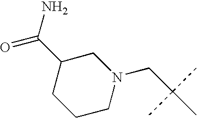 | CH | 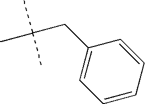 |
| C.3 | 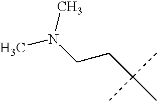 | CH | 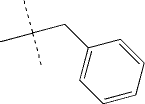 |
| C.4 | 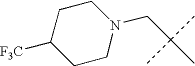 | CH | 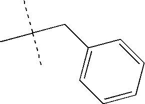 |
| C.5 | 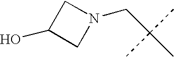 | CH | 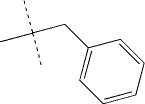 |
| C.6 | 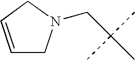 | CH | 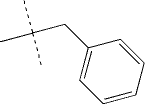 |
| C.7 | 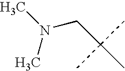 | CH | 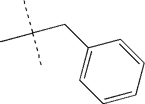 |
| C.8 | 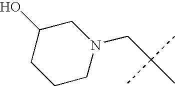 | CH | 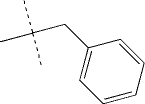 |
| C.9 | 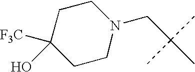 | CH | 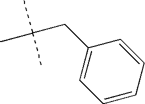 |
| C.10 | 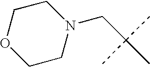 | CH | 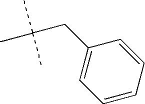 |
| C.11 | 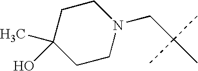 | CH | 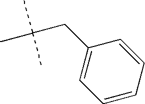 |
| C.12 | 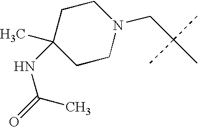 | CH | 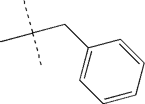 |
| C.13 | 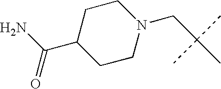 | CH | 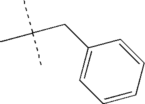 |
| C.14 | 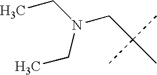 | CH | 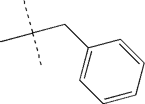 |
| C.15 | 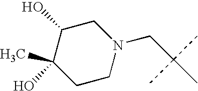 | CH | 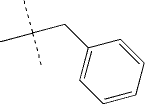 |
| C.16 | 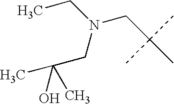 | CH | 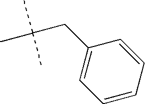 |
| C.17 | 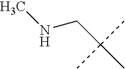 | CH | 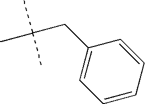 |
| C.18 | 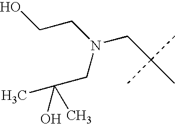 | CH | 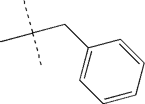 |
| C.19 | 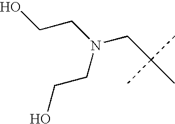 | CH | 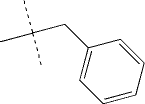 |
| C.20 | 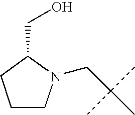 | CH | 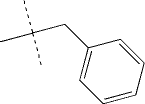 |
| C.21 | 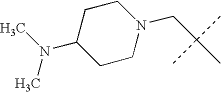 | CH | 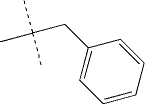 |
| C.22 | 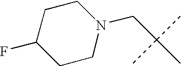 | CH | 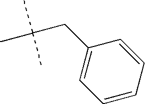 |
| C.23 | 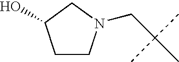 | CH | 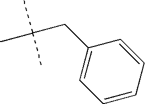 |
| C.24 | 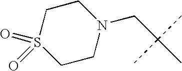 | CH | 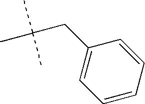 |
| C.25 | 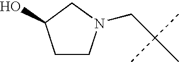 | CH | 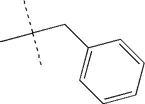 |
| C.26 | 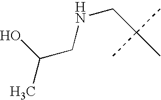 | CH | 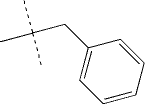 |
| C.27 | 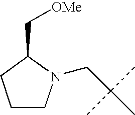 | CH | 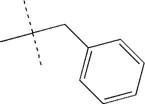 |
| C.28 | 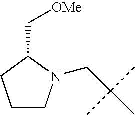 | CH | 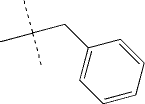 |
| C.29 | 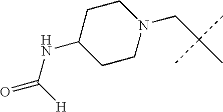 | CH | 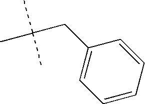 |
| C.30 | 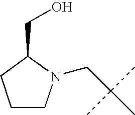 | CH | 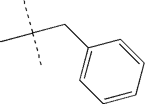 |
| C.31 | 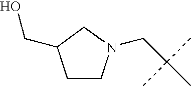 | CH | 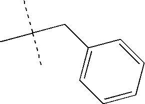 |
| C.32 | 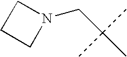 | CH | 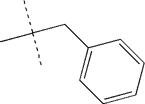 |
| C.33 | 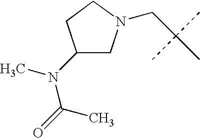 | CH | 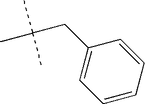 |
| C.34 | 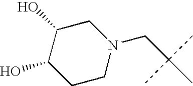 | CH | 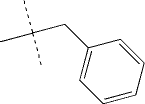 |
| C.35 | 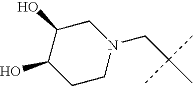 | CH | 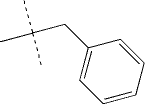 |
| C.36 | 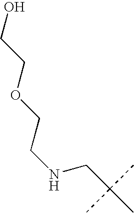 | CH | 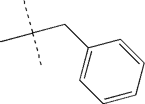 |
| C.37 | 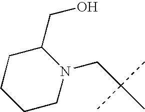 | CH | 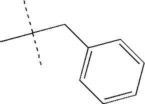 |
| C.38 | 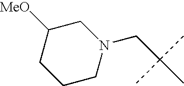 | CH | 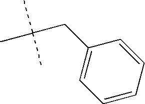 |
| C.39 | 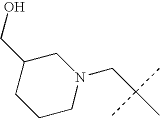 | CH | 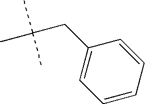 |
| C.40 | 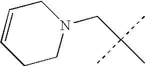 | CH | 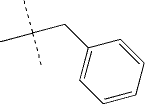 |
| C.41 | 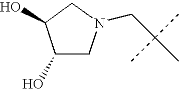 | CH | 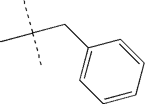 |
| C.42 | 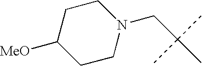 | CH | 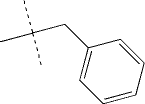 |
| C.43 | 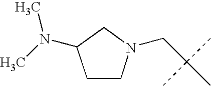 | CH | 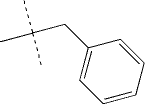 |
| C.44 | 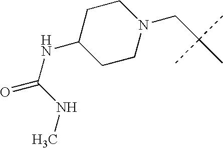 | CH | 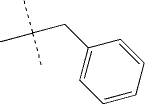 |
| C.45 | 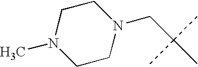 | CH | 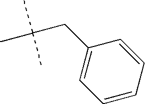 |
| C.46 | 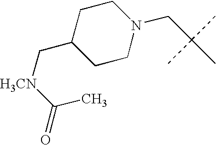 | CH | 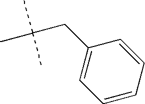 |
| C.47 | 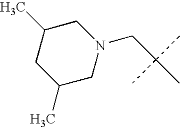 | CH | 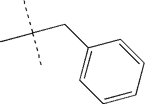 |
| C.48 | 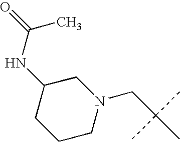 | CH | 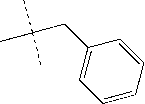 |
| C.49 | 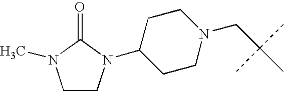 | CH | 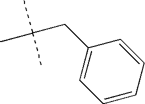 |
| C.50 | 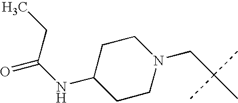 | CH | 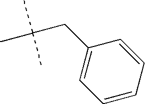 |
| C.51 | 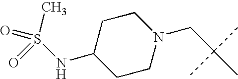 | CH | 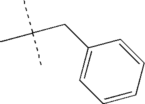 |
| C.52 | 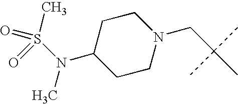 | CH | 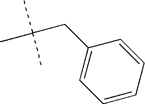 |
| C.53 | 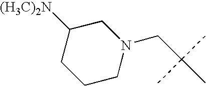 | CH | 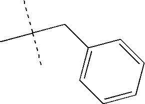 |
| C.54 | 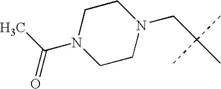 | CH | 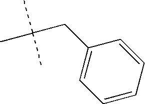 |
| C.55 | 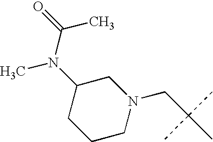 | CH | 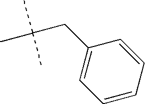 |
| C.56 | 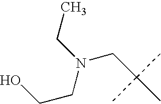 | CH | 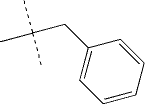 |
| C.57 | 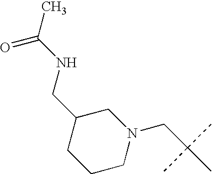 | CH | 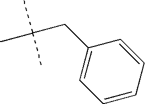 |
| C.58 | 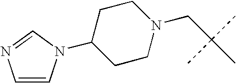 | CH | 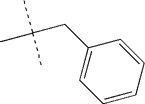 |
| C.59 | 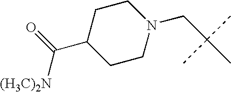 | CH | 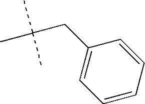 |
| C.60 | 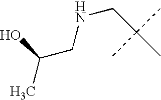 | CH | 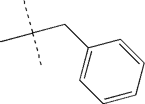 |
| C.61 | 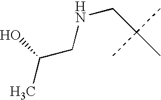 | CH | 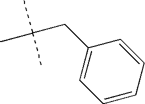 |
| C.62 | 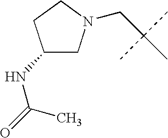 | CH | 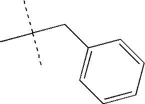 |
| C.63 | 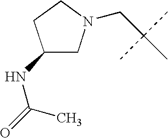 | CH | 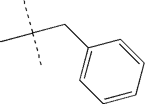 |
| C.64 | 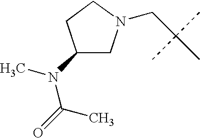 | CH | 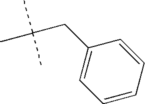 |
| C.65 | 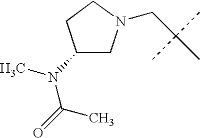 | CH | 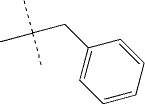 |
| C.66 | 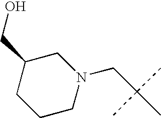 | CH | 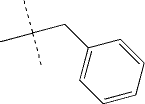 |
| C.67 | 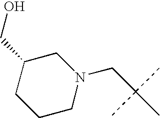 | CH | 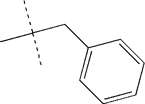 |
| C.68 | 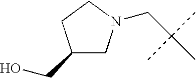 | CH | 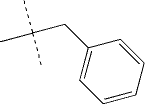 |
| C.69 | 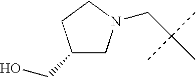 | CH | 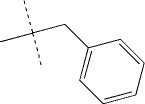 |
| C.70 | 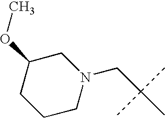 | CH | 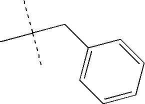 |
| C.71 | 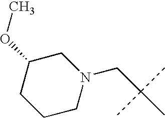 | CH | 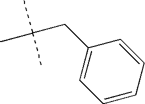 |
| C.72 | 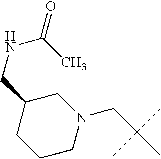 | CH | 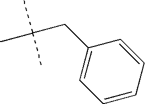 |
| C.73 | 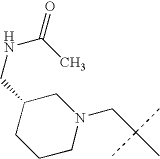 | CH | 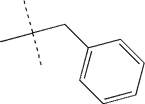 |
| C.74 | 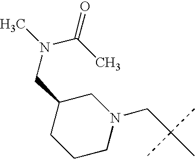 | CH | 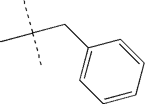 |
| C.75 | 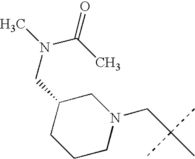 | CH | 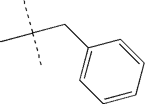 |
| C.76 | 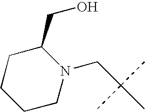 | CH | 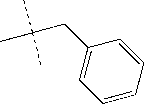 |
| C.77 | 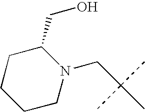 | CH | 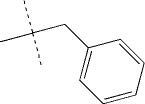 |
| C.78 | 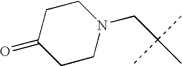 | CH | 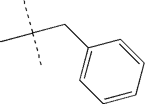 |
| C.79 | 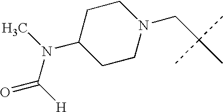 | CH | 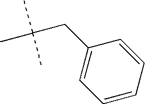 |
| C.80 | 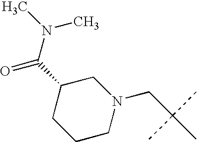 | CH | 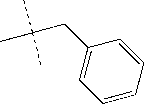 |
| C.81 | 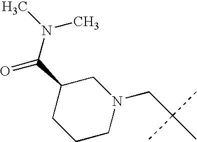 | CH | 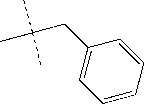 |
| C.82 | 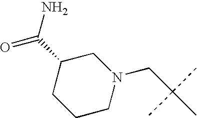 | CH | 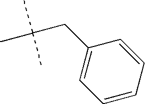 |
| C.83 | 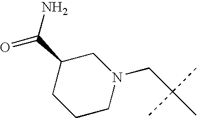 | CH | 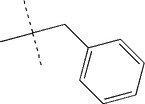 |
| C.84 | 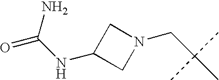 | CH | 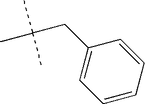 |
| C.85 | 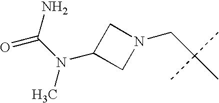 | CH | 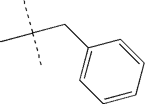 |
| C.86 | 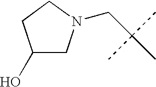 | CH | 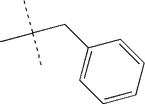 |
| C.87 | 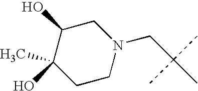 | CH | 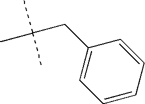 |
| C.88 | 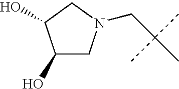 | CH | 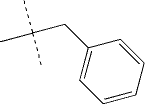 |
| C.89 | 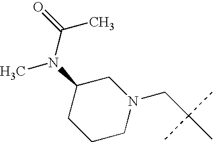 | CH | 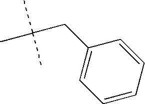 |
| C.90 | 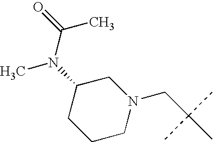 | CH | 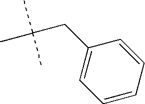 |
| C.91 | 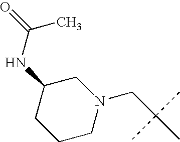 | CH | 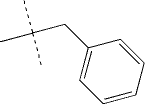 |
| C.92 | 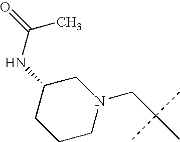 | CH | 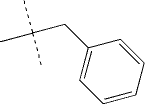 |
| C.93 | 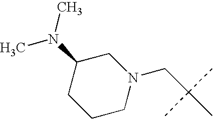 | CH | 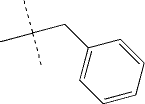 |
| C.94 | 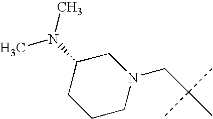 | CH | 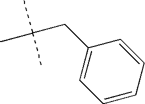 |
| C.95 | 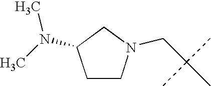 | CH | 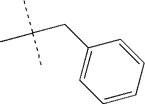 |
| C.96 | 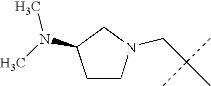 | CH | 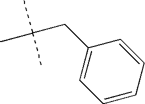 |
| C.97 | 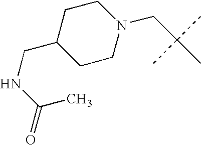 | CH | 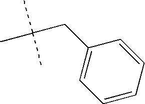 |
| C.98 | 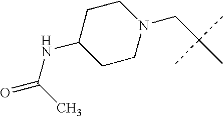 | CH | 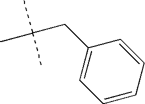 |
| C.99 | 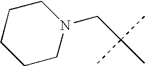 | CH | 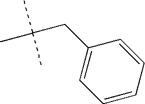 |
| C.100 | 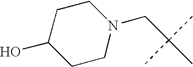 | CH | 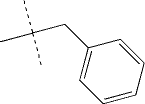 |
| C.101 | 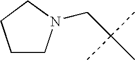 | CH | 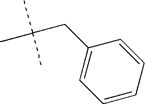 |
| C.102 | 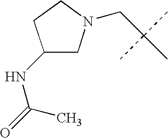 | CH | 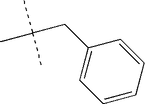 |
| C.103 | 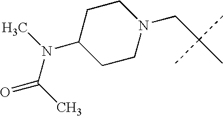 | CH | 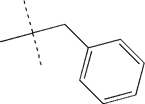 |
| C.104 | 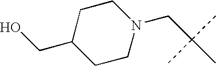 | CH | 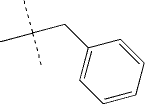 |
| C.105 | 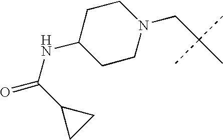 | CH | 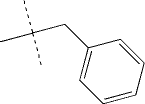 |
| C.106 | 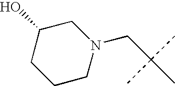 | CH | 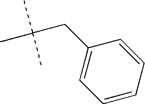 |
| C.107 | 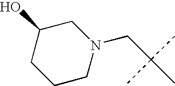 | CH | 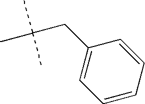 |
| C.108 | 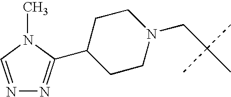 | CH | 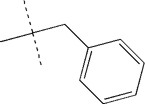 |
| C.109 | 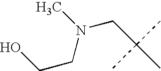 | CH | 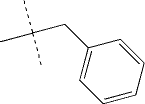 |
| C.110 | 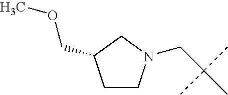 | CH | 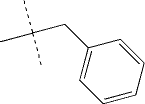 |
| C.111 | 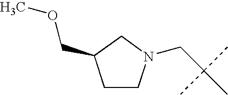 | CH | 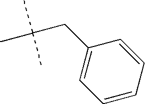 |
| C.112 | 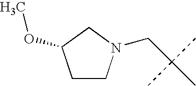 | CH | 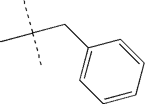 |
| C.113 | 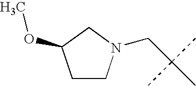 | CH | 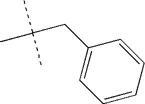 |
| C.114 | 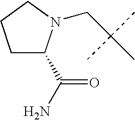 | CH | 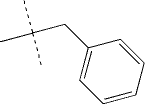 |
| C.115 | 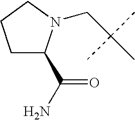 | CH | 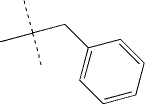 |
| C.116 | 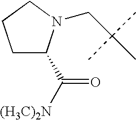 | CH | 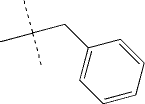 |
| C.117 | 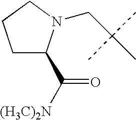 | CH | 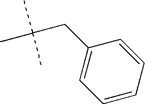 |
| C.118 | 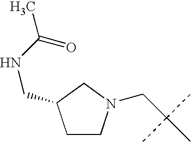 | CH | 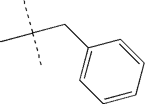 |
| C.119 | 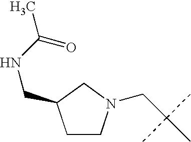 | CH | 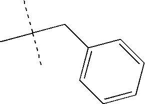 |
| C.120 | 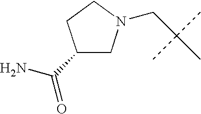 | CH | 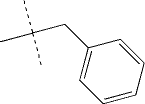 |
| C.121 | 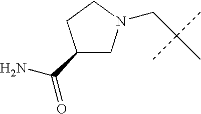 | CH | 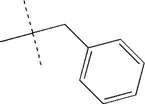 |
| C.122 | 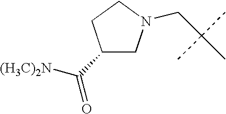 | CH | 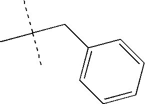 |
| C.123 | 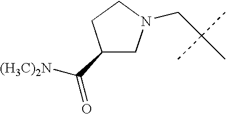 | CH | 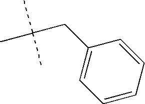 |
| C.124 | 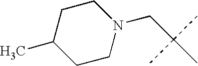 | CH | 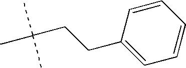 |
| C.125 | 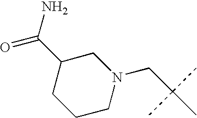 | CH | 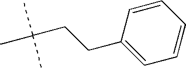 |
| C.126 | 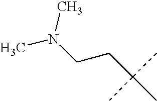 | CH | 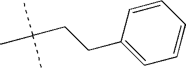 |
| C.127 | 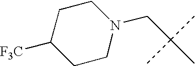 | CH | 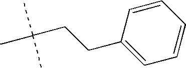 |
| C.128 | 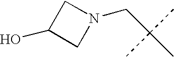 | CH | 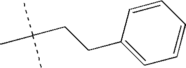 |
| C.129 | 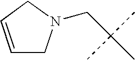 | CH | 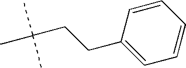 |
| C.130 | 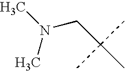 | CH | 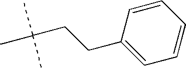 |
| C.131 | 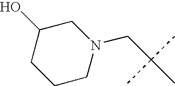 | CH | 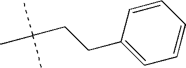 |
| C.132 | 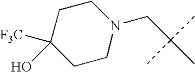 | CH | 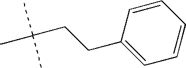 |
| C.133 | 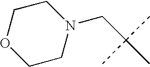 | CH | 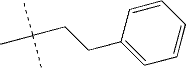 |
| C.134 | 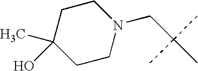 | CH | 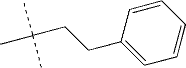 |
| C.135 | 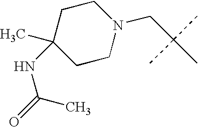 | CH | 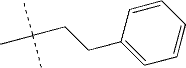 |
| C.136 | 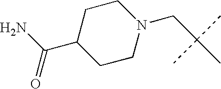 | CH | 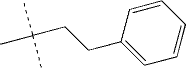 |
| C.137 | 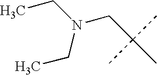 | CH | 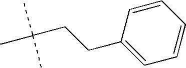 |
| C.138 | 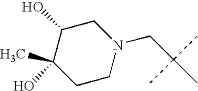 | CH | 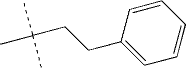 |
| C.139 | 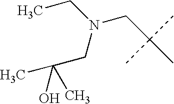 | CH | 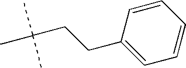 |
| C.140 | 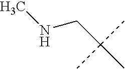 | CH | 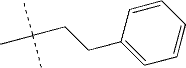 |
| C.141 | 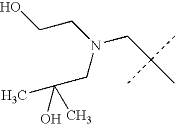 | CH | 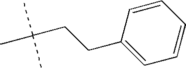 |
| C.142 | 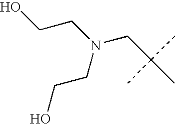 | CH | 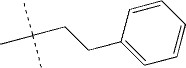 |
| C.143 | 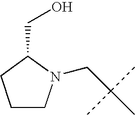 | CH | 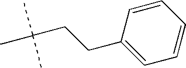 |
| C.144 | 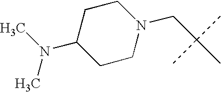 | CH | 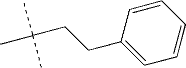 |
| C.145 | 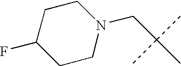 | CH | 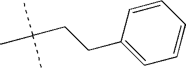 |
| C.146 | 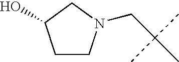 | CH | 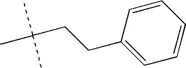 |
| C.147 | 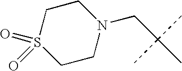 | CH | 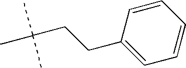 |
| C.148 | 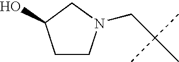 | CH | 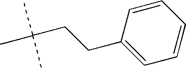 |
| C.149 | 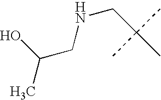 | CH | 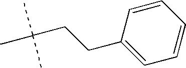 |
| C.150 | 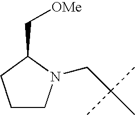 | CH | 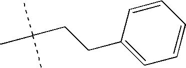 |
| C.151 | 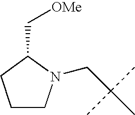 | CH | 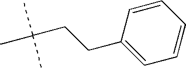 |
| C.152 | 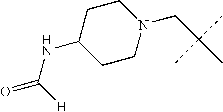 | CH | 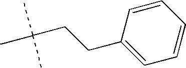 |
| C.153 | 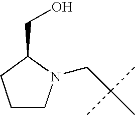 | CH | 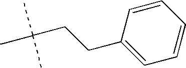 |
| C.154 | 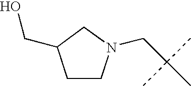 | CH | 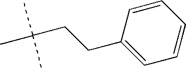 |
| C.155 | 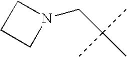 | CH | 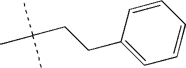 |
| C.156 | 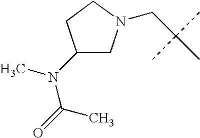 | CH | 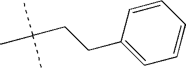 |
| C.157 | 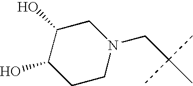 | CH | 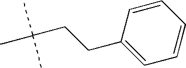 |
| C.158 | 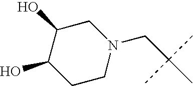 | CH | 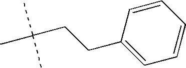 |
| C.159 | 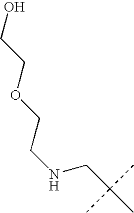 | CH | 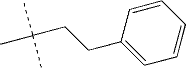 |
| C.160 | 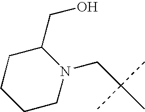 | CH | 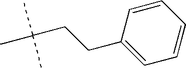 |
| C.161 | 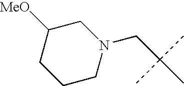 | CH | 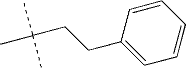 |
| C.162 | 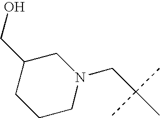 | CH | 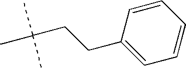 |
| C.163 | 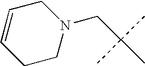 | CH | 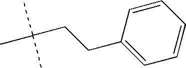 |
| C.164 | 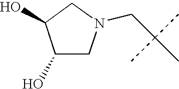 | CH | 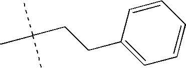 |
| C.165 | 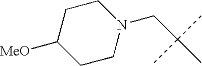 | CH | 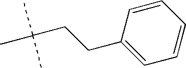 |
| C.166 | 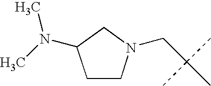 | CH | 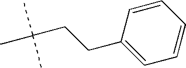 |
| C.167 | 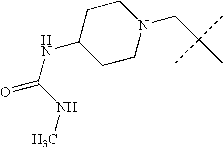 | CH | 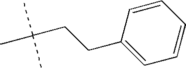 |
| C.168 | 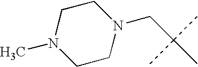 | CH | 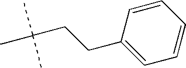 |
| C.169 | 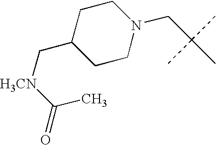 | CH | 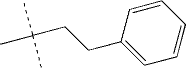 |
| C.170 | 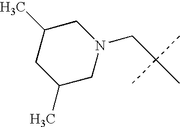 | CH | 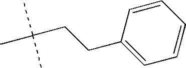 |
| C.171 | 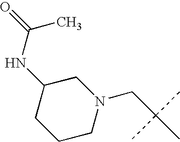 | CH | 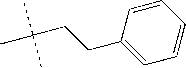 |
| C.172 | 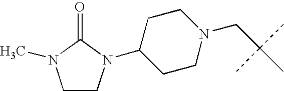 | CH | 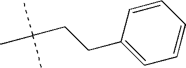 |
| C.173 | 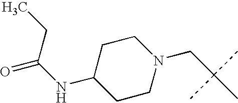 | CH | 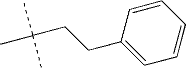 |
| C.174 | 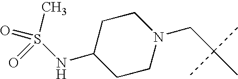 | CH | 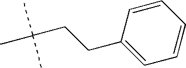 |
| C.175 | 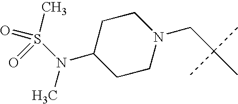 | CH | 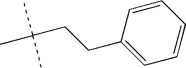 |
| C.176 | 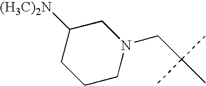 | CH | 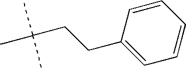 |
| C.177 | 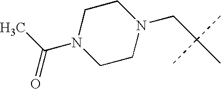 | CH | 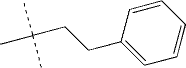 |
| C.178 | 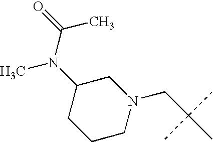 | CH | 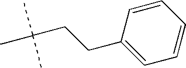 |
| C.179 | 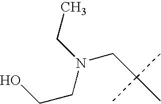 | CH | 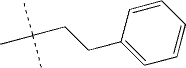 |
| C.180 | 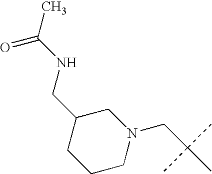 | CH | 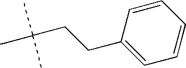 |
| C.181 | 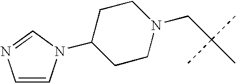 | CH | 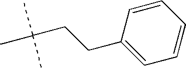 |
| C.182 | 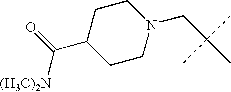 | CH | 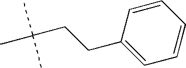 |
| C.183 | 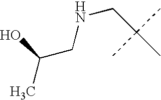 | CH | 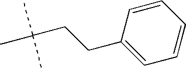 |
| C.184 | 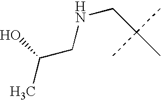 | CH | 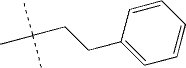 |
| C.185 | 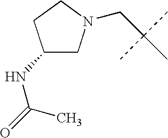 | CH | 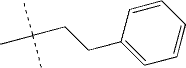 |
| C.186 | 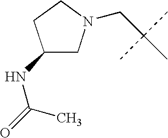 | CH | 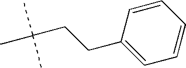 |
| C.187 | 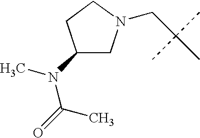 | CH | 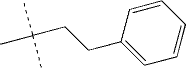 |
| C.188 | 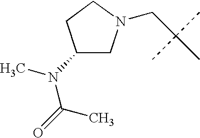 | CH | 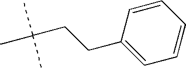 |
| C.189 | 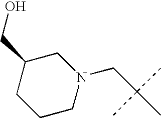 | CH | 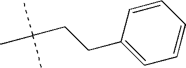 |
| C.190 | 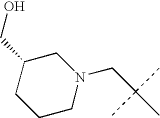 | CH | 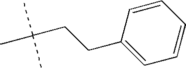 |
| C.191 | 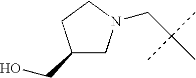 | CH | 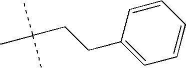 |
| C.192 | 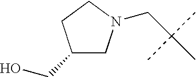 | CH | 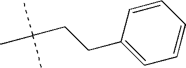 |
| C.193 | 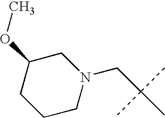 | CH | 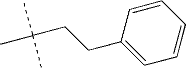 |
| C.194 | 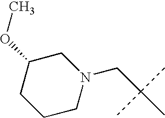 | CH | 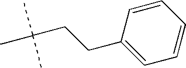 |
| C.195 | 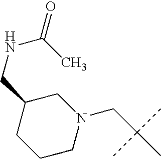 | CH | 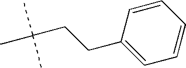 |
| C.196 | 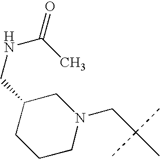 | CH | 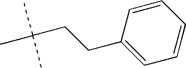 |
| C.197 | 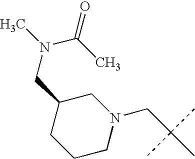 | CH | 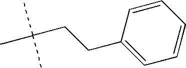 |
| C.198 | 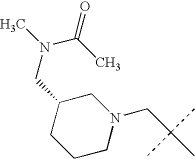 | CH | 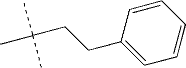 |
| C.199 | 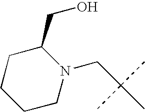 | CH | 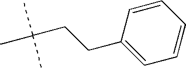 |
| C.200 | 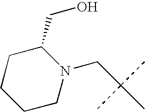 | CH | 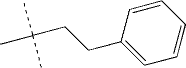 |
| C.201 | 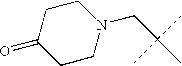 | CH | 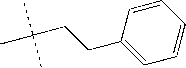 |
| C.202 | 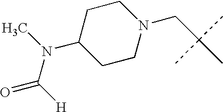 | CH | 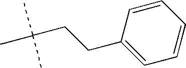 |
| C.203 | 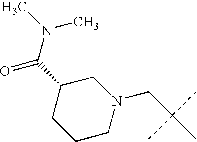 | CH | 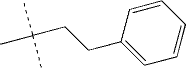 |
| C.204 | 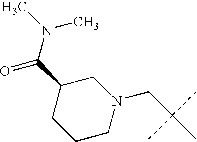 | CH | 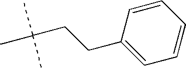 |
| C.205 | 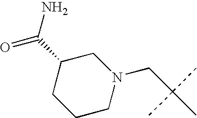 | CH | 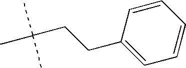 |
| C.206 | 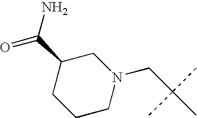 | CH | 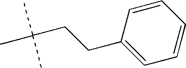 |
| C.207 | 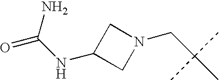 | CH | 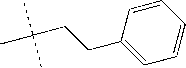 |
| C.208 | 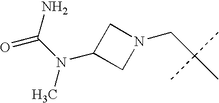 | CH | 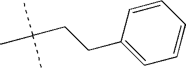 |
| C.209 | 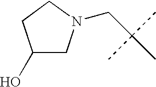 | CH | 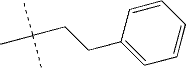 |
| C.210 | 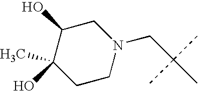 | CH | 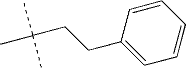 |
| C.211 | 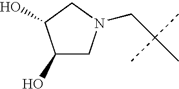 | CH | 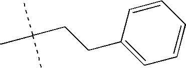 |
| C.212 | 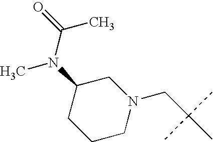 | CH | 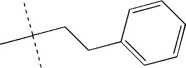 |
| C.213 | 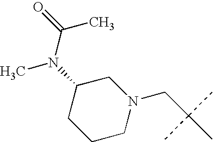 | CH | 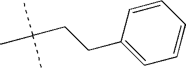 |
| C.214 | 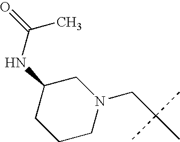 | CH | 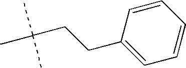 |
| C.215 | 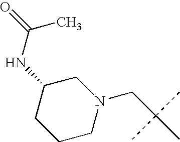 | CH | 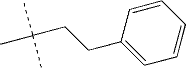 |
| C.216 | 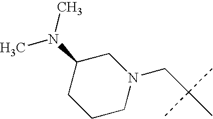 | CH | 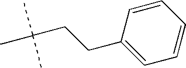 |
| C.217 | 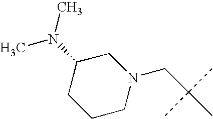 | CH | 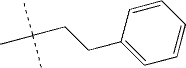 |
| C.218 | 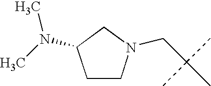 | CH | 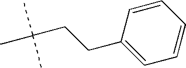 |
| C.219 | 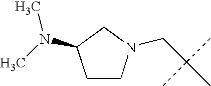 | CH | 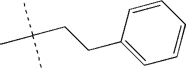 |
| C.220 | 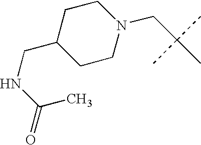 | CH | 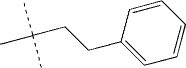 |
| C.221 | 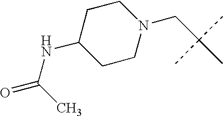 | CH | 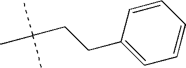 |
| C.222 | 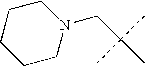 | CH | 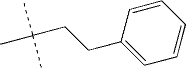 |
| C.223 | 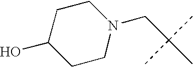 | CH | 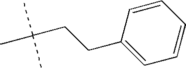 |
| C.224 | 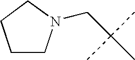 | CH | 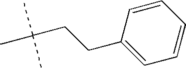 |
| C.225 | 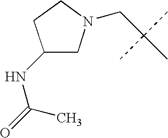 | CH | 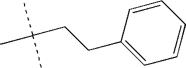 |
| C.226 | 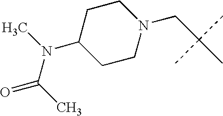 | CH | 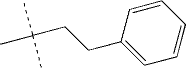 |
| C.227 | 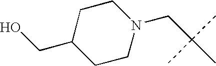 | CH | 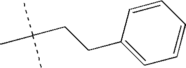 |
| C.228 | 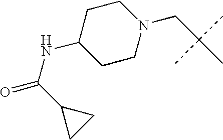 | CH | 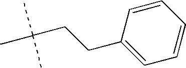 |
| C.229 | 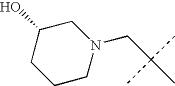 | CH | 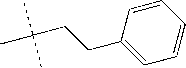 |
| C.230 | 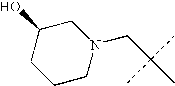 | CH | 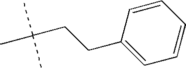 |
| C.231 | 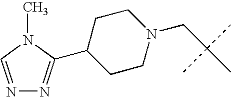 | CH | 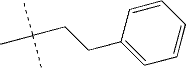 |
| C.232 | 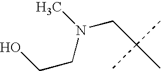 | CH | 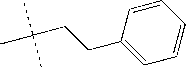 |
| C.233 | 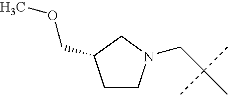 | CH | 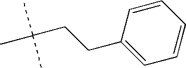 |
| C.234 | 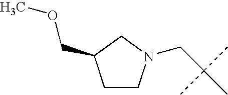 | CH | 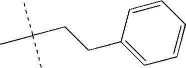 |
| C.235 | 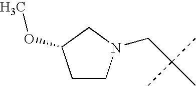 | CH | 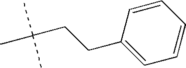 |
| C.236 | 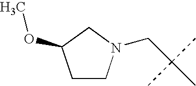 | CH | 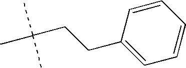 |
| C.237 | 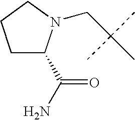 | CH | 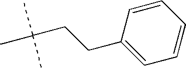 |
| C.238 | 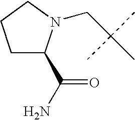 | CH | 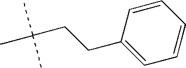 |
| C.239 | 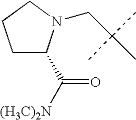 | CH | 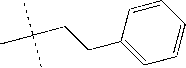 |
| C.240 | 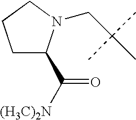 | CH | 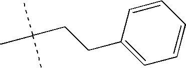 |
| C.241 | 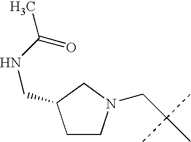 | CH | 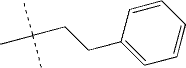 |
| C.242 | 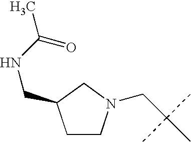 | CH | 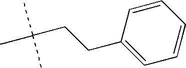 |
| C.243 | 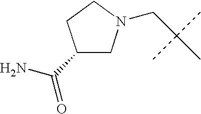 | CH | 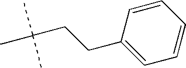 |
| C.244 | 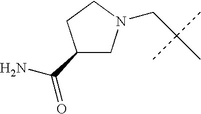 | CH | 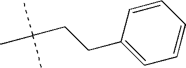 |
| C.245 | 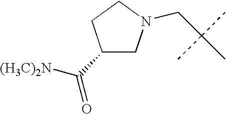 | CH | 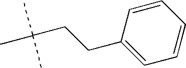 |
| C.246 | 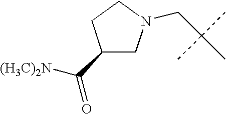 | CH | 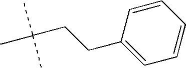 |
| C.247 | 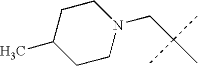 | CH | 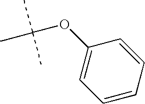 |
| C.248 | 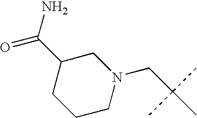 | CH | 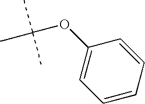 |
| C.249 | 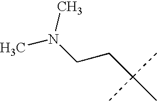 | CH | 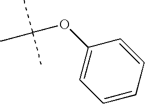 |
| C.250 | 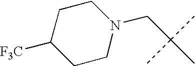 | CH | 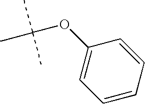 |
| C.251 | 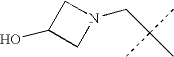 | CH | 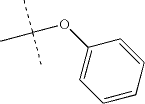 |
| C.252 | 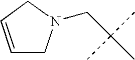 | CH | 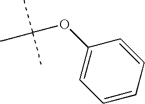 |
| C.253 | 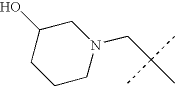 | CH | 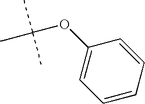 |
| C.254 | 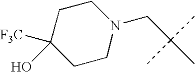 | CH | 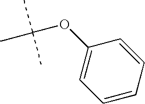 |
| C.255 | 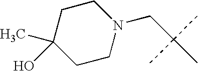 | CH | 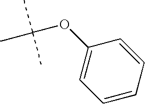 |
| C.256 | 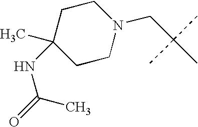 | CH | 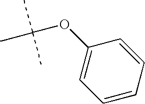 |
| C.257 | 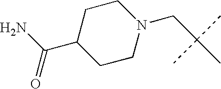 | CH | 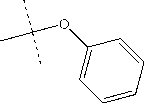 |
| C.258 | 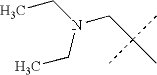 | CH | 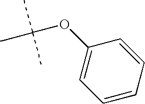 |
| C.259 | 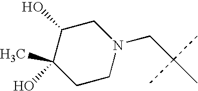 | CH | 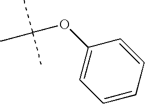 |
| C.260 | 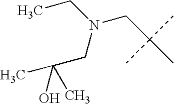 | CH | 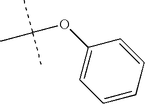 |
| C.261 | 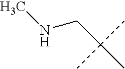 | CH | 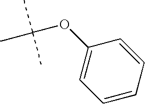 |
| C.262 | 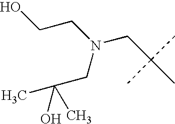 | CH | 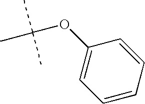 |
| C.263 | 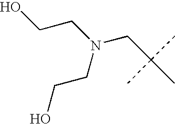 | CH | 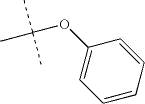 |
| C.264 | 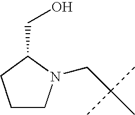 | CH | 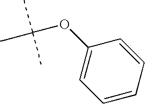 |
| C.265 | 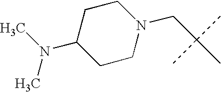 | CH | 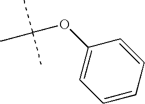 |
| C.266 | 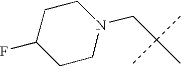 | CH | 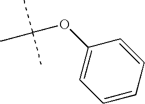 |
| C.267 | 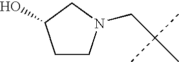 | CH | 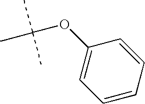 |
| C.268 | 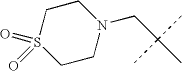 | CH | 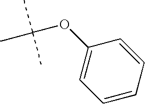 |
| C.269 | 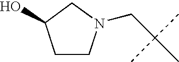 | CH | 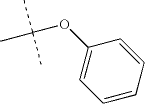 |
| C.270 | 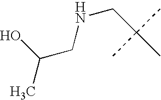 | CH | 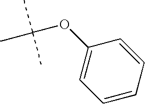 |
| C.271 | 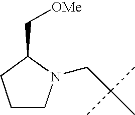 | CH | 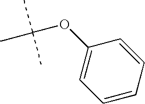 |
| C.272 | 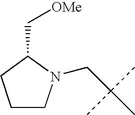 | CH | 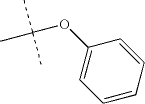 |
| C.273 | 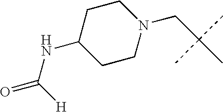 | CH | 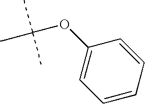 |
| C.274 | 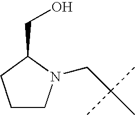 | CH | 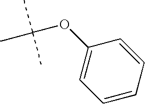 |
| C.275 | 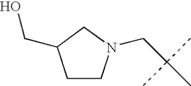 | CH | 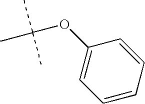 |
| C.276 | 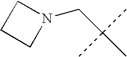 | CH | 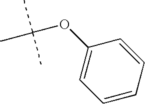 |
| C.277 | 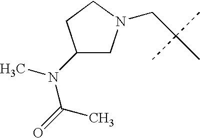 | CH | 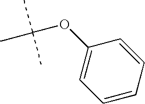 |
| C.278 | 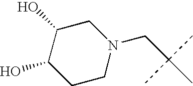 | CH | 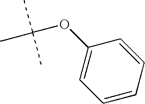 |
| C.279 | 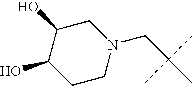 | CH | 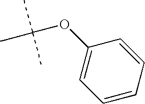 |
| C.280 | 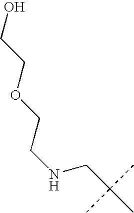 | CH | 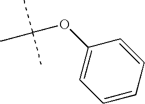 |
| C.281 | 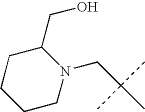 | CH | 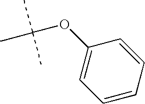 |
| C.282 | 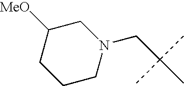 | CH | 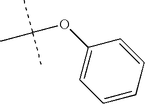 |
| C.283 | 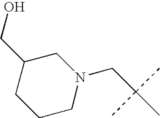 | CH | 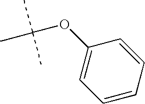 |
| C.284 | 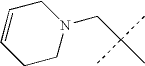 | CH | 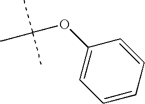 |
| C.285 | 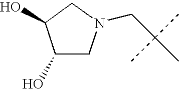 | CH | 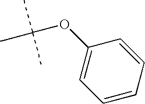 |
| C.286 | 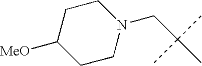 | CH | 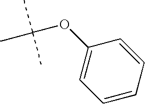 |
| C.287 | 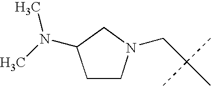 | CH | 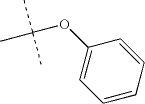 |
| C.288 | 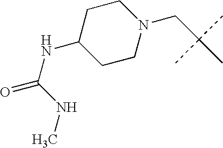 | CH | 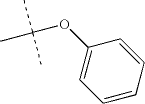 |
| C.289 | 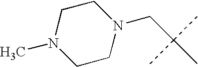 | CH | 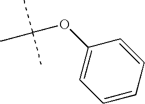 |
| C.290 | 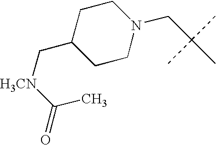 | CH | 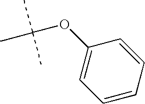 |
| C.291 | 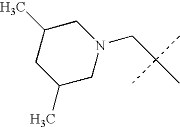 | CH | 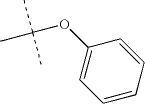 |
| C.292 | 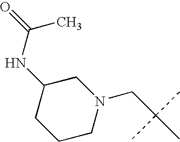 | CH | 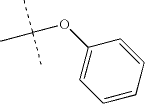 |
| C.293 | 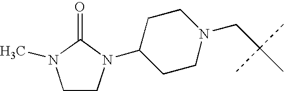 | CH | 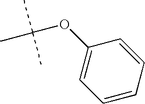 |
| C.294 | 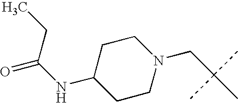 | CH | 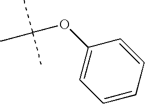 |
| C.295 | 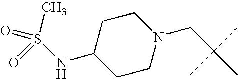 | CH | 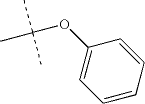 |
| C.296 | 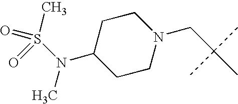 | CH | 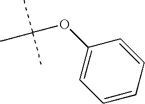 |
| C.297 | 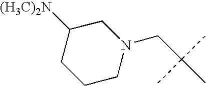 | CH | 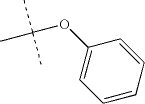 |
| C.298 | 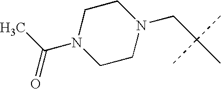 | CH | 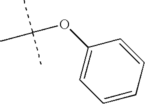 |
| C.299 | 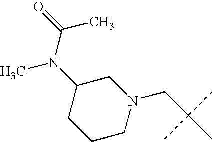 | CH | 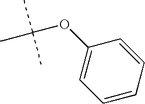 |
| C.300 | 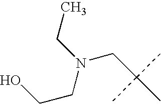 | CH | 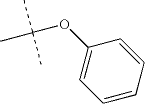 |
| C.301 | 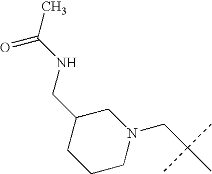 | CH | 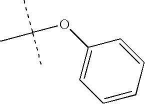 |
| C.302 | 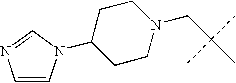 | CH | 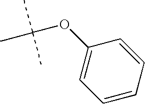 |
| C.303 | 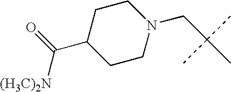 | CH | 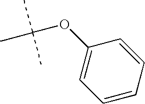 |
| C.304 | 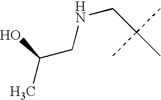 | CH | 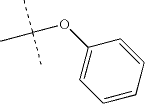 |
| C.305 | 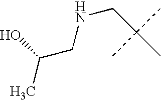 | CH | 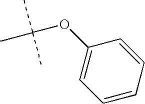 |
| C.306 | 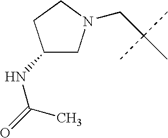 | CH | 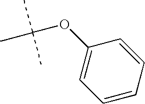 |
| C.307 | 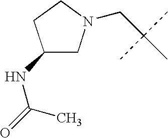 | CH | 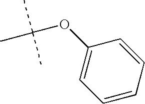 |
| C.308 | 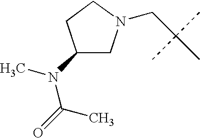 | CH | 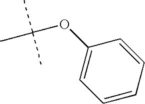 |
| C.309 | 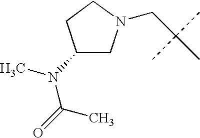 | CH | 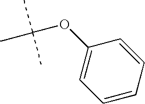 |
| C.310 | 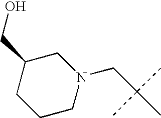 | CH | 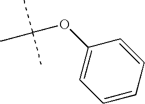 |
| C.311 | 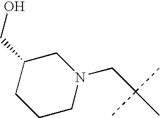 | CH | 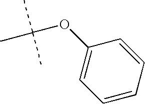 |
| C.312 | 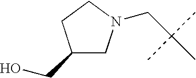 | CH | 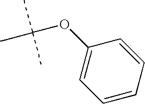 |
| C.313 | 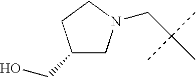 | CH | 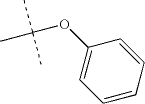 |
| C.314 | 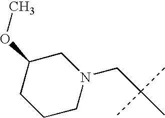 | CH | 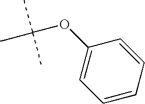 |
| C.315 | 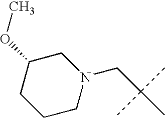 | CH | 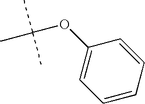 |
| C.316 | 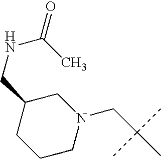 | CH | 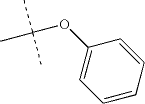 |
| C.317 | 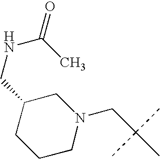 | CH | 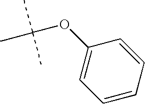 |
| C.318 | 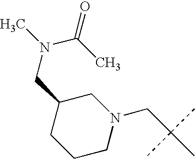 | CH | 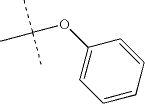 |
| C.319 | 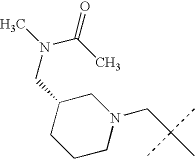 | CH | 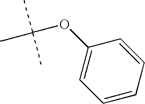 |
| C.320 | 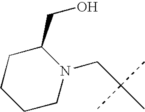 | CH | 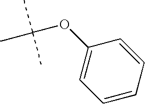 |
| C.321 | 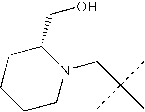 | CH | 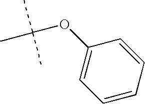 |
| C.322 | 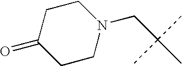 | CH | 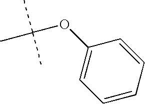 |
| C.323 | 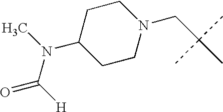 | CH | 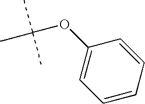 |
| C.324 | 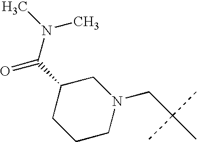 | CH | 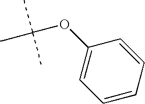 |
| C.325 | 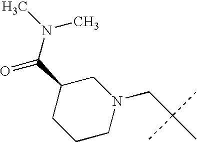 | CH | 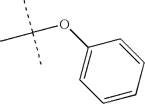 |
| C.326 | 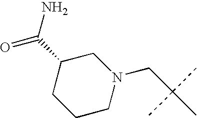 | CH | 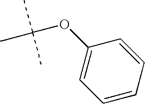 |
| C.327 | 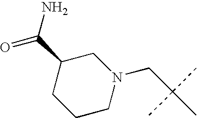 | CH | 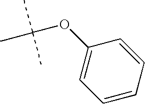 |
| C.328 | 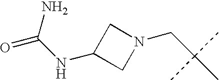 | CH | 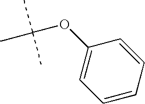 |
| C.329 | 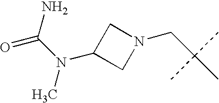 | CH | 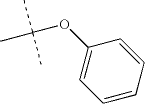 |
| C.330 | 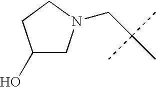 | CH | 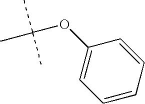 |
| C.331 | 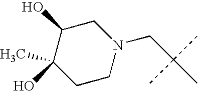 | CH | 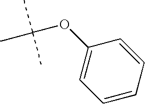 |
| C.332 | 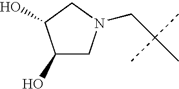 | CH | 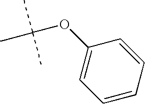 |
| C.333 | 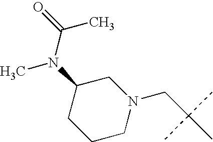 | CH | 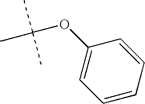 |
| C.334 | 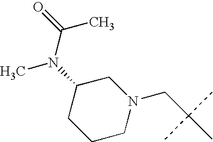 | CH | 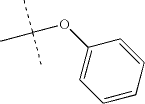 |
| C.335 | 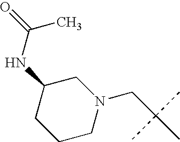 | CH | 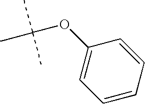 |
| C.336 | 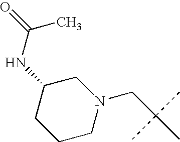 | CH | 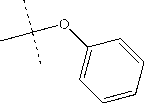 |
| C.337 | 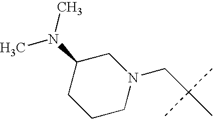 | CH | 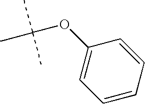 |
| C.338 | 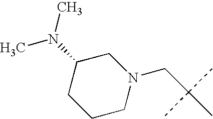 | CH | 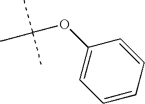 |
| C.339 | 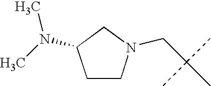 | CH | 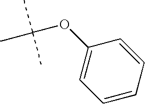 |
| C.340 | 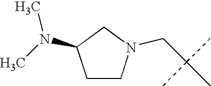 | CH | 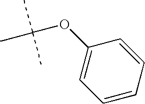 |
| C.341 | 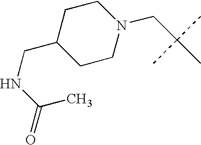 | CH | 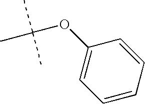 |
| C.342 | 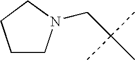 | CH | 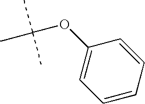 |
| C.343 | 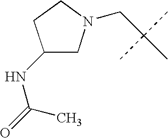 | CH | 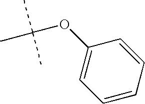 |
| C.344 | 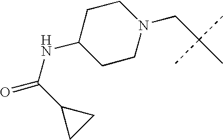 | CH | 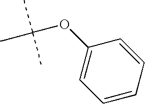 |
| C.345 | 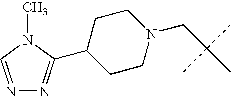 | CH | 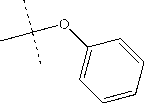 |
| C.346 | 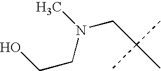 | CH | 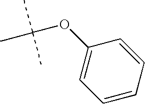 |
| C.347 | 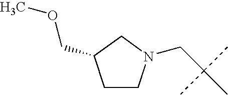 | CH | 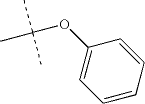 |
| C.348 | 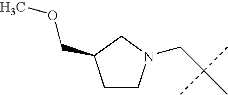 | CH | 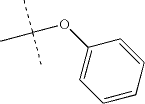 |
| C.349 | 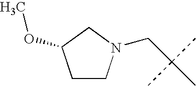 | CH | 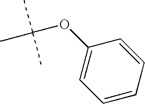 |
| C.350 | 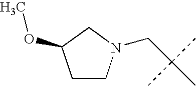 | CH | 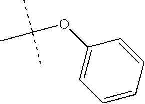 |
| C.351 | 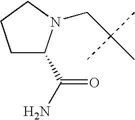 | CH | 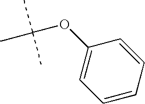 |
| C.352 | 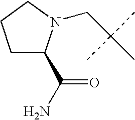 | CH | 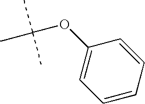 |
| C.353 | 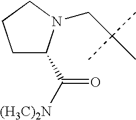 | CH | 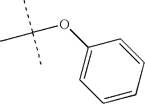 |
| C.354 | 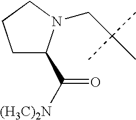 | CH | 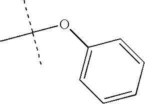 |
| C.355 | 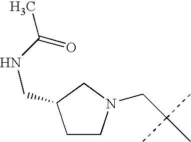 | CH | 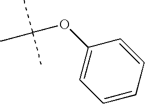 |
| C.356 | 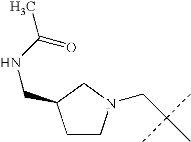 | CH | 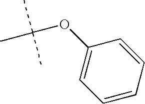 |
| C.357 | 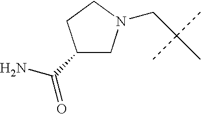 | CH | 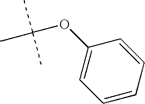 |
| C.358 | 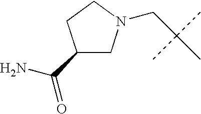 | CH | 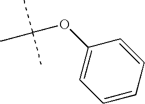 |
| C.359 | 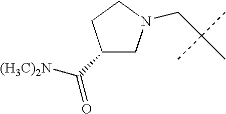 | CH | 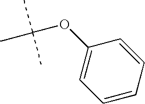 |
| C.360 | 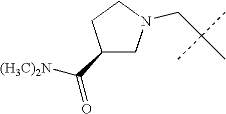 | CH | 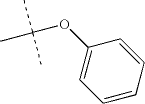 |
| C.361 | 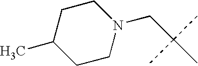 | CH | 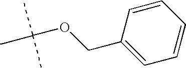 |
| C.362 | 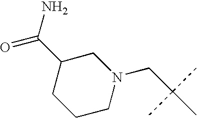 | CH | 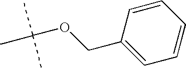 |
| C.363 | 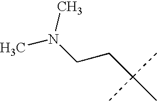 | CH | 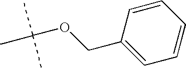 |
| C.364 | 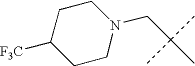 | CH | 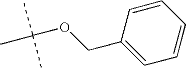 |
| C.365 | 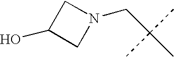 | CH | 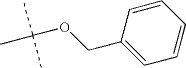 |
| C.366 | 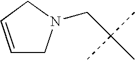 | CH | 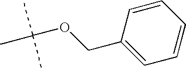 |
| C.367 | 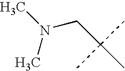 | CH | 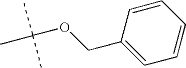 |
| C.368 | 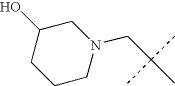 | CH | 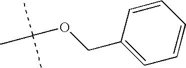 |
| C.369 | 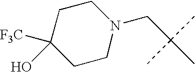 | CH | 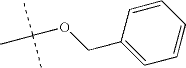 |
| C.370 | 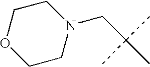 | CH | 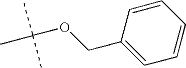 |
| C.371 | 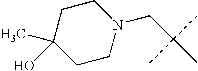 | CH | 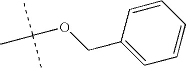 |
| C.372 | 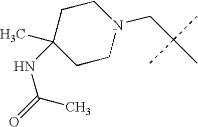 | CH | 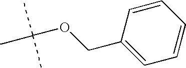 |
| C.373 | 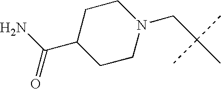 | CH | 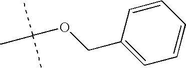 |
| C.374 | 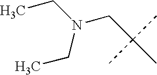 | CH | 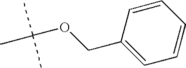 |
| C.375 | 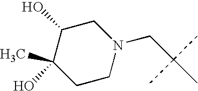 | CH | 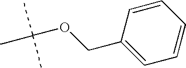 |
| C.376 | 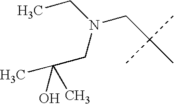 | CH | 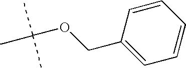 |
| C.377 | 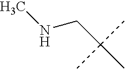 | CH | 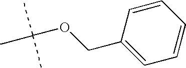 |
| C.378 | 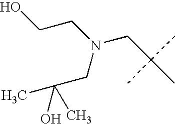 | CH | 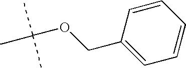 |
| C.379 | 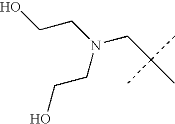 | CH | 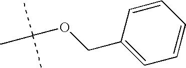 |
| C.380 | 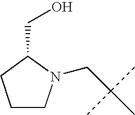 | CH | 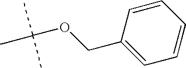 |
| C.381 | 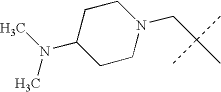 | CH | 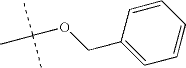 |
| C.382 | 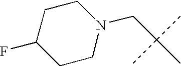 | CH | 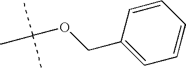 |
| C.383 | 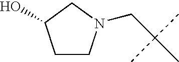 | CH | 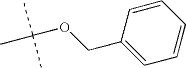 |
| C.384 | 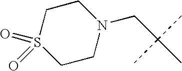 | CH | 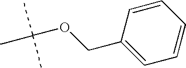 |
| C.385 | 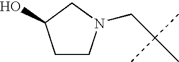 | CH | 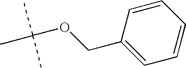 |
| C.386 | 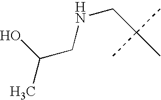 | CH | 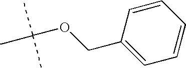 |
| C.387 | 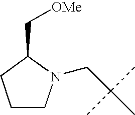 | CH | 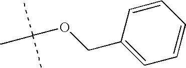 |
| C.388 | 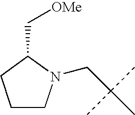 | CH | 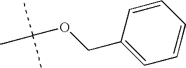 |
| C.389 | 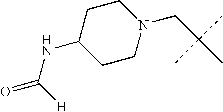 | CH | 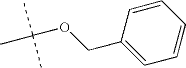 |
| C.390 | 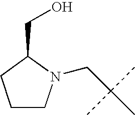 | CH | 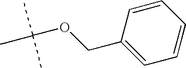 |
| C.391 | 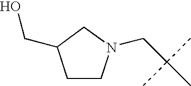 | CH | 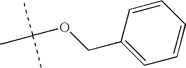 |
| C.392 | 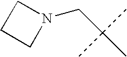 | CH | 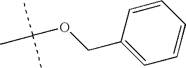 |
| C.393 | 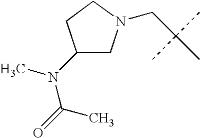 | CH | 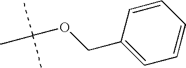 |
| C.394 | 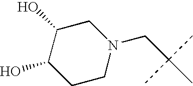 | CH | 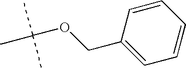 |
| C.395 | 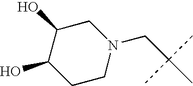 | CH | 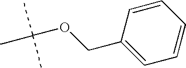 |
| C.396 | 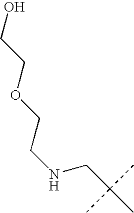 | CH | 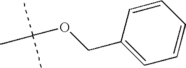 |
| C.397 | 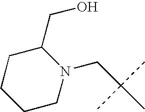 | CH | 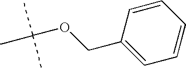 |
| C.398 | 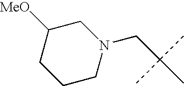 | CH | 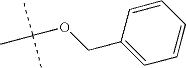 |
| C.399 | 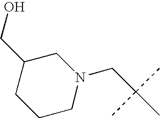 | CH | 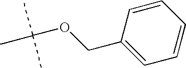 |
| C.400 | 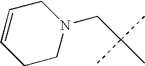 | CH | 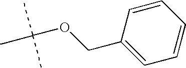 |
| C.401 | 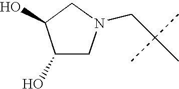 | CH | 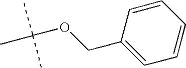 |
| C.402 | 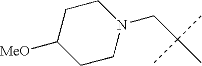 | CH | 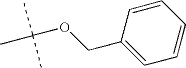 |
| C.403 | 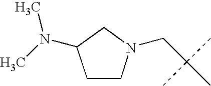 | CH | 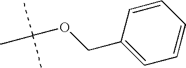 |
| C.404 | 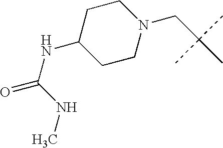 | CH | 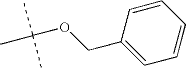 |
| C.405 | 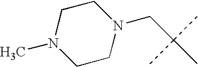 | CH | 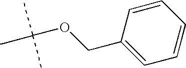 |
| C.406 | 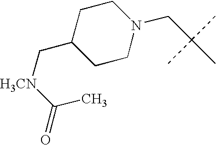 | CH | 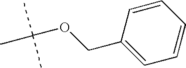 |
| C.407 | 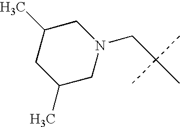 | CH | 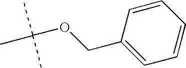 |
| C.408 | 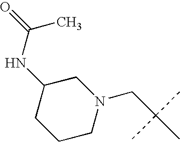 | CH | 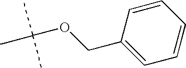 |
| C.409 | 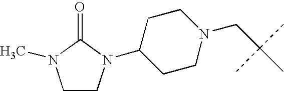 | CH | 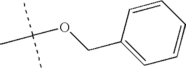 |
| C.410 | 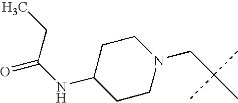 | CH | 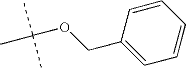 |
| C.411 | 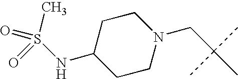 | CH | 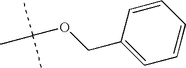 |
| C.412 | 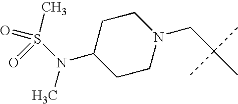 | CH | 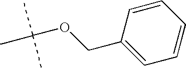 |
| C.413 | 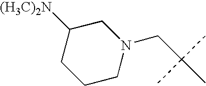 | CH | 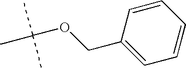 |
| C.414 | 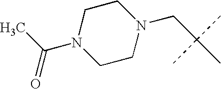 | CH | 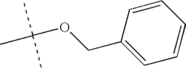 |
| C.415 | 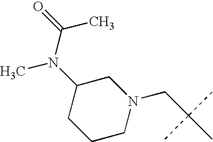 | CH | 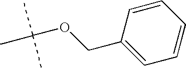 |
| C.416 | 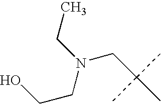 | CH | 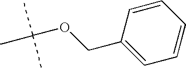 |
| C.417 | 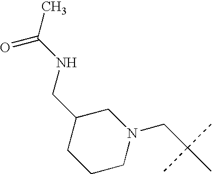 | CH | 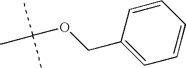 |
| C.418 | 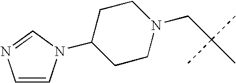 | CH | 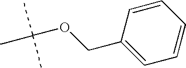 |
| C.419 | 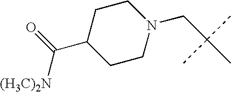 | CH | 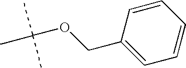 |
| C.420 | 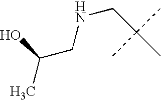 | CH | 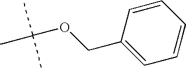 |
| C.421 | 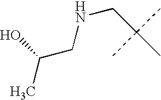 | CH | 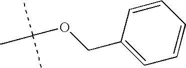 |
| C.422 | 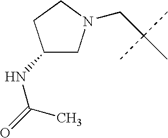 | CH | 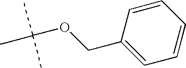 |
| C.423 | 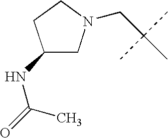 | CH | 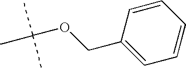 |
| C.424 | 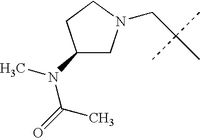 | CH | 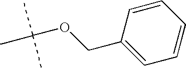 |
| C.425 | 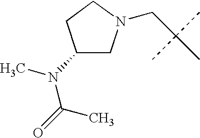 | CH | 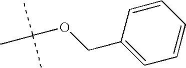 |
| C.426 | 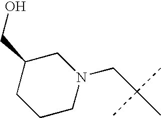 | CH | 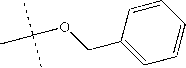 |
| C.427 | 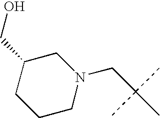 | CH | 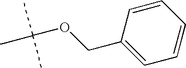 |
| C.428 | 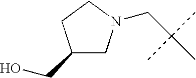 | CH | 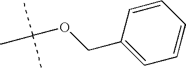 |
| C.429 | 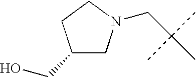 | CH | 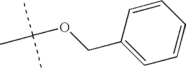 |
| C.430 | 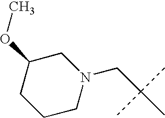 | CH | 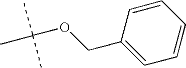 |
| C.431 | 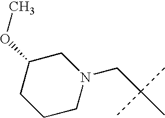 | CH | 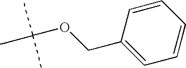 |
| C.432 | 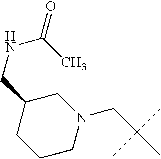 | CH | 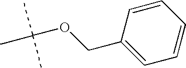 |
| C.433 | 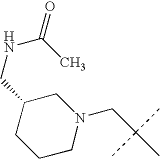 | CH | 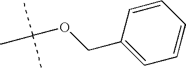 |
| C.434 | 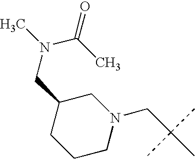 | CH | 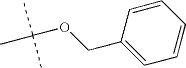 |
| C.435 | 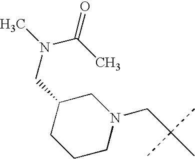 | CH | 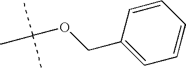 |
| C.436 | 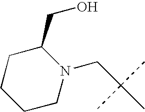 | CH | 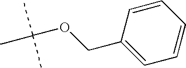 |
| C.437 | 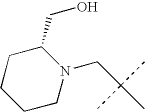 | CH | 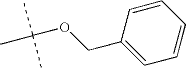 |
| C.438 | 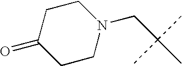 | CH | 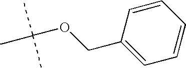 |
| C.439 | 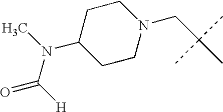 | CH | 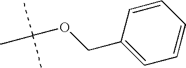 |
| C.440 | 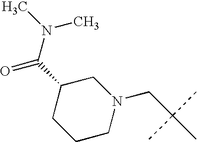 | CH | 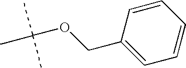 |
| C.441 | 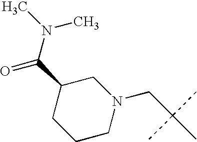 | CH | 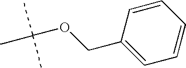 |
| C.442 | 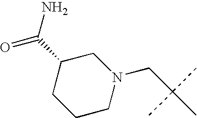 | CH | 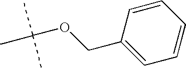 |
| C.443 | 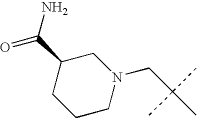 | CH | 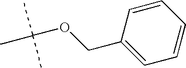 |
| C.444 | 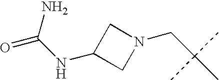 | CH | 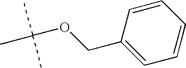 |
| C.445 | 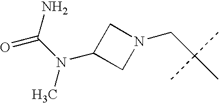 | CH | 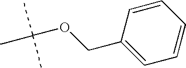 |
| C.446 | 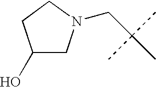 | CH | 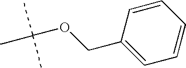 |
| C.447 | 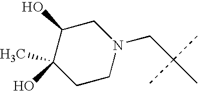 | CH | 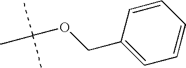 |
| C.448 | 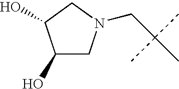 | CH | 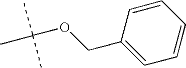 |
| C.449 | 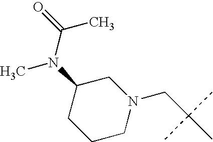 | CH | 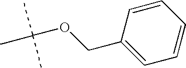 |
| C.450 | 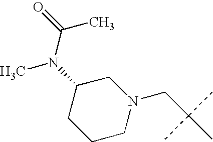 | CH | 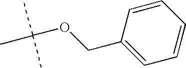 |
| C.451 | 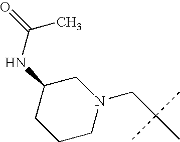 | CH | 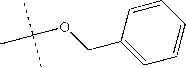 |
| C.452 | 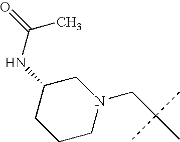 | CH | 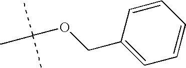 |
| C.453 | 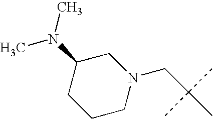 | CH | 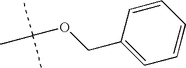 |
| C.454 | 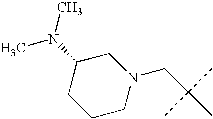 | CH | 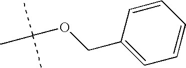 |
| C.455 | 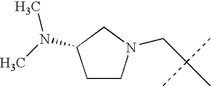 | CH | 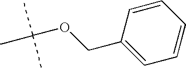 |
| C.456 | 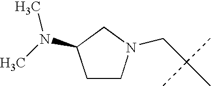 | CH | 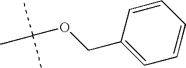 |
| C.457 | 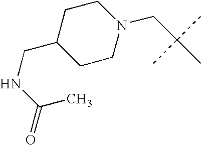 | CH | 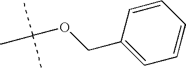 |
| C.458 | 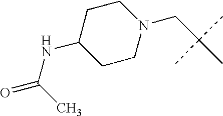 | CH | 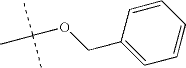 |
| C.459 | 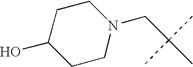 | CH | 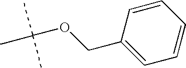 |
| C.460 | 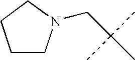 | CH | 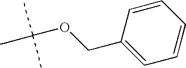 |
| C.461 | 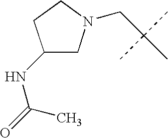 | CH | 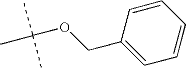 |
| C.462 | 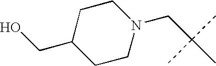 | CH | 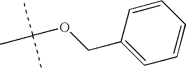 |
| C.463 | 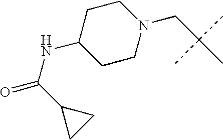 | CH | 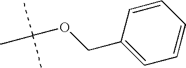 |
| C.464 | 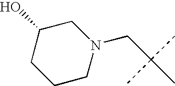 | CH | 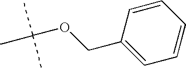 |
| C.465 | 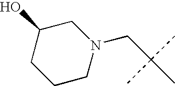 | CH | 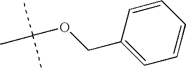 |
| C.466 | 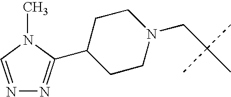 | CH | 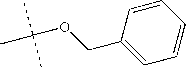 |
| C.467 | 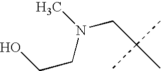 | CH | 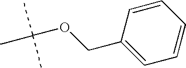 |
| C.468 | 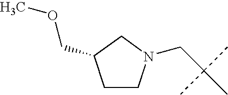 | CH | 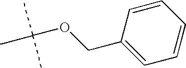 |
| C.469 | 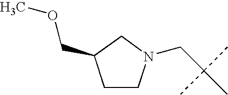 | CH | 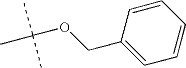 |
| C.470 | 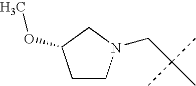 | CH | 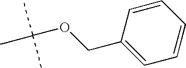 |
| C.471 | 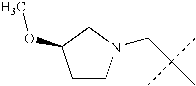 | CH | 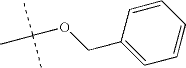 |
| C.472 | 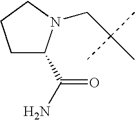 | CH | 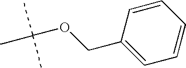 |
| C.473 | 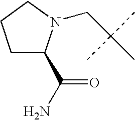 | CH | 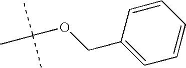 |
| C.474 | 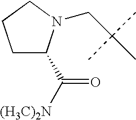 | CH | 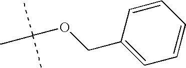 |
| C.475 | 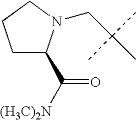 | CH | 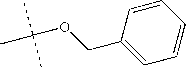 |
| C.476 | 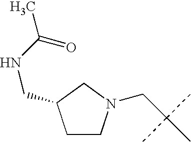 | CH | 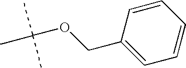 |
| C.477 | 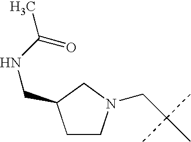 | CH | 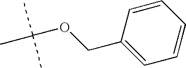 |
| C.478 | 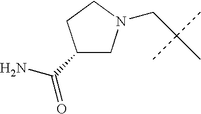 | CH | 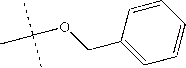 |
| C.479 | 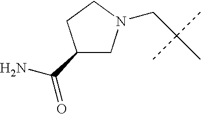 | CH | 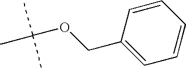 |
| C.480 | 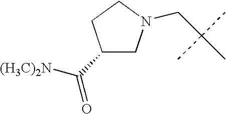 | CH | 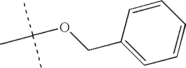 |
| C.481 | 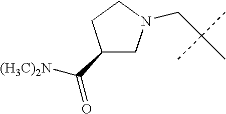 | CH | 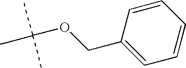 |
| C.482 | 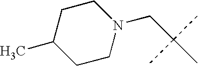 | CH | 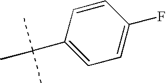 |
| C.483 | 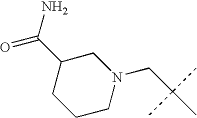 | CH | 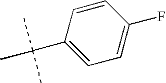 |
| C.484 | 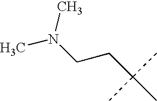 | CH | 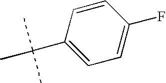 |
| C.485 | 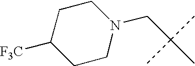 | CH | 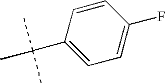 |
| C.486 | 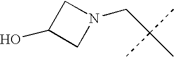 | CH | 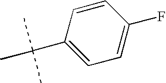 |
| C.487 | 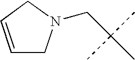 | CH | 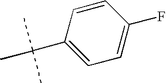 |
| C.488 | 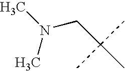 | CH | 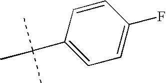 |
| C.489 | 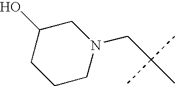 | CH | 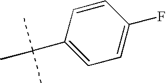 |
| C.490 | 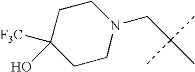 | CH | 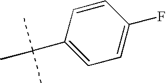 |
| C.491 | 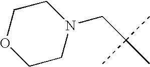 | CH | 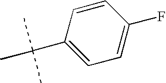 |
| C.492 | 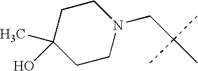 | CH | 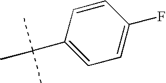 |
| C.493 | 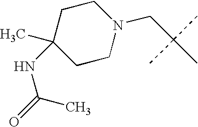 | CH | 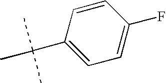 |
| C.494 | 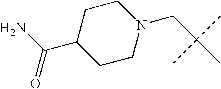 | CH | 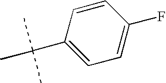 |
| C.495 | 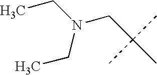 | CH | 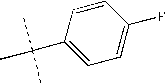 |
| C.496 | 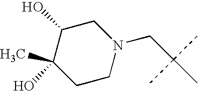 | CH | 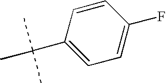 |
| C.497 | 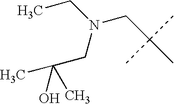 | CH | 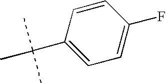 |
| C.498 | 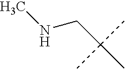 | CH | 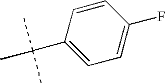 |
| C.499 | 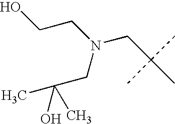 | CH | 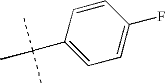 |
| C.500 | 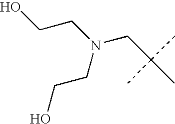 | CH | 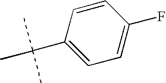 |
| C.501 | 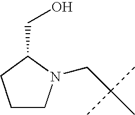 | CH | 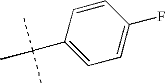 |
| C.502 | 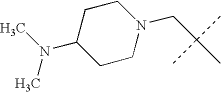 | CH | 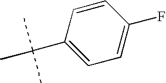 |
| C.503 | 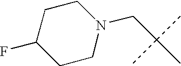 | CH | 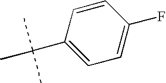 |
| C.504 | 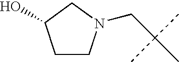 | CH | 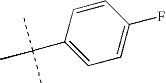 |
| C.505 | 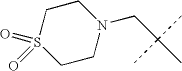 | CH | 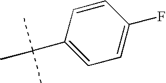 |
| C.506 | 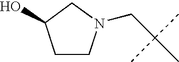 | CH | 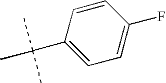 |
| C.507 | 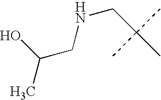 | CH | 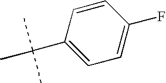 |
| C.508 | 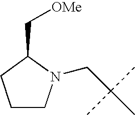 | CH | 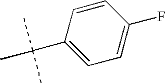 |
| C.509 | 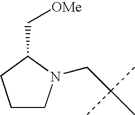 | CH | 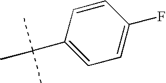 |
| C.510 | 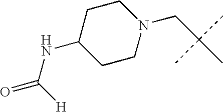 | CH | 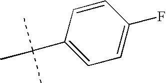 |
| C.511 | 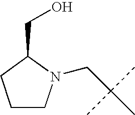 | CH | 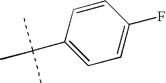 |
| C.512 | 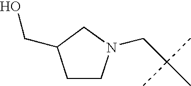 | CH | 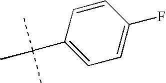 |
| C.513 | 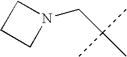 | CH | 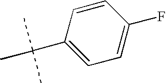 |
| C.514 | 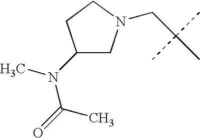 | CH | 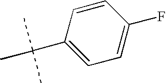 |
| C.515 | 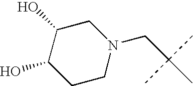 | CH | 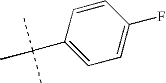 |
| C.516 | 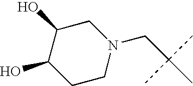 | CH | 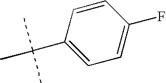 |
| C.517 | 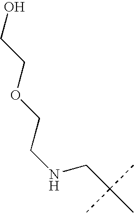 | CH | 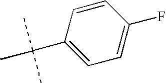 |
| C.518 | 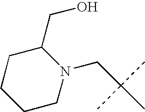 | CH | 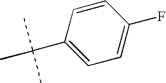 |
| C.519 | 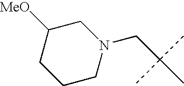 | CH | 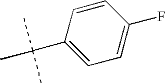 |
| C.520 | 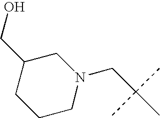 | CH | 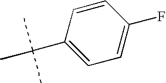 |
| C.521 | 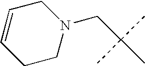 | CH | 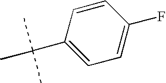 |
| C.522 | 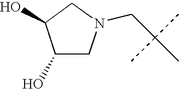 | CH | 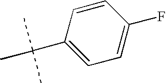 |
| C.523 | 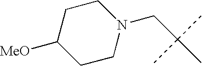 | CH | 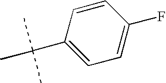 |
| C.524 | 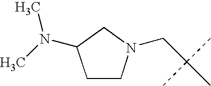 | CH | 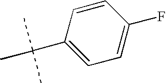 |
| C.525 | 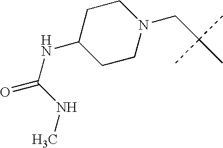 | CH | 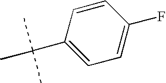 |
| C.526 | 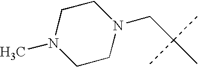 | CH | 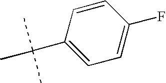 |
| C.527 | 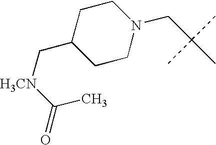 | CH | 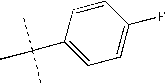 |
| C.528 | 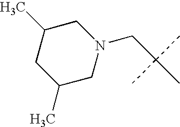 | CH | 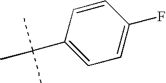 |
| C.529 | 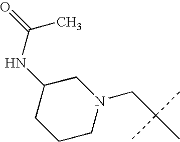 | CH | 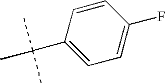 |
| C.530 | 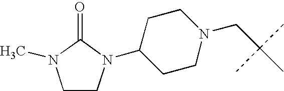 | CH | 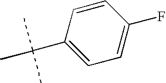 |
| C.531 | 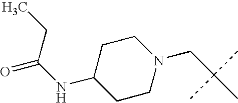 | CH | 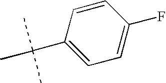 |
| C.532 | 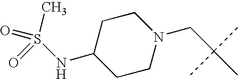 | CH | 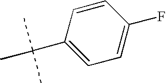 |
| C.533 | 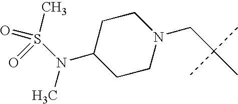 | CH | 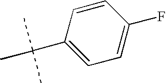 |
| C.534 | 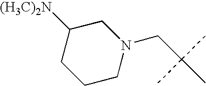 | CH | 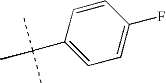 |
| C.535 | 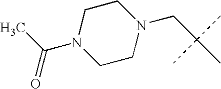 | CH | 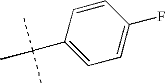 |
| C.536 | 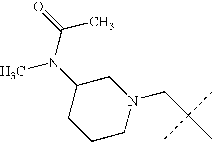 | CH | 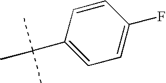 |
| C.537 | 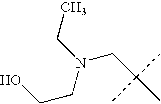 | CH | 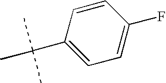 |
| C.538 | 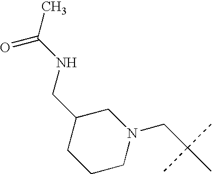 | CH | 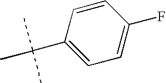 |
| C.539 | 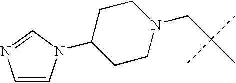 | CH | 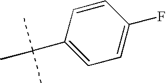 |
| C.540 | 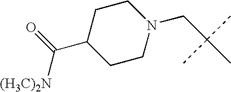 | CH | 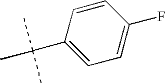 |
| C.541 | 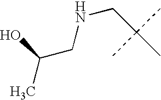 | CH | 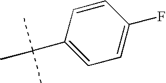 |
| C.542 | 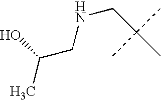 | CH | 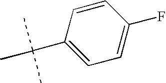 |
| C.543 | 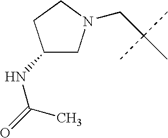 | CH | 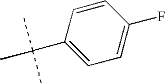 |
| C.544 | 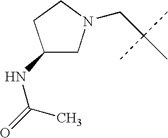 | CH | 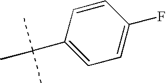 |
| C.545 | 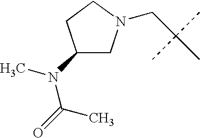 | CH | 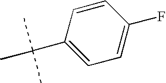 |
| C.546 | 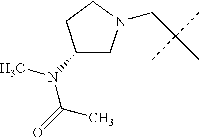 | CH | 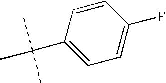 |
| C.547 | 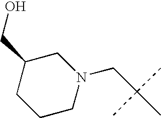 | CH | 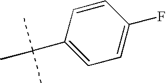 |
| C.548 | 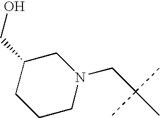 | CH | 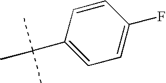 |
| C.549 | 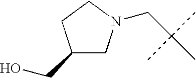 | CH | 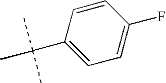 |
| C.550 | 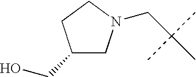 | CH | 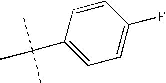 |
| C.551 | 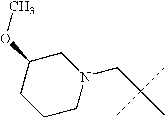 | CH | 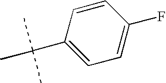 |
| C.552 | 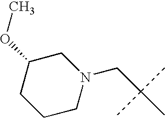 | CH | 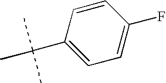 |
| C.553 | 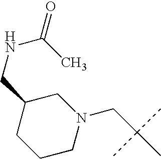 | CH | 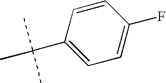 |
| C.554 | 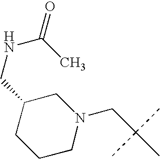 | CH | 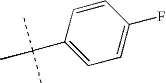 |
| C.555 | 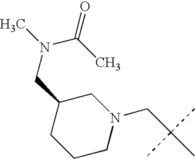 | CH | 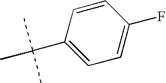 |
| C.556 | 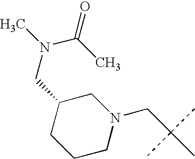 | CH | 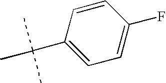 |
| C.557 | 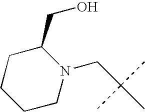 | CH | 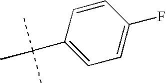 |
| C.558 | 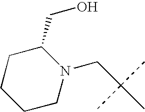 | CH | 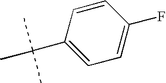 |
| C.559 | 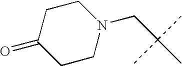 | CH | 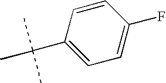 |
| C.560 | 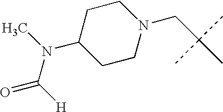 | CH | 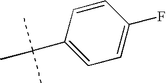 |
| C.561 | 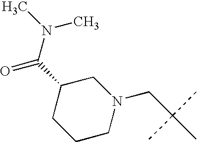 | CH | 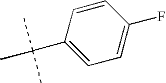 |
| C.562 | 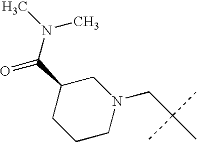 | CH | 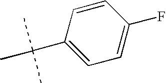 |
| C.563 | 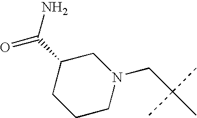 | CH | 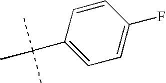 |
| C.564 | 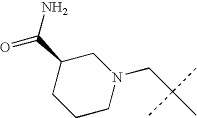 | CH | 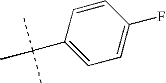 |
| C.565 | 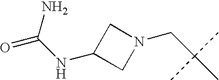 | CH | 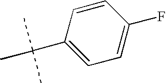 |
| C.566 | 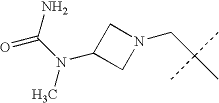 | CH | 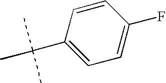 |
| C.567 | 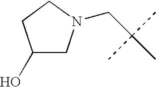 | CH | 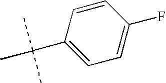 |
| C.568 | 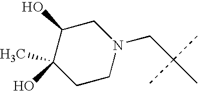 | CH | 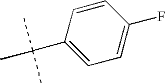 |
| C.569 | 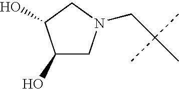 | CH | 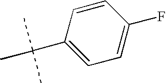 |
| C.570 | 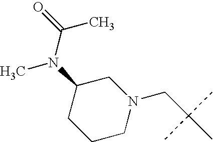 | CH | 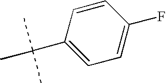 |
| C.571 | 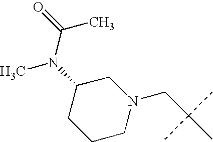 | CH | 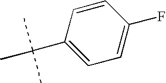 |
| C.572 | 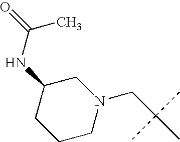 | CH | 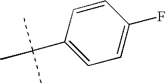 |
| C.573 | 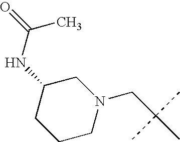 | CH | 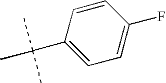 |
| C.574 | 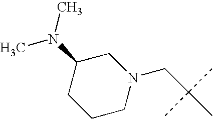 | CH | 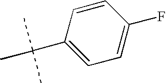 |
| C.575 | 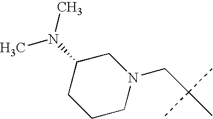 | CH | 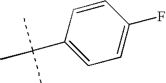 |
| C.576 | 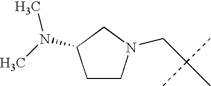 | CH | 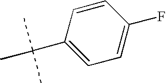 |
| C.577 | 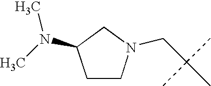 | CH | 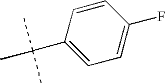 |
| C.578 | 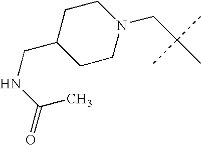 | CH | 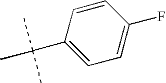 |
| C.579 | 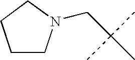 | CH | 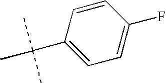 |
| C.580 | 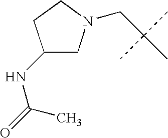 | CH | 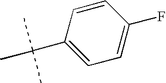 |
| C.581 | 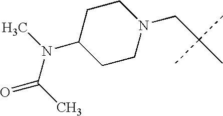 | CH | 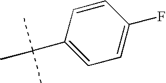 |
| C.582 | 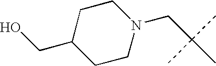 | CH | 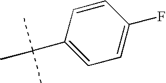 |
| C.583 | 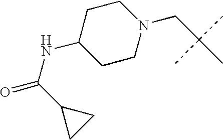 | CH | 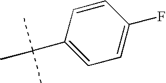 |
| C.584 | 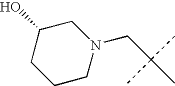 | CH | 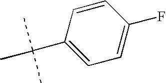 |
| C.585 | 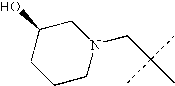 | CH | 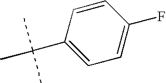 |
| C.586 | 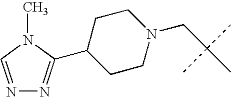 | CH | 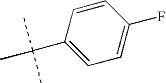 |
| C.587 | 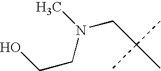 | CH | 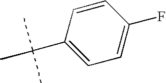 |
| C.588 | 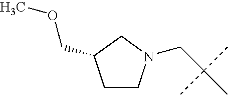 | CH | 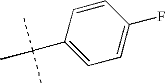 |
| C.589 | 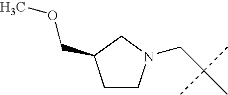 | CH | 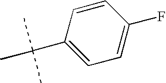 |
| C.590 | 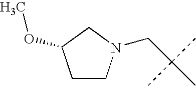 | CH | 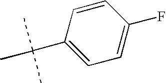 |
| C.591 | 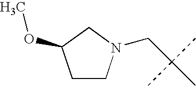 | CH | 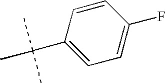 |
| C.592 | 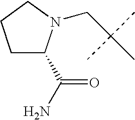 | CH | 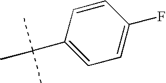 |
| C.593 | 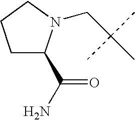 | CH | 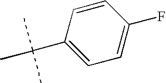 |
| C.594 | 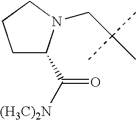 | CH | 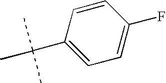 |
| C.595 | 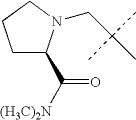 | CH | 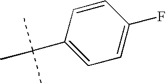 |
| C.596 | 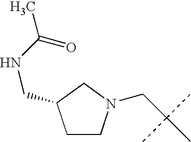 | CH | 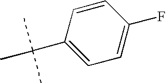 |
| C.597 | 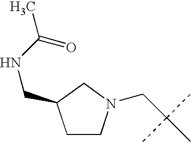 | CH | 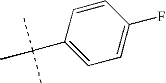 |
| C.598 | 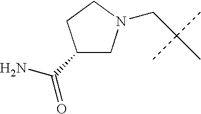 | CH | 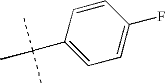 |
| C.599 | 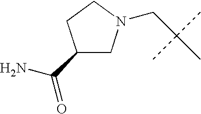 | CH | 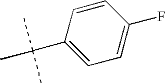 |
| C.600 | 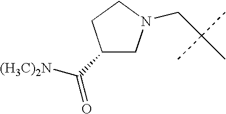 | CH | 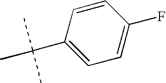 |
| C.601 | 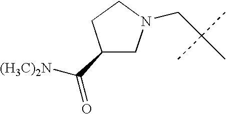 | CH | 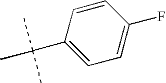 |

Some test methods for determining an MCH-receptor antagonistic activity will now be described. In addition, other test methods known to the skilled man may be used, e.g. by inhibiting the MCH-receptor-mediated inhibition of cAMP production, as described by Hoogduijn M et al. in “Melanin-concentrating hormone and its receptor are expressed and functional in human skin”, Biochem. Biophys. Res Commun. 296 (2002) 698-701 and by biosensory measurement of the binding of MCH to the MCH receptor in the presence of antagonistic substances by plasmon resonance, as described by Karlsson OP and Lofas S. in “Flow-Mediated On-Surface Reconstitution of G-Protein Coupled Receptors for Applications in Surface Plasmon Resonance Biosensors”, Anal. Biochem. 300 (2002),132-138. Other methods of testing antagonistic activity to MCH receptors are contained in the references and patent documents mentioned hereinbefore, and the description of the test methods used is hereby incorporated in this application.

MCH-1 Receptor Binding Test

Method: MCH binding to hMCH-1R transfected cells

Species: Human

Test cell: hMCH-1R stably transfected into CHO/Galphal 6 cells

Results: IC50 values

Membranes from CHO/Galphal6 cells stably transfected with human hMCH-1R are resuspended using a syringe (needle 0.6×25 mm) and diluted in test buffer (50 mM HEPES, 10 mM MgCl₂, 2 mM EGTA, pH 7.00; 0.1% bovine serum albumin (protease-free), 0.021% bacitracin, 1 μg/ml aprotinin, 1 μg/ml leupeptin and 1 μM phosphoramidone) to a concentration of 5 to 15 μg/ml.

200 microlitres of this membrane fraction (contains 1 to 3 μg of protein) are incubated for 60 minutes at ambient temperature with 100 pM of ¹²⁵I-tyrosyl melanin concentrating hormone (¹²⁵I-MCH commercially obtainable from NEN) and increasing concentrations of the test compound in a final volume of 250 microlitres. After the incubation the reaction is filtered using a cell harvester through 0.5% PEI treated fibreglass filters (GF/B, Unifilter Packard). The membrane-bound radioactivity retained on the filter is then determined after the addition of scintillator substance (Packard Microscint 20) in a measuring device (TopCount of Packard).

The non-specific binding is defined as bound radioactivity in the presence of 1 micromolar MCH during the incubation period.

The analysis of the concentration binding curve is carried out on the assumption of one receptor binding site.

Standard:

Non-labelled MCH competes with labelled ¹²⁵I-MCH for the receptor binding with an IC50 value of between 0.06 and 0.15 nM.

The KD value of the radioligand is 0.156 nM.

MCH-1 Receptor-Coupled Ca²⁺ Mobilisation Test

Method: Calcium mobilisation test with human MCH (FLIPR³⁸⁴)

Species: Human

Test cells: CHO/Galpha 16 cells stably transfected with hMCH-R1

Results: 1st measurement:: % stimulation of the reference (MCH 10⁻⁶M)

2nd measurement: pKB value

Reagents:

| | |
|---|---|
| HBSS (10x) | (GIBCO) |
| HEPES buffer (1M) | (GIBCO) |
| Pluronic F-127 | (Molecular Probes) |
| Fluo-4 | (Molecular Probes) |
| Probenecid | (Sigma) |
| MCH | (Bachem) |
| bovine serum albumin | (Serva) |
| (protease-free) | |
| DMSO | (Serva) |
| Ham's F12 | (BioWhittaker) |
| FCS | (BioWhittaker) |
| L-Glutamine | (GIBCO) |
| Hygromycin B | (GIBCO) |
| PENStrep | (BioWhittaker) |
| Zeocin | (Invitrogen) |

Clonal CHO/Galpha16 hMCH-R1 cells are cultivated in Ham's F12 cell culture medium (with L-glutamine; BioWhittaker; Cat.No.: BE12-615F). This contains per 500 ml 10% FCS, 1% PENStrep, 5 ml L-glutamine (200 mM stock solution), 3 ml hygromycin B (50 mg/ml in PBS) and 1.25 ml zeocin (100 μg/ml stock solution). One day before the experiment the cells are plated on a 384-well microtitre plate (black-walled with a transparent base, made by Costar) in a density of 2500 cells per cavity and cultivated in the above medium overnight at 37° C., 5% CO₂ and 95% relative humidity. On the day of the experiment the cells are incubated with cell culture medium to which 2 mM Fluo-4 and 4.6 mM Probenicid have been added, at 37° C. for 45 minutes. After charging with fluorescent dye the cells are washed four times with Hanks buffer solution (1×HBSS, 20 mM HEPES), which has been combined with 0.07% Probenicid. The test substances are diluted in Hanks buffer solution, combined with 2.5% DMSO. The background fluorescence of non-stimulated cells is measured in the presence of substance in the 384-well microtitre plate five minutes after the last washing step in the FLIPR³⁸⁴ apparatus (Molecular Devices; excitation wavelength: 488 nm; emission wavelength: bandpass 510 to 570 nm). To stimulate the cells MCH is diluted in Hanks buffer with 0.1% BSA, pipetted into the 384-well cell culture plate 35 minutes after the last washing step and the MCH-stimulated fluorescence is then measured in the FLIPR³⁸⁴ apparatus.

Data Analysis:

1st measurement: The cellular Ca²⁺ mobilisation is measured as the peak of the relative fluorescence minus the background and is expressed as the percentage of the maximum signal of the reference (MCH 10⁻⁶M). This measurement serves to identify any possible agonistic effect of a test substance.

2nd measurement: The cellular Ca²⁺ mobilisation is measured as the peak of the relative fluorescence minus the background and is expressed as the percentage of the maximum signal of the reference (MCH 10⁻⁶M, signal is standardised to 100%). The EC50 values of the MCH dosage activity curve with and without test substance (defined concentration) are determined graphically by the GraphPad Prism 2.01 curve program. MCH antagonists cause the MCH stimulation curve to shift to the right in the graph plotted.

The inhibition is expressed as a pKB value:
pKB=log(EC_{50(testsubstance+MCH)}/EC_{50(MCH)}−1)−log c_{(testsubstance)}

The compounds according to the invention, including their salts, exhibit an MCH-receptor antagonistic activity in the tests mentioned above. Using the MCH-1 receptor binding test described above an antagonistic activity is obtained in a dosage range from about 10⁻¹⁰ to 10⁻⁵ M, particularly from 10⁻⁹ to 10⁻⁶ M.

The following IC50 values were determined using the MCH-1 receptor binding test described above:

| Compound | | |
|---|---|---|
| according to | | IC50 |
| Example no. | Name of substance | value |
| 3.30 | N-[1-(4-{3-[6-(4-Methoxy-phenyl)-pyridazin-3- | 79 nM |
| | yl]-propylamino}-benzyl)-pyrrolidin-3-yl]- | |
| | acetamide | |
| 9.1 | 1-(4-{3-[6-(4-Methoxy-phenyl)-pyridazin-3- | 174 nM |
| | ylamino]-propyl}-benzyl)-piperidin-4-ol | |
| 12.1 | (2-{2-Chloro-4-[2-(2-chloro-4-iodo-phenoxy)- | 172 nM |
| | ethoxy]-phenoxy}-ethyl)-diethyl-amine | |

Some examples of formulations will be described hereinafter, wherein the term “active substance” denotes one or more compounds according to the invention, including their salts. In the case of one of the combinations with one or more active substances described, the term “active substance” also includes the additional active substances.

### EXAMPLE A

Capsules for Powder Inhalation Containing 1 mg Active Substance

Composition:

1 capsule for powder inhalation contains:

| | |
|---|---|
| active substance | 1.0 mg |
| lactose | 20.0 mg |
| hard gelatine capsules | 50.0 mg |
| | 71.0 mg |

Method of Preparation:

The active substance is ground to the particle size required for inhalation. The ground active substance is homogeneously mixed with the lactose. The mixture is packed into hard gelatine capsules.

### EXAMPLE B

Inhalable solution for Respimat® containing 1 mg active substance

Composition:

1 spray contains:

| | | |
|---|---|---|
| active substance | 1.0 | mg |
| benzalkonium chloride | 0.002 | mg |
| disodium edetate | 0.0075 | mg |
| purified water ad | 15.0 | μl |

Method of Preparation:

The active substance and benzalkonium chloride are dissolved in water and packed into Respimat® cartridges.

### EXAMPLE C

Inhalable Solution for Nebulisers Containing 1 mg Active Substance

Composition:

| 1 vial contains: | | |
|---|---|---|
| active substance | 0.1 | g |
| sodium chloride | 0.18 | g |
| benzalkonium chloride | 0.002 | g |
| purified water ad | 20.0 | ml |

Method of Preparation:

The active substance, sodium chloride and benzalkonium chloride are dissolved in water.

### EXAMPLE D

Propellant Type Metered Dose Aerosol Containing 1 mg Active Substance

Composition:

| 1 spray contains: | | |
|---|---|---|
| active substance | 1.0 | mg |
| lecithin | 0.1% | |
| propellant gas ad | 50.0 | μl |

Method of Preparation:

The micronised active substance is homogeneously suspended in the mixture of lecithin and propellant gas. The suspension is transferred into a pressurised container with a metering valve.

### EXAMPLE E

Nasal Spray Containing 1 mg Active Substance

Composition:

| | | |
|---|---|---|
| active substance | 1.0 | mg |
| sodium chloride | 0.9 | mg |
| benzalkonium chloride | 0.025 | mg |
| disodium edetate | 0.05 | mg |
| purified water ad | 0.1 | ml |

Method of Preparation:

The active substance and the excipients are dissolved in water and transferred into a corresponding container.

### EXAMPLE F

Injectable Solution Containing 5 mg of Active Substance Per 5 ml

Composition:

| | | |
|---|---|---|
| active substance | 5 | mg |
| glucose | 250 | mg |
| human serum albumin | 10 | mg |
| glycofurol | 250 | mg |
| water for injections ad | 5 | ml |

Preparation:

Glycofurol and glucose are dissolved in water for injections (Wfl); human serum albumin is added; active ingredient is dissolved with heating; made up to specified volume with Wfl; transferred into ampoules under nitrogen gas.

### EXAMPLE G

Injectable solution containing 100 mg of active substance per 20 ml

Composition:

| | | |
|---|---|---|
| active substance | 100 | mg |
| monopotassium dihydrogen phosphate = KH₂PO₄ | 12 | mg |
| disodium hydrogen phosphate = Na₂HPO₄•2H₂O | 2 | mg |
| sodium chloride | 180 | mg |
| human serum albumin | 50 | mg |
| Polysorbate 80 | 20 | mg |
| water for injections ad | 20 | ml |

Preparation:

Polysorbate 80, sodium chloride, monopotassium dihydrogen phosphate and disodium hydrogen phosphate are dissolved in water for injections (Wfl); human serum albumin is added; active ingredient is dissolved with heating; made up to specified volume with Wfl; transferred into ampoules.

### EXAMPLE H

Lyophilisate Containing 10 mg of Active Substance

Composition:

| | |
|---|---|
| Active substance | 10 mg |
| Mannitol | 300 mg |
| human serum albumin | 20 mg |

Preparation:

Mannitol is dissolved in water for injections (Wfl); human serum albumin is added; active ingredient is dissolved with heating; made up to specified volume with Wfl; transferred into vials; freeze-dried.

Solvent for lyophilisate:

| | | |
|---|---|---|
| Polysorbate 80 = Tween 80 | 20 | mg |
| mannitol | 200 | mg |
| water for injections ad | 10 | ml |

Preparation:

Polysorbate 80 and mannitol are dissolved in water for injections (Wfl); transferred into ampoules.

### EXAMPLE I

Tablets Containing 20 mg of Active Substance

Composition:

| | |
|---|---|
| active substance | 20 mg |
| lactose | 120 mg |
| maize starch | 40 mg |
| magnesium stearate | 2 mg |
| Povidone K 25 | 18 mg |

Preparation:

Active substance, lactose and maize starch are homogeneously mixed; granulated with an aqueous solution of Povidone; mixed with magnesium stearate; compressed in a tablet press; weight of tablet 200 mg.

### EXAMPLE J

Capsules Containing 20 mg Active Substance

Composition:

| | |
|---|---|
| active substance | 20 mg |
| maize starch | 80 mg |
| highly dispersed silica | 5 mg |
| magnesium stearate | 2.5 mg |

Preparation:

Active substance, maize starch and silica are homogeneously mixed; mixed with magnesium stearate; the mixture is packed into size 3 hard gelatine capsules in a capsule filling machine.

### EXAMPLE K

Suppositories Containing 50 mg of Active Substance

Composition:

| | |
|---|---|
| active substance | 50 mg |
| hard fat (Adeps solidus) q.s. ad | 1700 mg |

Preparation:

Hard fat is melted at about 38° C.; ground active substance is homogeneously dispersed in the molten hard fat; after cooling to about 35° C. it is poured into chilled moulds.

### EXAMPLE L

Injectable Solution Containing 10 mg of Active Substance Per 1 ml

Composition:

| | | |
|---|---|---|
| active substance | 10 | mg |
| mannitol | 50 | mg |
| human serum albumin | 10 | mg |
| water for injections ad | 1 | ml |

Preparation:

Mannitol is dissolved in water for injections (Wfl); human serum albumin is added; active ingredient is dissolved with heating; made up to specified volume with Wfl; transferred into ampoules under nitrogen gas.

1. A (Hetero)aryl compound comprised of of general formula I 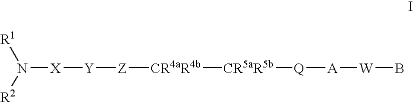
wherein
R¹, R² independently of one another denote H, C₁₋₈-alkyl or C₃₋₇-cycloalkyl, while the alkyl or cycloalkyl group may be mono- or polysubstituted by identical or different groups R”, and a —CH₂— group in position 3 or 4 of a 5-, 6- or 7-membered cycloalkyl group may be replaced by —O—, —S— or —NR³—, or
R² denotes a C₁₋₃-alkylene bridge which is linked to the group Y, wherein the alkylene bridge may be sustituted with one or more C₁₋₃-alkyl-groups, and R¹ is defined as hereinbefore or denotes a group selected from C₁₋₄-alkyl-CO—, C₁₋₄-alkyl-O—CO—, (C₁₋₄-alkyl)NH—CO— and (C₁₋₄-alkyl)₂N—CO— wherein alkyl-groups may be mono- or polyfluorinated; or
R¹ and R² form a C₃₋₈-alkylene bridge, wherein a —CH₂— group not adjacent to the N atom of the R¹R²N group may be replaced by —CH═N—, —CH═CH—, —O—, —S— —SO—, —(SO₂)—, —CO—, —C(═CH₂)—, —C(═N—OH)—, —C(═N—(C₁₋₄-alkyl))- or —NR¹³—,
while in the alkylene bridge defined hereinbefore one or more H atoms may be replaced by identical or different groups R¹⁴, and
the alkylene bridge defined hereinbefore may be substituted by one or two identical or different carbo- or heterocyclic groups Cy in such a way that the bond between the alkylene bridge and the group Cy is made
via a single or double bond,
via a common C atom forming a spirocyclic ring system,
via two common adjacent C and/or N atoms forming a fused bicyclic ring system or
via three or more C and/or N atoms forming a bridged ring system;
X denotes a C₁₋₄-alkylene bridge, while in the definition C₂₋₄-alkylene one or two C atoms may be monosubstituted by R¹⁰, or
a C₃₋₄-alkylene bridge, wherein a —CH₂—CH₂— group not directly adjacent to the N atom of the R¹R²N— group is replaced by —CH₂—O— or —CH₂—NR⁴—,
while the meanings given for X hereinbefore may comprise one, two or three identical or different C₁₋₄-alkyl substituents, while two alkyl groups may be joined together forming a 3 to 7-membered cyclic group; and
R⁴ denotes H or C₁₋₃-alkyl; and
R¹⁰ denotes hydroxy, hydroxy-C₁₋₃-alkyl, C₁₋₄-alkoxy or C₁₋₄-alkoxy-C₁₋₃-alkyl; and
Y is a 5- or 6-membered unsaturated or aromatic carbocyclic group which may contain 1, 2, 3 or 4 heteroatoms selected from N, O and/or S; and which cyclic group may be mono- or polysubstituted by identical or different substituents R²⁰;
Q, Z independently of one another denote a group selected from —CR^{3a}R^{3b}—, —O— and —NR^{N}—,
R^{N} independently of one another denote H, C₁₋₄-alkyl, formyl, C₁₋₃-alkylcarbonyl or C₁₋₃-alkylsulfonyl; and
R^{3a}, R^{3b}, R^{4a},
R^{4b}, R^{5a}, R^{5b} independently of one another denote H or C₁₋₄-alkyl; and
A is a 5- or 6-membered unsaturated or aromatic carbocyclic group which may contain 1, 2, 3 or 4 heteroatoms selected from N, O and/or S; which cyclic group may be mono- or polysubstituted by identical or different substituents R²⁰; and
B denotes a group Cy; and
W denotes a single bond, —CH₂—, —O—, —NR^{N}—, —O—CH₂—, —NR^{N}—CH₂—, —CH₂—O—, —CH₂—NR^{N}—N or —CH₂—CH₂—;
or
B is selected from the group consisting of halogen, CN, C₁₋₆-alkyl, C₁₋₆-alkoxy, C₂₋₆-alkenyl, C₂₋₆-alkynyl, C₃₋₆-alkenyloxy, C₃₋₆-alkynyloxy, C₃₋₇-cycloalkyl-C₁₋₃-alkyl, C₃₋₇-cycloalkenyl-C₁₋₃-alkyl, C₁₋₆-alkylcarbonyl, C₁₋₆-alkylamino or di-(C₁₋₆-alkyl)-amino, wherein one or more C atoms independently of one another may be mono- or polysubstituted by halogen and/ or monosubstituted by hydroxy, C₁₋₄-alkoxy or cyano and/ or cyclic groups may be mono- or polysubstituted by identical or different groups R²⁰; and
W denotes a single bond; and
Cy denotes a carbo- or heterocyclic group selected from one of the following meanings
a saturated 3- to 7-membered carbocyclic group,
an unsaturated 4- to 7-membered carbocyclic group,
a phenyl group,
a saturated 4- to 7-membered or unsaturated 5- to 7-membered heterocyclic group with an N, O or S atom as heteroatom,
a saturated or unsaturated 5- to 7-membered heterocyclic group with two or more N atoms or with one or two N atoms and an O or S atom as heteroatoms,
an aromatic heterocyclic 5- or 6-membered group with one or more identical or different heteroatoms selected from N, O and/or S.
while the above-mentioned saturated 6- or 7-membered groups may also be present as bridged ring systems with an imino, (C₁₋₄-alkyl)-imino, methylene, ethylene, (C₁₋₄-alkyl)-methylene or di-(C₁₋₄-alkyl)-methylene bridge, and
while the above-mentioned cyclic groups may be mono- or polysubstituted at one or more C atoms by identical or different groups R²⁰, or in the case of a phenyl group may also additionally be monosubstituted by nitro, and/or one or more NH groups may be substituted by R²¹, and
while in the above-mentioned saturated or unsaturated carbo- or heterocyclic groups a —CH₂-group may be replaced by a —C(═O)— group;
R¹¹ denotes halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N—, R¹⁸R¹⁹N—CO— or Cy, while in the above-mentioned groups one or more C atoms may be substituted independently of one another by substituents selected from halogen, OH, CN, CF₃, C₁₋₃-alkyl, C₁₋₃-alkoxy, hydroxy-C₁₋₃-alkyl;
R¹³ has one of the meanings given for R¹⁷,
R¹⁴ denotes halogen, cyano, C₁₋₆-alkyl, C₂₋₆-alkenyl, C₂₋₆-alkynyl, R¹⁵—O—, R¹⁵—O—CO— R¹⁵—CO—, R¹⁵—CO—O—, R¹⁶R¹⁷N—, HCO—NR¹⁵—, R¹⁸R¹⁹N—CO—, R¹⁵—O—C₁₋₃—alkyl, R¹⁵—O—CO—C₁₋₃-alkyl, R¹⁻⁵—SO₂—NH, R¹⁵—SO₂—N(C₁₋₃-alkyl)-, R¹⁵—O—CO—NH—C₁₋₃-alkyl, R¹⁵—SO₂—NH—C₁₋₃-alkyl, R¹⁵—CO—C₁₋₃-alkyl, R¹⁵—CO—O—C₁₋₃-alkyl, R¹⁶R¹⁷N—C₁₋₃-alkyl, R¹⁸R¹⁹N—CO—C₁₋₃-alkyl or Cy-C₁₋₃-alkyl,
R¹⁵ denotes H, C₁₋₄-alkyl, C₃₋₇-cycloalkyl, C₃₋₇-cycloalkyl-C₁₋₃-alkyl, phenyl, phenyl-C₁₋₃-alkyl, pyridinyl or pyridinyl-C₁₋₃-alkyl,
R¹⁶ denotes H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl or cyclo-C₃₋₆-alkyleneimino-C₂₋₆-alkyl,
R¹⁷ has one of the meanings given for R¹⁶ or denotes phenyl, phenyl-C₁₋₃-alkyl, pyridinyl, C₁₋₄-alkylcarbonyl, C₃₋₇-cycloalkylcarbonyl, hydroxycarbonyl-C₁₋₃-alkyl, C₁₋₄-alkoxycarbonyl, C₁₋₄-alkylaminocarbonyl, C₁₋₄-alkoxycarbonyl-C₁₋₃-alkyl, C₁₋₄-alkylcarbonylamino-C₂₋₃-alkyl, N—(C₁₋₄-alkylcarbonyl)-N—(C₁₋₄-alkyl)-amino-C₂₋₃-alkyl, C₁₋₄-alkylsulphonyl, C₁₋₄-alkylsulphonylamino-C₂₋₃-alkyl or N—(C₁₋₄-alkylsulphonyl)-N(—C₁₋₄-alkyl)-amino-C₂₋₃-alkyl;
R¹⁸, R¹⁹ independently of one another denote H or C₁₋₆-alkyl wherein R¹⁸, R¹⁹ may be linked to form a C₃₋₆-alkylene bridge, wherein a —CH₂— group not adjacent to an N atom may be replaced by —O—, —S—, —SO—, —(SO₂)—, —CO—, —C(═CH₂)— or —NR¹³—;
R²⁰ denotes halogen, hydroxy, cyano, nitro, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²—C₁₋₃-alkyl or has one of the meanings given for R²²; and
R²¹ denotes C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulphonyl, aminosulphonyl, C₁₋₄-alkylaminosulphonyl, di-C₁₋₄-alkylaminosulphonyl or cyclo-C₃₋₆-alkylene-imino-sulphonyl,
R²² denotes pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC-, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylamino-carbonyl, di-(C₁₋₄-alkyl)-aminocarbonyl, cyclo-C₃₋₆-alkyl-amino-carbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulphonyl, C₁₋₄-alkyl-sulphinyl, C₁₋₄-alkyl-sulphonylamino, C₁₋₄-alkyl-sulphonyl-N-(C₁₋₄-alkyl)amino, amino, C₁₋₄-alkylamino, di-(C₁₋₄-alkyl)-amino, C₁₋₄-alkyl-carbonyl-amino, C₁₋₄-alkyl-carbonyl-N—(C₁₋₄-alkyl)amino, cyclo-C₃₋₆-alkyleneimino, phenyl-C₁₋₃-alkylamino, N—(C₁₋₄-alkyl)-phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazin-yl)carbonyl, aminocarbonylamino or C₁₋₄-alkylaminocarbonylamino,
while in the above-mentioned groups and radicals, particularly in A, B, Q, W, X, Y, Z, R^{N}, R^{3a}, R^{3b}, R⁴, R^{4a}, R^{4b}, R^{5a}, R^{5b}, R¹⁰, R¹¹, R¹³ to R²², in each case one or more C atoms may additionally be mono- or polysubstituted by F and/or in each case one or two C atoms independently of one another may additionally be monosubstituted by Cl or Br and/or in each case one or more phenyl rings may additionally comprise independently of one another one, two or three substituents selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl- and di-(C₁₋₃-alkyl)-amino-C₁₋₃-alkyl and/or may be monosubstituted by nitro, and the H atom of any carboxy group present or an H atom bound to an N atom may in each case be replaced by a group which can be cleaved in vivo,
the tautomers, the diastereomers, the enantiomers, the mixtures thereof and the salts thereof;
with the proviso that the following compounds (D1) and (D2) are not included:
(D1) 2-[[[4-[[3-(2-fluorophenyl)propyl]amino]phenyl]methyl]amino]-propanamide; and
(D2) 2-[[[4-[[3-(3-fluorophenyl)propyl]amino]phenyl]methyl]amino]-propanamide.

2. A compound according to claim 1, characterised in that the groups R¹, R² are selected independently of one another from the group comprising H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N-(C₁₋₄-alkyl)-pyrrolidin-3-yl, pyrrolidinyl-C₁₋₃-alkyl, N—(C₁₋₄-alkyl)-pyrrolidinyl-C₁₋₃-alkyl, piperidin-3-yl, piperidin-4-yl, N—(C₁₋₄-alkyl)-piperidin-3-yl, N—(C₁₋₄-alkyl)-piperidin-4-yl, piperidinyl-C₁₋₃-alkyl, N—(C₁₋₄-alkyl)-piperidinyl-C₁₋₃-alkyl, tetrahydropyran-3-yl, tetrahydropyran-4-yl, tetrahydrofuran-2-ylmethyl, tetrahydrofuran-3-ylmethyl, phenyl-C₁₋₃-alkyl or pyridyl-C₁₋₃-alkyl, while in the above-mentioned groups and radicals one or more C atoms independently of one another may be mono- or polysubstituted by F, C₁₋₃-alkyl or hydroxy-C₁₋₃-alkyl, and/or one or two C atoms independently of one another may be monosubstituted by Cl, Br, OH, CF₃ or CN, and the above-mentioned cyclic groups may be mono- or polysubstituted at one or more C atoms by identical or different radicals R²⁰ in the case of a phenyl group may also additionally be monosubstituted by nitro, and/or one or more NH groups may be substituted by R²¹ wherein R²⁰ and R²¹ are defined as in claim 1.

3. A compound according to claim 1, characterised in that R¹ and R² together with the N atom to which they are bound form a heterocyclic group which is selected from the meanings azetidine, pyrrolidine, piperidine, azepan, 2,5-dihydro-1H-pyrrole, 1,2,3,6-tetrahydro-pyridine, 2,3,4,7-tetrahydro-1H-azepine, 2,3,6,7-tetrahydro-1H-azepine, piperazine in which the free imine function is substituted by R¹³, piperidin-4-one morpholine, thiomorpholine, 1-oxo-thiomorpholin-4-yl and 1,1-dioxo-thiomorpholin-4-yl;
while one or more H atoms may be replaced by identical or different groups R¹⁴, and/or
the heterocyclic groups specified may be substituted by one or two identical or different carbo- or heterocyclic groups Cy in such a way that the bond between the alkylene bridge and the group Cy is made
via a single or double bond,
via a common C atom forming a spirocyclic ring system,
via two common adjacent C and/or N atoms forming a fused bicyclic ring system or
via three or more C and/or N atoms forming a bridged ring system;
and the groups R¹³, R¹⁴ and the group Cy are defined as in claim 1.

4. A compound according to claim 1, characterised in that the group R² denotes a C₁₋₃-alkylene bridge which is linked to the group Y, wherein the alkylene bridge may be sustituted with one or more C₁₋₃-alkyl-groups, and R¹ is defined as in claim 2 or denotes a group selected from C₁₋₄-alkyl-CO—, C₁₋₄-alkyl-O—CO—, (C₁₋₄-alkyl)NH—CO—and (C₁₋₄-alkyl)₂N—CO— wherein alkyl-groups may be mono- or polyfluorinated.

5. A compound according to claim 1, characterised in that X denotes a —CH₂—, —CH₂—CH₂—, —CH₂—CH₂—O— or —CH₂—CH₂—NR⁴— bridging group, wherein one or two hydrogen atoms may be replaced by identical or different C₁₋₃-alkyl-groups, while two alkyl-groups may linked together to form a 3 to 6-membered cycloalkyl group; and wherein R⁴ is defined as in claim 1.

6. A compound according to claim 1, characterised in that the group Y denotes a phenyl, pyridyl, pyridazinyl, pyrimidinyl or pyrazinyl group which may be mono- or polysubstituted by identical or different substituents R²⁰, while R²⁰ is defined as in claim 1.

7. A compound according to claim 1, characterised in that the groups Q, Z independently of one another denote a group selected from —CH₂—, —O— and —NR^{N}—, with the proviso that Q and Z do not both at the same time denote —CH₂—.

8. A compound according to claim 1, characterised in that the groups Q, Z denote —CH₂—.

9. A compound according to claim 1, characterised in that the group A denotes a phenyl, pyridyl, pyridazinyl, pyrimidinyl or pyrazinyl group which may be mono- or polysubstituted by identical or different substituents R²⁰, while R²⁰ is defined as in claim 1.

10. A compound according to claim 1, characterised in that the group B is selected from the group consisting of phenyl, pyridyl, pyridazinyl, pyrazinyl, pyrimidinyl, pyrrolyl, pyrazolyl, imidazolyl, triazolyl, tetrazolyl, and thienyl, wherein said group B may be mono- or polysubstituted by identical or different substituents R²⁰, while R²⁰ is defined as in claim 1, and
the group W denotes a single bond, —CH₂—, —O—, —NR^{N}—, —O—CH₂—, —NR^{N}CH₂—, —CH₂—O— or —CH₂—NR^{N}— wherein R^{N} denotes H or C₁₋₄-alkyl, or the group W denotes —CH₂—H₂—.

11. A compound according to claim 1, characterised in that the group B is selected from the group consisting of halogen, CN, C₁₋₄-alkyl, C₁₋₆-alkoxy, C₁₋₄-alkylcarbonyl, C₁₋₄-alkylamino or di-(C₁₋₄-alkyl)-amino, wherein one or more C-atoms of said groups may additionally be mono- or polysubstituted by F; and
the group W denotes a single bond.

12. Physiologically acceptable salts of the compounds according to claim 1.

13. A pharmaceutical composition, comprised of at least one compound according to claim 1, optionally together with one or more physiologically acceptable excipients.

14. A pharmaceutical composition, comprised of at least one compound according to claim 1, optionally together with one or more inert carriers and/or diluents.

15. A method of influencing eating behaviour of a mammal, said method comprised of the steps of administering to said mammal a therapeutically effective amount at least one compound according to claim 1, including the compounds (D1) and (D2) explicitly excluded in claim 1 or one of the physiologically acceptable salts thereof, for influencing the eating behaviour of a mammal.

16. A method of for reducing the body weight and/ or for preventing an increase in the body weight of a mammal, said method comprised of the steps of administering to said mammal a thereapeutically effective amount of one or more compounds according to claim 1, including the compounds (D1) and (D2) explicitly excluded in claim 1 or one of the physiologically acceptable salts thereof.

17. A pharmaceutical composition comprised of a MCH-receptor-antagonistic compound according to claim 1, including the compounds (D1) and (D2) explicitly excluded in claim 1 or one of the physiologically acceptable salts thereof, for preparing a pharmaceutical composition with an MCH-receptor-antagonistic activity.

18. A method of preventing and/or treating symptoms and/or diseases which are caused by MCH or otherwise causally connected with MCH said method comprised of the steps of administering to a patient in need of such treatment a therapeutically effective amount of a compound according to claim 1, including the compounds (D1) and (D2) explicitly excluded in claim 1 or a pharmaceutically acceptable amount thereof.

19. A method of treating metabolic disorders and/or eating disorders, particularly obesity, bulimia, bulimia nervosa, cachexia, anorexia, anorexia nervosa and hyperphagia said method comprised of the steps of administering to a patient in need therof a therapeutically effective amount of a compound according to claim 1 including the compounds (D1) and (D2) explicitly excluded in claim 1 or a pharmaceutically acceptable salt therof.

20. The method of claim 19 wherein the metabolic disorder is selected from the list consisitng of obesity, bulimia, bulimia nervosa, cachexia, anorexia, anorexia nervosa and hyperphagia.

21. A method of treating and/or preventing diseases and/or disorders associated with obesity, said method comprised of the steps of administering to a patient in need therof a therapeutically effecctive amount of a compound according to claim 1, including the compounds (D1) and (D2) explicitly excluded in claim 1 or a physiologically acceptable salts thereof.

22. The method of claim 21 wherein the disease or dosorder associated with obesity is selected from particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis and gonitis,

23. A method of preventing and/or treating a disease or condition selected from hyperlipidaemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia and hormonal disorders, said method comprised of the steps of administering to a patient in need therof a thereapuetically effective amount of a compound according to claim 1 including the compounds (D1) and (D2) explicitly excluded in claim 1 or one of the physiologically acceptable salts thereof.

24. A method of treating or preventing and/or treating micturition disorders, said method comprised of the steps of administering to a patient in need therof a therapeutically effective amount of a compound according to claim 1 including the compounds (D1) and (D2) explicitly excluded in claim 1 or one of the physiologically acceptable salts thereof.

25. The method of claim 24 wherein the micturition disorder is selected from the list consisting of such as for example urinary incontinence, hyperactive urinary bladder, urgency, nycturia and enuresis.

26. A method of treating or preventing dependencies and/or withdrawal symptoms said method comprised of the steps of administering to a patient in need therorf a therapeutically effective amount of a compound according to claim 1 including the compounds (D1) and (D2) explicitly excluded in claim 1 or one of the physiologically acceptable salts thereof.

27. Process for preparing a composition or a pharmaceutical composition of a compound according to claim 1, wherein said pharmacuetical composition is incorporated in one or more inert carriers and/or diluents by a non-chemical method.

28. Pharmaceutical composition, containing
a first active substance which is selected from the compounds according to claim 1, including the compounds (D1) and (D2) explicitly excluded in claim 1 or one of the physiologically acceptable salts thereof, and
a second active substance selected from the group consisting of active substances for the treatment of diabetes, active substances for the treatment of diabetic complications, active substances for the treatment of obesity, active substances for the treatment of high blood pressure, active substances for the treatment of hyperlipidaemia, including arteriosclerosis, active substances for the treatment of arthritis, active substances for the treatment of anxiety states and active substances for the treatment of depression,
optionally together with one or more inert carriers and/or diluents.

29. Process for preparing (hetero)aryl compounds of formula (1-3) 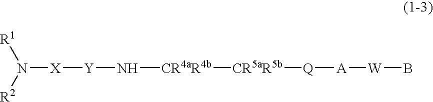
wherein R¹, R², X, Y, R^{4a}, R^{4b}, R^{5a}, R^{5b}, Q, A, W and B are defined in claim 1,
by reacting a compound of general formula (1-1) 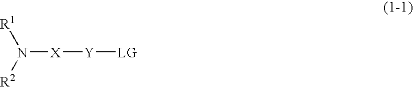
wherein R¹, R², X and Y are defined as hereinbefore,
with a compound of general formula (1-2) 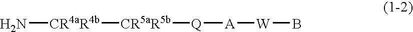
wherein R⁴¹, R^{4b}, R⁵¹, R^{5b}, Q, A, W and B are defined as hereinbefore,
in the presence of a palladium catalyst with or without ligands and/or copper iodide and in the presence of a base.
